SEQUENCE LISTING (208 sequences)

SEQ ID NO: 1 - protein, 231 aa, Escherichia coli [sequence omitted]

SEQ ID NO: 2 - DNA, 696 bp, Escherichia coli
  source 1..696: mol_type = genomic DNA
atgaaaatctccccctcgttaatgtgtatggatctgctgaaatttaaagaacagatcgaatttatcgacagccatgccgattacttccacatcgatatcatggacggtcactttgtccccaatctgacactctcaccgttcttcgtaagtcaggttaaaaaactggcaactaaaccgctcgactgtcatctgatggtgacgcggccgcaggattacattgctcaactggcgcgtgcgggagcagatttcatcactctgcatccggaaaccatcaacggccaggcgttccgcctgattgatgaaatccgccgtcatgacatgaaagtggggctgatccttaacccggagacgccagttgaggccatgaaatactatatccataaggccgataaaattacggtcatgactgtcgatcccggctttgccggacaaccgttcattcctgaaatgctggataaacttgccgaactgaaggcatggcgtgaacgagaaggtctggagtacgaaattgaggtggacggttcctgcaaccaggcaacttacgaaaaactgatggcggcaggggcggatgtctttatcgtcggcacttccggcctgtttaatcatgcggaaaatatcgacgaagcatggagaattatgaccgcgcagattctggctgcaaaaagcgaggtacagcctcatgcaaaaacagcataa

SEQ ID NO: 3 - protein, 222 aa, Escherichia coli
MSTPRQILAAIFDMDGLLIDSEPLWDRAELDVMASLGVDISRRNELPDTLGLRIDMVVDLWYARQPWNGPSRQEVVERVIARAISLVEETRPLLPGVREAVALCKEQGLLVGLASASPLHMLEKVLTMFDLRDSFDALASAEKLPYSKPHPQVYLDCAAKLGVDPLTCVALEDSVNGMIASKAARMRSIVVPAPEAQNDPRFVLADVKLSSLTELTAKDLLG

SEQ ID NO: 4 - protein, 222 aa, Escherichia coli
MSTPRQILAAIFDMDGLLIDSEPLWDRAELDVMASLGVDISRRNELPDTLGLRIDMVVDLWYARQPWNGPSRQEVVERVIARAISLVEETRPLLPGVREAVALCKEQGLLVGLASASPLHMLEKVLTMFDLRDSFDALASAEKLPYSKPHPQVYLDCAAKLGVDPLTCVALEDSVNGMIASKAARMRSIVVPAPEAQNDPRFVLANVKLSSLTELTAKDLLG

SEQ ID NO: 5 - DNA, 669 bp, Escherichia coli
  source 1..669: mol_type = genomic DNA
atgtcaaccccgcgtcagattcttgctgcaatttttgatatggatggattacttatcgactcagaacctttatgggatcgagccgaactggatgtgatggcaagcctgggggtggatatctcccgtcgtaacgagctgccggacaccttaggtttacgcatcgatatggtggtcgatctttggtacgcccggcaaccgtggaatgggccaagccgtcaggaagtagtagaacgggttattgcccgtgccatttcactggttgaagagacacgtccattattaccaggcgtgcgcgaagccgttgcgttatgcaaagaacaaggtttattggtgggactggcctccgcgtcaccactacatatgctggaaaaagtgttgaccatgtttgacttacgcgacagtttcgatgccctcgcctcggccgaaaaactgccttacagcaagccgcatccgcaagtatatctcgactgcgcagcaaaactgggcgttgaccctctgacctgcgtagcgctggaagattcggtaaatggcatgatcgcctctaaagcagcccgcatgcgttccatcgtcgttcctgcgccagaagcgcaaaatgatccacgttttgtattagcagacgtcaaactttcatcgctgacagaactcaccgcaaaagaccttctcggttga

SEQ ID NO: 6 - DNA, 1476 bp, Escherichia coli
  source 1..1476: mol_type = genomic DNA
atggcggtaacgcaaacagcccaggcctgtgacctggtcattttcggcgcgaaaggcgaccttgcgcgtcgtaaattgctgccttccctgtatcaactggaaaaagccggtcagctcaacccggacacccggattatcggcgtagggcgtgctgactgggataaagcggcatataccaaagttgtccgcgaggcgctcgaaactttcatgaaagaaaccattgatgaaggtttatgggacaccctgagtgcacgtctggatttttgtaatctcgatgtcaatgacactgctgcattcagccgtctcggcgcgatgctggatcaaaaaaatcgtatcaccattaactactttgccatgccgcccagcacttttggcgcaatttgcaaagggcttggcgaggcaaaactgaatgctaaaccggcacgcgtagtcatggagaaaccgctggggacgtcgctggcgacctcgcaggaaatcaatgatcaggttggcgaatacttcgaggagtgccaggtttaccgtatcgaccactatcttggtaaagaaacggtgctgaacctgttggcgctgcgttttgctaactccctgtttgtgaataactgggacaatcgcaccattgatcatgttgagattaccgtggcagaagaagtggggatcgaagggcgctggggctattttgataaagccggtcagatgcgcgacatgatccagaaccacctgctgcaaattctttgcatgattgcgatgtctccgccgtctgacctgagcgcagacagcatccgcgatgaaaaagtgaaagtactgaagtctctgcgccgcatcgaccgctccaacgtacgcgaaaaaaccgtacgcgggcaatatactgcgggcttcgcccagggcaaaaaagtgccgggatatctggaagaagagggcgcgaacaagagcagcaatacagaaactttcgtggcgatccgcgtcgacattgataactggcgctgggccggtgtgccattctacctgcgtactggtaaacgtctgccgaccaaatgttctgaagtcgtggtctatttcaaaacacctgaactgaatctgtttaaagaatcgtggcaggatctgccgcagaataaactgactatccgtctgcaacctgatgaaggcgtggatatccaggtactgaataaagttcctggccttgaccacaaacataacctgcaaatcaccaagctggatctgagctattcagaaacctttaatcagacgcatctggcggatgcctatgaacgtttgctgctggaaaccatgcgtggtattcaggcactgtttgtacgtcgcgacgaagtggaagaagcctggaaatgggtagactccattactgaggcgtgggcgatggacaatgatgcgccgaaaccgtatcaggccggaacctggggacccgttgcctcggtggcgatgattacccgtgatggtcgttcctggaatgagtttgagtaa

SEQ ID NO: 7 - DNA, 963 bp, Escherichia coli
  source 1..963: mol_type = genomic DNA
atgattaagaaaatcggtgtgttgacaagcggcggtgatgcgccaggcatgaacgccgcaattcgcggggttgttcgttctgcgctgacagaaggtctggaagtaatgggtatttatgacggctatctgggtctgtatgaagaccgtatggtacagctagaccgttacagcgtgtctgacatgatcaaccgtggcggtacgttcctcggttctgcgcgtttcccggaattccgcgacgagaacatccgcgccgtggctatcgaaaacctgaaaaaacgtggtatcgacgcgctggtggttatcggcggtgacggttcctacatgggtgcaatgcgtctgaccgaaatgggcttcccgtgcatcggtctgccgggcactatcgacaacgacatcaaaggcactgactacactatcggtttcttcactgcgctgagcaccgttgtagaagcgatcgaccgtctgcgtgacacctcttcttctcaccagcgtatttccgtggtggaagtgatgggccgttattgtggagatctgacgttggctgcggccattgccggtggctgtgaattcgttgtggttccggaagttgaattcagccgtgaagacctggtaaacgaaatcaaagcgggtatcgcgaaaggtaaaaaacacgcgatcgtggcgattaccgaacatatgtgtgatgttgacgaactggcgcatttcatcgagaaagaaaccggtcgtgaaacccgcgcaactgtgctgggccacatccagcgcggtggttctccggtgccttacgaccgtattctggcttcccgtatgggcgcttacgctatcgatctgctgctggcaggttacggcggtcgttgtgtaggtatccagaacgaacagctggttcaccacgacatcatcgacgctatcgaaaacatgaagcgtccgttcaaaggtgactggctggactgcgcgaaaaaactgtattaa

SEQ ID NO: 8 - DNA, 450 bp, Escherichia coli
  source 1..450: mol_type = genomic DNA
atgaaaaagattgcatttggctgtgatcatgtcggtttcattttaaaacatgaaatagtggcacatttagttgagcgtggcgttgaagtgattgataaaggaacctggtcgtcagagcgtactgattatccacattacgccagtcaagtcgcactggctgttgctggcggagaggttgatggcgggattttgatttgtggtactggcgtcggtatttcgatagcggcgaacaagtttgccggaattcgcgcggtcgtctgtagcgaaccttattccgcgcaactttcgcggcagcataacgacaccaacgtgctggcttttggttcacgagtggttggcctcgaactggcaaaaatgattgtggatgcgtggctgggcgcacagtacgaaggcggtcgtcatcaacaacgcgtggaggcgattacggcaatagagcagcggagaaattga

SEQ ID NO: 9 - DNA, 1176 bp, Escherichia coli
  source 1..1176: mol_type = genomic DNA
atgcaaaaactcattaactcagtgcaaaactatgcctggggcagcaaaacggcgttgactgaactttatggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggatgattttgccttctcgctgcatgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaa

SEQ ID NO: 10 - protein, 489 aa, Bacillus subtilis
MKTNQQPKAVIVIFGATGDLAKRKLYPSIHRLYQNGQIGEEFAVVGVGRRPWSNEDLRQTVKTSISSSADKHIDDFTSHFYYHPFDVTNPGSYQELNVLLNQLEDTYQIPNNRMFYLAMAPEFFGTIAKTLKSEGVTATTGWSRLVIEKPFGHDLPSAQALNKEIREAFTEDQIYRIDHYLGKQMVQNIEVIRFANAIFEPLWTNRYISNIQITSSESLGVEDRARYYEKSGALRDMVQNHIMQMVALLAMEPPIKLNTEEIRSEKVKVLRALRPIAKDEVDEYFVRGQYHAGEIDGVPVPAYTDEDNVAPDSNTETFVAGKLLIDNFRWAGVPFYIRTGKRMKEKSTKIVVQFKDIPMNLYYGNENNMNPNLLVIHIQPDEGITLYLNAKKLGGAAHAQPIKLDYCSNCNDELNTPEAYEKLIHDCLLGDATNFAHWDEVALSWSFVDSISETWAANKTLSPNYESGSMGPKESDDLLVKDGLHWWNI

SEQ ID NO: 11 - protein, 503 aa, Lactococcus lactis
MTEQKQALFTIFGATGDLAKRKLYPSLFRLFKKGELADNFAVIGTARRPWTNEYYREVVLESIKDLMNSKTEAENFASHFYYQSHDVSDSSHYVNLKDLGEKLRKQYKTAGNQVFFLAMAPQFFGTIAEHLKSENILTGEGFERIVIEKPFGTSYDTAKSLNDSLAKVFSEEQIFRIDHYLGKEMIQAVSAVRFANPIFESLWNNQHIDNVQITFAEFIGVEDRGGYYETSGALKDMIQNHVLQVLSLIAMEKPEKFDESYIVKEKVKALNAIRQYSSEEALENFVRGQYIAGRFDGEDYLGYREEDSVATDSRTETFAAGKFVIDNERWSGVPFYVRSGKRMTEKGTRINIVFKKDKDNLFAENCDDQSVQNVLTIYIQPTEGFSLSVNGKAAGQGFHLEPLRLNFRHDSEFLGNSPEAYEKLFLDVLNGDGTNFSHWEEAARAWELIDVIREAWDKETSELPTYAARTMGPKAAFDLLEKNGHEWAWQPDLWYQERGYYNK

SEQ ID NO: 12 - protein, 319 aa, Bacillus subtilis
MKRIGVLTSGGDSPGMNAAVRAVVRKAIYHDVEVYGIYNGYAGLISGKIEKLELGSVGDIIHRGGTKLYTARCPEFKTVEGREKGIANLKKLGIEGLVVIGGDGSYMGAKKLTEHGFPCVGVPGTIDNDIPGTDFTIGFDTALNTVIDAIDKIRDTATSHERTYVIEVMGRHAGDIALWAGLAGGAESILIPEADYDMHEIIARLKRGHERGKKHSIIIVAEGVGSGVEFGKRIEEETNLETRVSVLGHIQRGGSPSAADRVLASRLGAYAVELLLEGKGGRCVGIQNNKLVDHDIIEILETKHTVEQNMYQLSKELSI

SEQ ID NO: 13 - protein, 340 aa, Lactococcus lactis
MKRIAVLTSGGDAPGMNAAIRAVVRKAISEGIEVYGINHGYAGMVAGDIFPLTSASVGDKIGRGGTFLYSARYPEFAQVEGQLAGIEQLKKFGIEGVVVIGGDGSYHGAMRLTEHGFPAVGLPGTIDNDIVGTDFTIGFDTAVSTVVDALDKIRDTSSSHNRTFVVEVMGRNAGDIALNAGIAAGADDICIPEKEFKFENVVNNINKGYEKGKNHHIIVLAEGVMTGEEFATKLKEAGYKGDLRVSVLGHIQRGGSPTARDRVLASRMGARAVELLRDGIGGVAVGIRNEELVESPILGTAEEGALFSLTTEGGIKVNNPHKAGLELYRLNSALNNLNLN

SEQ ID NO: 14 - protein, 149 aa, Bacillus subtilis
MKVAIASDHGGVHIRNEIKELMDELQIEYIDMGCDCGSGSVDYPDYAFPVAEKVVSGEVDRGILICGTGIGMSISANKVKGIRCALAHDTFSAKATREHNDTNILAMGERVIGPGLAREIAKIWLTTEFTGGRHQTRIGKISDYEEKNL

SEQ ID NO: 15 - protein, 224 aa, Lactococcus lactis
MDNLKKQVGIKAAEFVKSGMVVGLGTGSTAAYFVEELGRRIAEEQLEITGVTTSNVTSSQARALGIPLASIDEVDYVDLTVDGADEIDSSLNGIKGGGAALLMEKIVATYSKDYIWIVDESKLSENLGSFKIPVEVIPYGSQQVFKKFEAAGYAPTWRLNEENERLITDMHHFIIDLHISQIKEPEKLAEELDLMVGVVEHGLFNNMVKKVIVAGNEGVRIINK

SEQ ID NO: 16 - protein, 315 aa, Bacillus subtilis
MTTEPLFFKPVFKERIWGGTALADFGYTIPSQRTGECWAFAAHQNGQSVVQNGMYKGFTLSELWEHHRHLFGQLEGDRFPLLTKILDADQDLSVQVHPNDEYANIHENGELGKTECWYIIDCQKDAEIIYGHNATTKEELTTMIERGEWDELLRRVKVKPGDFFYVPSGTVHAIGKGILALETQQNSDTTYRLYDYDRKDAEGKLRELHLKKSIEVIEVPSIPERHTVHHEQIEDLLTTTLIECAYFSVGKWNLSGSASLKQQKPFLLISVIEGEGRMISGEYVYPFKKGDHMLLPYGLGEFKLEGYAECIVSHL

SEQ ID NO: 17 - protein, 316 aa, Bacillus subtilis
MTQSPIFLTPVFKEKIWGGTALRDRFGYSIPSESTGECWAISAHPKGPSTVANGPYKGKTLIELWEEHREVFGGVEGDRFPLLTKLLDVKEDTSIKVHPDDYYAGENEEGELGKTECWYIIDCKENAEIIYGHTARSKTELVTMINSGDWEGLLRRIKIKPGDFYYVPSGTLHALCKGALVLETQQNSDATYRVYDYDRLDSNGSPRELHFAKAVNAATVPHVDGYIDESTESRKGITIKTFVQGEYFSVYKWDINGEAEMAQDESFLICSVIEGSGLLKYEDKTCPLKKGDHFILPAQMPDFTIKGTCTLIVSHI

SEQ ID NO: 18 - protein, 315 aa, Bacillus subtilis
MTHPLFLEPVFKERLWGGTKLRDAFGYAIPSQKTGECWAVSAHAHGSSSVKNGPLAGKTLDQVWKDHPEIFGFPDGKVFPLLVKLLDANMDLSVQVHPDDDYAKLHENGDLGKTECWYIIDCKDDAELILGHHASTKEEFKQRIESGDWNGLLRRIKIKPGDFFYVPSGTLHALCKGTLVLEIQQNSDTTYRVYDYDRCNDQGQKRTLHIEKAMEVITIPHIDKVHTPEVKEVGNAEIIVYVQSDYFSVYKWKISGRAAFPSYQTYLLGSVLSGSGRIINNGIQYECNAGSHFILPAHFGEFTIEGTCEFMISHP

SEQ ID NO: 19 - protein, 315 aa, Lactococcus lactis
MKEPLFLNSVLQEKIWGGDHLKEFGYDLPSDKVGEYWAISAHPHGVSTIANGEFKGQKLDQLYASHRELFGDSKKEVFPLLTKILDANDWLSVQVHPDDEYGQKHEGELGKTECWYIISAEPGAEIIYGHNAKSREELAEMIKSGDWDHLLRKVKVKTGDFFHVPSGTMHAIGAGIVILETQQSSDTTYRVYDFDRKDDQGNLRELHIQQSIDVLNIPGDKVPENQVKTEKFADAEITTLVKSDFFDVYKWQIHGDHEFTKVADYTLVSVLDGQGKLTVDGNEYPVEKGAHFILPSNIEKWNLSGQLEIIASNPA

SEQ ID NO: 20 - protein, 491 aa, Escherichia coli
MAVTQTAQACDLVIFGAKGDLARRKLLPSLYQLEKAGQLNPDTRIIGVGRADWDKAAYTKVVREALETFMKETIDEGLWDTLSARLDFCNLDVNDTAAFSRLGAMLDQKNRITINYFAMPPSTFGAICKGLGEAKLNAKPARVVMEKPLGTSLATSQEINDQVGEYFEECQVYRIDHYLGKETVLNLLALRFANSLFVNNWDNRTIDHVEITVAEEVGIEGRWGYFDKAGQMRDMIQNHLLQILCMIAMSPPSDLSADSIRDEKVKVLKSLRRIDRSNVREKTVRGQYTAGFAQGKKVPGYLEEEGANKSSNTETFVAIRVDIDNWRWAGVPFYLRTGKRLPTKCSEVVVYFKTPELNLFKESWQDLPQNKLTIRLQPDEGVDIQVLNKVPGLDHKHNLQITKLDLSYSETFNQTHLADAYERLLLETMRGIQALFVRRDEVEEAWKWVDSITEAWAMDNDAPKPYQAGTWGPVASVAMITRDGRSWNEFE

SEQ ID NO: 21 - protein, 320 aa, Escherichia coli
MIKKIGVLTSGGDAPGMNAAIRGVVRSALTEGLEVMGIYDGYLGLYEDRMVQLDRYSVSDMINRGGTFLGSARFPEFRDENIRAVAIENLKKRGIDALVVIGGDGSYMGAMRLTEMGFPCIGLPGTIDNDIKGTDYTIGFFTALSTVVEAIDRLRDTSSSHQRISVVEVMGRYCGDLTLAAAIAGGCEFVVVPEVEFSREDLVNEIKAGIAKGKKHAIVAITEHMCDVDELAHFIEKETGRETRATVLGHIQRGGSPVPYDRILASRMGAYAIDLLLAGYGGRCVGIQNEQLVHHDIIDAIENMKRPFKGDWLDCAKKLY

SEQ ID NO: 22 - DNA, 930 bp, Escherichia coli
  source 1..930: mol_type = genomic DNA
atggtacgtatctatacgttgacacttgcgccctctctcgatagcgcaacaattaccccgcaaatttatcccgaaggaaaactgcgctgtaccgcaccggtgttcgaacccgggggcggcggcatcaacgtcgcccgcgccattgcccatcttggaggcagtgccacagcgatcttcccggcgggtggcgcgaccggcgaacacctggtttcactgttggcggatgaaaatgtccccgtcgctactgtagaagccaaagactggacccggcagaatttacacgtacatgtggaagcaagcggtgagcagtatcgttttgttatgccaggcgcggcattaaatgaagatgagtttcgccagcttgaagagcaagttctggaaattgaatccggggccatcctggtcataagcggaagcctgccgccaggtgtgaagctggaaaaattaacccaactgatttccgctgcgcaaaaacaagggatccgctgcatcgtcgacagttctggcgaagcgttaagtgcagcactggcaattggtaacatcgagttggttaagcctaaccaaaaagaactcagtgcgctggtgaatcgcgaactcacccagccggacgatgtccgcaaagccgcgcaggaaatcgttaatagcggcaaggccaaacgggttgtcgtttccctgggtccacaaggagcgctgggtgttgatagtgaaaactgtattcaggtggtgccaccaccggtgaaaagccagagtaccgttggcgctggtgacagcatggtcggcgcgatgacactgaaactggcagaaaatgcctctcttgaagagatggttcgttttggcgtagctgcggggagtgcagccacactcaatcagggaacacgtctgtgctcccatgacgatacgcaaaaaatttacgcttacctttcccgctaa

SEQ ID NO: 23 - protein, 309 aa, Escherichia coli
MVRIYTLTLAPSLDSATITPQIYPEGKLRCTAPVFEPGGGGINVARAIAHLGGSATAIFPAGGATGEHLVSLLADENVPVATVEAKDWTRQNLHVHVEASGEQYRFVMPGAALNEDEFRQLEEQVLEIESGAILVISGSLPPGVKLEKLTQLISAAQKQGIRCIVDSSGEALSAALAIGNIELVKPNQKELSALVNRELTQPDDVRKAAQEIVNSGKAKRVVVSLGPQGALGVDSENCIQVVPPPVKSQSTVGAGDSMVGAMTLKLAENASLEEMVRFGVAAGSAATLNQGTRLCSHDDTQKIYAYLSR

SEQ ID NO: 24 - protein, 149 aa, Escherichia coli
MKKIAFGCDHVGFILKHEIVAHLVERGVEVIDKGTWSSERTDYPHYASQVALAVAGGEVDGGILICGTGVGISIAANKFAGIRAVVCSEPYSAQLSRQHNDTNVLAFGSRVVGLELAKMIVDAWLGAQYEGGRHQQRVEAITAIEQRRN

SEQ ID NO: 25 - protein, 391 aa, Escherichia coli
MQKLINSVQNYAWGSKTALTELYGMENPSSQPMAELWMGAHPKSSSRVQNAAGDIVSLRDVIESDKSTLLGEAVAKRFGELPFLFKVLCAAQPLSIQVHPNKHNSEIGFAKENAAGIPMDAAERNYKDPNHKPELVFALTPFLAMNAFREFSEIVSLLQPVAGAHPAIAHFLQQPDAERLSELFASLLNMQGEEKSRALAILKSALDSQQGEPWQTIRLISEFYPEDSGLFSPLLLNVVKLNPGEAMFLFAETPHAYLQGVALEVMANSDNVLRAGLTPKYIDIPELVANVKFEAKPANQLLTQPVKQGAELDFPIPVDDFAFSLHDLSDKETTISQQSAAILFCVEGDATLWKGSQQLQLKPGESAFIAANESPVTVKGHGRLARVYNKL

SEQ ID NO: 26 - DNA, 1470 bp, Bacillus subtilis
  source 1..1470: mol_type = genomic DNA
gtgaaaacaaaccaacaaccaaaagcagtaattgtcatattcggtgcaactggagatttagcaaaacgaaaattgtatccgtctattcaccgtttatatcaaaacggacaaatcggagaagagtttgcagtggtaggagttggaagaagaccttggtctaatgaggatcttcgccaaactgttaaaacatccatttcctcatctgcagataagcatatagatgatttcacgtctcatttttactatcacccgtttgacgtgacaaaccctggttcttatcaagagctaaacgtattgcttaaccagctggaagatacatatcaaattcctaacaacagaatgttctacttggcaatggctcctgaattcttcggaacgattgcaaaaacattaaaatcagagggtgtaacagctacaaccggctggtcccgccttgtcatcgaaaaaccgttcggccatgatctgccaagcgcacaggcattgaataaagaaatccgcgaagcatttacggaagatcaaatttacagaatcgaccattatctaggcaaacaaatggttcagaacattgaagtgattcgatttgccaatgcgattttcgaaccgctttggacaaaccgctacatttcaaacattcaaatcacatctagcgaatcactaggcgttgaagaccgcgcaagatattacgaaaaatcaggcgcccttcgcgacatggtgcaaaaccatattatgcagatggttgcccttcttgcaatggagccgcctatcaaattgaacacagaagaaatccgcagcgagaaagtgaaggtgctgagagcactgcgtcctattgcaaaagacgaagtggatgaatactttgtgcgcggacaatatcatgctggtgaaattgacggtgtaccggttcctgcttatacagatgaagataatgtcgctcctgactccaatacagaaacctttgttgccggcaagctcttgatcgacaacttcagatgggctggtgttccattctacatcagaaccggaaaacgaatgaaagaaaagtccacaaaaattgtcgttcaatttaaggacattccgatgaacctgtactacggtaatgaaaacaacatgaatccgaacttgcttgtcattcatattcagcctgacgaaggcattacgctttacttaaatgctaaaaagcttggcggagcagcacacgcacagccaatcaaactcgattattgcagcaattgcaatgacgagttgaacacccctgaagcatatgaaaaactaattcacgactgtcttcttggcgatgcaacaaactttgcacactgggatgaagttgccctttcttggagctttgtcgactctatttctgaaacatgggcagcaaacaaaaccttatctcctaactacgaatcaggctcaatgggaccgaaagaatctgatgatcttttggtgaaagacggcttacactggtggaacatataa

SEQ ID NO: 27 - DNA, 960 bp, Bacillus subtilis
  source 1..960: mol_type = genomic DNA
atgaaacgaataggggtattaacgagcggcggggattccccgggaatgaacgcagcagttcgcgcagtagtcagaaaagcgatctatcatgacgttgaagtttacggtatttacaacggatacgcgggattgatcagcggaaagattgaaaagcttgaactcggatcagtaggcgatattatacatcgtggagggactaagctttatacggcgagatgtcctgaattcaaaacagttgaaggccgtgaaaaagggatagcaaacttgaagaagcttggtattgaaggccttgttgttatcggtggagacggttcctatatgggtgcgaaaaaattaacggaacacgggtttccatgtgtaggtgtaccgggtacaattgataatgacattccgggcactgattttacaatcggtttcgatacagctttaaatacagtaattgacgcaattgataagattcgcgatacagcgacttctcatgaacgtacatatgtaatcgaagtaatgggccgtcatgccggcgatatcgcattgtgggccggtcttgcagggggcgcagaatcgatcttaatccctgaggcagactatgacatgcacgaaatcattgcccgcttaaaacgcggccacgaacgcggcaagaagcacagtattattattgttgccgaaggtgtaggcagcggtgttgaattcgggaaacgcattgaagaagaaacaaatcttgaaactagggtatctgtattgggccatatccagcgcggaggttctccgagtgctgctgaccgtgtgttggcaagccgtctcggcgcatatgcagttgaactgctgcttgaaggaaaaggcggacgctgtgtaggtatacaaaacaataagcttgtagaccatgatattatagaaatacttgagacaaaacacacagttgagcaaaacatgtatcagctttcaaaagaactgtctatctaa

SEQ ID NO: 28 - DNA, 450 bp, Bacillus subtilis
  source 1..450: mol_type = genomic DNA
atgaaagtagccattgcatcggatcatggcggcgttcacattcgaaatgaaatcaaagagttaatggacgaattgcaaattgaatatattgatatgggctgtgactgcggcagcggctctgtcgattatccggattatgcttttccggtggccgaaaaagtggttagcggcgaagttgacagaggcattttaatttgcgggacaggcatcggcatgagcatttccgctaataaagtaaaagggattcgctgcgcgctggcgcacgataccttcagcgcgaaggcgacgagggagcataatgacacaaacatccttgcgatgggtgaacgggtgatcggacctggtttggctcgggaaatcgcaaaaatctggctgactactgagtttaccgggggaagacaccaaacgcgtattggaaaaatctccgattatgaagagaaaaacctgtag

SEQ ID NO: 29 - DNA, 948 bp, Bacillus subtilis
  source 1..948: mol_type = genomic DNA
atgacgactgaaccgttatttttcaagcctgttttcaaagaaagaatttggggcgggaccgctttagctgattttggctataccattccgtcacaacgaacaggggagtgctgggcttttgccgcgcatcaaaatggtcaaagcgttgttcaaaacggaatgtataaggggttcacgctcagcgaattatgggaacatcacagacatttattcggacagcttgaaggggaccgtttccctctgcttacaaaaatattagatgctgaccaggacttatctgttcaggtgcatccgaatgatgaatatgccaacatacatgaaaacggtgagcttggaaaaacagaatgctggtacattattgattgccaaaaagatgccgagattatttatggccacaatgcaacaacaaaggaagaactaactaccatgatagagcgtggagaatgggatgagctcttgcgccgtgtaaaggtaaagccgggggattttttctatgtgccaagcggtactgttcatgcgattggaaaaggaattcttgctttggagacgcagcagaactcagacacaacctacagattatatgattatgaccgaaaagatgcagaaggcaagctgcgcgagcttcatctgaaaaagagcattgaagtgatagaggtcccgtctattccagaacggcatacagttcaccatgaacaaattgaggatttgcttacaacgacattgattgaatgcgcttacttttcggtggggaaatggaacttatcaggatcagcaagcttaaagcagcaaaaaccattccttcttatcagtgtgattgaaggggagggccgtatgatctctggtgagtatgtctatcctttcaaaaaaggagatcatatgttgctgccttacggtcttggagaatttaaactcgaaggatatgcagaatgtatcgtctcccatctgtaa

SEQ ID NO: 30 - DNA, 951 bp, Bacillus subtilis
  source 1..951: mol_type = genomic DNA
atgacgcaatcaccgatttttctaacgcctgtgtttaaagaaaaaatctggggcggaaccgctttacgagatagatttggatacagtattccttcagaatcaacgggggaatgctgggccatttccgctcatccaaaaggaccgagcactgttgcaaatggcccgtataaaggaaagacattgatcgagctttgggaagagcaccgtgaagtattcggcggcgtagagggggatcggtttccgcttctgacaaagctgctggatgtgaaggaagatacgtcaattaaagttcaccctgatgattactatgccggagaaaacgaagagggagaactcggcaagacggaatgctggtacattatcgactgtaaggaaaacgcagaaatcatttacgggcatacggcccgctcaaaaaccgaacttgtcacaatgatcaacagcggtgactgggagggcctgctgcgaagaatcaaaattaaaccgggtgatttctattatgtgccgagcggaacgctgcacgcattgtgcaagggggcccttgttttagagactcagcaaaattcagatgccacataccgggtgtacgattatgaccgtcttgatagcaacggaagtccgagagagcttcattttgccaaagcggtcaatgccgccacggttccccatgtggacgggtatatagatgaatcgacagaatcaagaaaaggaataaccattaaaacatttgtccaaggggaatatttttcggtttataaatgggacatcaatggcgaagctgaaatggctcaggatgaatcctttctgatttgcagcgtgatagaaggaagcggtttgctcaagtatgaggacaaaacatgtccgctcaaaaaaggtgatcactttattttgccggctcaaatgcccgattttacgataaaaggaacttgtacccttatcgtgtctcatatttaa

SEQ ID NO: 31 - DNA, 948 bp, Bacillus subtilis
  source 1..948: mol_type = genomic DNA
atgacgcatccattatttttagagcctgtctttaaagaaagactatggggagggacgaagcttcgtgacgcttttggctacgcaataccctcacaaaaaacaggtgagtgctgggccgtttctgcacatgcccatggctcgtcgtctgtaaaaaatggcccgctggcaggaaagacacttgatcaagtatggaaagatcatccagagatattcgggtttccggatggtaaggtgtttccgctgctggtaaagctgctggacgccaatatggatctctccgtgcaagtccatcctgatgatgattatgcaaaactgcacgaaaatggcgaccttggtaaaacggagtgctggtatatcattgattgcaaagatgacgccgaactaattttgggacatcatgcaagcacaaaggaagagttcaaacaacgaatagaaagcggtgattggaacgggctgctgaggcgaatcaaaatcaagccaggagatttcttttatgtgccaagcggtacactccatgctttatgtaagggaacccttgtccttgaaatccagcaaaactctgatacaacatatcgcgtatacgattatgaccgctgtaatgaccagggccaaaaaagaactcttcatatagaaaaagccatggaagtcataacgataccgcatatcgataaagtgcatacaccggaagtaaaagaagttggtaacgctgagatcattgtttatgtgcaatcagattatttctcagtgtacaaatggaagattagcggccgagctgcttttccttcatatcaaacctatttgctggggagtgttctgagcggatcaggacgaatcataaataatggtattcagtatgaatgcaatgcaggctcacactttattctgcctgcgcattttggagaatttacaatagaaggaacatgtgaattcatgatatctcatccttaa

SEQ ID NO: 32 - DNA, 1023 bp, Lactococcus lactis
  source 1..1023: mol_type = genomic DNA
atgaaacgcattgcagttttgacttctggtggtgatgccccaggaatgaatgcggctattcgtgcagttgttcgcaaagcaatttctgaaggtatcgaagtttacggtatcaatcacggatatgcgggcatggttgcgggagatattttcccgcttacgtcagcttcagttggtgataaaatcggtcgtggtggtacattcttgtattcagcacgctacccagaatttgctcaagtagaaggacaacttgctgggattgagcaacttaaaaaattcggtatcgaaggtgtcgttgtaatcggtggtgatggttcttatcatggagctatgcgtcttacagaacatggtttcccagctgttggacttccaggaacaatcgataacgatatcgtaggaactgattttacaattggatttgatacagctgtttcaacagttgtagatgccttggataaaattcgtgatacttcatcatcacataaccgtactttcgttgtagaagtaatgggacgtaatgctggagatatcgctttgaatgctggtatcgctgctggtgcagatgatatttgtattccagaaaaagaatttaagtttgaaaacgtagttaacaacattaacaaaggctacgaaaaaggtaaaaatcaccacatcatcgttcttgctgaaggtgtaatgactggtgaagaatttgctacaaaacttaaagaagctggttataaaggagaccttcgcgtttctgtccttggacacatccaacgtggtggttcaccaacagctcgtgaccgtgttctagcttcacgtatgggtgctcgtgccgttgaattgcttcgtgatggaatcggtggcgtagccgttggtatccgtaatgaagaacttgtagaaagtccaattctcggaacagctgaagaaggagcactctttagtttgactactgaaggtggaatcaaagtaaataacccgcataaagctggtcttgaactttaccgtcttaactcagcactcaacaatcttaaccttaactaa

SEQ ID NO: 33 - DNA, 1512 bp, Lactococcus lactis
  source 1..1512: mol_type = genomic DNA
atgaccgaacaaaaacaagcacttttcaccatcttcggagcaactggtgacctcgctaaaagaaagctctacccttcactctttcgccttttcaaaaaaggtgagctagctgacaattttgcagtcattggtaccgctcgccgtccatggacaaacgaatattatcgtgaagttgttttagagtctatcaaagatttaatgaactcaaaaacagaagccgaaaatttcgccagtcatttttattatcaaagtcacgatgttagcgacagctcacattacgttaacttaaaagatttgggtgaaaaattgcgtaaacagtataaaactgctggcaatcaagtcttctttttagcaatggctcctcaattttttggcactatcgctgaacaccttaaatcagaaaatattttgacaggtgagggttttgagagaatcgtcattgaaaaaccatttggaacaagctacgatacggcaaaatcacttaatgacagcctcgcaaaagtatttagtgaagaacaaattttccgaatcgaccactaccttggaaaagaaatgatccaggccgtttctgccgttcgttttgccaatccaatctttgaatcgctttggaataatcaacacattgataacgttcagattacttttgccgaatttatcggtgttgaagaccgtggcggttactacgaaacttctggtgctttaaaagatatgattcaaaaccatgtcttacaagtactcagcctcattgccatggaaaaacctgaaaaatttgatgaatcttatattgtaaaagaaaaagttaaagcccttaatgcaatccgtcaatattcttctgaggaagctttagaaaacttcgttcgtggtcaatatatcgcaggacgttttgacggtgaagattacctaggctatcgcgaagaagattctgttgcaacagacagtcgaactgaaactttcgctgctggaaaatttgtcattgataatgagcgttggtcaggcgtccctttctatgttcgctcaggaaaacgtatgactgaaaaaggaactcgtattaatatcgttttcaaaaaagataaagacaatctctttgcagaaaattgcgatgaccaatccgttcaaaatgttttgaccatttatattcaaccaactgaagggttttcactttcagtaaatggaaaagcggctggtcaaggcttccatttagagcctttgcggttaaatttccgacacgatagtgaatttctcggaaattctcctgaagcatatgaaaaacttttcctagatgtccttaatggagatggaacaaatttctcacattgggaagaagcggctcgcgcctgggaacttattgatgttattcgagaagcttgggataaagaaacttctgagcttccaacttatgcggctcgcacaatgggacctaaggctgcatttgacttactagaaaaaaatggtcacgaatgggcttggcaacctgatttatggtatcaagaacgtggttactacaataaataa

SEQ ID NO: 34 - DNA, 675 bp, Lactococcus lactis
  source 1..675: mol_type = genomic DNA
atggataatttaaaaaaacaagtcggcataaaagctgctgaatttgttaaatcaggaatggtcgttggtttaggaactgggtcaacagcagcctattttgtcgaagaattgggtcgaagaattgccgaagaacaattggaaattacgggtgtaacaacgtccaatgtaacaagtagccaagccagagctcttggaattcctttagcctctattgacgaagtagattatgttgatttaacagttgatggcgcagatgaaattgattcttcactaaatggtattaaaggtggtggagcagcacttctaatggaaaaaattgttgcaacctactcaaaagactatatttggattgttgatgaaagtaaattatcagaaaatctaggatcctttaaaattcctgtagaagttattccttatggctcacaacaagtttttaaaaaattcgaagcggctggctatgctccaacttggcgtctaaatgaggaaaacgagagattgataacggatatgcatcactttattattgaccttcatatctctcaaattaaagaaccagaaaaacttgctgaagagcttgatttaatggttggagttgttgaacacggcctctttaataacatggttaaaaaagtgattgttgctggcaacgaaggcgtaagaataataaataagtaa

SEQ ID NO: 35 - DNA, 948 bp, Lactococcus lactis
  source 1..948: mol_type = genomic DNA
atgaaagaaccattgtttttgaactcagttttgcaagaaaaaatctggggcggcgaccatttgaaagagtttggctatgatttgccatcagacaaagttggtgaatattgggctatttctgctcatccacatggtgtgtcaacaattgctaatggcgaatttaaaggtcaaaaacttgaccaattatacgcaagtcaccgcgaattgtttggtgatagtaaaaaagaagtttttcccttactaactaaaattttagatgccaatgactggctttctgtgcaagttcatccagatgatgaatatggacaaaaacatgaaggtgaacttggaaaaactgaatgttggtacattatttcagctgaaccaggtgctgaaattatctatggacataatgctaaatcacgtgaagaattagcagaaatgattaaatctggtgattgggatcatttgttacgtaaggtaaaagtgaaaacaggagatttcttccatgttccgtcaggaacaatgcacgcaatcggtgctggaattgttattcttgaaacacaacaatcttctgatacaacttaccgtgtttatgatttcgaccgtaaagatgaccaaggaaatctacgtgaattacatattcaacaatcaattgatgtattgaatattccgggcgacaaagttcctgaaaatcaagttaaaactgaaaaatttgctgatgcagaaattacaactcttgtgaaatcagatttctttgatgtttataaatggcaaattcatggtgaccatgaatttaccaaagttgctgattacactttagtttctgtacttgatggtcaaggaaaattaacagttgatggaaatgaatatccagttgaaaaaggagctcatttcatcttaccaagcaacattgaaaaatggaatttgtctggtcaattagaaattattgccagcaatcctgcctaa

SEQ ID NO: 36 - protein, 271 aa, Escherichia coli
MSVKVIVTDMDGTFLNDAKTYNQPRFMAQYQELKKRGIKFVVASGNQYYQLISFFPELKDEISFVAENGALVYEHGKQLFHGELTRHESRIVIGELLKDKQLNFVACGLQSAYVSENAPEAFVALMAKHYHRLKPVKDYQEIDDVLFKFSLNLPDEQIPLVIDKLHVALDGIMKPVTSGFGFIDLIIPGLHKANGISRLLKRWDLSPQNVVAIGDSGNDAEMLKMARYSFAMGNAAENIKQIARYATDDNNHEGALNVIQAVLDNTSPFNS

SEQ ID NO: 37 - DNA, 816 bp, Escherichia coli
  source 1..816: mol_type = genomic DNA
atgagcgtaaaagttatcgtcacagacatggacggtacttttcttaacgacgccaaaacgtacaaccaaccacgttttatggcgcaatatcaggaactgaaaaagcgcggcattaagttcgttgttgccagcggtaatcagtattaccagcttatttcattctttcctgagctaaaggatgagatctcttttgtcgcggaaaacggcgcactggtttacgaacatggcaagcagttgttccacggcgaactgacccgacatgaatcgcggattgttattggcgagttgctaaaagataagcaactcaattttgtcgcctgcggtctgcaaagtgcatatgtcagcgaaaatgcccccgaagcatttgtcgcactgatggcaaaacactaccatcgcctgaaacctgtaaaagattatcaggagattgacgacgtactgttcaagttttcgctcaacctgccggatgaacaaatcccgttagtgatcgacaaactgcacgtagcgctcgatggcattatgaaacccgttaccagtggttttggctttatcgacctgattattcccggtctacataaagcaaacggtatttcgcggttactgaaacgctgggatctgtcaccgcaaaatgtggtagcgattggcgacagcggtaacgatgcggagatgctgaaaatggcgcgttattcctttgcgatgggcaatgctgcggaaaacattaaacaaatcgcccgttacgctaccgatgataataatcatgaaggcgcgctgaatgtgattcaggcggtgctggataacacatccccttttaacagctga

SEQ ID NO: 38 - protein, 464 aa, Escherichia coli
MPDAKKQGRSNKAMTFFVCFLAALAGLLFGLDIGVIAGALPFIADEFQITSHTQEWVVSSMMFGAAVGAVGSGWLSFKLGRKKSLMIGAILFVAGSLFSAAAPNVEVLILSRVLLGLAVGVASYTAPLYLSEIAPEKIRGSMISMYQLMITIGILGAYLSDTAFSYTGAWRWMLGVIIIPAILLLIGVFFLPDSPRWFAAKRRFVDAERVLLRLRDTSAEAKRELDEIRESLQVKQSGWALFKENSNFRRAVFLGVLLQVMQQFTGMNVIMYYAPKIFELAGYTNTTEQMWGTVIVGLTNVLATFIAIGLVDRWGRKPTLTLGFLVMAAGMGVLGTMMHIGIHSPSAQYFAIAMLLMFIVGFAMSAGPLIWVLCSEIQPLKGRDFGITCSTATNWIANMIVGATFLTMLNTLGNANTFWVYAALNVLFILLTLWLVPETKHVSLEHIERNLMKGRKLREIGAHD

SEQ ID NO: 39 - DNA, 1395 bp, Escherichia coli
  source 1..1395: mol_type = genomic DNA
atgcctgacgctaaaaaacaggggcggtcaaacaaggcaatgacgtttttcgtctgcttccttgccgctctggcgggattactctttggcctggatatcggtgtaattgctggcgcactgccgtttattgcagatgaattccagattacttcgcacacgcaagaatgggtcgtaagctccatgatgttcggtgcggcagtcggtgcggtgggcagcggctggctctcctttaaactcgggcgcaaaaagagcctgatgatcggcgcaattttgtttgttgccggttcgctgttctctgcggctgcgccaaacgttgaagtactgattctttcccgcgttctactggggctggcggtgggtgtggcctcttataccgcaccgctgtacctctctgaaattgcgccggaaaaaattcgtggcagtatgatctcgatgtatcagttgatgatcactatcgggatcctcggtgcttatctttctgataccgccttcagctacaccggtgcatggcgctggatgctgggtgtgattatcatcccggcaattttgctgctgattggtgtcttcttcctgccagacagcccacgttggtttgccgccaaacgccgttttgttgatgccgaacgcgtgctgctacgcctgcgtgacaccagcgcggaagcgaaacgcgaactggatgaaatccgtgaaagtttgcaggttaaacagagtggctgggcgctgtttaaagagaacagcaacttccgccgcgcggtgttccttggcgtactgttgcaggtaatgcagcaattcaccgggatgaacgtcatcatgtattacgcgccgaaaatcttcgaactggcgggttataccaacactaccgagcaaatgtgggggaccgtgattgtcggcctgaccaacgtacttgccacctttatcgcaatcggccttgttgaccgctggggacgtaaaccaacgctaacgctgggcttcctggtgatggctgctggcatgggcgtactcggtacaatgatgcatatcggtattcactctccgtcggcgcagtatttcgccatcgccatgctgctgatgtttattgtcggttttgccatgagtgccggtccgctgatttgggtactgtgctccgaaattcagccgctgaaaggccgcgattttggcatcacctgctccactgccaccaactggattgccaacatgatcgttggcgcaacgttcctgaccatgctcaacacgctgggtaacgccaacaccttctgggtgtatgcggctctgaacgtactgtttatcctgctgacattgtggctggtaccggaaaccaaacacgtttcgctggaacatattgaacgtaatctgatgaaaggtcgtaaactgcgcgaaataggcgctcacgattaa

SEQ ID NO: 40 - protein, 321 aa, Escherichia coli
MTKYALVGDVGGTNARLALCDIASGEISQAKTYSGLDYPSLEAVIRVYLEEHKVEVKDGCIAIACPITGDWVAMTNHTWAFSIAEMKKNLGFSHLEIINDFTAVSMAIPMLKKEHLIQFGGAEPVEGKPIAVYGAGTGLGVAHLVHVDKRWVSLPGEGGHVDFAPNSEEEAIILEILRAEIGHVSAERVLSGPGLVNLYRAIVKADNRLPENLKPKDITERALADSCTDCRRALSLFCVIMGRFGGNLALNLGTFGGVFIAGGIVPRFLEFFKASGFRAAFEDKGRFKEYVHDIPVYLIVHDNPGLLGSGAHLRQTLGHIL

SEQ ID NO: 41 - DNA, 966 bp, Escherichia coli
  source 1..966: mol_type = genomic DNA
atgacaaagtatgcattagtcggtgatgtgggcggcaccaacgcacgtcttgctctgtgtgatattgccagtggtgaaatctcgcaggctaagacctattcagggcttgattaccccagcctcgaagcggtcattcgcgtttatcttgaagaacataaggtcgaggtgaaagacggctgtattgccatcgcttgcccaattaccggtgactgggtggcgatgaccaaccatacctgggcgttctcaattgccgaaatgaaaaagaatctcggttttagccatctggaaattattaacgattttaccgctgtatcgatggcgatcccgatgctgaaaaaagagcatctgattcagtttggtggcgcagaaccggtcgaaggtaagcctattgcggtttacggtgccggaacggggcttggggttgcgcatctggtccatgtcgataagcgttgggtaagcttgccaggcgaaggcggtcacgttgattttgcgccgaatagtgaagaagaggccattatcctcgaaatattgcgtgcggaaattggtcatgtttcggcggagcgcgtgctttctggccctgggctggtgaatttgtatcgcgcaattgtgaaagctgacaaccgcctgccagaaaatctcaagccaaaagatattaccgaacgcgcgctggctgacagctgcaccgattgccgccgcgcattgtcgctgttttgcgtcattatgggccgttttggcggcaatctggcgctcaatctcgggacatttggcggcgtgtttattgcgggcggtatcgtgccgcgcttccttgagttcttcaaagcctccggtttccgtgccgcatttgaagataaagggcgctttaaagaatatgtccatgatattccggtgtatctcatcgtccatgacaatccgggccttctcggttccggtgcacatttacgccagaccttaggtcacattctgtaa

SEQ ID NO: 42 - DNA, 104 bp, Escherichia coli
  source 1..104: mol_type = genomic DNA
aattgtgagcggataacaattgacattgtgagcggataacaagatactgagcacatcagcaggacgcactgaccgaattcattaaagaggagaaaagatatacc

SEQ ID NO: 43 - DNA, 101 bp, Escherichia coli
  source 1..101: mol_type = genomic DNA
tccctatcagtgatagagattgacatccctatcagtgatagagatactgagcacatcagcaggacgcactgaccgaattcattaaagaggagaaaggtacc

SEQ ID NO: 44 - DNA, 87 bp, Escherichia coli
  source 1..87: mol_type = genomic DNA
taatacgactcactataggggaattgtgagcggataacaattcccctctagaaataattttgtttaactttaagaaggagatatacc

SEQ ID NO: 45 - DNA, 91 bp, Escherichia coli
  source 1..91: mol_type = genomic DNA
gttgacactctatcgttgatagagttattttaccactccctatcagtgatagagaaaagaattcaaaagatctaaagaggagaaaggatct

SEQ ID NO: 46 - DNA, 351 bp, Escherichia coli
  source 1..351: mol_type = genomic DNA
gtaataattttataaatgcgttcaaaataataatcaagtactaatagtgatattttaaggtctgatttttacgtgataattcaggagacacagaatgcgcataaaaataacagcataaaacaccttaccaccacccaagaatttcatattgtattgtttttcaatgaaaaaatattattcgcgtaatatctcacgataaataacattaggattttgttatttaaacacgagtcctttgcacttgcttactttatcgataaatcctacttttttaatgcgatccaatcattttaaggagtttaaaatggataagaagcaagtgaattcattaaagaggagaaaagatatacc

SEQ ID NO: 47 - DNA, 1641 bp, Escherichia coli
  source 1..1641: mol_type = genomic DNA
atggcaatccacaatcgtgcaggccaacctgcacaacagagtgatttgattaacgtcgcccaactgacggcgcaatattatgtactgaaaccagaagcagggaatgcggagcacgcggtgaaattcggtacttccggtcaccgtggcagtgcagcgcgccacagctttaacgagccgcacattctggcgatcgctcaggcaattgctgaagaacgtgcgaaaaacggcatcactggcccttgctatgtgggtaaagatactcacgccctgtccgaacctgcattcatttccgttctggaagtgctggcagcgaacggcgttgatgtcattgtgcaggaaaacaatggcttcaccccgacgcctgccgtttccaatgccatcctggttcacaataaaaaaggtggcccgctggcagacggtatcgtgattacaccgtcccataacccgccggaagatggtggaatcaaatacaatccgccaaatggtggcccggctgataccaacgtcactaaagtggtggaagacagggccaacgcactgctggccgatggcctgaaaggcgtgaagcgtatctccctcgacgaagcgatggcatccggtcatgtgaaagagcaggatctggtgcagccgttcgtggaaggtctggccgatatcgttgatatggccgcgattcagaaagcgggcctgacgctgggcgttgatccgctgggcggttccggtatcgaatactggaagcgtattggcgagtattacaacctcaacctgactatcgttaacgatcaggtcgatcaaaccttccgctttatgcaccttgataaagacggcgcgatccgtatggactgctcctccgagtgtgcgatggcgggcctgctggcactgcgtgataagttcgatctggcgtttgctaacgacccggattatgaccgtcacggtatcgtcactccggcaggtttgatgaatccgaaccactacctggcggtggcaatcaattacctgttccagcatcgtccgcagtggggcaaagatgttgccgtcggtaaaacgctggtttcatctgcgatgatcgaccgtgtggtcaacgacttgggccgtaaactggtagaagtcccggtaggtttcaaatggtttgtcgatggtctgttcgacggcagcttcggctttggcggcgaagagagtgcaggggcttccttcctgcgtttcgacggcacgccgtggtccaccgacaaagacggcatcatcatgtgtctgctggcggcggaaatcaccgctgtcaccggtaagaacccgcaggaacactacaacgaactggcaaaacgctttggtgcgccgagctacaaccgtttgcaggcagctgcgacttccgcacaaaaagcggcgctgtctaagctgtctccggaaatggtgagcgccagcaccctggcaggtgacccgatcaccgcgcgcctgactgctgctccgggcaacggtgcttctattggcggtctgaaagtgatgactgacaacggctggttcgccgcgcgtccgtcaggcacggaagacgcatataagatctactgcgaaagcttcctcggtgaagaacatcgcaagcagattgagaaagaagcggttgagattgttagcgaagttctgaaaaacgcgtaa

SEQ ID NO: 48 - protein, 546 aa, Escherichia coli
MAIHNRAGQPAQQSDLINVAQLTAQYYVLKPEAGNAEHAVKFGTSGHRGSAARHSFNEPHILAIAQAIAEERAKNGITGPCYVGKDTHALSEPAFISVLEVLAANGVDVIVQENNGFTPTPAVSNAILVHNKKGGPLADGIVITPSHNPPEDGGIKYNPPNGGPADTNVTKVVEDRANALLADGLKGVKRISLDEAMASGHVKEQDLVQPFVEGLADIVDMAAIQKAGLTLGVDPLGGSGIEYWKRIGEYYNLNLTIVNDQVDQTFRFMHLDKDGAIRMDCSSECAMAGLLALRDKFDLAFANDPDYDRHGIVTPAGLMNPNHYLAVAINYLFQHRPQWGKDVAVGKTLVSSAMIDRVVNDLGRKLVEVPVGFKWFVDGLFDGSFGFGGEESAGASFLRFDGTPWSTDKDGIIMCLLAAEITAVTGKNPQEHYNELAKRFGAPSYNRLQAAATSAQKAALSKLSPEMVSASTLAGDPITARLTAAPGNGASIGGLKVMTDNGWFAARPSGTEDAYKIYCESFLGEEHRKQIEKEAVEIVSEVLKNA

SEQ ID NO: 49 - DNA, 1536 bp, Bacillus subtilis
  source 1..1536: mol_type = genomic DNA
atgagtaaaaaaccagctgcactcatcattcttgatgggttcggattacgtaacgaaacagtagggaacgcagtcgctttagcgaaaaaaccgaattttgaccgctattggaaccagtatcctcatcaaactttgactgcttcaggcgaggctgtaggtcttcctgaagggcaaatggggaactccgaagtaggtcacttaaatatcggtgcgggacgtattgtgtaccaaagcttaacacgcgtaaatgttgccattcgtgaaggagagttcgaacgcaatcaaacattccttgacgcgatcagcaacgcgaaagaaaacaacaaagccttgcacctgttcggtcttttatctgacggaggcgtgcacagccatatcaatcatttattcgcactgttaaagcttgcgaaaaaagaagggctgacaaaggtttatatccatggcttccttgacggccgtgatgtaggtccgcagacagcgaaaacgtacatcaaccagttgaacgatcaaatcaaggaaatcggtgtaggtgaaatcgccagcatttccggacgttactactctatggaccgcgacaaacgctgggaccgtgtagaaaaagcgtaccgcgcaatggcgtacggcgaaggcccatcttaccgcagcgccctggatgttgttgatgattcatatgcaaatggtatctacgatgaattcgtgattccatctgtcatcacaaaagaaaacggtgagcctgttgcgaaaatccaagacggcgattctgtgattttctataatttcagaccggaccgcgccatccagatttccaacacgttcacaaacaaagacttccgtgacttcgaccgcggcgagaattatccaaagaacctgtatttcgtctgcctgactcacttcagtgaaacagttgacggctatgtagcgtttaaaccgataaatcttgataacacagtcggagaagtattatctcagcacgggttaaaacagcttcgaattgcagaaactgaaaagtatccgcacgtcacgttctttatgagcggcggccgtgaagctgaattcccgggtgaagagcgtattctcatcaactcgcctaaagttgcaacgtatgacttgaagcctgagatgagtgcgtatgaagtgaaagacgcgcttgtcaaagaaattgaagctgacaagcatgacgcgatcattcttaacttcgcaaaccctgatatggtcggccactccggaatggttgaaccaacaattaaagcaattgaagcagtggacgaatgcttgggcgaagtcgttgacgcgatccttgctaaaggcggacacgctatcattaccgctgatcacggtaatgctgacattctgattacagaatcaggcgaaccgcacactgcgcatacaacaaacccagtccctgtgattgtaacgaaagaaggcattacgctgcgtgaaggcggaatcctaggcgaccttgcaccaacgttattagaccttcttggtgttgaaaaaccgaaagaaatgacaggaacatctttaattcaaaaataa

SEQ ID NO: 50 - protein, 511 aa, Bacillus subtilis
MSKKPAALIILDGFGLRNETVGNAVALAKKPNFDRYWNQYPHQTLTASGEAVGLPEGQMGNSEVGHLNIGAGRIVYQSLTRVNVAIREGEFERNQTFLDAISNAKENNKALHLFGLLSDGGVHSHINHLFALLKLAKKEGLTKVYIHGFLDGRDVGPQTAKTYINQLNDQIKEIGVGEIASISGRYYSMDRDKRWDRVEKAYRAMAYGEGPSYRSALDVVDDSYANGIYDEFVIPSVITKENGEPVAKIQDGDSVIFYNFRPDRAIQISNTFTNKDFRDFDRGENYPKNLYFVCLTHFSETVDGYVAFKPINLDNTVGEVLSQHGLKQLRIAETEKYPHVTFFMSGGREAEFPGEERILINSPKVATYDLKPEMSAYEVKDALVKEIEADKHDAIILNFANPDMVGHSGMVEPTIKAIEAVDECLGEVVDAILAKGGHAIITADHGNADILITESGEPHTAHTTNPVPVIVTKEGITLREGGILGDLAPTLLDLLGVEKPKEMTGTSLIQK

SEQ ID NO: 51 - DNA, 666 bp, Lactococcus lactis
  source 1..666: mol_type = genomic DNA
atgtttaaagcagtattgtttgatttagatggtgtaattacagataccgcagagtatcattttagagcttggaaagctttggctgaagaaattggcattaatggtgttgaccgccaatttaatgagcaattaaaaggggtctcacgagaagactcgcttcagaaaattctagatttagctgataaaaaagtatcagctgaggaatttaaagaacttgctaagagaaaaaatgataactatgtgaaaatgattcaggatgtgtcgccagccgatgtctatcctggaattttacaattactcaaagatttacgttcaaataaaatcaaaattgctttagcatcggcttctaagaatggtccatttttattagagagaatgaatttaactggatattttgatgcaattgctgatccggctgaagttgcagcatcaaaaccagcaccagatatttttattgcagcagcacatgcagtgggtgttgccccctctgaatcaattgggttagaggattctcaagctggaattcaagccatcaaagattcaggggctttaccaattggtgtagggcgcccagaagatttgggagatgatatcgtcattgtgcctgatacttcacactatacattagaatttttgaaagaagtttggcttcaaaagcaaaaatga

SEQ ID NO: 52 - protein, 221 aa, Lactococcus lactis
MFKAVLFDLDGVITDTAEYHFRAWKALAEEIGINGVDRQFNEQLKGVSREDSLQKILDLADKKVSAEEFKELAKRKNDNYVKMIQDVSPADVYPGILQLLKDLRSNKIKIALASASKNGPFLLEKMNLTGYFDAIADPAEVAASKPAPDIFIAAAHAVGVAPSESIGLEDSQAGIQAIKDSGALPIGVGRPEDLGDDIVIVPDTSYYTLEFLKEVWLQKQK

SEQ ID NO: 53 - DNA, 352 bp, Escherichia coli
  source 1..352: mol_type = genomic DNA
gtaataattttataaatgcgttcaaaataataatcaagtactaatagtgatattttaaggtctgatttttacgtgataattcaggagacacagaatgcgcataaaaataacagcataaaacaccttaccaccacccaagaatttcatattgtattgtttttcaatgaaaaaatattattcgcgtaatatctcacgataaataacattaggattttgttatttaaacacgagtcctttgcacttgcttactttatcgataaatcctacttttttaatgcgatccaatcattttaaggagtttaaaatggataagaagcaagtcgaattcattaaagaggagaaaggtaccatg

SEQ ID NO: 54 - DNA, 329 bp, Escherichia coli
  source 1..329: mol_type = genomic DNA
tttgcagtgcaactaattccatgtatattactacccatatatagcgtctataaaatttaataaataatgacgccctagttaaacttaaagtgcctggttcaactatcaaaaatcgctcaccctttttcacctgtttaaaatatgttcagcaacccatcttgatggcgacctcctctccgcgatgatttcaataacatattctgtgttggcatatgaaattttgaggattaccctacacttataggagttaccttacaggggttccttcaatttgtgttgatttacgcgagataacgctcgaattcattaaagaggagaaaggtaccatg

SEQ ID NO: 55 - DNA, 253 bp, Escherichia coli
  source 1..253: mol_type = genomic DNA
tatccgaatgtaagaaagttggcgtaaatcaggtagttggcgtaaacttatttgacgtgtaccgcggtaagggtgttgcggaggggtataagagcctcgccataagcctgatcctgcaagataccagccgtacactcgaagaagaggagattgccgctaccgtcgccaaatgtgtagaggcattaaaagagcgattccaggcatcattgagggattgaacctcgaattcattaaagaggagaaaggtaccatg

SEQ ID NO: 56 - DNA, 174 bp, Escherichia coli
  source 1..174: mol_type = genomic DNA
tcattgaatctttattagttttgtttttcacgcttgttaccactattagtgtgataggaacagccagaatagcggaacacatagccggtgctatacttaatctcgttaattactgggacataacatcaagaggatatgaaattcgaattcattaaagaggagaaaggtaccatg

SEQ ID NO: 57 - protein, 1368 aa, synthetic construct
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 58 - DNA, 115 bp, synthetic construct
  source 1..115: mol_type = other DNA
tagatctattaaattgtgagcggataacaattgacattgtgagcggataacaagatactgagcacatcagcaggacgcactgaccgaattcattaaagaggagaaaggtaccatg

SEQ ID NO: 59 - DNA, 110 bp, synthetic construct
  source 1..110: mol_type = other DNA
atatttatctgcaaaattttaaataaagctccaataaatcatattgttaatttcttcactttccgctgattcggtgccagactgaaatcagcctataggaggaaatgatg

SEQ ID NO: 60 - DNA, 7100 bp, synthetic construct
  source 1..7100: mol_type = other DNA
taatacgactcactataggggaattgtgagcggataacaattcccctctagaaataattttgtttaactttaagaaggagatataccatgggccatcatcatcatcatcatcatcatcatcacagcagcggccatatcgaaggtcgtcatatgaaaatctccccctcgttaatgtgtatggatctgctgaaatttaaagaacagatcgaatttatcgacagccatgccgattacttccacatcgatatcatggacggtcactttgtccccaatctgacactctcaccgttcttcgtaagtcaggttaaaaaactggcaactaaaccgctcgactgtcatctgatggtgacgcggccgcaggattacattgctcaactggcgcgtgcgggagcagatttcatcactctgcatccggaaaccatcaacggccaggcgttccgcctgattgatgaaatccgccgtcatgacatgaaagtggggctgatccttaacccggagacgccagttgaggccatgaaatactatatccataaggccgataaaattacggtcatgactgtcgatcccggctttgccggacaaccgttcattcctgaaatgctggataaacttgccgaactgaaggcatggcgtgaacgagaaggtctggagtacgaaattgaggtggacggttcctgcaaccaggcaacttacgaaaaactgatggcggcaggggcggatgtctttatcgtcggcacttccggcctgtttaatcatgcggaaaatatcgacgaagcatggagaattatgaccgcgcagattctggctgcaaaaagcgaggtacagcctcatgcaaaaacagcataaggatccggctgctaacaaaaggagatatacatatgtcaaccccgcgtcagattcttgctgcaatttttgatatggatggattacttatcgactcagaacctttatgggatcgagccgaactggatgtgatggcaagcctgggggtggatatctcccgtcgtaacgagctgccggacaccttaggtttacgcatcgatatggtggtcgatctttggtacgcccggcaaccgtggaatgggccaagccgtcaggaagtagtagaacgggttattgcccgtgccatttcactggttgaagagacacgtccattattaccaggcgtgcgcgaagccgttgcgttatgcaaagaacaaggtttattggtgggactggcctccgcgtcaccactacatatgctggaaaaagtgttgaccatgtttgacttacgcgacagtttcgatgccctcgcctcggccgaaaaactgccttacagcaagccgcatccgcaagtatatctcgactgcgcagcaaaactgggcgttgaccctctgacctgcgtagcgctggaagattcggtaaatggcatgatcgcctctaaagcagcccgcatgcgttccatcgtcgttcctgcgccagaagcgcaaaatgatccacgttttgtattagcagacgtcaaactttcatcgctgacagaactcaccgcaaaagaccttctcggttgaggatccggctgctaacaaagcccgaaaggaagctgagttggctgctgccaccgctgagcaataactagcataaccccttggggcctctaaacgggtcttgaggggttttttgctgaaaggaggaactatatccggatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaat

SEQ ID NO: 61 - DNA, 7247 bp, synthetic construct
  source 1..7247: mol_type = other DNA
taatacgactcactataggggaattgtgagcggataacaattcccctctagaaataattttgtttaactttaagaaggagatataccatgggccatcatcatcatcatcatcatcatcatcacagcagcggccatatcgaaggtcgtcatatgaaaatctccccctcgttaatgtgtatggatctgctgaaatttaaagaacagatcgaatttatcgacagccatgccgattacttccacatcgatatcatggacggtcactttgtccccaatctgacactctcaccgttcttcgtaagtcaggttaaaaaactggcaactaaaccgctcgactgtcatctgatggtgacgcggccgcaggattacattgctcaactggcgcgtgcgggagcagatttcatcactctgcatccggaaaccatcaacggccaggcgttccgcctgattgatgaaatccgccgtcatgacatgaaagtggggctgatccttaacccggagacgccagttgaggccatgaaatactatatccataaggccgataaaattacggtcatgactgtcgatcccggctttgccggacaaccgttcattcctgaaatgctggataaacttgccgaactgaaggcatggcgtgaacgagaaggtctggagtacgaaattgaggtggacggttcctgcaaccaggcaacttacgaaaaactgatggcggcaggggcggatgtctttatcgtcggcacttccggcctgtttaatcatgcggaaaatatcgacgaagcatggagaattatgaccgcgcagattctggctgcaaaaagcgaggtacagcctcatgcaaaaacagcataaggatccggctgctaacaaaaggagatatacatatgagcgtaaaagttatcgtcacagacatggacggtacttttcttaacgacgccaaaacgtacaaccaaccacgttttatggcgcaatatcaggaactgaaaaagcgcggcattaagttcgttgttgccagcggtaatcagtattaccagcttatttcattctttcctgagctaaaggatgagatctcttttgtcgcggaaaacggcgcactggtttacgaacatggcaagcagttgttccacggcgaactgacccgacatgaatcgcggattgttattggcgagttgctaaaagataagcaactcaattttgtcgcctgcggtctgcaaagtgcatatgtcagcgaaaatgcccccgaagcatttgtcgcactgatggcaaaacactaccatcgcctgaaacctgtaaaagattatcaggagattgacgacgtactgttcaagttttcgctcaacctgccggatgaacaaatcccgttagtgatcgacaaactgcacgtagcgctcgatggcattatgaaacccgttaccagtggttttggctttatcgacctgattattcccggtctacataaagcaaacggtatttcgcggttactgaaacgctgggatctgtcaccgcaaaatgtggtagcgattggcgacagcggtaacgatgcggagatgctgaaaatggcgcgttattcctttgcgatgggcaatgctgcggaaaacattaaacaaatcgcccgttacgctaccgatgataataatcatgaaggcgcgctgaatgtgattcaggcggtgctggataacacatccccttttaacagctgaggatccggctgctaacaaagcccgaaaggaagctgagttggctgctgccaccgctgagcaataactagcataaccccttggggcctctaaacgggtcttgaggggttttttgctgaaaggaggaactatatccggatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaat

SEQ ID NO: 62 - DNA, 3657 bp, synthetic construct
  source 1..3657: mol_type = other DNA
aattgtgagcggataacaattgacattgtgagcggataacaagatactgagcacatcagcaggacgcactgaccgaattcattaaagaggagaaaagatataccatgaaaatctccccctcgttaatgtgtatggatctgctgaaatttaaagaacagatcgaatttatcgacagccatgccgattacttccacatcgatatcatggacggtcactttgtccccaatctgacactctcaccgttcttcgtaagtcaggttaaaaaactggcaactaaaccgctcgactgtcatctgatggtgacgcggccgcaggattacattgctcaactggcgcgtgcgggagcagatttcatcactctgcatccggaaaccatcaacggccaggcgttccgcctgattgatgaaatccgccgtcatgacatgaaagtggggctgatccttaacccggagacgccagttgaggccatgaaatactatatccataaggccgataaaattacggtcatgactgtcgatcccggctttgccggacaaccgttcattcctgaaatgctggataaacttgccgaactgaaggcatggcgtgaacgagaaggtctggagtacgaaattgaggtggacggttcctgcaaccaggcaacttacgaaaaactgatggcggcaggggcggatgtctttatcgtcggcacttccggcctgtttaatcatgcggaaaatatcgacgaagcatggagaattatgaccgcgcagattctggctgcaaaaagcgaggtacagcctcatgcaaaaacagcataaggatccggctgctaacaaaaggagatatacatatgtcaaccccgcgtcagattcttgctgcaatttttgatatggatggattacttatcgactcagaacctttatgggatcgagccgaactggatgtgatggcaagcctgggggtggatatctcccgtcgtaacgagctgccggacaccttaggtttacgcatcgatatggtggtcgatctttggtacgcccggcaaccgtggaatgggccaagccgtcaggaagtagtagaacgggttattgcccgtgccatttcactggttgaagagacacgtccattattaccaggcgtgcgcgaagccgttgcgttatgcaaagaacaaggtttattggtgggactggcctccgcgtcaccactacatatgctggaaaaagtgttgaccatgtttgacttacgcgacagtttcgatgccctcgcctcggccgaaaaactgccttacagcaagccgcatccgcaagtatatctcgactgcgcagcaaaactgggcgttgaccctctgacctgcgtagcgctggaagattcggtaaatggcatgatcgcctctaaagcagcccgcatgcgttccatcgtcgttcctgcgccagaagcgcaaaatgatccacgttttgtattagcagacgtcaaactttcatcgctgacagaactcaccgcaaaagaccttctcggttgagatccggctgctaaggtacctaatctagaggcatcaaataaaacgaaaggctcagtcgaaagactgggcctttcgttttatctgttgtttgtcggtgaacgctctcctgagtaggacaaatccgccgccctagacctaggcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgactagtgcttggattctcaccaataaaaaacgcccggcggcaaccgagcgttctgaacaaatccagatggagttctgaggtcattactggatctatcaacaggagtccaagcgagctcgtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcacctcgag

SEQ ID NO: 63 - DNA, 6840 bp, synthetic construct
  source 1..6840: mol_type = other DNA
gttgacactctatcgttgatagagttattttaccactccctatcagtgatagagaaaagaattcaaaagatctaaagaggagaaaggatctatggataagaaatactcaataggcttagctatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatgccattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgactaactcgagtaaggatctccaggcatcaaataaaacgaaaggctcagtcgaaagactgggcctttcgttttatctgttgtttgtcggtgaacgctctctactagagtcacactggctcaccttcgggtgggcctttctgcgtttatacctagggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgttactagtgcttggattctcaccaataaaaaacgcccggcggcaaccgagcgttctgaacaaatccagatggagttctgaggtcattactggatctatcaacaggagtccaagcgagctcgatatcaaatcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcattttagcttccttagctcctgaaaatctcgataactcaaaaaatacgcccggtagtgatcttatttcattatggtgaaagttggaacctcttacgtgccgatcaacgtctcattttcgccagatatcgacgtcttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctaattttt

SEQ ID NO: 64 - DNA, 3883 bp, synthetic construct
  source 1..3883: mol_type = other DNA
aattgtgagcggataacaattgacattgtgagcggataacaagatactgagcacatcagcaggacgcactgaccgaattcattaaagaggagaaaagatataccatgaaaatctccccctcgttaatgtgtatggatctgctgaaatttaaagaacagatcgaatttatcgacagccatgccgattacttccacatcgatatcatggacggtcactttgtccccaatctgacactctcaccgttcttcgtaagtcaggttaaaaaactggcaactaaaccgctcgactgtcatctgatggtgacgcggccgcaggattacattgctcaactggcgcgtgcgggagcagatttcatcactctgcatccggaaaccatcaacggccaggcgttccgcctgattgatgaaatccgccgtcatgacatgaaagtggggctgatccttaacccggagacgccagttgaggccatgaaatactatatccataaggccgataaaattacggtcatgactgtcgatcccggctttgccggacaaccgttcattcctgaaatgctggataaacttgccgaactgaaggcatggcgtgaacgagaaggtctggagtacgaaattgaggtggacggttcctgcaaccaggcaacttacgaaaaactgatggcggcaggggcggatgtctttatcgtcggcacttccggcctgtttaatcatgcggaaaatatcgacgaagcatggagaattatgaccgcgcagattctggctgcaaaaagcgaggtacagcctcatgcaaaaacagcataaggatccggctgctaacaaaaggagatatacatatgtcaaccccgcgtcagattcttgctgcaatttttgatatggatggattacttatcgactcagaacctttatgggatcgagccgaactggatgtgatggcaagcctgggggtggatatctcccgtcgtaacgagctgccggacaccttaggtttacgcatcgatatggtggtcgatctttggtacgcccggcaaccgtggaatgggccaagccgtcaggaagtagtagaacgggttattgcccgtgccatttcactggttgaagagacacgtccattattaccaggcgtgcgcgaagccgttgcgttatgcaaagaacaaggtttattggtgggactggcctccgcgtcaccactacatatgctggaaaaagtgttgaccatgtttgacttacgcgacagtttcgatgccctcgcctcggccgaaaaactgccttacagcaagccgcatccgcaagtatatctcgactgcgcagcaaaactgggcgttgaccctctgacctgcgtagcgctggaagattcggtaaatggcatgatcgcctctaaagcagcccgcatgcgttccatcgtcgttcctgcgccagaagcgcaaaatgatccacgttttgtattagcagacgtcaaactttcatcgctgacagaactcaccgcaaaagaccttctcggttgagatccggctgctaaggtacctaatctagaggcatcaaataaaacgaaaggctcagtcgaaagactgggcctttcgttttatctgttgtttgtcggtgaacgctctcctgagtaggacaaatccgccgccctagacctaggcgttcggctgcggcgagcggtatcagctcactcaaaggcggtaatacggttatccacagaatcaggggataacgcaggaaagaacagagcgcctgatgcggtattttctccttacgcatctgtgcggtatttcacaccgcatatgctggatccttgacagctagctcagtcctaggtataatactagtgttttagagctagaaatagcaagttaaaataaggctagtccgttatcaacttgaaaaagtggcaccgagtcggtgctttttttgaattctctagagtcgacctgcagaagcttagatctattaatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgactagtgcttggattctcaccaataaaaaacgcccggcggcaaccgagcgttctgaacaaatccagatggagttctgaggtcattactggatctatcaacaggagtccaagcgagctcgtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcacctcgag

SEQ ID NO: 65 - DNA, 3903 bp, synthetic construct
  source 1..3903: mol_type = other DNA
aattgtgagcggataacaattgacattgtgagcggataacaagatactgagcacatcagcaggacgcactgaccgaattcattaaagaggagaaaagatataccatgaaaatctccccctcgttaatgtgtatggatctgctgaaatttaaagaacagatcgaatttatcgacagccatgccgattacttccacatcgatatcatggacggtcactttgtccccaatctgacactctcaccgttcttcgtaagtcaggttaaaaaactggcaactaaaccgctcgactgtcatctgatggtgacgcggccgcaggattacattgctcaactggcgcgtgcgggagcagatttcatcactctgcatccggaaaccatcaacggccaggcgttccgcctgattgatgaaatccgccgtcatgacatgaaagtggggctgatccttaacccggagacgccagttgaggccatgaaatactatatccataaggccgataaaattacggtcatgactgtcgatcccggctttgccggacaaccgttcattcctgaaatgctggataaacttgccgaactgaaggcatggcgtgaacgagaaggtctggagtacgaaattgaggtggacggttcctgcaaccaggcaacttacgaaaaactgatggcggcaggggcggatgtctttatcgtcggcacttccggcctgtttaatcatgcggaaaatatcgacgaagcatggagaattatgaccgcgcagattctggctgcaaaaagcgaggtacagcctcatgcaaaaacagcataaggatccggctgctaacaaaaggagatatacatatgtcaaccccgcgtcagattcttgctgcaatttttgatatggatggattacttatcgactcagaacctttatgggatcgagccgaactggatgtgatggcaagcctgggggtggatatctcccgtcgtaacgagctgccggacaccttaggtttacgcatcgatatggtggtcgatctttggtacgcccggcaaccgtggaatgggccaagccgtcaggaagtagtagaacgggttattgcccgtgccatttcactggttgaagagacacgtccattattaccaggcgtgcgcgaagccgttgcgttatgcaaagaacaaggtttattggtgggactggcctccgcgtcaccactacatatgctggaaaaagtgttgaccatgtttgacttacgcgacagtttcgatgccctcgcctcggccgaaaaactgccttacagcaagccgcatccgcaagtatatctcgactgcgcagcaaaactgggcgttgaccctctgacctgcgtagcgctggaagattcggtaaatggcatgatcgcctctaaagcagcccgcatgcgttccatcgtcgttcctgcgccagaagcgcaaaatgatccacgttttgtattagcagacgtcaaactttcatcgctgacagaactcaccgcaaaagaccttctcggttgagatccggctgctaaggtacctaatctagaggcatcaaataaaacgaaaggctcagtcgaaagactgggcctttcgttttatctgttgtttgtcggtgaacgctctcctgagtaggacaaatccgccgccctagacctaggcgttcggctgcggcgagcggtatcagctcactcaaaggcggtaatacggttatccacagaatcaggggataacgcaggaaagaacagagcgcctgatgcggtattttctccttacgcatctgtgcggtatttcacaccgcatatgctggatccttgacagctagctcagtcctaggtataatactagtttcttcactttccgctgattgttttagagctagaaatagcaagttaaaataaggctagtccgttatcaacttgaaaaagtggcaccgagtcggtgctttttttgaattctctagagtcgacctgcagaagcttagatctattaatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgactagtgcttggattctcaccaataaaaaacgcccggcggcaaccgagcgttctgaacaaatccagatggagttctgaggtcattactggatctatcaacaggagtccaagcgagctcgtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcacctcgag

SEQ ID NO: 66 - DNA, 3904 bp, synthetic construct
  source 1..3904: mol_type = other DNA
gtaataattttataaatgcgttcaaaataataatcaagtactaatagtgatattttaaggtctgatttttacgtgataattcaggagacacagaatgcgcataaaaataacagcataaaacaccttaccaccacccaagaatttcatattgtattgtttttcaatgaaaaaatattattcgcgtaatatctcacgataaataacattaggattttgttatttaaacacgagtcctttgcacttgcttactttatcgataaatcctacttttttaatgcgatccaatcattttaaggagtttaaaatggataagaagcaagtgaattcattaaagaggagaaaagatataccatgaaaatctccccctcgttaatgtgtatggatctgctgaaatttaaagaacagatcgaatttatcgacagccatgccgattacttccacatcgatatcatggacggtcactttgtccccaatctgacactctcaccgttcttcgtaagtcaggttaaaaaactggcaactaaaccgctcgactgtcatctgatggtgacgcggccgcaggattacattgctcaactggcgcgtgcgggagcagatttcatcactctgcatccggaaaccatcaacggccaggcgttccgcctgattgatgaaatccgccgtcatgacatgaaagtggggctgatccttaacccggagacgccagttgaggccatgaaatactatatccataaggccgataaaattacggtcatgactgtcgatcccggctttgccggacaaccgttcattcctgaaatgctggataaacttgccgaactgaaggcatggcgtgaacgagaaggtctggagtacgaaattgaggtggacggttcctgcaaccaggcaacttacgaaaaactgatggcggcaggggcggatgtctttatcgtcggcacttccggcctgtttaatcatgcggaaaatatcgacgaagcatggagaattatgaccgcgcagattctggctgcaaaaagcgaggtacagcctcatgcaaaaacagcataaggatccggctgctaacaaaaggagatatacatatgtcaaccccgcgtcagattcttgctgcaatttttgatatggatggattacttatcgactcagaacctttatgggatcgagccgaactggatgtgatggcaagcctgggggtggatatctcccgtcgtaacgagctgccggacaccttaggtttacgcatcgatatggtggtcgatctttggtacgcccggcaaccgtggaatgggccaagccgtcaggaagtagtagaacgggttattgcccgtgccatttcactggttgaagagacacgtccattattaccaggcgtgcgcgaagccgttgcgttatgcaaagaacaaggtttattggtgggactggcctccgcgtcaccactacatatgctggaaaaagtgttgaccatgtttgacttacgcgacagtttcgatgccctcgcctcggccgaaaaactgccttacagcaagccgcatccgcaagtatatctcgactgcgcagcaaaactgggcgttgaccctctgacctgcgtagcgctggaagattcggtaaatggcatgatcgcctctaaagcagcccgcatgcgttccatcgtcgttcctgcgccagaagcgcaaaatgatccacgttttgtattagcagacgtcaaactttcatcgctgacagaactcaccgcaaaagaccttctcggttgagatccggctgctaaggtacctaatctagaggcatcaaataaaacgaaaggctcagtcgaaagactgggcctttcgttttatctgttgtttgtcggtgaacgctctcctgagtaggacaaatccgccgccctagacctaggcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgactagtgcttggattctcaccaataaaaaacgcccggcggcaaccgagcgttctgaacaaatccagatggagttctgaggtcattactggatctatcaacaggagtccaagcgagctcgtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcacctcgag

SEQ ID NO: 67 - DNA, 4233 bp, synthetic construct
  source 1..4233: mol_type = other DNA
tccctatcagtgatagagattgacatccctatcagtgatagagatactgagcacatcagcaggacgcactgaccgaattcattaaagaggagaaaggtacaatgcctgacgctaaaaaacaggggcggtcaaacaaggcaatgacgtttttcgtctgcttccttgccgctctggcgggattactctttggcctggatatcggtgtaattgctggcgcactgccgtttattgcagatgaattccagattacttcgcacacgcaagaatgggtcgtaagctccatgatgttcggtgcggcagtcggtgcggtgggcagcggctggctctcctttaaactcgggcgcaaaaagagcctgatgatcggcgcaattttgtttgttgccggttcgctgttctctgcggctgcgccaaacgttgaagtactgattctttcccgcgttctactggggctggcggtgggtgtggcctcttataccgcaccgctgtacctctctgaaattgcgccggaaaaaattcgtggcagtatgatctcgatgtatcagttgatgatcactatcgggatcctcggtgcttatctttctgataccgccttcagctacaccggtgcatggcgctggatgctgggtgtgattatcatcccggcaattttgctgctgattggtgtcttcttcctgccagacagcccacgttggtttgccgccaaacgccgttttgttgatgccgaacgcgtgctgctacgcctgcgtgacaccagcgcggaagcgaaacgcgaactggatgaaatccgtgaaagtttgcaggttaaacagagtggctgggcgctgtttaaagagaacagcaacttccgccgcgcggtgttccttggcgtactgttgcaggtaatgcagcaattcaccgggatgaacgtcatcatgtattacgcgccgaaaatcttcgaactggcgggttataccaacactaccgagcaaatgtgggggaccgtgattgtcggcctgaccaacgtacttgccacctttatcgcaatcggccttgttgaccgctggggacgtaaaccaacgctaacgctgggcttcctggtgatggctgctggcatgggcgtactcggtacaatgatgcatatcggtattcactctccgtcggcgcagtatttcgccatcgccatgctgctgatgtttattgtcggttttgccatgagtgccggtccgctgatttgggtactgtgctccgaaattcagccgctgaaaggccgcgattttggcatcacctgctccactgccaccaactggattgccaacatgatcgttggcgcaacgttcctgaccatgctcaacacgctgggtaacgccaacaccttctgggtgtatgcggctctgaacgtactgtttatcctgctgacattgtggctggtaccggaaaccaaacacgtttcgctggaacatattgaacgtaatctgatgaaaggtcgtaaactgcgcgaaataggcgctcacgattaaggctgctaacaaaaggagatatacatatgacaaagtatgcattagtcggtgatgtgggcggcaccaacgcacgtcttgctctgtgtgatattgccagtggtgaaatctcgcaggctaagacctattcagggcttgattaccccagcctcgaagcggtcattcgcgtttatcttgaagaacataaggtcgaggtgaaagacggctgtattgccatcgcttgcccaattaccggtgactgggtggcgatgaccaaccatacctgggcgttctcaattgccgaaatgaaaaagaatctcggttttagccatctggaaattattaacgattttaccgctgtatcgatggcgatcccgatgctgaaaaaagagcatctgattcagtttggtggcgcagaaccggtcgaaggtaagcctattgcggtttacggtgccggaacggggcttggggttgcgcatctggtccatgtcgataagcgttgggtaagcttgccaggcgaaggcggtcacgttgattttgcgccgaatagtgaagaagaggccattatcctcgaaatattgcgtgcggaaattggtcatgtttcggcggagcgcgtgctttctggccctgggctggtgaatttgtatcgcgcaattgtgaaagctgacaaccgcctgccagaaaatctcaagccaaaagatattaccgaacgcgcgctggctgacagctgcaccgattgccgccgcgcattgtcgctgttttgcgtcattatgggccgttttggcggcaatctggcgctcaatctcgggacatttggcggcgtgtttattgcgggcggtatcgtgccgcgcttccttgagttcttcaaagcctccggtttccgtgccgcatttgaagataaagggcgctttaaagaatatgtccatgatattccggtgtatctcatcgtccatgacaatccgggccttctcggttccggtgcacatttacgccagaccttaggtcacattctgtaaacgcgtgctagaggcatcaaataaaacgaaaggctcagtcgaaagactgggcctttcgttttatctgttgtttgtcggtgaacgctctcctgagtaggacaaatccgccgccctagacctaggtagcgcagaaacgtcctagaagatgccaggaggatacttagcagagagacaataaggccggagcgaagccgtttttccataggctccgcccccctgacgaacatcacgaaatctgacgctcaaatcagtggtggcgaaacccgacaggactataaagataccaggcgtttccccctgatggctccctcttgcgctctcctgttcccgtcctgcggcgtccgtgttgtggtggaggctttacccaaatcaccacgtcccgttccgtgtagacagttcgctccaagctgggctgtgtgcaagaaccccccgttcagcccgactgctgcgccttatccggtaactatcatcttgagtccaacccggaaagacacgacaaaacgccactggcagcagccattggtaactgagaattagtggatttagatatcgagagtcttgaagtggtggcctaacagaggctacactgaaaggacagtatttggtatctgcgctccactaaagccagttaccaggttaagcagttccccaactgacttaaccttcgatcaaaccgcctccccaggcggttttttcgtttacagagcaggagattacgacgatcgtaaaaggatctcaagaagatcctttacggattcccgacaccatcaccagataaaatatttctagatttcagtgcaatttatctcttcaaatgtagcacctgaagtcagccccatacgatataagttgttactagtgcttggattctcaccaataaaaaacgcccggcggcaaccgagcgttctgaacaaatccagatggagttctgaggtcattactggatctatcaacaggagtccaagcgagctctcgaaccccagagtcccgctcattaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacctgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctgcgtaacatgcgaaacgatcctcatcctgtctcttgatcagatcttgatcccctgcgccatcagatccttggcggcaagaaagccatccagtttactttgcagggcttcccaaccttaccagagggcgccccagctggcaattccgacgtctaagaaaccattattatcatgacattaacctataaaaataggcgtatcacgaggccctttcgtcttcacctcgag

SEQ ID NO: 68 - DNA, 12545 bp, synthetic construct
  source 1..12545: mol_type = other DNA
atggataagaaatactcaataggcttagatatcggcacaaatagcgtcggatgggcggtgatcactgatgattataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatcacattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgactgaagtatattttagatgaagattatttcttaatctagacatgagcggatacatatttgaatgtatttagaaaaataaacaaataggggttccgcgcacatttccccgaaaagtgccacctgcatcgatttattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgtttttttgggaattcgagctctaaggaggttataaaaaatggatattaatactgaaactgagatcaagcaaaagcattcactaaccccctttcctgttttcctaatcagcccggcatttcgcgggcgatattttcacagctatttcaggagttcagccatgaacgcttattacattcaggatcgtcttgaggctcagagctgggcgcgtcactaccagcagctcgcccgtgaagagaaagaggcagaactggcagacgacatggaaaaaggcctgccccagcacctgtttgaatcgctatgcatcgatcatttgcaacgccacggggccagcaaaaaatccattacccgtgcgtttgatgacgatgttgagtttcaggagcgcatggcagaacacatccggtacatggttgaaaccattgctcaccaccaggttgatattgattcagaggtataaaacgaatgagtactgcactcgcaacgctggctgggaagctggctgaacgtgtcggcatggattctgtcgacccacaggaactgatcaccactcttcgccagacggcatttaaaggtgatgccagcgatgcgcagttcatcgcattactgatcgttgccaaccagtacggccttaatccgtggacgaaagaaatttacgcctttcctgataagcagaatggcatcgttccggtggtgggcgttgatggctggtcccgcatcatcaatgaaaaccagcagtttgatggcatggactttgagcaggacaatgaatcctgtacatgccggatttaccgcaaggaccgtaatcatccgatctgcgttaccgaatggatggatgaatgccgccgcgaaccattcaaaactcgcgaaggcagagaaatcacggggccgtggcagtcgcatcccaaacggatgttacgtcataaagccatgattcagtgtgcccgtctggccttcggatttgctggtatctatgacaaggatgaagccgagcgcattgtcgaaaatactgcatacactgcagaacgtcagccggaacgcgacatcactccggttaacgatgaaaccatgcaggagattaacactctgctgatcgccctggataaaacatgggatgacgacttattgccgctctgttcccagatatttcgccgcgacattcgtgcatcgtcagaactgacacaggccgaagcagtaaaagctcttggattcctgaaacagaaagccgcagagcagaaggtggcagcatgacaccggacattatcctgcagcgtaccgggatcgatgtgagagctgtcgaacagggggatgatgcgtggcacaaattacggctcggcgtcatcaccgcttcagaagttcacaacgtgatagcaaaaccccgctccggaaagaagtggcctgacatgaaaatgtcctacttccacaccctgcttgctgaggtttgcaccggtgtggctccggaagttaacgctaaagcactggcctggggaaaacagtacgagaacgacgccagaaccctgtttgaattcacttccggcgtgaatgttactgaatccccgatcatctatcgcgacgaaagtatgcgtaccgcctgctctcccgatggtttatgcagtgacggcaacggccttgaactgaaatgcccgtttacctcccgggatttcatgaagttccggctcggtggtttcgaggccataaagtcagcttacatggcccaggtgcagtacagcatgtgggtgacgcgaaaaaatgcctggtactttgccaactatgacccgcgtatgaagcgtgaaggcctgcattatgtcgtgattgagcgggatgaaaagtacatggcgagttttgacgagatcgtgccggagttcatcgaaaaaatggacgaggcactggctgaaattggttttgtatttggggagcaatggcgatgacgcatcctcacgataatatccgggtaggcgcaatcactttcgtctactccgttacaaagcgaggctgggtatttcccggcctttctgttatccgaaatccactgaaagcacagcggctggctgaggagataaataataaacgaggggctgtatgcacaaagcatcttctgttgagttaagaacgagtatcgagatggcacatagccttgctcaaattggaatcaggtttgtgccaataccagtagaaacagacgaagaatccatgggtatggacagatctcaaaaaaagcaccgactcggtgccactttttcaagttgataacggactagccttattttaacttgctatttctagctctaaaacctggtaacaggattagcagatgtgtgaaattgttatccgctcacaattccacacattatacgagccggatgattaattgtcaacagctcatttcagaatatttgccagaaccgttatgatgtcggcgcaaaaaacattatccagaacgggagtgcgccttgagcgacacgaattatgcagtgatttacgacctgcacagccataccacagcttccgatggctgcctgacgccagaagcattggtgcaccgtgcagtcgatgataagctgtcaaaccagatcaattcgcgctaactcacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttgacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcaccattcgatggtgtcaacgtaaatgcatgccgcttcgccttccatgggtatggacagttttccctttgatatgtaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatacttactttgcatgtcactcaaaaattttgcctcaaaactggtgagctgaatttttgcagttaaagcatcgtgtagtgtttttcttagtccgttacgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcggttgcggccgcaatcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttacgtttccacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccctgcagggtttgcagtcagagtagaatagaagtatcaaaaaaagcaccgactcggtgccactttttcaagttgataacggactagccttattttaacttgctatgctgttttgaatggttccaacaagattattttataacttttataacaaataatcaaggagaaattcaaagaaatttatcagccataaaacaatacttaatactatagaatgataacaaaataaactactttttaaaagaattttgtgttataatctatttattattaagtattgggtaatattttttgaagagatattttgaaaaagaaaaattaaagcatattaaactaatttcggaggtcattaaaactattattgaaatcatcaaactcattatggatttaatttaaactttttattttaggaggcaaaa

SEQ ID NO: 69 - DNA, 2187 bp, synthetic construct
  source 1..2187: mol_type = other DNA
ttcccgtgcatcggtctgccgttttagagctagaaatagcaagttaaaataaggctagtccgttatcaacttgaaaaagtggcaccgagtcggtgctttttttgaattctctagagtcgacctgcagaagcttagatctattaccctgttatccctactcgagttcatgtgcagctccataagcaaaaggggatgataagtttatcaccaccgactatttgcaacagtgccgttgatcgtgctatgatcgactgatgtcatcagcggtggagtgcaatgtcatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaagatgccgctcgccagtcgattggctgagctcatgaagttcctattccgaagttccgcgaacgcgtaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatgctggatccttgacagctagctcagtcctaggtataatactagt

SEQ ID NO: 70 - DNA, 2187 bp, synthetic construct
  source 1..2187: mol_type = other DNA
aattgaatccggggccatccgttttagagctagaaatagcaagttaaaataaggctagtccgttatcaacttgaaaaagtggcaccgagtcggtgctttttttgaattctctagagtcgacctgcagaagcttagatctattaccctgttatccctactcgagttcatgtgcagctccataagcaaaaggggatgataagtttatcaccaccgactatttgcaacagtgccgttgatcgtgctatgatcgactgatgtcatcagcggtggagtgcaatgtcatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaagatgccgctcgccagtcgattggctgagctcatgaagttcctattccgaagttccgcgaacgcgtaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatgctggatccttgacagctagctcagtcctaggtataatactagt

SEQ ID NO: 71 - DNA, 2187 bp, synthetic construct
  source 1..2187: mol_type = other DNA
gtcgcactggctgttgctgggttttagagctagaaatagcaagttaaaataaggctagtccgttatcaacttgaaaaagtggcaccgagtcggtgctttttttgaattctctagagtcgacctgcagaagcttagatctattaccctgttatccctactcgagttcatgtgcagctccataagcaaaaggggatgataagtttatcaccaccgactatttgcaacagtgccgttgatcgtgctatgatcgactgatgtcatcagcggtggagtgcaatgtcatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaagatgccgctcgccagtcgattggctgagctcatgaagttcctattccgaagttccgcgaacgcgtaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatgctggatccttgacagctagctcagtcctaggtataatactagt

SEQ ID NO: 72 - DNA, 2187 bp, synthetic construct
  source 1..2187: mol_type = other DNA
gcagaagaagtggggatcgagttttagagctagaaatagcaagttaaaataaggctagtccgttatcaacttgaaaaagtggcaccgagtcggtgctttttttgaattctctagagtcgacctgcagaagcttagatctattaccctgttatccctactcgagttcatgtgcagctccataagcaaaaggggatgataagtttatcaccaccgactatttgcaacagtgccgttgatcgtgctatgatcgactgatgtcatcagcggtggagtgcaatgtcatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaagatgccgctcgccagtcgattggctgagctcatgaagttcctattccgaagttccgcgaacgcgtaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatgctggatccttgacagctagctcagtcctaggtataatactagt

SEQ ID NO: 73 - DNA, 2187 bp, synthetic construct
  source 1..2187: mol_type = other DNA
tcggccctcgatagccagcagttttagagctagaaatagcaagttaaaataaggctagtccgttatcaacttgaaaaagtggcaccgagtcggtgctttttttgaattctctagagtcgacctgcagaagcttagatctattaccctgttatccctactcgagttcatgtgcagctccataagcaaaaggggatgataagtttatcaccaccgactatttgcaacagtgccgttgatcgtgctatgatcgactgatgtcatcagcggtggagtgcaatgtcatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaagatgccgctcgccagtcgattggctgagctcatgaagttcctattccgaagttccgcgaacgcgtaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatgctggatccttgacagctagctcagtcctaggtataatactagt

SEQ ID NO: 74 - DNA, 30 bp, synthetic construct
  source 1..30: mol_type = other DNA
gcacatcagcgttttagagctagaaatagc

SEQ ID NO: 75 - DNA, 30 bp, synthetic construct
  source 1..30: mol_type = other DNA
tcagtatcttactagtattatacctaggac

SEQ ID NO: 76 - DNA, 44 bp, synthetic construct
  source 1..44: mol_type = other DNA
gccatatcgaaggtcgtcatatgaaaatctccccctcgttaatg

SEQ ID NO: 77 - DNA, 50 bp, synthetic construct
  source 1..50: mol_type = other DNA
atctccttttgttagcagccggatccttatgctgtttttgcatgaggctg

SEQ ID NO: 78 - DNA, 52 bp, synthetic construct
  source 1..52: mol_type = other DNA
ggctgctaacaaaaggagatatacatatgtcaaccccgcgtcagattcttgc

SEQ ID NO: 79 - DNA, 46 bp, synthetic construct
  source 1..46: mol_type = other DNA
gctttgttagcagccggatcctcaaccgagaaggtcttttgcggtg

SEQ ID NO: 80 - DNA, 49 bp, synthetic construct
  source 1..49: mol_type = other DNA
ggctgctaacaaaaggagatatacatatgagcgtaaaagttatcgtcac

SEQ ID NO: 81 - DNA, 43 bp, synthetic construct
  source 1..43: mol_type = other DNA
gctttgttagcagccggatcctcagctgttaaaaggggatgtg

SEQ ID NO: 82 - DNA, 42 bp, synthetic construct
  source 1..42: mol_type = other DNA
ggatccggctgctaacaaagcccgaaaggaagctgagttggc

SEQ ID NO: 83 - DNA, 58 bp, synthetic construct
  source 1..58: mol_type = other DNA
ctgacctgaatcaattcagcaggaagtgattgttatactatttgcacattcgttggat

SEQ ID NO: 84 - DNA, 58 bp, synthetic construct
  source 1..58: mol_type = other DNA
tctgttgccggaagtcttcttgcacatcgaagtgatccaacgaatgtgcaaatagtat

SEQ ID NO: 85 - DNA, 58 bp, synthetic construct
  source 1..58: mol_type = other DNA
agacttccggcaacagatttcattttgcattccaaagttcagaggtagtctgatttcg

SEQ ID NO: 86 - DNA, 59 bp, synthetic construct
  source 1..59: mol_type = other DNA
tgtcatcggtttcagggtaaaggaatctgcctttttccgaaatcagactacctctgaac

SEQ ID NO: 87 - DNA, 60 bp, synthetic construct
  source 1..60: mol_type = other DNA
accctgaaaccgatgacagaagcaaaaatgcctgatgcgcttcgcttatcaggcctacat

SEQ ID NO: 88 - DNA, 57 bp, synthetic construct
  source 1..57: mol_type = other DNA
cctacaaaagtttgcaaattcaataaattgcagaattcatgtaggcctgataagcga

SEQ ID NO: 89 - DNA, 43 bp, synthetic construct
  source 1..43: mol_type = other DNA
caagcgagctcgatatcaaatcagaagaactcgtcaagaaggc

SEQ ID NO: 90 - DNA, 49 bp, synthetic construct
  source 1..49: mol_type = other DNA
caggagctaaggaagctaaaatgattgaacaagatggattgcacgcagg

SEQ ID NO: 91 - DNA, 36 bp, synthetic construct
  source 1..36: mol_type = other DNA
tttagcttccttagctcctgaaaatctcgataactc

SEQ ID NO: 92 - DNA, 26 bp, synthetic construct
  source 1..26: mol_type = other DNA
tttgatatcgagctcgcttggactcc

SEQ ID NO: 93 - DNA, 30 bp, synthetic construct
  source 1..30: mol_type = other DNA
tcggtctgccgttttagagctagaaatagc

SEQ ID NO: 94 - DNA, 30 bp, synthetic construct
  source 1..30: mol_type = other DNA
tgcacgggaaactagtattatacctaggac

SEQ ID NO: 95 - DNA, 30 bp, synthetic construct
  source 1..30: mol_type = other DNA
ctgttgctgggttttagagctagaaatagc

SEQ ID NO: 96 - DNA, 30 bp, synthetic construct
  source 1..30: mol_type = other DNA
ccagtgcgacactagtattatacctaggac

SEQ ID NO: 97 - DNA, 42 bp, synthetic construct
  source 1..42: mol_type = other DNA
cgctgtcagatgtaactctgtaaaacagatcaggaaggcgta

SEQ ID NO: 98 - DNA, 59 bp, synthetic construct
  source 1..59: mol_type = other DNA
tgtgatgttaatgaattaaaaacacccaaaatcgatgaattacgccttcctgatctgtt

SEQ ID NO: 99 - DNA, 60 bp, synthetic construct
  source 1..60: mol_type = other DNA
gggtgtttttaattcattaacatcacaaatgttttttgattgtgaagttttgcacggacg

SEQ ID NO: 100 - DNA, 59 bp, synthetic construct
  source 1..59: mol_type = other DNA
ctcatccatgcaagtagtggatgaatctcatcttccccgtccgtgcaaaacttcacaat

SEQ ID NO: 101 - DNA, 60 bp, synthetic construct
  source 1..60: mol_type = other DNA
ccactacttgcatggatgagtaatgattaatgtggatagagtttctttttgaggttggct

SEQ ID NO: 102 - DNA, 42 bp, synthetic construct
  source 1..42: mol_type = other DNA
agcggaaagcgtttcattagccaacctcaaaaagaaactcta

SEQ ID NO: 103 - DNA, 55 bp, synthetic construct
  source 1..55: mol_type = other DNA
atgaaacgctttccgctatttcttttattcaccctgctcacgctgtccaccgttc

SEQ ID NO: 104 - DNA, 22 bp, synthetic construct
  source 1..22: mol_type = other DNA
cggaacggtggacagcgtgagc

SEQ ID NO: 105 - DNA, 30 bp, synthetic construct
  source 1..30: mol_type = other DNA
tggggatcgagttttagagctagaaatagc

SEQ ID NO: 106 - DNA, 30 bp, synthetic construct
  source 1..30: mol_type = other DNA
cttcttctgcactagtattatacctaggac

SEQ ID NO: 107 - DNA, 60 bp, synthetic construct
  source 1..60: mol_type = other DNA
taatcgcacgggtggataagcgtttacagttttcgcaagctcgtaaaagcagtacagtgc

SEQ ID NO: 108 - DNA, 60 bp, synthetic construct
  source 1..60: mol_type = other DNA
cggtacttaagccagggtatacttgtaattttcttacggtgcactgtactgcttttacga

SEQ ID NO: 109 - DNA, 59 bp, synthetic construct
  source 1..59: mol_type = other DNA
accctggcttaagtaccgggttagttaacttaaggagaatgactatctgcgcttatcct

SEQ ID NO: 110 - DNA, 56 bp, synthetic construct
  source 1..56: mol_type = other DNA
gcgcaagatcatgttaccggtaaaataaccataaaggataagcgcagatagtcatt

SEQ ID NO: 111 - DNA, 60 bp, synthetic construct
  source 1..60: mol_type = other DNA
cggtaacatgatcttgcgcagattgtagaacaatttttacactttcaggcctcgtgcgga

SEQ ID NO: 112 - DNA, 51 bp, synthetic construct
  source 1..51: mol_type = other DNA
cagtcagtgtaataaaaaaagcctcgtgggtgaatccgcacgaggcctgaa

SEQ ID NO: 113 - DNA, 46 bp, synthetic construct
  source 1..46: mol_type = other DNA
aagaggagaaaagatataccatgaaaatctccccctcgttaatgtg

SEQ ID NO: 114 - DNA, 39 bp, synthetic construct
  source 1..39: mol_type = other DNA
ggtaccttagcagccggatctcaaccgagaaggtctttt

SEQ ID NO: 115 - DNA, 35 bp, synthetic construct
  source 1..35: mol_type = other DNA
gatccggctgctaaggtacctaatctagaggcatc

SEQ ID NO: 116 - DNA, 40 bp, synthetic construct
  source 1..40: mol_type = other DNA
ggtatatcttttctcctctttaatgaattcggtcagtgcg

SEQ ID NO: 117 - DNA, 30 bp, synthetic construct
  source 1..30: mol_type = other DNA
acaatcgtgcgttttagagctagaaatagc

SEQ ID NO: 118 - DNA, 30 bp, synthetic construct
  source 1..30: mol_type = other DNA
ggattgccatactagtattatacctaggac

SEQ ID NO: 119 - DNA, 59 bp, synthetic construct
  source 1..59: mol_type = other DNA
ctgaattattttactctgtgtaataaataaagggcgcttagatgccctgtacacggcga

SEQ ID NO: 120 - DNA, 48 bp, synthetic construct
  source 1..48: mol_type = other DNA
acgcgtggcaaggggggagagcctcgccgtgtacagggcatctaagcg

SEQ ID NO: 121 - DNA, 60 bp, synthetic construct
  source 1..60: mol_type = other DNA
ccttgccacgcgtgagaacgtaaaaaaagcacccatactcaggagcactctcaatttccg

SEQ ID NO: 122 - DNA, 54 bp, synthetic construct
  source 1..54: mol_type = other DNA
gccatctggctgccttagtctccccaacgtcttacggaaattgagagtgctcct

SEQ ID NO: 123 - DNA, 60 bp, synthetic construct
  source 1..60: mol_type = other DNA
ggcagccagatggctgccttttttacaggtgttattcagaattgatacgtgccggtaatg

SEQ ID NO: 124 - DNA, 60 bp, synthetic construct
  source 1..60: mol_type = other DNA
gtggaaggttctatcgtctacggcacaccgcgtaatttcagcattaccggcacgtatcaa

SEQ ID NO: 125 - DNA, 30 bp, synthetic construct
  source 1..30: mol_type = other DNA
aacctgcaaagttttagagctagaaatagc

SEQ ID NO: 126 - DNA, 30 bp, synthetic construct
  source 1..30: mol_type = other DNA
agcaaatgcaactagtattatacctaggac

SEQ ID NO: 127 - DNA, 51 bp, synthetic construct
  source 1..51: mol_type = other DNA
caacaccaacaccggccgtaaatgcagcatgatcgaacacatcatgcagtc

SEQ ID NO: 128 - DNA, 59 bp, synthetic construct
  source 1..59: mol_type = other DNA
tccgcaaaccttctcaatcaattccgccgggaattgcatcgactgcatgatgtgttcga

SEQ ID NO: 129 - DNA, 60 bp, synthetic construct
  source 1..60: mol_type = other DNA
gattgagaaggtttgcggaactatctaaaacgttgcagacaaaggacaaagcaacacatt

SEQ ID NO: 130 - DNA, 57 bp, synthetic construct
  source 1..57: mol_type = other DNA
aagggcgatcttgcgaccgccctttttttattaaatgtgttgctttgtcctttgtct

SEQ ID NO: 131 - DNA, 60 bp, synthetic construct
  source 1..60: mol_type = other DNA
cgcaagatcgcccttttttacgtatgacaaacacagaattgcctgatgcgctacgcttat

SEQ ID NO: 132 - DNA, 58 bp, synthetic construct
  source 1..58: mol_type = other DNA
tcgcttaaattcaatatattgcaccatcctcgtaggcctgataagcgtagcgcatcag

SEQ ID NO: 133 - DNA, 30 bp, synthetic construct
  source 1..30: mol_type = other DNA
atgacgaccttcgatatggccgctgctgtg

SEQ ID NO: 134 - DNA, 30 bp, synthetic construct
  source 1..30: mol_type = other DNA
aaattgtgaggttttagagctagaaatagc

SEQ ID NO: 135 - DNA, 30 bp, synthetic construct
  source 1..30: mol_type = other DNA
aatagatctaactagtattatacctaggac

SEQ ID NO: 136 - DNA, 57 bp, synthetic construct
  source 1..57: mol_type = other DNA
aatatcctacacacttttttaacaaaaactgagactagtacgactttttgcggctcc

SEQ ID NO: 137 - DNA, 54 bp, synthetic construct
  source 1..54: mol_type = other DNA
cccactattaaagcaagaatcctacgggaagtaacctggagccgcaaaaagtcg

SEQ ID NO: 138 - DNA, 59 bp, synthetic construct
  source 1..59: mol_type = other DNA
aggattcttgctttaatagtgggattaatttccacattaaaacagggattgatcgagct

SEQ ID NO: 139 - DNA, 59 bp, synthetic construct
  source 1..59: mol_type = other DNA
gcctttaataagcttagcaagagatgttaattttttcagtaagctcgatcaatccctgt

SEQ ID NO: 140 - DNA, 60 bp, synthetic construct
  source 1..60: mol_type = other DNA
tctcttgctaagcttattaaaggcttataacaccttcaggcggccagtccgcctgatttc

SEQ ID NO: 141 - DNA, 60 bp, synthetic construct
  source 1..60: mol_type = other DNA
ctaccgctaccagcgatttattcataatgattatccataaaatgaaatcaggcggactgg

SEQ ID NO: 142 - DNA, 50 bp, synthetic construct
  source 1..50: mol_type = other DNA
tcctcgctcactgactcgcttccttacgcatctgtgcggtatttcacacc

SEQ ID NO: 143 - DNA, 50 bp, synthetic construct
  source 1..50: mol_type = other DNA
gcagtcgaacgaccgagcgtggataacagggtaatagatctaagcttctg

SEQ ID NO: 144 - DNA, 22 bp, synthetic construct
  source 1..22: mol_type = other DNA
acgctcggtcgttcgactgcgg

SEQ ID NO: 145 - DNA, 34 bp, synthetic construct
  source 1..34: mol_type = other DNA
agcgagtcagtgagcgaggaagcggaatatatcc

SEQ ID NO: 146 - DNA, 23 bp, synthetic construct
  source 1..23: mol_type = other DNA
gttttagagctagaaatagcaag

SEQ ID NO: 147 - DNA, 38 bp, synthetic construct
  source 1..38: mol_type = other DNA
tcatgacggcactagtattatacctaggactgagctag

SEQ ID NO: 148 - DNA, 43 bp, synthetic construct
  source 1..43: mol_type = other DNA
catgaaagtggttttagagctagaaatagcaagttaaaataag

SEQ ID NO: 149 - DNA, 52 bp, synthetic construct
  source 1..52: mol_type = other DNA
cgccatcattggcggcaccagtttcttcggcggcaaggggcgcattttctct

SEQ ID NO: 150 - DNA, 56 bp, synthetic construct
  source 1..56: mol_type = other DNA
gttgttgatggtgccgatgatcaacccgccaatcaccacagagaaaatgcgcccct

SEQ ID NO: 151 - DNA, 60 bp, synthetic construct
  source 1..60: mol_type = other DNA
tcggcaccatcaacaacggtctgaatattttgcaggtacaaacctattaccaactggtgg

SEQ ID NO: 152 - DNA, 58 bp, synthetic construct
  source 1..58: mol_type = other DNA
caagggcgacagccgcgataattaatccgcccatcaccaccagttggtaataggtttg

SEQ ID NO: 153 - DNA, 60 bp, synthetic construct
  source 1..60: mol_type = other DNA
ggctgtcgcccttgaccgtcttatcagtaagtaaggaattgaaccgtcgtagcgggcgtg

SEQ ID NO: 154 - DNA, 55 bp, synthetic construct
  source 1..55: mol_type = other DNA
tgctgtccgcagacaaaagcggatatgcgttgcccccatatccacgcccgctacg

SEQ ID NO: 155 - DNA, 60 bp, synthetic construct
  source 1..60: mol_type = other DNA
tgtctgcggacagcagaaggtgaaacgctacactgcgaaaaaaagcggaccgcagaagtc

SEQ ID NO: 156 - DNA, 44 bp, synthetic construct
  source 1..44: mol_type = other DNA
aggccgggagcaatgacttctgcggtccgctttttttcgcagtg

SEQ ID NO: 157 - DNA, 60 bp, synthetic construct
  source 1..60: mol_type = other DNA
ttgctcccggcctggtgtcgggtatcggcgaaatgattgacgagcaactcaggcgcttta

SEQ ID NO: 158 - DNA, 57 bp, synthetic construct
  source 1..57: mol_type = other DNA
ccggaaatcccatcaccagaccatgacagcgagcgttaaagcgcctgagttgctcgt

SEQ ID NO: 159 - DNA, 23 bp, synthetic construct
  source 1..23: mol_type = other DNA
tgagcgggactctggggttcgag

SEQ ID NO: 160 - DNA, 27 bp, synthetic construct
  source 1..27: mol_type = other DNA
gcgaaacgatcctcatcctgtctcttg

SEQ ID NO: 161 - DNA, 37 bp, synthetic construct
  source 1..37: mol_type = other DNA
tgaggatcgtttcgcatgttacgcagcagcaacgatg

SEQ ID NO: 162 - DNA, 36 bp, synthetic construct
  source 1..36: mol_type = other DNA
ccagagtcccgctcattaggtggcggtacttgggtc

SEQ ID NO: 163 - DNA, 30 bp, synthetic construct
  source 1..30: mol_type = other DNA
gtttaaacagactagtattatacctaggac

SEQ ID NO: 164 - DNA, 30 bp, synthetic construct
  source 1..30: mol_type = other DNA
tgcagactctgttttagagctagaaatagc

SEQ ID NO: 165 - DNA, 37 bp, synthetic construct
  source 1..37: mol_type = other DNA
ggactgtattgcgctcttcgtgcgtcgcgtctgttaa

SEQ ID NO: 166 - DNA, 60 bp, synthetic construct
  source 1..60: mol_type = other DNA
gcctggcggttcgactttagcctgtattgttagcgccagtttttaacagacgcgacgcac

SEQ ID NO: 167 - DNA, 43 bp, synthetic construct
  source 1..43: mol_type = other DNA
cgaaccgccaggctagactttagttccacaacactaaacctat

SEQ ID NO: 168 - DNA, 54 bp, synthetic construct
  source 1..54: mol_type = other DNA
ccgggaaatgtatttccccaacttataggtttagtgttgtggaactaaagtcta

SEQ ID NO: 169 - DNA, 59 bp, synthetic construct
  source 1..59: mol_type = other DNA
ggggaaatacatttcccgggttcttttaaaaatcagtcacaagtaaggtagggttatga

SEQ ID NO: 170 - DNA, 60 bp, synthetic construct
  source 1..60: mol_type = other DNA
ccgaaagcgatacccggggatgctaaaatgcctgaaatcataaccctaccttacttgtga

SEQ ID NO: 171 - DNA, 60 bp, synthetic construct
  source 1..60: mol_type = other DNA
cgggtatcgctttcggtaaagctctgcttctgaaagaagacgaaattgtcattgaccgga

SEQ ID NO: 172 - DNA, 45 bp, synthetic construct
  source 1..45: mol_type = other DNA
cctggtcggcagaaatttttttccggtcaatgacaatttcgtctt

SEQ ID NO: 173 - DNA, 25 bp, synthetic construct
  source 1..25: mol_type = other DNA
cggatccatctccttttgttagcag

SEQ ID NO: 174 - DNA, 25 bp, synthetic construct
  source 1..25: mol_type = other DNA
gctgctaacaaagcccgaaaggaag

SEQ ID NO: 175 - DNA, 41 bp, synthetic construct
  source 1..41: mol_type = other DNA
aacaaaaggagatggatccggtgcggtgcaaaggttttctg

SEQ ID NO: 176 - DNA, 40 bp, synthetic construct
  source 1..40: mol_type = other DNA
tttcgggctttgttagcagctcactgaataataacatcgc

SEQ ID NO: 177 - DNA, 40 bp, synthetic construct
  source 1..40: mol_type = other DNA
aacaaaaggagatggatccgatgtaccaggttgttgcgtc

SEQ ID NO: 178 - DNA, 40 bp, synthetic construct
  source 1..40: mol_type = other DNA
tttcgggctttgttagcagcttacgataaatagagtttac

SEQ ID NO: 179 - DNA, 40 bp, synthetic construct
  source 1..40: mol_type = other DNA
aacaaaaggagatggatccgatggctattaaactcattgc

SEQ ID NO: 180 - DNA, 40 bp, synthetic construct
  source 1..40: mol_type = other DNA
tttcgggctttgttagcagcttaattcagcacatacttct

SEQ ID NO: 181 - DNA, 42 bp, synthetic construct
  source 1..42: mol_type = other DNA
aacaaaaggagatggatccgatgctctatatctttgatttag

SEQ ID NO: 182 - DNA, 39 bp, synthetic construct
  source 1..39: mol_type = other DNA
tttcgggctttgttagcagcttagcataacaccttcgcg

SEQ ID NO: 183 - DNA, 33 bp, synthetic construct
  source 1..33: mol_type = other DNA
ctcgaggtgaagacgaaagggcctcgtgatacg

SEQ ID NO: 184 - DNA, 41 bp, synthetic construct
  source 1..41: mol_type = other DNA
cgaattcattaaagaggagaaaggtaccatgggtcatcacc

SEQ ID NO: 185 - DNA, 40 bp, synthetic construct
  source 1..40: mol_type = other DNA
ttcgtcttcacctcgagtatccgaatgtaagaaagttggc

SEQ ID NO: 186 - DNA, 40 bp, synthetic construct
  source 1..40: mol_type = other DNA
cctctttaatgaattcgaggttcaatccctcaatgatgcc

SEQ ID NO: 187 - DNA, 48 bp, synthetic construct
  source 1..48: mol_type = other DNA
cgtcttcacctcgagtcattgaatctttattagttttgtttttcacgc

SEQ ID NO: 188 - DNA, 46 bp, synthetic construct
  source 1..46: mol_type = other DNA
cctctttaatgaattcgaatttcatatcctcttgatgttatgtccc

SEQ ID NO: 189 - DNA, 29 bp, synthetic construct
  source 1..29: mol_type = other DNA
cacctcgagtttgcagtgcaactaattcc

SEQ ID NO: 190 - DNA, 30 bp, synthetic construct
  source 1..30: mol_type = other DNA
cctctttaatgaattcgagcgttatctcgc

SEQ ID NO: 191 - DNA, 42 bp, synthetic construct
  source 1..42: mol_type = other DNA
gccctttcgtcttcacctcgaggtaataattttataaatgcg

SEQ ID NO: 192 - DNA, 44 bp, synthetic construct
  source 1..44: mol_type = other DNA
cctctttaatgaattcgacttgcttcttatccattttaaactcc

SEQ ID NO: 193 - DNA, 30 bp, synthetic construct
  source 1..30: mol_type = other DNA
atagccagcagttttagagctagaaatagc

SEQ ID NO: 194 - DNA, 30 bp, synthetic construct
  source 1..30: mol_type = other DNA
cgagggccgaactagtattatacctaggac

SEQ ID NO: 195 - DNA, 30 bp, synthetic construct
  source 1..30: mol_type = other DNA
tccgctgattgttttagagctagaaatagc

SEQ ID NO: 196 - DNA, 35 bp, synthetic construct
  source 1..35: mol_type = other DNA
aagtgaagaaactagtattatacctaggactgagc

SEQ ID NO: 197 - DNA, 57 bp, synthetic construct
  source 1..57: mol_type = other DNA
ggtatatcttttctcctctttaatgaattcacttgcttcttatccattttaaactcc

SEQ ID NO: 198 - DNA, 30 bp, synthetic construct
  source 1..30: mol_type = other DNA
gaaaggtaccgttttagagctagaaatagc

SEQ ID NO: 199 - DNA, 30 bp, synthetic construct
  source 1..30: mol_type = other DNA
tcctctttaaactagtattatacctaggac

SEQ ID NO: 200 - DNA, 25 bp, synthetic construct
  source 1..25: mol_type = other DNA
actagtattatacctaggactgagc

SEQ ID NO: 201 - DNA, 20 bp, synthetic construct
  source 1..20: mol_type = other DNA
ttcccgtgcatcggtctgcc

SEQ ID NO: 202 - DNA, 20 bp, synthetic construct
  source 1..20: mol_type = other DNA
gccgtcatgacatgaaagtg

SEQ ID NO: 203 - DNA, 20 bp, synthetic construct
  source 1..20: mol_type = other DNA
gcagaagaagtggggatcga

SEQ ID NO: 204 - DNA, 20 bp, synthetic construct
  source 1..20: mol_type = other DNA
gtcgcactggctgttgctgg

SEQ ID NO: 205 - DNA, 20 bp, synthetic construct
  source 1..20: mol_type = other DNA
tcggccctcgatagccagca

SEQ ID NO: 206 - DNA, 20 bp, synthetic construct
  source 1..20: mol_type = other DNA
tgcatttgctaacctgcaaa

SEQ ID NO: 207 - DNA, 20 bp, synthetic construct
  source 1..20: mol_type = other DNA
ctgtttaaactgcagactct

SEQ ID NO: 208 - DNA, 20 bp, synthetic construct
  source 1..20: mol_type = other DNA
atggcaatccacaatcgtgc

(19) United States
(12) Patent Application Publication
(10) Pub. No.: US 2026/0078422 A1
(43) Pub. Date: Mar. 19, 2026

(54) MICROORGANISMS FOR THE PRODUCTION OF LOW-CALORIE SUGARS

(71) Applicant: The Regents of the University of California, Oakland, CA (US)

(72) Inventors: Shota ATSUMI, Davis, CA (US); Justin Bloomfield SIEGEL, Davis, CA (US); Angela ZHANG, Davis, CA (US); Jayce Elizabeth TAYLOR, Davis, CA (US); Jake Nicholas GONZALES, Davis, CA (US); Dileep Sai Kumar PALUR, Davis, CA (US); Timothy Anderson COULTHER, Davis, CA (US); Amiruddin Bin Johan LECHNER, Davis, CA (US); Pamela Ruth DENISH, Davis, CA (US)

(21) Appl. No.: 19/109,749

(22) PCT Filed: Sep. 7, 2023
(86) PCT No.: PCT/US2023/073656

§ 371 Date: Mar. 7, 2025

Related U.S. Application Data
(60) Provisional application No. 63/405,208, filed on Sep. 9, 2022.
(60) Provisional application No. 63/450,582, filed on Mar. 7, 2023.

(51) Int. Cl. C12P 19/24; C12N 1/20; C12N 9/12; C12N 9/16; C12N 9/90; C12R 1/19

(52) U.S. Cl. CPC C12P 19/24; C12N 1/20; C12N 9/1205; C12N 9/16; C12N 9/90; C12Y 207/01002; C12Y 301/03; C12Y 501/00; C12Y 503/01008; C12R 2001/19

(57) ABSTRACT

The present disclosure relates to microorganisms useful in the biosynthesis of psicose. Also provided are methods of producing the disclosed microorganism and methods of producing psicose.

### CROSS-REFERENCE TO RELATED APPLICATIONS

This application claims priority to U.S. Provisional Patent Application Ser. No. 63/405,208, filed Sep. 9, 2022, and U.S. Provisional Patent Application Ser. No. 63/450,582, filed Mar. 7, 2023, the contents of each of which are incorporated by reference in their entirety, and to each of which priority is claimed.

### SEQUENCE LISTING

The instant application contains a Sequence Listing which has been submitted electronically in XML format and is hereby incorporated by reference in its entirety. Said XML copy, created on Sep. 5, 2023, is named 081906-1401075-250410PC_SL and is 284,563 bytes in size.

### TECHNICAL FIELD

The presently disclosed subject matter relates to compositions and methods for producing a low-calorie sugar in microorganisms.

### BACKGROUND

Current industrial D-psicose (allulose) production is through a 2-step, in vitro enzymatic synthesis starting with the conversion of glucose to fructose via xylose-isomerase (EC 5.3.1.5). This reaction has a ΔG° of −0.1 KJ/mol, making it reversible. Isomerization is followed by the epimerization of fructose to psicose via either D-tagatose-3-epimerase (EC 5.1.3.31) or D-psicose-3-epimerase (EC 5.1.3.30). A major draw back to the in vitro synthesis is that this reaction has a predicted ΔG° of +5 KJ/mol, making it also thermodynamically unfavorable. Since both reactions are reversible, this in vitro system eventually results in a mixture of glucose, fructose, and psicose, which increases the cost of downstream separation and purification processes.

### SUMMARY

In one aspect, the present disclosure relates to a recombinant microorganism comprising an exogenous epimerase and an exogenous phosphatase, wherein the recombinant microorganism produces an increased amount of psicose as compared to a naturally occurring microorganism.

In some embodiments, the epimerase is an allulose-6-phosphate 3-epimerase (AlsE). In some embodiments, the epimerase is an E. coli AlsE. In some embodiments, the epimerase comprises an amino acid sequence at least about 80% identical to the amino acid sequence set forth in SEQ ID NO: 1. In some embodiments, the epimerase comprises the amino acid sequence set forth in SEQ ID NO: 1. In some embodiments, the epimerase consists of the amino acid sequence set forth in SEQ ID NO: 1.

In some embodiments, the phosphatase is a hexitol phosphatase B (HxpB). In some embodiments, the phosphatase is an E. coli HxpB. In some embodiments, the phosphatase comprises an amino acid sequence at least about 80% identical to the amino acid sequence set forth in SEQ ID NO: 3 or SEQ ID NO: 4. In some embodiments, the phosphatase comprises the amino acid sequence set forth in SEQ ID NO: 3 or SEQ ID NO: 4. In some embodiments, the phosphatase consists of the amino acid sequence set forth in SEQ ID NO: 3 or SEQ ID NO: 4.

In some embodiments, the recombinant microorganism further comprises exogenous galactose:H⁺ symporter (GalP) and glucokinase (Glk). In some embodiments, the GalP is an E. coli GalP and the Glk is E. coli Glk. In some embodiments, the GalP comprises an amino acid sequence at least about 80% identical to the amino acid sequence set forth in SEQ ID NO: 38 and the Glk comprises an amino acid sequence at least about 80% identical to the amino acid sequence set forth in SEQ ID NO: 40. In some embodiments, the GalP comprises the amino acid sequence set forth in SEQ ID NO: 38 and the Glk comprises the amino acid sequence set forth in SEQ ID NO: 40.

In some embodiments, the recombinant microorganism further comprises a mutation of a gene encoding for an enzyme of the pentose phosphate pathway compared to a naturally occurring microorganism. In some embodiments, the enzyme of the pentose phosphate pathway is glucose-6-phosphate 1-dehydrogenase (Zwf). In some embodiments, the recombinant microorganism further comprises a mutation of a gene encoding for an enzyme of the glycolysis compared to a naturally occurring microorganism.

In some embodiments, the enzyme of the glycolysis is phosphofructokinase-1 (PfkA), phosphofructokinase-2 (PfkB), or pyruvate kinase (PykF). In some embodiments, the enzyme of the glycolysis is phosphofructokinase-1 (PfkA).

In some embodiments, the recombinant microorganism further comprises a mutation of a gene encoding for an enzyme of allose degradation pathway. In some embodiments, the enzyme of allose degradation pathway is allose-6-phosphate isomerase (RpiB). In some embodiments, the recombinant microorganism further comprises a mutation of a gene encoding for an enzyme of mannose biosynthesis pathway. In some embodiments, the enzyme of mannose biosynthesis pathway is mannose-6-phosphate isomerase (ManA).

In some embodiments, the microorganism further comprises an exogenous nuclease and a sgRNA. In some embodiments, the nuclease is a dCas9. In some embodiments, the sgRNA targets a gene encoding for an enzyme of the glycolysis. In some embodiments, the enzyme of the glycolysis is phosphofructokinase-2 (PfkB). In some embodiments, the exogenous epimerase and the exogenous phosphatase are expressed by a stationary phase promoter. In some embodiments, the exogenous nuclease is expressed by an inducible promoter.

In some embodiments, the microorganism further comprising a mutation of a gene encoding for an enzyme of glycogen biosynthesis selected from the group consisting of phosphoglucomutase (Pgm), UDP-glucose pyrophosphorylase, glycogen synthase, glycogen branching enzyme, and glycogenin. In some embodiments, the enzyme of glycogen biosynthesis is phosphoglucomutase (Pgm).

In another aspect, the present disclosure relates to a microorganism comprising a recombinant polynucleotide encoding an epimerase and a phosphatase, wherein expression of the epimerase and the phosphatase results in an increased production of psicose as compared to a microorganism lacking the recombinant polynucleotide.

In some embodiments, the epimerase is an allulose-6-phosphate 3-epimerase (AlsE). In some embodiments, the epimerase is an E. coli AlsE. In some embodiments, the epimerase comprises an amino acid sequence at least about 80% identical to the amino acid sequence set forth in SEQ ID NO: 1. In some embodiments, the epimerase comprises the amino acid sequence set forth in SEQ ID NO: 1. In some embodiments, the epimerase consists of the amino acid sequence set forth in SEQ ID NO: 1.

In some embodiments, the phosphatase is a hexitol phosphatase B (HxpB). In some embodiments, the phosphatase is an E. coli HxpB. In some embodiments, the phosphatase comprises an amino acid sequence at least about 80% identical to the amino acid sequence set forth in SEQ ID NO: 3 or SEQ ID NO: 4. In some embodiments, the phosphatase comprises the amino acid sequence set forth in SEQ ID NO: 3 or SEQ ID NO: 4. In some embodiments, the phosphatase consists of the amino acid sequence set forth in SEQ ID NO: 3 or SEQ ID NO: 4.

In some embodiments, the microorganism further comprises a mutation of a gene encoding for an enzyme of the pentose phosphate pathway. In some embodiments, the enzyme of the pentose phosphate pathway is glucose-6-phosphate 1-dehydrogenase (Zwf). In some embodiments, the microorganism further comprises a mutation of a gene encoding for an enzyme of the glycolysis. In some embodiments, the enzyme of the glycolysis is phosphofructokinase-1 (PfkA), phosphofructokinase-2 (PfkB), or pyruvate kinase (PykF). In some embodiments, the enzyme of the glycolysis is phosphofructokinase-1 (PfkA).

In some embodiments, the microorganism further comprises a mutation of a gene encoding for an enzyme of allose degradation pathway. In some embodiments, the enzyme of allose degradation pathway is allose-6-phosphate isomerase (RpiB). In some embodiments, the microorganism further comprises a mutation of a gene encoding for an enzyme of mannose biosynthesis pathway. In some embodiments, the enzyme of mannose biosynthesis pathway is mannose-6-phosphate isomerase (ManA).

In some embodiments, the microorganism further comprises an exogenous nuclease and a sgRNA. In some embodiments, the nuclease is a dCas9. In some embodiments, the sgRNA targets a gene encoding for an enzyme of the glycolysis. In some embodiments, the enzyme of the glycolysis is phosphofructokinase-2 (PfkB). In some embodiments, the exogenous epimerase and the exogenous phosphatase are expressed by a stationary phase promoter. In some embodiments, the exogenous nuclease is expressed by an inducible promoter.

In some embodiments, the microorganism further comprising a mutation of a gene encoding for an enzyme of glycogen biosynthesis selected from the group consisting of phosphoglucomutase (Pgm), UDP-glucose pyrophosphorylase, glycogen synthase, glycogen branching enzyme, and glycogenin. In some embodiments, the enzyme of glycogen biosynthesis is phosphoglucomutase (Pgm).

In one aspect, the present disclosure relates to a microorganism comprising a recombinant polynucleotide encoding an epimerase and a phosphatase; a mutation of a gene encoding for an enzyme of the pentose phosphate pathway; a mutation of a gene encoding for an enzyme of the glycolysis; a mutation of a gene encoding for an enzyme of allose degradation pathway; and a mutation of a gene encoding for an enzyme of mannose biosynthesis pathway; and optionally a recombinant polynucleotide encoding GalP, Glk, or both.

In a further aspect, the present disclosure relates to a microorganism comprising a recombinant polynucleotide encoding an allulose-6-phosphate 3-epimerase (AlsE) and a hexitol phosphatase B (HxpB); a mutation of glucose-6-phosphate 1-dehydrogenase (Zwf); a mutation of phosphofructokinase-1 (PfkA); a mutation of allose-6-phosphate isomerase (RpiB); a mutation of mannose-6-phosphate isomerase (ManA); and optionally a recombinant polynucleotide encoding GalP, Glk, or both.

Further, in one aspect, the present disclosure relates to a microorganism comprising: a recombinant polynucleotide encoding an allulose-6-phosphate 3-epimerase (AlsE); a recombinant polynucleotide encoding a hexitol phosphatase B (HxpB); a mutation of glucose-6-phosphate 1-dehydrogenase (Zwf); a mutation of phosphofructokinase-1 (PfkA); a mutation of allose-6-phosphate isomerase (RpiB); a mutation of mannose-6-phosphate isomerase (ManA); and optionally a recombinant polynucleotide encoding GalP, Glk, or both.

In some embodiments, the recombinant polynucleotide is stably integrated into the genome. In some embodiments, the comprising an increased content of intracellular fructose-6-phosphate as compared to a naturally occurring microorganism. In some embodiments, the microorganism is E. coli, Bacillus subtilis or Lactococcus lactis.

In some embodiments, the mutation is a deletion. In some embodiments, the mutation reduces or eliminates expression or activity of the enzyme.

In one aspect, the present disclosure also relates to a method for producing psicose comprising culturing the microorganism disclosed herein under conditions suitable for converting a substrate to psicose. In some embodiments, the substrate comprises glucose. In some embodiments, the psicose has a purity value of at least about 80%, at least about 85%, at least about 90%, at least about 95%, or at least about 99%. In some embodiments, the purity value is 100%. In some embodiments, the purity value is determined by the formula:

$$\% purity = \frac{[ allu lose ]}{[ allulose ] + [ mannose ] + [ glucose ]} \times 100$$

Moreover, the present disclosure related to psicose produced by a method comprising culturing the microorganism disclosed herein. In some embodiments, the psicose has a purity value of at least about 80%, at least about 85%, at least about 90%, at least about 95%, or at least about 99%. In some embodiments, the purity value is 100%. In some embodiments, the purity value is determined by the formula disclosed herein.

Additionally, the present disclosure relates to methods of making a food product comprising psicose, the method comprising culturing the microorganism disclosed herein under conditions suitable for converting a substrate to psicose; purifying the psicose; and combining the psicose with a food product to form a food comprising psicose. In some embodiments, the food product is a chewing-gum, a sugar confectionary, a chocolate, or a savory good. In some embodiments, the food item is a beverage, yogurt, ice cream, a baked good, or a nutritional bar.

### BRIEF DESCRIPTION OF THE DRAWINGS

FIG. 1 illustrates psicose production capability of E. coli. AL3601 is MG1655 with a Z1 fragment (lacI^{q} tetR spec^{R}) and T7 RNA polymerase gene (P_{lacUV5}:T7RNAP) (see Table 1). When grown on M9P media (M9 minimum media with 5 g/L yeast extract) with 10 g/L glucose at 30° C., AL3601 was not observed to produce psicose. However, with the deletion of pfkA, 0.15 g/L psicose was produced.

FIG. 2 illustrates a pathway for the biosynthetic production of psicose. Glucose is imported and phosphorylated to glucose-6-phosphate (G6P) by the phosphotransferase system (PTS) or GalP/Glk. G6P is then isomerized to fructose-6-phosphate (F6P) by Glucose-6-phosphate isomerase. F6P is epimerized to psicose-6-phosphate by D-allulose-6-phosphate 3-epimerase (AlsE), which is then dephosphorylated to free psicose by Hexitol phosphatase B (HxpB). Finally, free psicose can diffuse across the cell membrane into the supernatant. Competing pathways include the pentose phosphate pathway, catalyzed by glucose-6-phosphate dehydrogenase (Zwf), glycolysis, which is catalyzed by phosphofructokinases A and B (PfkA and B), the allose degradation pathway, catalyzed by allose-6-phosphate isomerase (RpiB), and the mannose biosynthesis pathway, catalyzed by mannose-6-phosphate isomerase (ManA).

FIG. 3 shows a comparison of psicose production using phosphatases HxpB and YbiV. E. coli encodes the phosphatases HxpB and YbiV. Two plasmids, pAL1946 and pAL1947 (Table 2), were constructed to overexpress alsE and either hxpB or ybiV respectively under an inducible promoter, P_{T7}. AL3601 with pAL1946 or pAL1947 was cultured in M9P media with 10 g/L glucose at 30° C. and induced with 1 mM IPTG. The cultures containing pAL1946 generated 1.0 g/L of psicose after 24 hr. while the cultures containing pAL1947 produced 0.4 g/L. Cultures induced using IPTG reached a lower culture density than their uninduced counterparts.

FIG. 4 shows psicose production in AL3601 and triple knock out (TKO) strain. The genes pfkA, zwf, and rpiB, were deleted in AL3601, generating AL3729 (Table 1). Plasmid pAL1946, containing P_{T7}:alsE-hxpB, was introduced into AL3601 and AL3729. Cultures were grown in M9P media supplemented with 10 g/L glucose at 30° C. and induced with 25 mM IPTG. The uninduced TKO strains generated 1.5 g/L of psicose after 24 hr, while the induced TKO strains produced 0.6 g/L.

FIG. 5 shows a comparison of promoters P_{LlacO1} and P_{T7} for psicose production. Genes alsE and hxpB were expressed under either P_{T7} or P_{LlacO1}. PAL1946 (Table 2), containing P_{T7}:alsE-hxpB, was introduced into AL3601 (Table 1), while pAL2001, containing P_{LlacO1}:alsE-hxpB, was introduced into strain AL1050 (Table 2). Cultures were grown in M9P media supplemented with 10 g/L glucose at 30° C. and induced with 1 mM IPTG. The cultures using P_{LlacO1} generated 0.5 g/L of psicose after 24 hr, while the cultures using P_{T7} produced 0.5 g/L.

FIG. 6 shows a comparison of promoters P_{LlacO1} and P_{T7} in TKO strain. pAL1946 (Table 2), containing P_{T7}:alsE-hxpB, was introduced into AL3729 (AL3601+TKO, Table 1), and pAL2001 (Table 2) containing P_{LlacO1}:alsE-hxpB was introduced into AL3756 (AL1050+TKO, Table 1). Cultures were grown in M9P media supplemented with 10 g/L glucose at 30° C. and induced with 1 mM IPTG for 24 hours. The uninduced TKO strains using P_{T7} generated 1.8 g/L of psicose, while the induced strains produced 0.6 g/L. The uninduced TKO strains using P_{LlacO1} generated 0.6 g/L of psicose, and the induced strains produced 1.4 g/L.

FIG. 7 shows GC/MS analysis to identify the side product. Gas chromatography mass spectrometry (GC-MS) analysis was used to identify the side product in the psicose production. The analysis identified the side product is mannose. The left-side image above shows the GC elution peak, in green, of the side product compared to a mannose standard, in brown. Both side product and the mannose standard elute at approximately 667.5-668.0 seconds post-injection. The right-side image shows the mass spectrum taken of the side product peak at 667.567 seconds (top), compared to a mass spectrum of mannose (bottom).

FIG. 8 shows identification and elimination of mannose side product. To reduce mannose production, manA was deleted from AL3756, generating the quadruple knockout (QKO) strain AL3990 (Table 1). The manA gene encodes for the enzyme mannose-6-phosphate isomerase (ManA), which catalyzes the reversible isomerization of mannose-6-phosphate and F6P. The production plasmid containing P_{LlacO1}:alsE-hxpB was introduced into the QKO and TKO strains. Cultures were grown in M9P media supplemented with 10 g/L glucose at 30° C. and induced with 1 mM IPTG. After 24 hours, the QKO strains generated 3.5 g/L psicose and 0.7 g/L mannose, while the TKO strains produced 2.3 g/L of psicose and 2.5 g/L of mannose. D indicates gene deletion.

FIG. 9) shows psicose production in TKO and QKO strains. Psicose production was compared in AL3756 (ΔpfkA Δzwf ΔrpiB) and AL3990 (ΔpfkA Δzwf ΔrpiB ΔmanA) strains (Table 1). Each strain was transformed with pAL2001, containing P_{LlacO1}:alsE-hxpB. Cultures were grown in M9P media supplemented with 10 g/L glucose at 30° C. and induced with 1 mM IPTG. After 24 hours, the induced QKO strains generated 3.5 g/L with a yield of 34%. The TKO strains produced 2.3 g/L of psicose, with a yield of 25%.

FIG. 10 shows psicose production in AL3990 with pAL2001 (Table 1 & 2) from 15 g/L glucose. AL3990 (ΔpfkA Δzwf ΔrpiB ΔmanA) (Table 1) was transformed with pAL2001, containing P_{LlacO1}:alsE-hxpB. Cultures were grown in M9P media supplemented with 15 g/L glucose at 30° C. and induced with 1 mM IPTG. After 24 hours, the induced strains generated 2.3 g/L with a yield of 40%.

FIG. 11 shows dynamic regulation of carbon flux using CRISPRi at high density. CRISPRi was used to knock down pfkB which is responsible for converting F6P to fructose-1,6-bisphosphate in glycolysis in the QKO strain. The gRNA was designed to target the promoter region of pfkB. Cells were grown in M9P media supplemented with 10 g/L glucose at 37° C. until reaching an OD₆₀₀ of 1.0. The cells were then centrifuged and resuspended in 3.0 mL of M9P media supplemented with 10 g/L of glucose, 1 mM IPTG and 100 ng/ml aTC. After 24 hours growing at 30° C., the induced strains containing the gRNA sequence for the pfkB promoter produced 1.7 g/L of psicose, with a yield of 46%. FIG. 11 (left) shows produced psicose production. FIG. 11 (middle) shows consumed glucose. FIG. 11 (right) shows variation of optical density.

FIG. 12 shows inducer-free production of psicose using the stationary phase-active promoter P_{gadB}. Plasmids pAL2001 (Table 2), containing P_{LlacO1}:alsE-hxpB, and pAL2247 (Table 2), containing P_{gadB}:alsE-hxpB, were individually introduced into QKO strain AL3990 (Table 1). Cells were grown in M9P media supplemented with 30 g/L glucose at 30° C. and induced with 1 mM IPTG (for P_{LlacO1} strain) for 24 hours. The induced cultures containing pAL2001 generated 6.8 g/L of psicose, with a yield of 57%, while the cultures containing P_{gadB}:alsE-hxpB produced 8.8 g/L, with a yield of 63%. FIG. 12 (right) shows produced psicose. FIG. 12 (middle) shows consumed glucose. FIG. 12 (left) shows variation of optical density.

FIG. 13 shows the effect of sugar symporter GalP on psicose production. Galactose proton symporter GalP can be used to supplement glucose import. Once glucose is transported across the cellular membrane by GalP, it can be phosphorylated by glucokinase Glk and assimilated into central carbon metabolism. Plasmid pAL2274, containing P_{LtetO1}:galP-glk, was co-introduced with either pAL2001 (P_{LlacO1}:alsE-hxpB) or pAL2247 (P_{gadB}:alsE-hxpB) into QKO strain AL3990 (Table 1 & 2). Cultures were grown in MOP media supplemented with 30 g/L glucose at 30° C. and induced with 1 mM IPTG (for P_{LlacO1} strain) for 24 hours. P_{LtetO1} was not induced since the full induction of galP-glk impairs cell growth. Highest titer and yield were achieved by cultures containing pAL2247 and pAL2274, which generated 10.7 g/L. of psicose with a yield of 61%.

FIGS. 14A-14C show strategies for the biosynthesis of D-psicose. FIG. 14A shows the current industrial method for D-psicose production leading to limited yield (˜50%) due to a positive ΔG′°. FIG. 14B shows the proposed biosynthetic pathway of D-psicose. The dephosphorylation step thermodynamically drives production forwards due to a large negative αG′^{m} under cellular reactant concentrations of 1 mM. FIG. 14C shows the proposed pathway for the biosynthetic production of D-psicose in E. coli. Deleted steps are in blue. Overexpressed steps are in red. PTS, the phosphotransferase system; AlsE, D-allulose 6-phosphate 3-epimerase; HxpB, hexitol phosphatase B.

FIGS. 15A-15D show D-psicose production capability in E. coli. Cells were grown in M9P media with 10 g L⁻¹ glucose at 37° C. to OD₆₀₀ ˜0.4, then grown at 30° C. for 24 h. At OD₆₀₀ ˜0.4, 1 mM IPTG was added (FIGS. 15B-15D). FIG. 15A shows D-psicose production in MG1655 and AL3601 (Table 5) with and without the deletion of pfkA and/or alsE. FIG. 15B shows various sugar phosphatases with AlsE were tested for D-psicose in AL3601. FIG. 15C shows the operon of alsE and hxpB was expressed under P_{T7} and PL_{lacO1} in AL3601 and AL1050 (Table 5) respectively. ΔOD₆₀₀ indicates the difference in OD₆₀₀ at 0 h and 24 h. FIG. 15D shows comparison of the effect of gene deletions on the production of D-psicose. Error bars indicate s.d. (n=3 biological replicates).

FIGS. 16A-16D show enhancing D-psicose production capability in E. coli. FIG. 16A shows cells grown in M9P media with various concentrations of glucose to OD₆₀₀ ˜0.4 at 37° C. then grown at 30° C. for 24 h. At OD₆₀₀ ˜0.4. 1 mM IPTG was added for the P_{LlacO1} constructs. The operon of alsE and hxpB was expressed under P_{LlacO1} (pAL2001) and P_{gadB} (pAL2247) in AL3756 and AL3990 respectively (Strain 1 and 2, Table 4). FIG. 16B shows Strain 1 and 2 were grown in M9P media with 40 g L⁻¹ glucose at 37° C. to OD₆₀₀ of ˜0 (no culturing at 37° C.), ˜0.4, or ˜1, then grown at 30° C. for 24 h. When the temperature was shifted to 30° C., 1 mM IPTG was added for the P_{LlacO1} constructs. FIG. 16C shows the operon of galP and glk were expressed under P_{LlacO1} (pAL2264, Table 6). Strain 4 (AL3990 with pAL2264 and pAL2247, Table 4) was grown in M9P media with 40 g L⁻¹ glucose to OD₆₀₀ ˜1, then grown at 30° C. for 24 h. At OD₆₀₀ ˜1, 1 mM IPTG was added to induce P_{LlacO1}:galP-glk. Specific titer (g⁻¹ L⁻¹ OD₆₀₀⁻¹) indicates titer per final OD₆₀₀. FIG. 16D shows comparison of the effect of gene deletions on the production of D-psicose, ptsG, ptsH, and/or pgm were deleted in Strain 4. D-psicose production was done as described in FIG. 16C. Error bars indicate s.d. (n=3 biological replicates).

FIGS. 17A and 17B show dynamic control of glycolysis with CRISPRi. FIG. 17A CRISPRi was used to knock down pfkB. The sgRNA targeting the promoter region of pfkB or without targeting sequence was expressed from a constitutive promoter. dcas9 was expressed from the aTc-inducible Pₜₑₜ. Strain 5, 6, and 7 (Table 4) were grown in M9P media with 40 g L⁻¹ glucose at 37° C. to OD₆₀₀ ˜1, after which 1 mM IPTG and 100 ng/ml aTc were added and cells were grown at 30° C. for 24 h. FIG. 17B shows high cell density D-psicose production. Strain 7 was grown in M9P media with 40 g L⁻¹ glucose at 37° C. until an OD₆₀₀ of ˜1, after which they were induced with 1 mM IPTG and 100 ng mL⁻¹ aTc and grown for another 30 min. Cultures were then spun down and resuspended in M9P media with 40 g L⁻¹, 1 mM IPTG and 100 ng mL⁻¹ aTC to an OD₆₀₀ of ˜8 and grown at 30° C. for 24 h. Error bars indicate s.d. (n=3 biological replicates).

FIGS. 18A-18C show GC-MS identification of D-mannose side product. FIG. 18A shows the GC elution peak: the side product (green) and a mannose standard (brown). FIGS. 18B and 18C show the mass spectrum: the side product peak (FIG. 18B) and a mannose standard (FIG. 18C).

FIG. 19 shows the effect of manA knockout on D-mannose production. To reduce mannose production, manA was knocked out in AL3756 (Table 5), generating AL3990 (Table 5). Production plasmid pAL2001 (Table 6) containing P_{LlacO1}:alsE-hxpB was introduced into AL3756 and AL3990, generating Strain 1 and 2 (Table 4). Cultures were grown in M9P media supplemented with 10 g/L glucose at 30° C. and induced with 1 mM IPTG. After 24 h, Strain 1 generated 2.5 g L⁻¹ of D-mannose, while Strain 2 generated 0.7 g L⁻¹. Error bars indicate s.d. (n=3 biological replicates).

FIGS. 20A-20C show characterization of the stationary phase promoters. Fluorescence and OD₆₀₀ of strains with sfGFP expressed under promoters P_{gadB}, P_{cbpA2}, P_{dps}, and P_{ihfA4} were monitored for timing and activity in comparison to P_{LlacO1} induced with 1 mM IPTG. Error bars indicate s.d. (n=3 biological replicates).

FIGS. 21A and 21B show inhibition of fluorescence by CRISPRi. FIG. 21A shows three sgRNAs were designed for targeting P_{LlacO1}:sfgfp. Figure discloses SEQ ID NO: 58. FIG. 21B shows the dcas9 gene cloned under the aTc-inducible promoter Pₜₑₜ, generating plasmid pAL1952 (Table 6). Constitutively expressed sgRNA A (pAL2066), B (pAL2173), and C (pAL2174) or sgRNA without targeting sequence (pAL2063) were individually cloned onto a plasmid containing P_{LlacO1}:sfgfp (Table 6). AL1050 with the CRISPRi system was cultured at 30° C. and fluorescence was measured at 0 and 4 hr after induction with 1 mM IPTG and with or without 100 ng mL⁻¹ aTc. Error bars indicate s.d. (n=3 biological replicates).

FIGS. 22A and 22B show growth inhibition with CRISPRi. The effect on growth of two different CRISPRi systems was tested in AL4186 (Table 5). In the first CRISPRi system, dcas9 and sgRNA are on a medium copy (p15A ori) and a high copy (ColE ori) plasmid, respectively. In the second CRISPRi system, both dcas9 and sgRNA are on the same plasmid (p15A ori). FIG. 22A shows sgRNA designed to target the middle of the pfkB promoter region, P_{pfkB1} and P_{pfkB2}. Figure discloses SEQ ID NO: 59. FIG. 22B shows cells were grown at 30° C. and OD₆₀₀ was measured at 0 and 24 h. ΔOD₆₀₀ indicates the difference in OD₆₀₀ at 0 h and 24 h. Error bars indicate s.d. (n=3 biological replicates).

FIGS. 23A-23C show glucose consumption and psicose production over time. D-glucose consumption and D-psicose production by Strain 7 (Table 4) was monitored in M9P media containing glucose at 3 g L⁻¹ (FIG. 23A), 5 g L⁻¹ (FIG. 23B), and 10 g L⁻¹ (FIG. 23C) at 30° C. for 10 h. Error bars indicate s.d. (n=3 biological replicates).

FIG. 24 shows high cell density D-psicose production in strain AL4186. Strain AL4186 (MG1655 ΔpfkA Δzwf ΔrpiB ΔmanA Δpgm) was transformed with plasmids pAL2247 (P_{gadB}:alsE-hxpB), pAL2264 (P_{LlacO1}:galP-glk), and pAL2188 (Pₜₑₜ:dcas9 pTargetF-pfkB). Cultures were grown in M9P media with 40 g L⁻¹ glucose at 37° C. until an OD₆₀₀ of ˜1, after which they were induced with 1 mM IPTG and 100 ng mL⁻¹ aTc and grown for another 30 min. Cultures were then spun down and resuspended in M9P media with 40 g L⁻¹ glucose, 1 mM IPTG, and 100 ng mL⁻¹ aTC to an OD₆₀₀ of ˜10 and grown at 30° C. for 8 h, with samples taken at 0, 4, and 8 h. Over the course of 8 hr. cultures produced an average of 15.3 g L⁻¹ of D-psicose with a specific titer of 1.4 g L⁻¹ OD₆₀₀⁻¹, yield of 43%, and productivity of 1.9 g L⁻¹ hr⁻¹. Although D-psicose titers produced at 0-4 hr and 4-8 hr were similar, at 7.3 and 8.0 g L⁻¹, the yield at 4-8 hr (53%) was higher than that at 0-4 h (35%). Error bars indicate s.d. (n=3 biological replicates).

FIG. 25 shows the HPLC chromatogram of the high cell density experiment with Strain 7 (see Table 4) cultured for 24 h. The elution of D-glucose occurs at ˜3.36 min, and the elution of D-psicose occurs at ˜5.45 min. FIGS. 26A and 26B show the biochemical features of phosphates. FIG. 26A shows the AlphaFold predicted structure of HxpB with P6P (199 Å) located in the active site pocket (429 Å), with volumes calculated using MoloVol and CAVER, respectively. FIG. 26B shows ASP173 interacts with the magnesium ion, which in turn positions the phosphate in POP for nucleophilic attack by ASP15. Residues GLU22, TRP25, LEU52, SER117 are predicted to form hydrogen bonds (shown as dotted yellow lines) with the hydroxyl groups and position the P6P for hydrolysis.

### DETAILED DESCRIPTION

The market for rare sugars as a foodstuff, dietary supplement, and health aid is expanding. Among rare sugars, D-psicose has attracted particular attention. However, the current method of producing D-psicose is costly, inefficient, and thermodynamically unfavorable, limiting its potential for wide-spread use. Significantly, the present disclosure addressed various obstacles in D-psicose production including thermodynamic barriers, limited yield, the requirement for purified enzymes and the addition of cofactors. Another significant finding disclosed herein was that E. coli naturally possesses a thermodynamically favorable pathway for D-psicose production allowing the improvement of D-psicose production by increasing the expression of native genes and eliminating competing pathways without the introduction of heterologous genes. The present disclosed subject matter facilitates the industrial-scale production of D-psicose without the need for costly enzyme purification or difficult feedstock and product separation.

The present disclosure is based, in part, on the discovery that microorganisms including specific genetic modifications (e.g., gene deletion) can be made for producing low-calorie sugars. In certain embodiments, the low-calorie sugar is psicose. For clarity and not by way of limitation, the detailed description of the presently disclosed subject matter is divided into the following subsections:
1. Definitions;
2. Microorganisms Producing Psicose;
3. Methods for Producing and Generating Microorganisms;
4 Methods for Producing Psicose; and
5. Food Products.

### 1. Definitions

The terms used in this specification generally have their ordinary meanings in the art, within the context of this invention and in the specific context where each term is used. Certain terms are discussed below, or elsewhere in the specification, to provide additional guidance to the practitioner in describing the methods and compositions of the invention and how to make and use them.

As used herein, the use of the word “a” or “an” when used in conjunction with the term “comprising” in the claims and/or the specification can mean “one,” but it is also consistent with the meaning of “one or more,” “at least one.” and “one or more than one.”

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The present disclosure also contemplates other embodiments “comprising.” “consisting of”, and “consisting essentially of,” the embodiments or elements presented herein, whether explicitly set forth or not.

As used herein, the term “microorganism” refers to any organism that exists as a microscopic cell that is included within the domains of archaea, bacteria, or eukarya, the latter including yeast and filamentous fungi, protozoa, algae, or higher Protista. In certain embodiments, the term includes prokaryotic or eukaryotic cells or organisms having a microscopic size including, but without any limitation, bacteria, archaea, and eubacteria of all species as well eukaryotic microorganisms such as yeast and fungi. In certain embodiments, the term microorganism includes cells that can be cultured for the production of a chemical (e.g., sugar). In certain embodiments, the microorganism is a prokaryotic microorganism. In certain embodiments, the prokaryotic microorganism is a bacterium.

As used herein, the terms “bacterium,” “bacteria,” or “eubacteria,” refers to a domain of prokaryotic organisms. In certain embodiments. bacteria include gram-negative bacteria. gram-positive bacteria, proteobacteria, cyanobacteria, spirochetes, and related species, planctomyces, bacteroides, chlamydia, green sulfur bacteria, green non-sulfur bacteria, radioresistant micrococci, and thermotoga and Thermosipho thermophiles.

As used herein, the term “gram-negative bacteria” includes cocci, nonenteric rods, and enteric rods. The genera of gram-negative bacteria include, for example, and without any limitation, Neisseria, Spirillum, Pasteurella, Brucella, Yersinia, Francisella, Haemophilus, Bordetella, Escherichia, Salmonella, Shigella, Klebsiella, Proteus, Vibrio, Pseudomonas, Bacteroides, Acetobacter, Aerobacter, Agrobacterium, Azotobacter, Spirilla, Serratia, Vibrio, Rhizobium, Chlamydia, Rickettsia, Treponema, and Fusobacterium.

As used herein, the term “gram-positive bacteria” includes cocci, nonsporulating rods, and sporulating rods. The genera of gram-positive bacteria include, for example, and without any limitation, Actinomyces, Bacillus, Clostridium, Corynebacterium, Erysipelothrix, Lactobacillus, Listeria, Mycobacterium, Myxococcus, Nocardia, Staphylococcus, Streptococcus, and Streptomyces.

As used herein, the term “recombinant microorganism” refers to a microorganism that contains one or more recombinant polynucleotides.

The term “exogenous,” as used herein, refers to molecules that are not naturally found in and/or produced by a given yeast, bacterium, organism, microorganism, or cell in nature. The term “endogenous,” as used herein, refers to molecules that are naturally found in and/or produced by a given yeast, bacterium, organism, microorganism, or cell in nature.

The term “nucleic acid molecule,” “nucleotide sequence,” or “polynucleotide,” as used herein, refers to a single or double-stranded covalently-linked sequence of nucleotides in which the 3′ and 5′ ends on each nucleotide are joined by phosphodiester bonds. The nucleic acid molecule can include deoxyribonucleotide bases or ribonucleotide bases and can be manufactured synthetically in vitro or isolated from natural sources.

As used herein, “recombinant polynucleotide” refers to a polynucleotide wherein the exact nucleotide sequence of the polynucleotide is foreign to (i.e., not naturally found in) a given host. In certain embodiments, a recombinant polynucleotide sequence is naturally found in a given host, but in an unnatural (e.g., greater than or less than expected) amount, or additionally if the sequence of a polynucleotide comprises two or more subsequences that are not found in the same relationship to each other in nature. For example, but without any limitation, a recombinant polynucleotide could have two or more sequences from unrelated polynucleotides or from endogenous nucleotides arranged to make a new polynucleotide. In certain embodiments, the present disclosure provides the introduction of a recombinant polynucleotide into a microorganism, wherein the polynucleotide encodes for a polypeptide that is not normally found in the microorganism. With reference to the microorganism's genome, then, the polynucleotide sequence that encodes the polypeptide is recombinant or heterologous.

A “gene,” as used herein, refers to a DNA region (including exons and introns) encoding a gene product, as well as all DNA regions which regulate the production of the gene product. whether or not such regulatory sequences are adjacent to coding and/or transcribed sequences. In certain non-limiting embodiments, a gene includes promoter sequences, terminators, translational regulatory sequences such as ribosome binding sites and internal ribosome entry sites, enhancers, silencers, insulators, boundary elements, replication origins, matrix attachment sites, and locus control regions.

The terms “polypeptide.” “peptide,” “amino acid sequence” and “protein,” used interchangeably herein, refer to a molecule formed from the linking of at least two amino acids. The link between one amino acid residue and the next is an amide bond and is sometimes referred to as a peptide bond. A polypeptide can be obtained by a suitable method known in the art, including isolation from natural sources, expression in a recombinant expression system, chemical synthesis, or enzymatic synthesis. The terms can apply to amino acid polymers in which one or more amino acid residues is an artificial chemical mimetic of a corresponding naturally occurring amino acid, as well as to naturally occurring amino acid polymers and non-naturally occurring amino acid polymers.

The term “amino acid,” as used herein, can be naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, gamma-carboxyglutamate, and O-phosphoserine. Amino acid analogs and derivatives can refer to compounds that have the same basic chemical structure as a naturally occurring amino acid, i.e., a carbon that is bound to a hydrogen, a carboxyl group, an amino group, and an R group, e.g., homoserine, norleucine, methionine sulfoxide, and methionine methyl sulfonium. Such analogs can have modified R groups (e.g., norleucine) or modified peptide backbones, but retain the same basic chemical structure as a naturally occurring amino acid. Amino acid mimetics mean chemical compounds that have a structure that is different from the general chemical structure of amino acid, but that function in a manner similar to a naturally occurring amino acid. Non-limiting examples of amino acids include tryptophan, phenylalanine, histidine, glycine, cysteine, alanine, tyrosine, serine, methionine, asparagine, leucine, asparagine. threonine, isoleucine, proline, glutamic acid, aspartic acid, hydroxyl proline, arginine, cystine, glutamine, lysine, valine, ornithine, taurine, and combinations thereof.

The term “isolated,” as used herein, refers to a material that is removed from at least one component with which it is naturally associated (e.g., removed from its original environment).

As used herein, the terms “reduce” and “reduction” refer to a measurable lessening of an end-point (e.g., enzymatic activity, production of compound, expression of a protein) by at least about 10%, at least about 50%, at least about 75%, or at least about 90%. In certain embodiments, the reduction can be from about 10% to about 100%.

As used herein, the term “increase.” “elevate” and “elevation” refers to a measurable augmentation of an end-point (e.g., enzymatic activity, production of compound, expression of a protein) by at least about 10%, at least about 50%, at least about 75%, or at least about 90%. In certain embodiments, the increase can be from about 10% to about 100%. In certain embodiments, the increase can be at least about 10-fold, about 100-fold, or about 1000-fold or more. In certain embodiments, the increase can be about 100-fold or more, about 1000-fold or more, or about 10,000-fold or more.

Techniques for determining nucleic acid and amino acid sequence identity are known in the art. Typically, such techniques include determining the nucleotide sequence of the mRNA for a gene and/or determining the amino acid sequence encoded thereby and comparing these sequences to a second nucleotide or amino acid sequence. Genomic sequences can also be determined and compared in this fashion. In general, identity refers to an exact nucleotide-to-nucleotide or amino acid-to-amino acid correspondence of two polynucleotides or polypeptide sequences, respectively. Two or more sequences (polynucleotide or amino acid) can be compared by determining their percent identity. The percent identity of two sequences, whether nucleic acid or amino acid sequences, is the number of exact matches between two aligned sequences divided by the length of the shorter sequences and multiplied by 100. Unless indicated otherwise, percent identity is determined for two sequences when compared and aligned for maximum correspondence over a comparison window or designated region as measured using a BLAST or BLAST 2.0 sequence comparison algorithms with default parameters. See. e.g., the NCBI web site at ncbi.nlm.nih.gov/BLAST. For example, BLASTN and BLASTP can be used using the following default parameters: genetic code=standard; filter-none; strand=both: cutoff=60); expect=10); Matrix=BLOSUM62; Descriptions=50 sequences; sort by=HIGH SCORE; Databases=non-redundant. GenBank+EMBL+DDBJ+PDB+GenBank CDS translations+Swiss protein+Spupdate+PIR. Details of these programs can be found on the GenBank website.

A “mutation” in a gene can include, for example, nucleotide changes, deletions of one or more nucleotides (which can include the entire coding sequence and/or promoter or other regulatory sequences) and insertions of one or more nucleotides, which can occur in the coding sequence of the gene or its regulatory components (e.g., the gene's promoter). Mutations can include, for example, mutations that reduce or eliminate function (e.g., nonsense mutations) as well as alterations of the genome that reduce or knockout expression of a gene product.

As used herein, the term “yield” refers to the amount of a product that is recovered from a process or chemical reaction. For example, but without any limitation, the yield refers to the amount of allulose recovered from the culturing of one of the presently disclosed microorganisms. In certain embodiments, yield is expressed as a fraction or a percentage based on the raw materials used or as a ratio of the final product to the starting materials without considering any side reactions. The term “yield coefficient,” as used herein. is a measure of the amount of product produced to the raw materials consumed. In certain embodiments, the yield coefficient refers to the production of allulose to the substrate (e.g., glucose).

### 2. Microorganisms Producing Psicose

The present disclosure provides genetically engineered microorganisms. In certain embodiments, the presently disclosed microorganisms can produce an increased amount of psicose, e.g., an increased amount compared to a control microorganism that is naturally-occurring.

D-Psicose, also known as D-allulose, is a natural but rare monosaccharide. It is a ketohexose that has the same empirical formula as common monosaccharides such as glucose and fructose. It is epimeric with fructose at the 3-position. Its enantiomer, L-psicose, is unknown in nature but has been synthesized. D-psicose has the following formula:

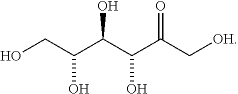 [SMILES: O=C(CO)[C@H](O)[C@H](O)[C@H](O)CO]

D-Psicose is 70% as sweet as sucrose (e.g., table sugar), but it has only about 10% of the nutritional energy (e.g., calorie) value. As a result, D-psicose can be used as a replacement for sucrose and artificial sweeteners.

The present disclosure provides genetically engineered microorganisms that have increased production of psicose as compared to naturally-occurring microorganisms. FIG. 2 illustrates the biochemical pathways modulated in an exemplary microorganism of the present disclosure. In this exemplary microorganism, glucose is imported and phosphorylated to glucose-6-phosphate (G6P) by the phosphotransferase system (PTS) or GalP/Glk. G6P is then isomerized to fructose-6-phosphate (F6P) by Glucose-6-phosphate isomerase. Next, F6P is epimerized to psicose-6-phosphate by D-allulose-6-phosphate 3-epimerase (AlsE), which is then dephosphorylated to free psicose by Hexitol phosphatase B (HxpB). Finally, free psicose can diffuse across the cell membrane into the supernatant. In some embodiments, the presently disclosed microorganisms also include gene editing (e.g., knockout) of certain competing pathways including the pentose phosphate pathway, catalyzed by glucose-6-phosphate dehydrogenase (Zwf); glycolysis, which is catalyzed by phosphofructokinases A and B (PfkA and PfkB); the allose degradation pathway, catalyzed by allose-6-phosphate isomerase (RpiB); and the mannose biosynthesis pathway, catalyzed by mannose-6-phosphate isomerase (ManA)

### 2.1. Psicose Production Enzymes

In certain embodiments, the presently disclosed microorganisms include overexpression of at least one gene encoding an enzyme catalyzing reactions for the production of psicose. In certain embodiments, the presently disclosed microorganisms include a recombinant polynucleotide encoding at least one enzyme catalyzing reactions for the production of psicose. In certain embodiments, the enzyme is an epimerase, for example an epimerase that converts fructose-6-phosphate (F6P) to psicose-6-phosphate. As used herein, the term “epimerase” refers to a class of enzymes that catalyze the inversion of asymmetric groups in a substrate with several centers of asymmetry. Non-limiting examples of epimerase include D-allulose-6-phosphate 3-epimerase, methylmalonyl-CoA epimerase, UDPgalactose 4-epimerase, UDPglucose 4-epimerase, UDPglucuronate 4-epimerase, UDPglucuronate 5′-epimerase, ribose-5-phosphate epimerase, GDPmannose 3,5-epimerase, L-ribulosephosphate 4-epimerase, UDP-N-acetylglucosamine 2-epimerase, UDP-N-acetylglucosamine 4-epimerase, UDPgalactose 4-epimerase, UDPglucose 4-epimerase, UDPglucuronate 4-epimerase, UDPglucuronate 5′-epimerase, GDPmannose 3,5-epimerase, methylmalonyl-CoA epimerase, ribose-5-phosphate epimerase, and UDP-N-acetylglucosamine 2-epimerase.

In certain embodiments, the epimerase is a D-allulose-6-phosphate 3-epimerase (AlsE) (UniProt No. P32719). AlsE catalyzes the reversible epimerization of D-allulose 6-phosphate to D-fructose 6-phosphate. In certain embodiments, AlsE is an E. coli AlsE. In certain embodiments, AlsE comprises an amino acid sequence that is at least about 80%, at least about 85%, at least about 90%, at least about 95%, at least about 96%, at least about 97%, at least about 98%, at least about 99%, or at least about 100% identical to the amino acid sequence set forth in SEQ ID NO: 1. In certain embodiments, AlsE comprises the amino acid sequence set forth in SEQ ID NO: 1. In certain embodiments, AlsE consists of the amino acid sequence set forth in SEQ ID NO: 1. SEQ ID NO: 1 is provided below:

[SEQ ID NO: 1]
MKISPSIMCMDLLKFKEQIEFIDSHADYFHIDIMDGHFVPNLTLSPFFV
SQVKKLATKPLDCHEMVTRPQDYIAQLARAGADFITLHPETINGQAFRL
IDEIRRHDMKVGLILNPETPVEAMKYYIHKADKITVMTVDPGFAGQPFI
PEMLDKLAELKAWREREGLEYEIEVDGSCNQATYEKIMAAGADVFIVGT
SGLFNHAENIDEAWRIMTAQILAAKSEVQPHAKTA

In certain embodiments, the gene alsE is encoded by a nucleotide sequence that is at least about 80%, at least about 85%, at least about 90%, at least about 95%, at least about 96%, at least about 97%, at least about 98%, at least about 99%, or at least about 100% identical to the nucleotide sequence set forth in SEQ ID NO: 2. In certain embodiments, alsE comprises the nucleotide sequence set forth in SEQ ID NO: 2. In certain embodiments, alsE consists of the nucleotide sequence set forth in SEQ ID NO: 2. SEQ ID NO: 2 is provided below:

[SEQ ID NO: 2]
ATGAAAATCTCCCCCTCGTTAATGTGTATGGATCTGCTGAAATTTAAAG
AACAGATCGAATTTATCGACAGCCATGCCGATTACTTCCACATCGATAT
CATGGACGGTCACTTTGTCCCCAATCTGACACTCTCACCGTTCTTCGTA
AGTCAGGTTAAAAAACTGGCAACTAAACCGCTCGACTGTCATCTGATGG
TGACGCGGCCGCAGGATTACATTGCTCAACTGGCGCGTGCGGGAGCAGA
TTTCATCACTCTGCATCCGGAAACCATCAACGGCCAGGCGTTCCGCCTG
ATTGATGAAATCCGCCGTCATGACATGAAAGTGGGGCTGATCCTTAACC
CGGAGACGCCAGTTGAGGCCATGAAATACTATATCCATAAGGCCGATAA
AATTACGGTCATGACTGTCGATCCCGGCTTTGCCGGACAACCGTTCATT
CCTGAAATGCTGGATAAACTTGCCGAACTGAAGGCATGGCGTGAACGAG
AAGGTCTGGAGTACGAAATTGAGGTGGACGGTTCCTGCAACCAGGCAAC
TTACGAAAAACTGATGGCGGCAGGGGCGGATGTCTTTATCGTCGGCACT
TCCGGCCTGTTTAATCATGCGGAAAATATCGACGAAGCATGGAGAATTA
TGACCGCGCAGATTCTGGCTGCAAAAAGCGAGGTACAGCCTCATGCAAA
AACAGCATAA

In certain embodiments, the enzyme is a phosphatase, for example a phosphatase that dephosphorylates psicose-6-phosphate to free psicose. As used herein, the term “phosphatase” refers to a class of enzymes that catalyze the removal of a phosphate group from an organic compound. In certain embodiments, the phosphatase catalyzes the removal of a phosphate group from a sugar. In certain embodiments, the sugar is a hexose.

In certain embodiments, the phosphatase is a Hexitol phosphatase B (HxpB) (UniProt No. P77247 or UniProt No. Q7ADF8). HxpB catalyzes the dephosphorylation of D-psicose 6-phosphate. In certain embodiments, HxpB is an E. coli HxpB. In certain embodiments, HxpB comprises an amino acid sequence that is at least about 80%, at least about 85%, at least about 90%, at least about 95%, at least about 96%, at least about 97%, at least about 98%, at least about 99%, or at least about 100% identical to the amino acid sequence set forth in SEQ ID NO: 3. In certain embodiments, HxpB comprises the amino acid sequence set forth in SEQ ID NO: 3. In certain embodiments, HxpB consists of the amino acid sequence set forth in SEQ ID NO: 3. SEQ ID NO: 3 is provided below:

(SEQ ID NO: 3]
MSTPRQILAAIFDMDGLLIDSEPLWDRAELDVMASLGVDISRRNELPDT
LGLRIDMVVDLWYARQPWNGPSRQEVVERVIARAISLVEETRPLLPGVR
EAVALCKEQGLIVGLASASPLHMLEKVLTMEDLRDSEDALASAEKLPYS
KPHPQVYLDCAAKLGVDPLTCVALEDSVNGMIASKAARMRSIVVPAPEA
QNDPREVLADVKESSLTELTAKDLLG

In certain embodiments, HxpB comprises the amino acid sequence set forth in SEQ ID NO: 4. In certain embodiments, HxpB consists of the amino acid sequence set forth in SEQ ID NO: 4. SEQ ID NO: 4 is provided below:

[SEQ ID NO: 4]
MSTPRQILAAIFDMDGLLIDSEPLWDRAELDVMASLGVDISRRNELPDT
LGLRIDMVVDLWYARQPWNGPSRQEVVERVIARAISLVEETRPLLPGVR
EAVALCKEQGLLVGLASASPLHMLEKVLTMEDLRDSEDALASAEKLPYS
KPHPQVYLDCAAKLGVDPITCVALEDSVNGMIASKAARMRSIVVPAPEA
QNDPRFVLANVKLSSLTELTAKDLLG

In certain embodiments, the gene hxpB is encoded by a nucleotide sequence that is at least about 80%, at least about 85%, at least about 90%, at least about 95%, at least about 96%, at least about 97%, at least about 98%, at least about 99%, or at least about 100% identical to the nucleotide sequence set forth in SEQ ID NO: 5. In certain embodiments, hxpB comprises the nucleotide sequence set forth in SEQ ID NO: 5. In certain embodiments, hxpB consists of the nucleotide sequence set forth in SEQ ID NO: 5. SEQ ID NO: 5 is provided below:

[SEQ ID NO: 5]
ATGTCAACCCCGCGTCAGATTCTTGCTGCAATTTTTGATATGGATGGAT
TACTTATCGACTCAGAACCTTTATGGGATCGAGCCGAACTGGATGTGAT
GGCAAGCCTGGGGGTGGATATCTCCCGTCGTAACGAGCTGCCGGACACC
TTAGGTTTACGCATCGATATGGTGGTCGATCTTTGGTACGCCCGGCAAC
CGTGGAATGGGCCAAGCCGTCAGGAAGTAGTAGAACGGGTTATTGCCCG
TGCCATTTCACTGGTTGAAGAGACACGTCCATTATTACCAGGCGTGCGC
GAAGCCGTTGCGTTATGCAAAGAACAAGGTTTATTGGTGGGACTGGCCT
CCGCGTCACCACTACATATGCTGGAAAAAGTGTTGACCATGTTTGACTT
ACGCGACAGTTTCGATGCCCTCGCCTCGGCCGAAAAACTGCCTTACAGC
AAGCCGCATCCGCAAGTATATCTCGACTGCGCAGCAAAACTGGGCGTTG
ACCCTCTGACCTGCGTAGCGCTGGAAGATTCGGTAAATGGCATGATCGC
CTCTAAAGCAGCCCGCATGCGTTCCATCGTCGTTCCTGCGCCAGAAGCG
CAAAATGATCCACGTTTTGTATTAGCAGACGTCAAACTTTCATCGCTGA
CAGAACTCACCGCAAAAGACCTTCTCGGTTGA

In certain embodiments, the phosphatase is a sugar phosphatase, e.g., YbiV, G6425 (EcoCyc) P75792 (UniProt) and catalyzes the dephosphorylation of D-psicose 6-phosphate. In certain embodiments, YbiV is an E. coli YbiV. In certain embodiments, YbiV comprises an amino acid sequence that is at least about 80%, at least about 85%, at least about 90%, at least about 95%, at least about 96%, at least about 97%, at least about 98%, at least about 99%, or at least about 100% identical to the amino acid sequence set forth in SEQ ID NO: 36. In certain embodiments, YbiV comprises the amino acid sequence set forth in SEQ ID NO: 36. In certain embodiments, YbiV consists of the amino acid sequence set forth in SEQ ID NO: 36. SEQ ID NO: 36 is provided below:

[SEQ ID NO: 36]
MSVKVIVTDMDGTFLNDAKTYNQPRFMAQYQELKKRGIKFVVASGNQ
YYQLISFFPELKDEISFVAENGALVYEHGKQLFHGELTRHESRIVIG
ELLKDKQLNFVACGLQSAYVSENAPEAFVALMAKHYHRLKPVKDYQE
IDDVLFKFSLNLPDEQIPLVIDKLHVALDGIMKPVTSGFGFIDLIIP
GLHKANGISRLLKRWDLSPQNVVAIGDSGNDAEMLKMARYSFAMGNA
AENIKQIARYATDDNNHEGALNVIQAVLDNTSPFNS

In certain embodiments, the gene ybiV is encoded by a nucleotide sequence that is at least about 80%, at least about 85%, at least about 90%, at least about 95%, at least about 96%, at least about 97%, at least about 98%, at least about 99%, or at least about 100% identical to the nucleotide sequence set forth in SEQ ID NO: 37. In certain embodiments, ybiV comprises the nucleotide sequence set forth in SEQ ID NO: 37. In certain embodiments, ybiV consists of the nucleotide sequence set forth in SEQ ID NO: 37. SEQ ID NO: 37 is provided below:

[SEQ ID NO: 37]
atgAGCGTAAAAGTTATCGTCACAGACATGGACGGTACTTTTCTTAA
CGACGCCAAAACGTACAACCAACCACGTTTTATGGCGCAATATCAGG
AACTGAAAAAGCGCGGCATTAAGTTCGTTGTTGCCAGCGGTAATCAG
TATTACCAGCTTATTTCATTCTTTCCTGAGCTAAAGGATGAGATCTC
TTTTGTCGCGGAAAACGGCGCACTGGTTTACGAACATGGCAAGCAGT
TGTTCCACGGCGAACTGACCCGACATGAATCGCGGATTGTTATTGGC
GAGTTGCTAAAAGATAAGCAACTCAATTTTGTCGCCTGCGGTCTGCA
AAGTGCATATGTCAGCGAAAATGCCCCCGAAGCATTTGTCGCACTGA
TGGCAAAACACTACCATCGCCTGAAACCTGTAAAAGATTATCAGGAG
ATTGACGACGTACTGTTCAAGTTTTCGCTCAACCTGCCGGATGAACA
AATCCCGTTAGTGATCGACAAACTGCACGTAGCGCTCGATGGCATTA
TGAAACCCGTTACCAGTGGTTTTGGCTTTATCGACCTGATTATTCCC
GGTCTACATAAAGCAAACGGTATTTCGCGGTTACTGAAACGCTGGGA
TCTGTCACCGCAAAATGTGGTAGCGATTGGCGACAGCGGTAACGATG
CGGAGATGCTGAAAATGGCGCGTTATTCCTTTGCGATGGGCAATGCT
GCGGAAAACATTAAACAAATCGCCCGTTACGCTACCGATGATAATAA
TCATGAAGGCGCGCTGAATGTGATTCAGGCGGTGCTGGATAACACAT
CCCCTTTTAACAGCtga

In certain embodiments, one or more transporter that transports glucose into the cell can be expressed (i.e., overexpressed) in the cell, thereby increasing glucose in the cell. In some embodiments, one or both of galactose:H⁺ symporter (GalP) and glucokinase (Glk) are expressed in the microorganism. For example, in some embodiments, GalP transports glucose into the cell where it is phosphorylated to glucose-6-phosphate by Glk and assimilated into central carbon metabolism.

In certain embodiments, the galactose:H⁺ symporter (GalP) is as described in EG12148 (EcoCyc) or POAEP1 (UniProt). In certain embodiments, GalP is an E. coli GalP. In certain embodiments, GalP comprises an amino acid sequence that is at least about 80%, at least about 85%, at least about 90%, at least about 95%, at least about 96%, at least about 97%, at least about 98%, at least about 99%, or at least about 100% identical to the amino acid sequence set forth in SEQ ID NO: 38. In certain embodiments, GalP comprises the amino acid sequence set forth in SEQ ID NO: 38. In certain embodiments, GalP consists of the amino acid sequence set forth in SEQ ID NO: 38. SEQ ID NO: 38 is provided below:

(SEQ ID NO: 38)
MPDAKKQGRSNKAMTFFVCFLAALAGLLFGLDIGVIAGALPFIADEF
QITSHTQEWVVSSMMFGAAVGAVGSGWLSFKLGRKKSLMIGAILFVA
GSLFSAAAPNVEVLILSRVLLGLAVGVASYTAPLYLSEIAPEKIRGS
MISMYQLMITIGILGAYLSDTAFSYTGAWRWMLGVIIIPAILLLIGV
FFLPDSPRWFAAKRRFVDAERVLLRLRDTSAEAKRELDEIRESLQVK
QSGWALFKENSNFRRAVFLGVLLQVMQQFTGMNVIMYYAPKIFELAG
YTNTTEQMWGTVIVGLTNVLATFIAIGLVDRWGRKPTLTLGFLVMAA
GMGVLGTMMHIGIHSPSAQYFAIAMLLMFIVGFAMSAGPLIWVLCSE
IQPLKGRDFGITCSTATNWIANMIVGATFLTMLNTLGNANTFWVYAA
LNVLFILLTLWLVPETKHVSLEHIERNLMKGRKLREIGAHD

In certain embodiments, the gene galP is encoded by a nucleotide sequence that is at least about 80%, at least about 85%, at least about 90%, at least about 95%, at least about 96%, at least about 97%, at least about 98%, at least about 99%, or at least about 100% identical to the nucleotide sequence set forth in SEQ ID NO: 39. In certain embodiments, galP comprises the nucleotide sequence set forth in SEQ ID NO: 39. In certain embodiments, galP consists of the nucleotide sequence set forth in SEQ ID NO: 39. SEQ ID NO: 39 is provided below:

(SEQ ID NO: 39)
atgCCTGACGCTAAAAAACAGGGGCGGTCAAACAAGGCAATGACGTT
TTTCGTCTGCTTCCTTGCCGCTCTGGCGGGATTACTCTTTGGCCTGG
ATATCGGTGTAATTGCTGGCGCACTGCCGTTTATTGCAGATGAATTC
CAGATTACTTCGCACACGCAAGAATGGGTCGTAAGCTCCATGATGTT
CGGTGCGGCAGTCGGTGCGGTGGGCAGCGGCTGGCTCTCCTTTAAAC
TCGGGCGCAAAAAGAGCCTGATGATCGGCGCAATTTTGTTTGTTGCC
GGTTCGCTGTTCTCTGCGGCTGCGCCAAACGTTGAAGTACTGATTCT
TTCCCGCGTTCTACTGGGGCTGGCGGTGGGTGTGGCCTCTTATACCG
CACCGCTGTACCTCTCTGAAATTGCGCCGGAAAAAATTCGTGGCAGT
ATGATCTCGATGTATCAGTTGATGATCACTATCGGGATCCTCGGTGC
TTATCTTTCTGATACCGCCTTCAGCTACACCGGTGCATGGCGCTGGA
TGCTGGGTGTGATTATCATCCCGGCAATTTTGCTGCTGATTGGTGTC
TTCTTCCTGCCAGACAGCCCACGTTGGTTTGCCGCCAAACGCCGTTT
TGTTGATGCCGAACGCGTGCTGCTACGCCTGCGTGACACCAGCGCGG
AAGCGAAACGCGAACTGGATGAAATCCGTGAAAGTTTGCAGGTTAAA
CAGAGTGGCTGGGCGCTGTTTAAAGAGAACAGCAACTTCCGCCGCGC
GGTGTTCCTTGGCGTACTGTTGCAGGTAATGCAGCAATTCACCGGGA
TGAACGTCATCATGTATTACGCGCCGAAAATCTTCGAACTGGGGGGT
TATACCAACACTACCGAGCAAATGTGGGGGACCGTGATTGTCGGCCT
GACCAACGTACTTGCCACCTTTATCGCAATCGGCCTTGTTGACCGCT
GGGGACGTAAACCAACGCTAACGCTGGGCTTCCTGGTGATGGCTGCT
GGCATGGGCGTACTCGGTACAATGATGCATATCGGTATTCACTCTCC
GTCGGCGCAGTATTTCGCCATCGCCATGCTGCTGATGTTTATTGTCG
GTTTTGCCATGAGTGCCGGTCCGCTGATTTGGGTACTGTGCTCCGAA
ATTCAGCCGCTGAAAGGCCGCGATTTTGGCATCACCTGCTCCACTGC
CACCAACTGGATTGCCAACATGATCGTTGGCGCAACGTTCCTGACCA
TGCTCAACACGCTGGGTAACGCCAACACCTTCTGGGTGTATGCGGCT
CTGAACGTACTGTTTATCCTGCTGACATTGTGGCTGGTACCGGAAAC
CAAACACGTTTCGCTGGAACATATTGAACGTAATCTGATGAAAGGTC
GTAAACTGCGCGAAATAGGCGCTCACGATtaa

In certain embodiments, the glucokinase (Glk) is as described in EG12957 (EcoCyc) or P0A6V8 (UniProt). In certain embodiments, Glk is an E. coli Glk. In certain embodiments, Glk comprises an amino acid sequence that is at least about 80%, at least about 85%, at least about 90%, at least about 95%, at least about 96%, at least about 97%, at least about 98%, at least about 99%, or at least about 100% identical to the amino acid sequence set forth in SEQ ID NO: 40. In certain embodiments, Glk comprises the amino acid sequence set forth in SEQ ID NO: 40. In certain embodiments, Glk consists of the amino acid sequence set forth in SEQ ID NO: 40. SEQ ID NO: 40 is provided below:

(SEQ ID NO: 40)
MTKYALVGDVGGTNARLALCDIASGEISQAKTYSGLDYPSLEAVIRV
YLEEHKVEVKDGCIAIACPITGDWVAMTNHTWAFSIAEMKKNLGFSH
LEIINDFTAVSMAIPMLKKEHLIQFGGAEPVEGKPIAVYGAGTGLGV
AHLVHVDKRWVSLPGEGGHVDFAPNSEEEAIILEILRAEIGHVSAER
VLSGPGLVNLYRAIVKADNRLPENLKPKDITERALADSCTDCRRALS
LFCVIMGRFGGNLALNLGTFGGVFIAGGIVPRFLEFFKASGFRAAFE
DKGRFKEYVHDIPVYLIVHDNPGLLGSGAHLRQTLGHIL

In certain embodiments, the gene glk is encoded by a nucleotide sequence that is at least about 80%, at least about 85%, at least about 90%, at least about 95%, at least about 96%, at least about 97%, at least about 98%, at least about 99%, or at least about 100% identical to the nucleotide sequence set forth in SEQ ID NO: 41. In certain embodiments, glk comprises the nucleotide sequence set forth in SEQ ID NO: 41. In certain embodiments, glk consists of the nucleotide sequence set forth in SEQ ID NO: 41. SEQ ID NO: 41 is provided below:

(SEQ ID NO: 41)
atgACAAAGTATGCATTAGTCGGTGATGTGGGCGGCACCAACGCACG
TCTTGCTCTGTGTGATATTGCCAGTGGTGAAATCTCGCAGGCTAAGA
CCTATTCAGGGCTTGATTACCCCAGCCTCGAAGCGGTCATTCGCGTT
TATCTTGAAGAACATAAGGTCGAGGTGAAAGACGGCTGTATTGCCAT
CGCTTGCCCAATTACCGGTGACTGGGTGGCGATGACCAACCATACCT
GGGCGTTCTCAATTGCCGAAATGAAAAAGAATCTCGGTTTTAGCCAT
CTGGAAATTATTAACGATTTTACCGCTGTATCGATGGCGATCCCGAT
GCTGAAAAAAGAGCATCTGATTCAGTTTGGTGGCGCAGAACCGGTCG
AAGGTAAGCCTATTGCGGTTTACGGTGCCGGAACGGGGCTTGGGGTT
GCGCATCTGGTCCATGTCGATAAGCGTTGGGTAAGCTTGCCAGGCGA
AGGCGGTCACGTTGATTTTGCGCCGAATAGTGAAGAAGAGGCCATTA
TCCTCGAAATATTGCGTGCGGAAATTGGTCATGTTTCGGCGGAGCGC
GTGCTTTCTGGCCCTGGGCTGGTGAATTTGTATCGCGCAATTGTGAA
AGCTGACAACCGCCTGCCAGAAAATCTCAAGCCAAAAGATATTACCG
AACGCGCGCTGGCTGACAGCTGCACCGATTGCCGCCGCGCATTGTCG
CTGTTTTGCGTCATTATGGGCCGTTTTGGCGGCAATCTGGCGCTCAA
TCTCGGGACATTTGGCGGCGTGTTTATTGCGGGCGGTATCGTGCCGC
GCTTCCTTGAGTTCTTCAAAGCCTCCGGTTTCCGTGCCGCATTTGAA
GATAAAGGGCGCTTTAAAGAATATGTCCATGATATTCCGGTGTATCT
CATCGTCCATGACAATCCGGGCCTTCTCGGTTCCGGTGCACATTTAC
GCCAGACCTTAGGTCACATTCTGtaa

Without being bound by any theory, the inventors of the present disclosure believe that any enzyme performing similar function to the enzyme described above can be used in the presently disclosed microorganisms. For example, but without any limitation, the presently disclosed microorganism can include any enzyme that catalyzes the reversible epimerization of D-fructose 6-phosphate to D-psicose 6-phosphate. In another non-limiting example, the presently disclosed microorganism can include any enzyme that dephosphorylates psicose-6-phosphate to free psicose.

### 2.2. Competing Pathways

In certain embodiments, the presently disclosed microorganisms include a mutation of one or more gene encoding one or more enzymes regulating biochemical pathways that can reduce the production of psicose. In certain embodiments, the presently disclosed microorganisms include a reduced expression of a gene encoding enzymes regulating biochemical pathways that can reduce the production of psicose. Physiologically, cells catalyze sugars through the pentose phosphate pathway and glycolysis to produce energy (e.g., ATP). The inventors of the present disclosure discovered that deletion or reduced expression of gene encoding enzymes of certain metabolic pathways results in increased psicose production.

In certain embodiments, the presently disclosed microorganisms include a mutation of a gene encoding an enzyme of the pentose phosphate pathway. In certain embodiments, the presently disclosed microorganisms include a reduced expression of a gene encoding an enzyme of the pentose phosphate pathway. In certain embodiments, the enzyme of the pentose phosphate pathway is selected from the group consisting of glucose-6-phosphate dehydrogenase, 6-phosphogluconolactonase, phosphogluconate dehydrogenase, phosphopentose isomerase, phosphopentose epimerase, transketolase, and transaldolase. In certain embodiments, the enzyme of the pentose phosphate pathway is glucose-6-phosphate dehydrogenase (Zwf) (Entrez Gene ID: 946370). Zwf catalyzes the oxidation of glucose 6-phosphate to 6-phosphogluconolactone. In certain embodiments, Zwf is an E. coli Zwf. A representative nucleotide sequence of the gene zwf is set forth in SEQ ID NO: 6 or at least 90%, 95%, 95%, or 99% identical to SEQ ID NO: 6. SEQ ID NO: 6 is provided below:

[SEQ ID NO: 6]
ATGGCGGTAACGCAAACAGCCCAGGCCTGTGACCTGGTCATTTTCGG
CGCGAAAGGCGACCTTGCGCGTCGTAAATTGCTGCCTTCCCTGTATC
AACTGGAAAAAGCCGGTCAGCTCAACCCGGACACCCGGATTATCGGC
GTAGGGCGTGCTGACTGGGATAAAGCGGCATATACCAAAGTTGTCCG
CGAGGCGCTCGAAACTTTCATGAAAGAAACCATTGATGAAGGTTTAT
GGGACACCCTGAGTGCACGTCTGGATTTTTGTAATCTCGATGTCAAT
GACACTGCTGCATTCAGCCGTCTCGGCGCGATGCTGGATCAAAAAAA
TCGTATCACCATTAACTACTTTGCCATGCCGCCCAGCACTTTTGGCG
CAATTTGCAAAGGGCTTGGCGAGGCAAAACTGAATGCTAAACCGGCA
CGCGTAGTCATGGAGAAACCGCTGGGGACGTCGCTGGCGACCTCGCA
GGAAATCAATGATCAGGTTGGCGAATACTTCGAGGAGTGCCAGGTTT
ACCGTATCGACCACTATCTTGGTAAAGAAACGGTGCTGAACCTGTTG
GCGCTGCGTTTTGCTAACTCCCTGTTTGTGAATAACTGGGACAATCG
CACCATTGATCATGTTGAGATTACCGTGGCAGAAGAAGTGGGGATCG
AAGGGCGCTGGGGCTATTTTGATAAAGCCGGTCAGATGCGCGACATG
ATCCAGAACCACCTGCTGCAAATTCTTTGCATGATTGCGATGTCTCC
GCCGTCTGACCTGAGCGCAGACAGCATCCGCGATGAAAAAGTGAAAG
TACTGAAGTCTCTGCGCCGCATCGACCGCTCCAACGTACGCGAAAAA
ACCGTACGCGGGCAATATACTGCGGGCTTCGCCCAGGGCAAAAAAGT
GCCGGGATATCTGGAAGAAGAGGGCGCGAACAAGAGCAGCAATACAG
AAACTTTCGTGGCGATCCGCGTCGACATTGATAACTGGCGCTGGGCC
GGTGTGCCATTCTACCTGCGTACTGGTAAACGTCTGCCGACCAAATG
TTCTGAAGTCGTGGTCTATTTCAAAACACCTGAACTGAATCTGTTTA
AAGAATCGTGGCAGGATCTGCCGCAGAATAAACTGACTATCCGTCTG
CAACCTGATGAAGGCGTGGATATCCAGGTACTGAATAAAGTTCCTGG
CCTTGACCACAAACATAACCTGCAAATCACCAAGCTGGATCTGAGCT
ATTCAGAAACCTTTAATCAGACGCATCTGGCGGATGCCTATGAACGT
TTGCTGCTGGAAACCATGCGTGGTATTCAGGCACTGTTTGTACGTCG
CGACGAAGTGGAAGAAGCCTGGAAATGGGTAGACTCCATTACTGAGG
CGTGGGCGATGGACAATGATGCGCCGAAACCGTATCAGGCCGGAACC
TGGGGACCCGTTGCCTCGGTGGCGATGATTACCCGTGATGGTCGTTC
CTGGAATGAGTTTGAGTAA

In certain embodiments, E. coli Zwf comprises an amino acid sequence that is at least about 80%, at least about 85%, at least about 90%, at least about 95%, at least about 96%, at least about 97%, at least about 98%, at least about 99%, or at least about 100% identical to the amino acid sequence set forth in SEQ ID NO: 20. In certain embodiments, E. coli Zwf comprises the amino acid sequence set forth in SEQ ID NO: 20. SEQ ID NO: 20 is provided below:

[SEQ ID NO: 20]
MAVTQTAQACDLVIFGAKGDLARRKLLPSLYQLEKAGQLNPDTRIIG
VGRADWDKAAYTKVVREALETFMKETIDEGLWDTLSARLDFCNLDVN
DTAAFSRLGAMLDQKNRITINYFAMPPSTFGAICKGLGEAKLNAKPA
RVVMEKPLGTSLATSQEINDQVGEYFEECQVYRIDHYLGKETVLNLL
ALRFANSLFVNNWDNRTIDHVEITVAEEVGIEGRWGYFDKAGQMRDM
IQNHLLQILCMIAMSPPSDLSADSIRDEKVKVLKSLRRIDRSNVREK
TVRGQYTAGFAQGKKVPGYLEEEGANKSSNTETFVAIRVDIDNWRWA
GVPFYLRTGKRLPTKCSEVVVYFKTPELNLFKESWQDLPQNKLTIRL
QPDEGVDIQVLNKVPGLDHKHNLQITKLDLSYSETFNQTHLADAYER
LLLETMRGIQALFVRRDEVEEAWKWVDSITEAWAMDNDAPKPYQAGT
WGPVASVAMITRDGRSWNEFE

In certain embodiments, Zwf is a Bacillus subtilis Zwf. A representative amino acid sequence of Bacillus subtilis Zwf is found as P54547 (Uniprot)/BSU23850 (KEGG) or as set forth in SEQ ID NO: 10 or at least 90%, 95%, 95%, or 99% identical to SEQ ID NO: 10. SEQ ID NO: 10 is provided below:

(SEQ ID NO: 10)
MKTNQQPKAVIVIFGATGDLAKRKLYPSIHRLYQNGQIGEEFAVVGV
GRRPWSNEDLRQTVKTSISSSADKHIDDFTSHFYYHPFDVTNPGSYQ
ELNVLLNQLEDTYQIPNNRMFYLAMAPEFFGTIAKTLKSEGVTATTG
WSRLVIEKPFGHDLPSAQALNKEIREAFTEDQIYRIDHYLGKQMVQN
IEVIRFANAIFEPLWTNRYISNIQITSSESLGVEDRARYYEKSGALR
DMVQNHIMQMVALLAMEPPIKLNTEEIRSEKVKVLRALRPIAKDEVD
EYFVRGQYHAGEIDGVPVPAYTDEDNVAPDSNTETFVAGKLLIDNFR
WAGVPFYIRTGKRMKEKSTKIVVQFKDIPMNLYYGNENNMNPNLLVI
HIQPDEGITLYLNAKKLGGAAHAQPIKLDYCSNCNDELNTPEAYEKL
IHDCLLGDATNFAHWDEVALSWSFVDSISETWAANKTLSPNYESGSM
GPKESDDLLVKDGLHWWNI

A representative nucleotide sequence of Bacillus subtilis zwf gene is set forth in SEQ ID NO: 26 or at least 90%, 95%, 95%, or 99% identical to SEQ ID NO: 26. SEQ ID NO: 26 is provided below:

(SEQ ID NO: 26)
gtgaaaacaaaccaacaaccaaaagcagtaattgtcatattcggtgc
aactggagatttagcaaaacgaaaattgtatccgtctattcaccgtt
tatatcaaaacggacaaatcggagaagagtttgcagtggtaggagtt
ggaagaagaccttggtctaatgaggatcttcgccaaactgttaaaac
atccatttcctcatctgcagataagcatatagatgatttcacgtctc
atttttactatcacccgtttgacgtgacaaaccctggttcttatcaa
gagctaaacgtattgcttaaccagctggaagatacatatcaaattcc
taacaacagaatgttctacttggcaatggctcctgaattcttcggaa
cgattgcaaaaacattaaaatcagagggtgtaacagctacaaccggc
tggtcccgccttgtcatcgaaaaaccgttcggccatgatctgccaag
cgcacaggcattgaataaagaaatccgcgaagcatttacggaagatc
aaatttacagaatcgaccattatctaggcaaacaaatggttcagaac
attgaagtgattcgatttgccaatgcgattttcgaaccgctttggac
aaaccgctacatttcaaacattcaaatcacatctagcgaatcactag
gcgttgaagaccgcgcaagatattacgaaaaatcaggcgcccttcgc
gacatggtgcaaaaccatattatgcagatggttgcccttcttgcaat
ggagccgcctatcaaattgaacacagaagaaatccgcagcgagaaag
tgaaggtgctgagagcactgcgtcctattgcaaaagacgaagtggat
gaatactttgtgcgcggacaatatcatgctggtgaaattgacggtgt
accggttcctgcttatacagatgaagataatgtcgctcctgactcca
atacagaaacctttgttgccggcaagctcttgatcgacaacttcaga
tgggctggtgttccattctacatcagaaccggaaaacgaatgaaaga
aaagtccacaaaaattgtcgttcaatttaaggacattccgatgaacc
tgtactacggtaatgaaaacaacatgaatccgaacttgcttgtcatt
catattcagcctgacgaaggcattacgctttacttaaatgctaaaaa
gcttggcggagcagcacacgcacagccaatcaaactcgattattgca
gcaattgcaatgacgagttgaacacccctgaagcatatgaaaaacta
attcacgactgtcttcttggcgatgcaacaaactttgcacactggga
tgaagttgccctttcttggagctttgtcgactctatttctgaaacat
gggcagcaaacaaaaccttatctcctaactacgaatcaggctcaatg
ggaccgaaagaatctgatgatcttttggtgaaagacggcttacactg
gtggaacatataa

In certain embodiments, Zwf is a Lactococcus lactis Zwf. A representative amino acid sequence of Lactococcus lactis Zwf is found as LLA12_RS12225: glucose-6-phosphate dehydrogenase, EC1.1.1.49, or as set forth in SEQ ID NO: 11 or at least 90%, 95%, 95%, or 99% identical to SEQ ID NO: 11. SEQ ID NO: 11 is provided below:

(SEQ ID NO: 11)
MTEQKQALFTIFGATGDLAKRKLYPSLFRLFKKGELADNFAVIGTAR
RPWTNEYYREVVLESIKDLMNSKTEAENFASHFYYQSHDVSDSSHYV
NLKDLGEKLRKQYKTAGNQVFFLAMAPQFFGTIAEHLKSENILTGEG
FERIVIEKPFGTSYDTAKSLNDSLAKVFSEEQIFRIDHYLGKEMIQA
VSAVRFANPIFESLWNNQHIDNVQITFAEFIGVEDRGGYYETSGALK
DMIQNHVLQVLSLIAMEKPEKFDESYIVKEKVKALNAIRQYSSEEAL
ENFVRGQYIAGRFDGEDYLGYREEDSVATDSRTETFAAGKFVIDNER
WSGVPFYVRSGKRMTEKGTRINIVFKKDKDNLFAENCDDQSVQNVLT
IYIQPTEGFSLSVNGKAAGQGFHLEPLRLNFRHDSEFLGNSPEAYEK
LFLDVLNGDGTNFSHWEEAARAWELIDVIREAWDKETSELPTYAART
MGPKAAFDLLEKNGHEWAWQPDLWYQERGYYNK

A representative nucleotide sequence of a Lactococcus lactis zwf gene is set forth in SEQ ID NO: 33 or at least 90%, 95%, 95%, or 99% identical to SEQ ID NO: 33. SEQ ID NO: 33 is provided below:

(SEQ ID NO: 33)
atgACCGAACAAAAACAAGCACTTTTCACCATCTTCGGAGCAACTGG
TGACCTCGCTAAAAGAAAGCTCTACCCTTCACTCTTTCGCCTTTTCA
AAAAAGGTGAGCTAGCTGACAATTTTGCAGTCATTGGTACCGCTCGC
CGTCCATGGACAAACGAATATTATCGTGAAGTTGTTTTAGAGTCTAT
CAAAGATTTAATGAACTCAAAAACAGAAGCCGAAAATTTCGCCAGTC
ATTTTTATTATCAAAGTCACGATGTTAGCGACAGCTCACATTACGTT
AACTTAAAAGATTTGGGTGAAAAATTGCGTAAACAGTATAAAACTGC
TGGCAATCAAGTCTTCTTTTTAGCAATGGCTCCTCAATTTTTTGGCA
CTATCGCTGAACACCTTAAATCAGAAAATATTTTGACAGGTGAGGGT
TTTGAGAGAATCGTCATTGAAAAACCATTTGGAACAAGCTACGATAC
GGCAAAATCACTTAATGACAGCCTCGCAAAAGTATTTAGTGAAGAAC
AAATTTTCCGAATCGACCACTACCTTGGAAAAGAAATGATCCAGGCC
GTTTCTGCCGTTCGTTTTGCCAATCCAATCTTTGAATCGCTTTGGAA
TAATCAACACATTGATAACGTTCAGATTACTTTTGCCGAATTTATCG
GTGTTGAAGACCGTGGCGGTTACTACGAAACTTCTGGTGCTTTAAAA
GATATGATTCAAAACCATGTCTTACAAGTACTCAGCCTCATTGCCAT
GGAAAAACCTGAAAAATTTGATGAATCTTATATTGTAAAAGAAAAAG
TTAAAGCCCTTAATGCAATCCGTCAATATTCTTCTGAGGAAGCTTTA
GAAAACTTCGTTCGTGGTCAATATATCGCAGGACGTTTTGACGGTGA
AGATTACCTAGGCTATCGCGAAGAAGATTCTGTTGCAACAGACAGTC
GAACTGAAACTTTCGCTGCTGGAAAATTTGTCATTGATAATGAGCGT
TGGTCAGGCGTCCCTTTCTATGTTCGCTCAGGAAAACGTATGACTGA
AAAAGGAACTCGTATTAATATCGTTTTCAAAAAAGATAAAGACAATC
TCTTTGCAGAAAATTGCGATGACCAATCCGTTCAAAATGTTTTGACC
ATTTATATTCAACCAACTGAAGGGTTTTCACTTTCAGTAAATGGAAA
AGCGGCTGGTCAAGGCTTCCATTTAGAGCCTTTGCGGTTAAATTTCC
GACACGATAGTGAATTTCTCGGAAATTCTCCTGAAGCATATGAAAAA
CTTTTCCTAGATGTCCTTAATGGAGATGGAACAAATTTCTCACATTG
GGAAGAAGCGGCTCGCGCCTGGGAACTTATTGATGTTATTCGAGAAG
CTTGGGATAAAGAAACTTCTGAGCTTCCAACTTATGCGGCTCGCACA
ATGGGACCTAAGGCTGCATTTGACTTACTAGAAAAAAATGGTCACGA
ATGGGCTTGGCAACCTGATTTATGGTATCAAGAACGTGGTTACTACA
ATAAAtaa

In certain embodiments, the presently disclosed microorganisms include a mutation of a gene encoding an enzyme of the glycogen biosynthesis. In certain embodiments, the presently disclosed microorganisms include a reduced expression of a gene encoding an enzyme of the glycogen biosynthesis. In certain embodiments, the enzyme of the glycogen biosynthesis is selected from the group consisting of phosphoglucomutase (Pgm), UDP-glucose pyrophosphorylase, glycogen synthase, glycogen branching enzyme, and glycogenin. In certain embodiments, the enzyme of the glycogen biosynthesis is phosphoglucomutase (Pgm) (Entrez Gene ID: 946370). Pgm (EC 5.4.2.2) is an enzyme that transfers a phosphate group on an α-D-glucose monomer from the 1 to the 6 position in the forward direction or the 6 to the 1 position in the reverse direction. In certain embodiments, Pgm is an E. coli Pgm. A representative nucleotide sequence of gene pgm is set forth in SEQ ID NO: 42 or at least 90%, 95%, 95%, or 99% identical to SEQ ID NO: 47. SEQ ID NO: 47 is provided below:

[SEQ ID NO: 47]
ATGGCAATCCACAATCGTGCAGGCCAACCTGCACAACAGAGTGATTT
GATTAACGTCGCCCAACTGACGGCGCAATATTATGTACTGAAACCAG
AAGCAGGGAATGCGGAGCACGCGGTGAAATTCGGTACTTCCGGTCAC
CGTGGCAGTGCAGCGCGCCACAGCTTTAACGAGCCGCACATTCTGGC
GATCGCTCAGGCAATTGCTGAAGAACGTGCGAAAAACGGCATCACTG
GCCCTTGCTATGTGGGTAAAGATACTCACGCCCTGTCCGAACCTGCA
TTCATTTCCGTTCTGGAAGTGCTGGCAGCGAACGGCGTTGATGTCAT
TGTGCAGGAAAACAATGGCTTCACCCCGACGCCTGCCGTTTCCAATG
CCATCCTGGTTCACAATAAAAAAGGTGGCCCGCTGGCAGACGGTATC
GTGATTACACCGTCCCATAACCCGCCGGAAGATGGTGGAATCAAATA
CAATCCGCCAAATGGTGGCCCGGCTGATACCAACGTCACTAAAGTGG
TGGAAGACAGGGCCAACGCACTGCTGGCCGATGGCCTGAAAGGCGTG
AAGCGTATCTCCCTCGACGAAGCGATGGCATCCGGTCATGTGAAAGA
GCAGGATCTGGTGCAGCCGTTCGTGGAAGGTCTGGCCGATATCGTTG
ATATGGCCGCGATTCAGAAAGCGGGCCTGACGCTGGGCGTTGATCCG
CTGGGGGGTTCCGGTATCGAATACTGGAAGCGTATTGGCGAGTATTA
CAACCTCAACCTGACTATCGTTAACGATCAGGTCGATCAAACCTTCC
GCTTTATGCACCTTGATAAAGACGGCGCGATCCGTATGGACTGCTCC
TCCGAGTGTGCGATGGGGGGCCTGCTGGCACTGCGTGATAAGTTCGA
TCTGGCGTTTGCTAACGACCCGGATTATGACCGTCACGGTATCGTCA
CTCCGGCAGGTTTGATGAATCCGAACCACTACCTGGCGGTGGCAATC
AATTACCTGTTCCAGCATCGTCCGCAGTGGGGCAAAGATGTTGCCGT
CGGTAAAACGCTGGTTTCATCTGCGATGATCGACCGTGTGGTCAACG
ACTTGGGCCGTAAACTGGTAGAAGTCCCGGTAGGTTTCAAATGGTTT
GTCGATGGTCTGTTCGACGGCAGCTTCGGCTTTGGCGGCGAAGAGAG
TGCAGGGGCTTCCTTCCTGCGTTTCGACGGCACGCCGTGGTCCACCG
ACAAAGACGGCATCATCATGTGTCTGCTGGCGGCGGAAATCACCGCT
GTCACCGGTAAGAACCCGCAGGAACACTACAACGAACTGGCAAAACG
CTTTGGTGCGCCGAGCTACAACCGTTTGCAGGCAGCTGCGACTTCCG
CACAAAAAGCGGCGCTGTCTAAGCTGTCTCCGGAAATGGTGAGCGCC
AGCACCCTGGCAGGTGACCCGATCACCGCGCGCCTGACTGCTGCTCC
GGGCAACGGTGCTTCTATTGGCGGTCTGAAAGTGATGACTGACAACG
GCTGGTTCGCCGCGCGTCCGTCAGGCACGGAAGACGCATATAAGATC
TACTGCGAAAGCTTCCTCGGTGAAGAACATCGCAAGCAGATTGAGAA
AGAAGCGGTTGAGATTGTTAGCGAAGTTCTGAAAAACGCGTAA

In certain embodiments, E. coli Pgm comprises an amino acid sequence that is at least about 80%, at least about 85%, at least about 90%, at least about 95%, at least about 96%, at least about 97%, at least about 98%, at least about 99%, or at least about 100% identical to the amino acid sequence set forth in SEQ ID NO: 48. In certain embodiments, E. coli Pgm comprises the amino acid sequence set forth in SEQ ID NO: 48. SEQ ID NO: 48 is provided below:

[SEQ ID NO: 48]
MAIHNRAGQPAQQSDLINVAQLTAQYYVLKPEAGNAEHAVKFGTSGH
RGSAARHSFNEPHILAIAQAIAEERAKNGITGPCYVGKDTHALSEPA
FISVLEVLAANGVDVIVQENNGFTPTPAVSNAILVHNKKGGPLADGI
VITPSHNPPEDGGIKYNPPNGGPADTNVTKVVEDRANALLADGLKGV
KRISLDEAMASGHVKEQDLVQPFVEGLADIVDMAAIQKAGLTLGVDP
LGGSGIEYWKRIGEYYNLNLTIVNDQVDQTFRFMHLDKDGAIRMDCS
SECAMAGLLALRDKFDLAFANDPDYDRHGIVTPAGLMNPNHYLAVAI
NYLFQHRPQWGKDVAVGKTLVSSAMIDRVVNDLGRKLVEVPVGFKWF
VDGLFDGSFGFGGEESAGASFLRFDGTPWSTDKDGIIMCLLAAEITA
VTGKNPQEHYNELAKRFGAPSYNRLQAAATSAQKAALSKLSPEMVSA
STLAGDPITARLTAAPGNGASIGGLKVMTDNGWFAARPSGTEDAYKI
YCESFLGEEHRKQIEKEAVEIVSEVLKNA

In certain embodiments, Pgm is a Bacillus subtilis Pgm. A representative nucleotide sequence of gene pgm is set forth in SEQ ID NO: 49 or at least 90%, 95%, 95%, or 99% identical to SEQ ID NO: 49. SEQ ID NO: 49 is provided below:

[SEQ ID NO: 49]
ATGAGTAAAAAACCAGCTGCACTCATCATTCTTGATGGGTTCGGATT
ACGTAACGAAACAGTAGGGAACGCAGTCGCTTTAGCGAAAAAACCGA
ATTTTGACCGCTATTGGAACCAGTATCCTCATCAAACTTTGACTGCT
TCAGGCGAGGCTGTAGGTCTTCCTGAAGGGCAAATGGGGAACTCCGA
AGTAGGTCACTTAAATATCGGTGCGGGACGTATTGTGTACCAAAGCT
TAACACGCGTAAATGTTGCCATTCGTGAAGGAGAGTTCGAACGCAAT
CAAACATTCCTTGACGCGATCAGCAACGCGAAAGAAAACAACAAAGC
CTTGCACCTGTTCGGTCTTTTATCTGACGGAGGCGTGCACAGCCATA
TCAATCATTTATTCGCACTGTTAAAGCTTGCGAAAAAAGAAGGGCTG
ACAAAGGTTTATATCCATGGCTTCCTTGACGGCCGTGATGTAGGTCC
GCAGACAGCGAAAACGTACATCAACCAGTTGAACGATCAAATCAAGG
AAATCGGTGTAGGTGAAATCGCCAGCATTTCCGGACGTTACTACTCT
ATGGACCGCGACAAACGCTGGGACCGTGTAGAAAAAGCGTACCGCGC
AATGGCGTACGGCGAAGGCCCATCTTACCGCAGCGCCCTGGATGTTG
TTGATGATTCATATGCAAATGGTATCTACGATGAATTCGTGATTCCA
TCTGTCATCACAAAAGAAAACGGTGAGCCTGTTGCGAAAATCCAAGA
CGGCGATTCTGTGATTTTCTATAATTTCAGACCGGACCGCGCCATCC
AGATTTCCAACACGTTCACAAACAAAGACTTCCGTGACTTCGACCGC
GGCGAGAATTATCCAAAGAACCTGTATTTCGTCTGCCTGACTCACTT
CAGTGAAACAGTTGACGGCTATGTAGCGTTTAAACCGATAAATCTTG
ATAACACAGTCGGAGAAGTATTATCTCAGCACGGGTTAAAACAGCTT
CGAATTGCAGAAACTGAAAAGTATCCGCACGTCACGTTCTTTATGAG
CGGCGGCCGTGAAGCTGAATTCCCGGGTGAAGAGCGTATTCTCATCA
ACTCGCCTAAAGTTGCAACGTATGACTTGAAGCCTGAGATGAGTGCG
TATGAAGTGAAAGACGCGCTTGTCAAAGAAATTGAAGCTGACAAGCA
TGACGCGATCATTCTTAACTTCGCAAACCCTGATATGGTCGGCCACT
CCGGAATGGTTGAACCAACAATTAAAGCAATTGAAGCAGTGGACGAA
TGCTTGGGCGAAGTCGTTGACGCGATCCTTGCTAAAGGCGGACACGC
TATCATTACCGCTGATCACGGTAATGCTGACATTCTGATTACAGAAT
CAGGCGAACCGCACACTGCGCATACAACAAACCCAGTCCCTGTGATT
GTAACGAAAGAAGGCATTACGCTGCGTGAAGGCGGAATCCTAGGCGA
CCTTGCACCAACGTTATTAGACCTTCTTGGTGTTGAAAAACCGAAAG
AAATGACAGGAACATCTTTAATTCAAAAATAA

In certain embodiments, Bacillus subtilis Pgm comprises an amino acid sequence that is at least about 80%, at least about 85%, at least about 90%, at least about 95%, at least about 96%, at least about 97%, at least about 98%, at least about 99%, or at least about 100% identical to the amino acid sequence set forth in SEQ ID NO: 50. In certain embodiments, Bacillus subtilis Pgm comprises the amino acid sequence set forth in SEQ ID NO: 50. SEQ ID NO: 50 is provided below:

(SEQ ID NO: 50)
MSKKPAALIILDGFGLRNETVGNAVALAKKPNFDRYWNQYPHQTITASGEAVGLPEGQMGNSEVGHL
NIGAGRIVYQSLTRVNVAIREGEFERNQTFLDAISNAKENNKALHLFGLLSDGGVHSHINHLFALLK
LAKKEGLTKVYIHGELDGRDVGPQTAKTYINQINDQIKEIGVGEIASISGRYYSMDRDKRWDRVEKA
YRAMAYGEGPSYRSALDVVDDSYANGIYDEFVIPSVITKENGEPVAKIQDGDSVIFYNFRPDRAIQI
SNTFINKDERDEDRGENYPKNLYFVCLTHESETVDGYVAFKPINLDNTVGEVLSQHGLKQLRIAETE
KYPHVTFFMSGGREAEFPGEERILINSPKVATYDIKPEMSAYEVKDALVKEIEADKHDAIILNFANP
DMVGHSGMVEPTIKAIEAVDECIGEVVDAILAKGGHAIITADHGNADILITESGEPHTAHTINPVPV
IVTKEGITLREGGILGDLAPTLIDLLGVEKPKEMTGTSLIQK

In certain embodiments, Pgm is a Lactococcus lactis Pgm. A representative nucleotide sequence of gene pgm is set forth in SEQ ID NO: 51 or at least 90%, 95%, 95%, or 99% identical to SEQ ID NO: 51. SEQ ID NO: 51 is provided below:

[SEQ ID NO: 51]
ATGTTTAAAGCAGTATTGTTTGATTTAGATGGTGTAATTACAGATACCGCAGAGTATCATTTTAGAG
CTTGGAAAGCTTTGGCTGAAGAAATTGGCATTAATGGTGTTGACCGCCAATTTAATGAGCAATTAAA
AGGGGTCTCACGAGAAGACTCGCTTCAGAAAATTCTAGATTTAGCTGATAAAAAAGTATCAGCTGAG
GAATTTAAAGAACTTGCTAAGAGAAAAAATGATAACTATGTGAAAATGATTCAGGATGTGTCGCCAG
CCGATGTCTATCCTGGAATTTTACAATTACTCAAAGATTTACGTTCAAATAAAATCAAAATTGCTTT
AGCATCGGCTTCTAAGAATGGTCCATTTTTATTAGAGAGAATGAATTTAACTGGATATTTTGATGCA
ATTGCTGATCCGGCTGAAGTTGCAGCATCAAAACCAGCACCAGATATTTTTATTGCAGCAGCACATG
CAGTGGGTGTTGCCCCCTCTGAATCAATTGGGTTAGAGGATTCTCAAGCTGGAATTCAAGCCATCAA
AGATTCAGGGGCTTTACCAATTGGTGTAGGGCGCCCAGAAGATTTGGGAGATGATATCGTCATTGTG
CCTGATACTTCACACTATACATTAGAATTTTTGAAAGAAGTTTGGCTTCAAAAGCAAAAATGA

In certain embodiments, Lactococcus lactis Pgm comprises an amino acid sequence that is at least about 80%, at least about 85%, at least about 90%, at least about 95%, at least about 96%, at least about 97%, at least about 98%, at least about 99%, or at least about 100% identical to the amino acid sequence set forth in SEQ ID NO: 52. In certain embodiments, Lactococcus lactis Pgm comprises the amino acid sequence set forth in SEQ ID NO: 52. SEQ ID NO: 52 is provided below:

(SEQ ID NO: 52)
MEKAVIFDLDGVITDTAEYHFRAWKALABEIGINGVDRQENEQLKGVSREDSLQKILDLADKKVSAE
EFKELAKRKNDNYVKMIQDVSPADVYPGILQLLKDLRSNKIKIALASASKNGPFLLEKMNLTGYFDA
IADPAEVAASKPAPDIFIAAAHAVGVAPSESIGLEDSQAGIQAIKDSGALPIGVGRPEDIGDDIVIV
PDTSYYTIEFLKEVWLQKQK

In certain embodiments, the presently disclosed microorganisms include a mutation of a gene encoding an enzyme of glycolysis. In certain embodiments, the presently disclosed microorganisms include a reduced expression of a gene encoding an enzyme of the glycolysis. In certain embodiments, the enzyme of the glycolysis is selected from the group consisting of phosphofructokinase A, phosphofructokinase B, fructose-biphosphate aldolase, triosephosphate isomerase, glyceraldehyde-3-phosphate dehydrogenase, phosphoglycerate kinase, phosphoglycerate mutase, enolase, and pyruvate kinase. In certain embodiments, the enzyme of glycolysis is phosphofructokinase B (PfkB). In certain embodiments, the enzyme of glycolysis is pyruvate kinase. In certain embodiments, the enzyme of glycolysis is phosphofructokinase A (PfkA) (Entrez Gene ID: 948412). PfkA catalyzes the phosphorylation of D-fructose 6-phosphate to fructose 1,6-bisphosphate by ATP, the first committing step of glycolysis. In certain embodiments, PfkA is an E. coli PfkA. A representative nucleotide sequence of gene pfkA is set forth in SEQ ID NO: 7. SEQ ID NO: 7 is provided below:

[SEQ ID NO: 7]
ATGATTAAGAAAATCGGTGTGTTGACAAGCGGCGGTGATGCGCCAGGCATGAACGCCGCAATTCGCG
GGGTTGTTCGTTCTGCGCTGACAGAAGGTCTGGAAGTAATGGGTATTTATGACGGCTATCTGGGTCT
GTATGAAGACCGTATGGTACAGCTAGACCGTTACAGCGTGTCTGACATGATCAACCGTGGCGGTACG
TTCCTCGGTTCTGCGCGTTTCCCGGAATTCCGCGACGAGAACATCCGCGCCGTGGCTATCGAAAACC
TGAAAAAACGTGGTATCGACGCGCTGGTGGTTATCGGGGGTGACGGTTCCTACATGGGTGCAATGCG
TCTGACCGAAATGGGCTTCCCGTGCATCGGTCTGCCGGGCACTATCGACAACGACATCAAAGGCACT
GACTACACTATCGGTTTCTTCACTGCGCTGAGCACCGTTGTAGAAGCGATCGACCGTCTGCGTGACA
CCTCTTCTTCTCACCAGCGTATTTCCGTGGTGGAAGTGATGGGCCGTTATTGTGGAGATCTGACGTT
GGCTGCGGCCATTGCCGGTGGCTGTGAATTCGTTGTGGTTCCGGAAGTTGAATTCAGCCGTGAAGAC
CTGGTAAACGAAATCAAAGCGGGTATCGCGAAAGGTAAAAAACACGCGATCGTGGCGATTACCGAAC
ATATGTGTGATGTTGACGAACTGGCGCATTTCATCGAGAAAGAAACCGGTCGTGAAACCCGCGCAAC
TGTGCTGGGCCACATCCAGCGCGGTGGTTCTCCGGTGCCTTACGACCGTATTCTGGCTTCCCGTATG
GGCGCTTACGCTATCGATCTGCTGCTGGCAGGTTACGGCGGTCGTTGTGTAGGTATCCAGAACGAAC
AGCTGGTTCACCACGACATCATCGACGCTATCGAAAACATGAAGCGTCCGTTCAAAGGTGACTGGCT
GGACTGCGCGAAAAAACTGTATTAA

In certain embodiments, E. coli PfkA comprises an amino acid sequence that is at least about 80%, at least about 85%, at least about 90%, at least about 95%, at least about 96%, at least about 97%, at least about 98%, at least about 99%, or at least about 100% identical to the amino acid sequence set forth in SEQ ID NO: 21. In certain embodiments, E. coli PfkA comprises the amino acid sequence set forth in SEQ ID NO: 21. SEQ ID NO: 21 is provided below:

[SEQ ID NO: 21)
MIKKIGVLTSGGDAPGMNAAIRGVVRSALTEGLEVMGIYDGYLGLYEDRMVQLDRYSVSDMINRGGT
FIGSARFPEERDENIRAVAIENIKKRGIDALVVIGGDGSYMGAMRLTEMGFPCIGLPGTIDNDIKGT
DYTIGFFTALSTVVEAIDRERDTSSSHQRISVVEVMGRYCGDLTLAAAIAGGCEFVVVPEVEFSRED
LVNEIKAGIAKGKKHAIVAITEHMCDVDELAHFIEKETGRETRATVIGHIQRGGSPVPYDRILASRM
GAYAIDLILAGYGGRCVGIQNEQLVHHDIIDAIENMKRPEKGDWIDCAKKLY

In certain embodiments, PfkA is a Bacillus subtilis PfkA. A representative amino acid sequence of Bacillus subtilis PfkA is found as 034529 (Uniprot)/BSU29190 (KEGG) or as set forth in SEQ ID NO: 12 or at least 90%, 95%, 95%, or 99% identical to SEQ ID NO:12. SEQ ID NO: 12 is provided below:

(SEQ ID NO: 12)
MKRIGVITSGGDSPGMNAAVRAVVRKAIYHDVEVYGIYNGYAGLISGKIEKLELGSVGDIIHRGGTK
LYTARCPEFKTVEGREKGIANIKKEGIEGLVVIGGDGSYMGAKKITEHGFPCVGVPGTIDNDIPGTD
FTIGEDTALNTVIDAIDKIRDTATSHERTYVIEVMGRHAGDIALWAGLAGGAESILIPEADYDMHEI
IARLKRGHERGKKHSIIIVAEGVGSGVEFGKRIEEEINLETRVSVLGHIQRGGSPSAADRVLASRLG
AYAVELLLEGKGGRCVGIQNNKEVDHDIIEILETKHTVEQNMYQLSKELSI

A representative nucleotide sequence of a Bacillus subtilis gene pfkA is set forth in SEQ ID NO: 27 or at least 90%, 95%, 95%, or 99% identical to SEQ ID NO: 27. SEQ ID NO: 27 is provided below:

(SEQ ID NO: 27)
atgaaacgaataggggtattaacgagcggcggggattccccgggaatgaacgcagcagttcgcgcag
tagtcagaaaagcgatctatcatgacgttgaagtttacggtatttacaacggatacgcgggattgat
cagcggaaagattgaaaagcttgaactcggatcagtaggcgatattatacatcgtggagggactaag
ctttatacggcgagatgtcctgaattcaaaacagttgaaggccgtgaaaaagggatagcaaacttga
agaagcttggtattgaaggccttgttgttatcggtggagacggttcctatatgggtgcgaaaaaatt
aacggaacacgggtttccatgtgtaggtgtaccgggtacaattgataatgacattccgggcactgat
tttacaatcggtttcgatacagctttaaatacagtaattgacgcaattgataagattcgcgatacag
cgacttctcatgaacgtacatatgtaatcgaagtaatgggccgtcatgccggcgatatcgcattgtg
ggccgatcttgcagggggcgcagaatcgatcttaatccctgaggcagactatgacatgcacgaaatc
attgcccgcttaaaacgcggccacgaacgcggcaagaagcacagtattattattgttgccgaaggtg
taggcagcggtgttgaattcgggaaacgcattgaagaagaaacaaatcttgaaactagggtatctgt
attgggccatatccagcgcggaggttctccgagtgctgctgaccgtgtgttggcaagccgtctcggc
gcatatgcagttgaactgctgcttgaaggaaaaggcggacgctgtgtaggtatacaaaacaataagc
ttgtagaccatgatattatagaaatacttgagacaaaacacacagttgagcaaaacatgtatcagct
ttcaaaagaactgtctatctaa

In certain embodiments, PfkA is a Lactococcus lactis PfkA. A representative amino acid sequence of Lactococcus lactis PfkA is found as LLA12_RS07020: ATP-dependent 6-phosphofructokinase, EC2.7.1.11, or as set forth in SEQ ID NO: 13 or at least 90%, 95%, 95%, or 99% identical to SEQ ID NO:13, which is provided below:

(SEQ ID NO: 13)
MKRIAV;TSGGDAPGMNAAIRAVVRKAISEGIEVYGINHGYAGMVAGDIFPLTSASVGDKIGRGGTF
LYSARYPEFAQVEGQLAGIEQLKKEGIEGVVVIGGDGSYHGAMRITEHGFPAVGLPGTIDNDIVGTD
FTIGFDTAVSTVVDALDKIRDTSSSHNRTFVVEVMGRNAGDIALNAGIAAGADDICIPEKEFKFENV
VNNINKGYEKGKNHHIIVLAEGVMTGEEFATKLKEAGYKGDLRVSVIGHIQRGGSPTARDRVLASRM
GARAVELLRDGIGGVAVGIRNEELVESPILGTAEEGALFSITTEGGIKVNNPHKAGLELYRINSALN
NLNLN

A representative nucleotide sequence of a Lactococcus lactis gene pfkA is set forth in SEQ ID NO: 32 or at least 90%, 95%, 95%, or 99% identical to SEQ ID NO: 32. SEQ ID NO: 32 is provided below:

(SEQ ID NO: 32)
atgAAACGCATTGCAGTTTTGACTTCTGGTGGTGATGCCCCAGGAATGAATGCGGCTATTCGTGCAG
TTGTTCGCAAAGCAATTTCTGAAGGTATCGAAGTTTACGGTATCAATCACGGATATGCGGGCATGGT
TGCGGGAGATATTTTCCCGCTTACGTCAGCTTCAGTTGGTGATAAAATCGGTCGTGGTGGTACATTC
TTGTATTCAGCACGCTACCCAGAATTTGCTCAAGTAGAAGGACAACTTGCTGGGATTGAGCAACTTA
AAAAATTCGGTATCGAAGGTGTCGTTGTAATCGGTGGTGATGGTTCTTATCATGGAGCTATGCGTCT
TACAGAACATGGTTTCCCAGCTGTTGGACTTCCAGGAACAATCGATAACGATATCGTAGGAACTGAT
TTTACAATTGGATTTGATACAGCTGTTTCAACAGTTGTAGATGCCTTGGATAAAATTCGTGATACTT
CATCATCACATAACCGTACTTTCGTTGTAGAAGTAATGGGACGTAATGCTGGAGATATCGCTTTGAA
TGCTGGTATCGCTGCTGGTGCAGATGATATTTGTATTCCAGAAAAAGAATTTAAGTTTGAAAACGTA
GTTAACAACATTAACAAAGGCTACGAAAAAGGTAAAAATCACCACATCATCGTTCTTGCTGAAGGTG
TAATGACTGGTGAAGAATTTGCTACAAAACTTAAAGAAGCTGGTTATAAAGGAGACCTTCGCGTTTC
TGTCCTTGGACACATCCAACGTGGTGGTTCACCAACAGCTCGTGACCGTGTTCTAGCTTCACGTATG
GGTGCTCGTGCCGTTGAATTGCTTCGTGATGGAATCGGTGGCGTAGCCGTTGGTATCCGTAATGAAG
AACTTGTAGAAAGTCCAATTCTCGGAACAGCTGAAGAAGGAGCACTCTTTAGTTTGACTACTGAAGG
TGGAATCAAAGTAAATAACCCGCATAAAGCTGGTCTTGAACTTTACCGTCTTAACTCAGCACTCAAC
AATCTTAACCTTAACTaa

In certain embodiments, PfkB is an E. coli PfkB. A representative nucleotide sequence of gene pfkB is set forth in SEQ ID NO: 22. SEQ ID NO: 22 is provided below:

[SEQ ID NO: 22]
atgGTACGTATCTATACGTTGACACTTGCGCCCTCTCTCGATAGCGCAACAATTACCCCGCAAATTT
ATCCCGAAGGAAAACTGCGCTGTACCGCACCGGTGTTCGAACCCGGGGGGGGCGGCATCAACGTCGC
CCGCGCCATTGCCCATCTTGGAGGCAGTGCCACAGCGATCTTCCCGGCGGGTGGCGCGACCGGCGAA
CACCTGGTTTCACTGTTGGCGGATGAAAATGTCCCCGTCGCTACTGTAGAAGCCAAAGACTGGACCC
GGCAGAATTTACACGTACATGTGGAAGCAAGCGGTGAGCAGTATCGTTTTGTTATGCCAGGCGCGGC
ATTAAATGAAGATGAGTTTCGCCAGCTTGAAGAGCAAGTTCTGGAAATTGAATCCGGGGCCATCCTG
GTCATAAGCGGAAGCCTGCCGCCAGGTGTGAAGCTGGAAAAATTAACCCAACTGATTTCCGCTGCGC
AAAAACAAGGGATCCGCTGCATCGTCGACAGTTCTGGCGAAGCGTTAAGTGCAGCACTGGCAATTGG
TAACATCGAGTTGGTTAAGCCTAACCAAAAAGAACTCAGTGCGCTGGTGAATCGCGAACTCACCCAG
CCGGACGATGTCCGCAAAGCCGCGCAGGAAATCGTTAATAGCGGCAAGGCCAAACGGGTTGTCGTTT
CCCTGGGTCCACAAGGAGCGCTGGGTGTTGATAGTGAAAACTGTATTCAGGTGGTGCCACCACCGGT
GAAAAGCCAGAGTACCGTTGGCGCTGGTGACAGCATGGTCGGCGCGATGACACTGAAACTGGCAGAA
AATGCCTCTCTTGAAGAGATGGTTCGTTTTGGCGTAGCTGCGGGGAGTGCAGCCACACTCAATCAGG
GAACACGTCTGTGCTCCCATGACGATACGCAAAAAATTTACGCTTACCTTTCCCGCtaa

In certain embodiments, E. coli PfkB comprises an amino acid sequence that is at least about 80%, at least about 85%, at least about 90%, at least about 95%, at least about 96%, at least about 97%, at least about 98%, at least about 99%, or at least about 100% identical to the amino acid sequence set forth in SEQ ID NO: 23. In certain embodiments, E. coli PfkB comprises the amino acid sequence set forth in SEQ ID NO: 23. SEQ ID NO: 23 is provided below:

[SEQ ID NO: 23]
MVRIYTITLAPSLDSATITPQIYPEGKLRCTAPVEEPGGGGINVARAIAHLGGSATAIFPAGGATGE
HIVSLLADENVPVATVEAKDWTRQNLHVHVEASGEQYRFVMPGAALNEDEFRQLEEQVLEIESGAIL
VISGSLPPGVKLEKLTQLISAAQKQGIRCIVDSSGEALSAALAIGNIELVKPNQKELSALVNRELTQ
PDDVRKAAQEIVNSGKAKRVVVSIGPQGALGVDSENCIQVVPPPVKSQSTVGAGDSMVGAMTLKLAE
NASLEEMVREGVAAGSAATLNQGTRICSHDDTQKIYAYLSR

In certain embodiments, the presently disclosed microorganisms do not include a deletion or a reduced expression of hexokinase. In certain embodiments, the presently disclosed microorganisms do not include a deletion, a disruption, or a reduced expression of glucokinase. In certain embodiments, the presently disclosed microorganisms do not include a deletion or a reduced expression of glucose-6-phosphate isomerase.

In certain embodiments, the presently disclosed microorganisms include a mutation of a gene encoding an enzyme of the allose degradation pathway. In certain embodiments, the presently disclosed microorganisms include a reduced expression of a gene encoding an enzyme of the allose degradation pathway. In certain embodiments, the enzyme of the allose degradation pathway is allose-6-phosphate isomerase (RpiB) (Entrez Gene ID: 948602). RpiB catalyzes the interconversion of ribulose-5-P and ribose-5-P, as well the interconversion of D-allose-6-phosphate (All6P) and D-allulose-6-phosphate. In certain embodiments, RpiB is an E. coli RpiB. A representative nucleotide sequence of gene rpiB is set forth in SEQ ID NO: 8 or at least 90%, 95%, 95%, or 99% identical to SEQ ID NO: 8. SEQ ID NO: 8 is provided below:

[SEQ ID NO: 8]
ATGAAAAAGATTGCATTTGGCTGTGATCATGTCGGTTTCATTTTAAAACATGAAATAGTGGCACATT
TAGTTGAGCGTGGCGTTGAAGTGATTGATAAAGGAACCTGGTCGTCAGAGCGTACTGATTATCCACA
TTACGCCAGTCAAGTCGCACTGGCTGTTGCTGGCGGAGAGGTTGATGGGGGGATTTTGATTTGTGGT
ACTGGCGTCGGTATTTCGATAGCGGCGAACAAGTTTGCCGGAATTCGCGCGGTCGTCTGTAGCGAAC
CTTATTCCGCGCAACTTTCGCGGCAGCATAACGACACCAACGTGCTGGCTTTTGGTTCACGAGTGGT
TGGCCTCGAACTGGCAAAAATGATTGTGGATGCGTGGCTGGGCGCACAGTACGAAGGCGGTCGTCAT
CAACAACGCGTGGAGGCGATTACGGCAATAGAGCAGCGGAGAAATTGA

In certain embodiments, E. coli RpiB comprises an amino acid sequence that is at least about 80%, at least about 85%, at least about 90%, at least about 95%, at least about 96%, at least about 97%, at least about 98%, at least about 99%, or at least about 100% identical to the amino acid sequence set forth in SEQ ID NO: 24. In certain embodiments, E. coli RpiB comprises the amino acid sequence set forth in SEQ ID NO: 24. SEQ ID NO: 24 is provided below:

[SEQ ID NO: 24]
MKKIAFGCDHVGFILKHEIVAHLVERGVEVIDKGTWSSERTDYPHYASQVALAVAGGEVDGGILICG
TGVGISIAANKFAGIRAVVCSEPYSAQLSRQHNDINVLAFGSRVVGLELAKMIVDAWLGAQYEGGRH
QQRVEAITAIEQRRN

In certain embodiments, RpiB is a Bacillus subtilis gene rpiB. A representative amino acid sequence of RpiB is found as A0A6M4JQ63 (Uniprot)/BSU36920 (KEGG) or is set forth in SEQ ID NO: 14 or at least 90%, 95%, 95%, or 99% identical to SEQ ID NO: 14. SEQ ID NO: 14 is provided below:

(SEQ ID NO: 14)
MKVAIASDHGGVHIRNEIKELMDELQIEYIDMGCDCGSGSVDYPDYAFPVAEKVVSGEVDRGILICG
IGIGMSISANKVKGIRCALAHDTESAKATREHNDTNILAMGERVIGPGLAREIAKIWITTEETGGRH
QTRIGKISDYEEKNL

A representative nucleotide sequence of Bacillus subtilis gene rpiB is set forth in SEQ ID NO: 28 or at least 90%, 95%, 95%, or 99% identical to SEQ ID NO: 28. SEQ ID NO: 28 is provided below:

(SEQ ID NO: 28)
atgaaagtagccattgcatcggatcatggcggcgttcacattcgaaatgaaatcaaagagttaatgg
acgaattgcasattgaatatattgatatgggctgtgactgcggcagcggctctgtcgattatccgga
ttatgcttttccggtggccgaaaaagtggttagcggcgaagttgacagaggcattttaatttgcggg
acaggcatcggcatgagcatttccgctaataaagtaaaagggattcgctgcgcgctggcgcacgata
ccttcagcgcgaaggcgacgagggagcataatgacacaaacatccttgcgatgggtgaacgggtgat
cggacctggtttggctcgggaaatcgcaaaaatctggctgactactgagtttaccgggggaagacac
caaacgcgtattggaaaaatctccgattatgaagagaaaaacctgtag

In certain embodiments, RpiB is a Lactococcus lactis RpiB. A representative amino acid sequence of gene rpiB is found as LLA12_RS12460: ribose-5-phosphate isomerase, EC5.3.1.6 or is set forth in SEQ ID NO: 15 or at least 90%, 95%, 95%, or 99% identical to SEQ ID NO: 15, which is provided below.

(SEQ ID NO: 15)
MDNIKKQVGIKAAEFVKSGMVVGLGTGSTAAYFVEELGRRIAEEQLEITGVTTSNVISSQARALGIP
LASIDEVDYVDLTVDGADEIDSSINGIKGGGAALLMEKIVATYSKDYIWIVDESKLSENLGSFKIPV
EVIPYGSQQVFKKFEAAGYAPTWRLNEENERLITDMHHFIIDLHISQIKEPEKLAEELDLMVGVVEH
GLENNMVKKVIVAGNEGVRIINK

A representative nucleotide sequence of a Lactococcus lactis gene rpiB is set forth in SEQ ID NO: 34 or at least 90%, 95%, 95%, or 99% identical to SEQ ID NO: 34. SEQ ID NO: 34 is provided below:

(SEQ ID NO: 34)
atgGATAATTTAAAAAAACAAGTCGGCATAAAAGCTGCTGAATTTGTTAAATCAGGAATGGTCGTTG
GTTTAGGAACTGGGTCAACAGCAGCCTATTTTGTCGAAGAATTGGGTCGAAGAATTGCCGAAGAACA
ATTGGAAATTACGGGTGTAACAACGTCCAATGTAACAAGTAGCCAAGCCAGAGCTCTTGGAATTCCT
TTAGCCTCTATTGACGAAGTAGATTATGTTGATTTAACAGTTGATGGCGCAGATGAAATTGATTCTT
CACTAAATGGTATTAAAGGTGGTGGAGCAGCACTTCTAATGGAAAAAATTGTTGCAACCTACTCAAA
AGACTATATTTGGATTGTTGATGAAAGTAAATTATCAGAAAATCTAGGATCCTTTAAAATTCCTGTA
GAAGTTATTCCTTATGGCTCACAACAAGTTTTTAAAAAATTCGAAGCGGCTGGCTATGCTCCAACTT
GGCGTCTAAATGAGGAAAACGAGAGATTGATAACGGATATGCATCACTTTATTATTGACCTTCATAT
CTCTCAAATTAAAGAACCAGAAAAACTTGCTGAAGAGCTTGATTTAATGGTTGGAGTTGTTGAACAC
GGCCTCTTTAATAACATGGTTAAAAAAGTGATTGTTGCTGGCAACGAAGGCGTAAGAATAATAAATA
AGtaa

In certain embodiments, the presently disclosed microorganisms include mutation of a gene encoding an enzyme of the mannose biosynthesis pathway. In certain embodiments, the presently disclosed microorganisms include a reduced expression of a gene encoding an enzyme of the mannose biosynthesis pathway. In certain embodiments, the enzyme of the mannose biosynthesis pathway is mannose-6-phosphate isomerase (ManA) (Entrez Gene ID: 944840). ManA is involved in the synthesis of the GDP-mannose and dolichol-phosphate-mannose required for a number of critical mannosyl transfer reactions. ManA also catalyzes the interconversion of fructose-6-phosphate and mannose-6-phosphate. In certain embodiments, ManA is an E. coli ManA. A representative nucleotide sequence of gene manA is set forth in SEQ ID NO: 9 or at least 90%, 95%, 95%, or 99% identical to SEQ ID NO: 9, which is provided below.

[SEQ ID NO: 9]
ATGCAAAAACTCATTAACTCAGTGCAAAACTATGCCTGGGGCAGCAAAACGGCGTTGACTGAACTTT
ATGGTATGGAAAATCCGTCCAGCCAGCCGATGGCCGAGCTGTGGATGGGCGCACATCCGAAAAGCAG
TTCACGAGTGCAGAATGCCGCCGGAGATATCGTTTCACTGCGTGATGTGATTGAGAGTGATAAATCG
ACTCTGCTCGGAGAGGCCGTTGCCAAACGCTTTGGCGAACTGCCTTTCCTGTTCAAAGTATTATGCG
CAGCACAGCCACTCTCCATTCAGGTTCATCCAAACAAACACAATTCTGAAATCGGTTTTGCCAAAGA
AAATGCCGCAGGTATCCCGATGGATGCCGCCGAGCGTAACTATAAAGATCCTAACCACAAGCCGGAG
CTGGTTTTTGCGCTGACGCCTTTCCTTGCGATGAACGCGTTTCGTGAATTTTCCGAGATTGTCTCCC
TACTCCAGCCGGTCGCAGGTGCACATCCGGCGATTGCTCACTTTTTACAACAGCCTGATGCCGAACG
TTTAAGCGAACTGTTCGCCAGCCTGTTGAATATGCAGGGTGAAGAAAAATCCCGCGCGCTGGCGATT
TTAAAATCGGCCCTCGATAGCCAGCAGGGTGAACCGTGGCAAACGATTCGTTTAATTTCTGAATTTT
ACCCGGAAGACAGCGGTCTGTTCTCCCCGCTATTGCTGAATGTGGTGAAATTGAACCCTGGCGAAGC
GATGTTCCTGTTCGCTGAAACACCGCACGCTTACCTGCAAGGCGTGGCGCTGGAAGTGATGGCAAAC
TCCGATAACGTGCTGCGTGCGGGTCTGACGCCTAAATACATTGATATTCCGGAACTGGTTGCCAATG
TGAAATTCGAAGCCAAACCGGCTAACCAGTTGTTGACCCAGCCGGTGAAACAAGGTGCAGAACTGGA
CTTCCCGATTCCAGTGGATGATTTTGCCTTCTCGCTGCATGACCTTAGTGATAAAGAAACCACCATT
AGCCAGCAGAGTGCCGCCATTTTGTTCTGCGTCGAAGGCGATGCAACGTTGTGGAAAGGTTCTCAGC
AGTTACAGCTTAAACCGGGTGAATCAGCGTTTATTGCCGCCAACGAATCACCGGTGACTGTCAAAGG
CCACGGCCGTTTAGCGCGTGTTTACAACAAGCTGTAA

In certain embodiments, E. coli ManA comprises an amino acid sequence that is at least about 80%, at least about 85%, at least about 90%, at least about 95%, at least about 96%, at least about 97%, at least about 98%, at least about 99%, or at least about 100% identical to the amino acid sequence set forth in SEQ ID NO: 25. In certain embodiments, E. coli ManA comprises the amino acid sequence set forth in SEQ ID NO: 25. SEQ ID NO: 25 is provided below:

[SEQ ID NO: 25]
MQKLINSVQNYAWGSKTALTELYGMENPSSQPMAELWMGAHPKSSSRVQNAAGDIVSIRDVIESDKS
TLIGEAVAKRFGELPFLEKVICAAQPLSIQVHENKHNSEIGFAKENAAGIPMDAAERNYKDPNHKPE
LVFALTPFLAMNAFREFSEIVSLLQPVAGAHPAIAHFLQQPDAERLSELFASLLNMQGEEKSRALAI
LKSALDSQQGEPWQTIRLISEFYPEDSGIFSPLLINVVKLNPGEAMFIFAETPHAYLQGVALEVMAN
SDNVIRAGITPKYIDIPELVANVKFEAKPANQLLTQPVKQGAELDFPIPVDDFAFSLHDLSDKETTI
SQQSAAILFCVEGDATLWKGSQQLQLKPGESAFIAANESPVTVKGHGRLARVYNKL

In certain embodiments, ManA is a Bacillus subtilis ManA. A representative amino acid sequence of ManA is found at 031646 (Uniprot)/BSU12020 (KEGG) or is set forth in SEQ ID NO: 16 or at least 90%, 95%, 95%, or 99% identical to SEQ ID NO: 16.

(SEQ ID NO: 16)
MTTEPLFFKPVFKERIWGGTALADEGYTIPSQRTGECWAFAAHQNGQSVVQNGMYKGFTLSELWEHH
RHLFGQLEGDRFPLITKILDADQDLSVQVHPNDEYANIHENGELGKTECWYIIDCQKDAEIIYGHNA
TTKEELTTMIERGEWDELIRRVKVKPGDFFYVPSGTVHAIGKGILALETQQNSDTTYRLYDYDRKDA
EGKLRELHLKKSIEVIEVPSIPERHTVHHEQIEDLLTTTLIECAYFSVGKWNLSGSASLKQQKPFLL
ISVIEGEGRMISGEYVYPEKKGDHMILPYGLGEFKLEGYAECIVSHI

A representative nucleotide sequence of a Bacillus subtilis gene manA is set forth in SEQ ID NO: 29 or at least 90%, 95%, 95%, or 99% identical to SEQ ID NO: 29.

(SEQ ID NO: 29)
atgacgactgaaccgttatttttcaagcctgttttcaaagaaagaatttggggggggaccgctttag
ctgattttggctataccattccgtcacaacgaacaggggagtgctgggcttttgccgcgcatcaaaa
tggtcaaagcgttgttcaaaacggaatgtataaggggttcacgctcagcgaattatgggaacatcac
agacatttattcggacagcttgaaggggaccgtttccctctgcttacaaaaatattagatgctgacc
aggacttatctgttcaggtgcatccgaatgatgaatatgccaacatacatgaaaacggtgagcttgg
aaaaacagaatgctggtacattattgattgccaaaaagatgccgagattatttatggccacaatgca
acaacaaaggaagaactaactaccatgatagagcgtggagaatgggatgagctcttgcgccgtgtaa
aggtaaagccgggggattttttctatgtgccaagcggtactgttcatgcgattggaaaaggaattct
tgctttggagacgcagcagaactcagacacaacctacagattatatgattatgaccgaaaagatgca
gaaggcaagctgcgcgagcttcatctgaaaaagagcattgaagtgatagaggtcccg
tctattccagaacggcatacagttcaccatgaacaaattgaggatttgcttacaacgacattgattg
aatgcgcttacttttcggtggggaaatggaacttatcaggatcagcaagcttaaagcagcaaaaacc
attccttcttatcagtgtgattgaaggggagggccgtatgatctctggtgagtatgtctatcctttc
aaaaaaggagatcatatgttgctgccttacggtcttggagaatttaaactcgaaggatatgcagaat
gtatcgtctcccatctgtaa

In certain embodiments, in Bacillus subtilis, optionally in combination with deletion of Bacillus subtilis ManA, one or both of the following are also deleted.

(i) YvyI of Bacillus subtilis. A representative amino acid sequence of YvyI is found at P39841 (Uniprot)/BSU35790 (KEGG) or is set forth in SEQ ID NO: 17 or at least 90%, 95%, 95%, or 99% identical to SEQ ID NO: 17. SEQ ID NO: 17 is provided below:

(SEQ ID NO: 17)
MTQSPIELTPVFKEKIWGGTALRDREGYSIPSESTGECWAISAHPKGPSTVANGPYKGKTLIELWEE
HREVEGGVEGDREPLLTKLIDVKEDTSIKVHPDDYYAGENEEGEIGKTECWYIIDCKENAEIIYGHT
ARSKTELVTMINSGDWEGLLRRIKIKPGDFYYVPSGTLHALCKGALVLETQQNSDATYRVYDYDRLD
SNGSPRELHFAKAVNAATVPHVDGYIDESTESRKGITIKTFVQGEYESVYKWDINGEAEMAQDESFL
ICSVIEGSGLIKYEDKTCPLKKGDHFILPAQMPDFTIKGTCTLIVSHI

A representative nucleotide sequence of Bacillus subtilis gene yvyI is set forth in SEQ ID NO: 30 or at least 90%, 95%, 95%, or 99% identical to SEQ ID NO: 30. SEQ ID NO: 30 is provided below:

(SEQ ID NO: 30)
atgacgcaatcaccgatttttctaacgcctgtgtttaaagaaaaaatctggggcggaaccgctttac
gagatagatttggatacagtattccttcagaatcaacgggggaatgctgggccatttccgctcatcc
aaaaggaccgagcactgttgcaaatggcccgtataaaggaaagacattgatcgagctttgggaagag
caccgtgaagtattcggcggcgtagagggggatcggtttccgcttctgacaaagctgctggatgtga
aggaagatacgtcaattaaagttcaccctgatgattactatgccggagaaaacgaagagggagaact
cggcaagacggaatgctggtacattatcgactgtaaggaaaacqcagaaatcatttacgggcatacg
gcccgctcaaaaaccgaacttgtcacaatgatcaacagcggtgactgggagggcctgctgcgaagaa
tcaaaattaaaccgggtgatttctattatgtgccgagcggaacgctgcacgcattgtgcaagggggc
ccttgttttagagactcagcaaaattcagatgccacataccgggtgtacgattatgaccgtcttgat
agcaacggaagtccgagagagcttcattttgccaaagcggtcaatgccgccacggttccccatgtgg
acgggtatatagatgaatcgacagaatcaagaaaaggaataaccattaaaacatttgtccaagggga
atatttttcggtttataaatgggacatcaatggcgaagctgaaatggctcaggatgaatcctttctg
atttgcagcgtgatagaaggaagcggtttgctcaagtatgaggacaaaacatgtccgctcaaaaaag
gtgatcactttattttgccggctcaaatgcccgattttacgataaaaggaacttgtacccttatcgt
gtctcatatttaa

(ii) GmuF of Bacillus subtilis. A representative amino acid sequence of GmuF is found at 005511 (Uniprot)/BSU05870 (KEGG) or is set forth in SEQ ID NO: 18 or at least 90%, 95%, 95%, or 99% identical to SEQ ID NO: 18. SEQ ID NO: 18 is provided below:

(SEQ ID NO: 18)
MTHPLFLEPVFKERLWGGTKLRDAFGYAIPSQKTGECWAVSAHAHGSSSVKNGPLAGKTIDQVWKDH
PEIEGEPDGKVEPILVKLLDANMDISVQVHPDDDYAKLKENGDIGKTECWYIIDCKDDAELILGHHA
STKEEFKQRIESGDWNGILRRIKIKPGDFFYVPSGTIHALCKGTIVLEIQQNSDTTYRVYDYDRQND
QGQKRTIHIEKAMEVITIPHIDKVHTPEVKEVGNAEIIVYVQSDYFSVYKWKISGRAAFPSYQTYLL
GSVISGSGRIINNGIQYECNAGSHFILPAHFGEFTIEGTCEFMISHP

A representative nucleotide sequence of a Bacillus subtilis gene gmuF is set forth in SEQ ID NO: 31 or at least 90%, 95%, 95%, or 99% identical to SEQ ID NO: 31. SEQ ID NO: 31 is provided below:

(SEQ ID NO: 31)
atgacgcatccattatttttagagcctgtctttaaagaaagactatggggagggacgaagcttcgtg
acgcttttggctacgcaataccctcacaaaaaacaggtgagtgctgggccgtttctgcacatgccca
tggctcgtcgtctgtaaaaaatggcccgctggcaggaaagacacttgatcaagtatggaaagatcat
ccagagatattcgggtttccggatggtaaggtgtttccgctgctggtaaagctgctggacgccaata
tggatctctccgtgcaagtccatcctgatgatgattatgcaaaactgcacgaaaatggcgaccttgg
taaaacggagtgctggtatatcattgattgcaaagatgacgccgaactaattttgggacatcatgca
agcacaaaggaagagttcaaacaacgaatagaaagcggtgattggaacgggctgctgaggcgaatca
aaatcaagccaggagatttcttttatgtgccaagcggtacactccatgctttatgtaagggaaccct
tgtccttgaaatccagcaaaactctgatacaacatatcgcgtatacgattatgaccgctgtaatgac
cagggccaaaaaagaactcttcatatagaaaaagccatggaagtcataacgataccgcatatcgata
aagtgcatacaccggaagtaaaagaagttggtaacgctgagatcattgtttatgtgcaatcagatta
tttctcagtgtacaaatggaagattagcggccgagctgcttttccttcatatcaaacctatttgctg
gggagtgttctgagcggatcaggacgaatcataaataatggtattcagtatgaatgcaatgcaggct
cacactttattctgcctgcgcattttggagaatttacaatagaaggaacatgtgaattcatgatatc
tcatccttaa

In certain embodiments, ManA is a Lactococcus lactis ManA. A representative amino acid sequence of Lactococcus lactis ManA is found as LLA12_RS03920: mannose-6-phosphate isomerase, EC5.3.1.8 or is set forth in SEQ ID NO: 19 or at least 90%, 95%, 95%, or 99% identical to SEQ ID NO: 19. SEQ ID NO: 19 is provided below:

(SEQ ID NO: 19)
MKEPLEINSVLQEKIWGGDHIKEFGYDLPSDKVGEYWAISAHPHGVSTIANGEFKGQKLDQLYASHR
ELFGDSKKEVFPLLTKILDANDWLSVQVHPDDEYGQKHEGELGKTECWYIISAEPGAEIIYGHNAKS
REELAEMIKSGDWDHLLRKVKVKTGDFFHVPSGTMHAIGAGIVILETQQSSDTTYRVYDEDRKDDQG
NIRELHIQQSIDVINIPGDKVPENQVKTEKFADAEITTLVKSDEEDVYKWQIHGDHEFTKVADYTLV
SVLDGQGKLTVDGNEYPVEKGAHFILPSNIEKWNLSGQLEIIASNPA

A representative nucleotide sequence of a Lactococcus lactis gene manA is set forth in SEQ ID NO: 35 or at least 90%, 95%, 95%, or 99% identical to SEQ ID NO: 35. SEQ ID NO: 35 is provided below:

(SEQ ID NO: 35)
atgAAAGAACCATTGTTTTTGAACTCAGTTTTGCAAGAAAAAATCTGGGGCGGCGACCATTTGAAAG
AGTTTGGCTATGATTTGCCATCAGACAAAGTTGGTGAATATTGGGCTATTTCTGCTCATCCACATGG
TGTGTCAACAATTGCTAATGGCGAATTTAAAGGTCAAAAACTTGACCAATTATACGCAAGTCACCGC
GAATTGTTTGGTGATAGTAAAAAAGAAGTTTTTCCCTTACTAACTAAAATTTTAGATGCCAATGACT
GGCTTTCTGTGCAAGTTCATCCAGATGATGAATATGGACAAAAACATGAAGGTGAACTTGGAAAAAC
TGAATGTTGGTACATTATTTCAGCTGAACCAGGTGCTGAAATTATCTATGGACATAATGCTAAATCA
CGTGAAGAATTAGCAGAAATGATTAAATCTGGTGATTGGGATCATTTGTTACGTAAGGTAAAAGTGA
AAACAGGAGATTTCTTCCATGTTCCGTCAGGAACAATGCACGCAATCGGTGCTGGAATTGTTATTCT
TGAAACACAACAATCTTCTGATACAACTTACCGTGTTTATGATTTCGACCGTAAAGATGACCAAGGA
AATCTACGTGAATTACATATTCAACAATCAATTGATGTATTGAATATTCCGGGCGACAAAGTTCCTG
AAAATCAAGTTAAAACTGAAAAATTTGCTGATGCAGAAATTACAACTCTTGTGAAATCAGATTTCTT
TGATGTTTATAAATGGCAAATTCATGGTGACCATGAATTTACCAAAGTTGCTGATTACACTTTAGTT
TCTGTACTTGATGGTCAAGGAAAATTAACAGTTGATGGAAATGAATATCCAGTTGAAAAAGGAGCTC
ATTTCATCTTACCAAGCAACATTGAAAAATGGAATTTGTCTGGTCAATTAGAAATTATTGCCAGCAA
TCCTGCCtaa

In certain embodiments, the deletion of a gene comprises a non-frameshift deletion, a frameshift deletion, or a combination thereof. In certain embodiments, the deletion of a gene can be achieved by an insertion (e.g., a non-frameshift insertion, a frameshift insertion, or a combination thereof). In certain embodiments, the deletion of a gene comprises a nonsense mutation.

### 2.3. Cells

The present disclosure provides recombinant microorganisms. Any culturable microorganism is suitable for use in the compositions and methods described herein. In certain embodiments, the microorganism is a bacterium. In certain embodiments, the microorganism is selected from the group consisting of Aceiobacter aceti, Achromobacter, Acidiphilium, Acinetobacter, Actinomadura, Actinoplanes, Aeropyrumpernix, Agrobacterium, Alcaligenes, Ananas comosus (M), Arthrobacter, Bacillus alcalophilus, Bacillus amyloliquefaciens, Bacillus brevis, Bacillus circulars, Bacillus clausii, Bacillus lentus, Bacillus lichenifirmis, Bacillus macerans, Bacillus stearothermophilus, Bacillus subtilis, Bifidobacterium, Brevibacillus brevis, Burkholderia cepacia, Candida cylindracea, Carica papaya (L). Cellulosimicrobium, Cephalosporium, Chaetomium erraticum, Chaetomium gracile, Clostridium, Clostridium butyricum, Clostridium acetobutylicum, Clostridium thermocellum, Corynebacterium (glutamicum), Corynebacterium efficiens, Escherichia coli, Enterococcus, Erwina chrysanthemi, Gliconobacter, Gluconacetobacter, Haloarcula, Humicola insolens, Kitasatospora setae, Klebsiella, Klebsiella oxytoca, Kocuria, Lactlactis, Lactobacillus, Lactobacillus fermentum, Lactobacillus sake, Lactococcus, Lactococcus lactis, Leuconostoc, Methylocystis, Methanolobus siciliae, Methanogenium organophilum, Methanobacterium bryantii, Microbacterium imperiale, Micrococcus lysodeikticus, Microlunatus, Mucorjavanicus, Mycobacterium, Myrothecium, Nitrobacter, Nitrosomonas, Nocardia, Papaya carica, Pediococcus, Pediococcus halophilus, Paracoccus pantotrophus, Propionibacterium, Pseudomonas, Pseudomonasfluorescens, Pseudomonas denitrificans, Pyrococcus, Pyrococcusfuriosus, Pyrococcus horikoshii, Rhizobium, Rhizomucor michei, Rhizomucor pusillus Lindt, Rhizopus, Rhizopus delemar, Rhizopus japonicus, Rhizopus niveus, Rhizopus oryzae, Rhizopus oligosporus, Rhodococcus, Sckroiina libertina, Sphingobacterium multivorum, Sphingobium, Sphingomonas, Streptococcus, Streptococcus thermophilus Y-1, Streptomyces, Streptomyces griseus, Streptomyces lividans, Streptomyces murinus, Streptomyces ruhiginosus, Streptomyces violaceoruber, Streptoverticillium mobaraense, Tetragenococcus, Thermus, Thiosphaera pantotropha, Trametes, Vibrio alginolyticus, Xanthomonas, Zymomonas, and Zymomonus mobilis. In certain embodiments, the microorganism is Escherichia coli (E. coli). In certain embodiments, the microorganism is Bacillus subtilis. In certain embodiments, the microorganism is Lactococcus lactis.

In certain embodiments, the E. coli is selected from the group consisting of Enterotoxigenic E. coli (ETEC), Enteropathogenic E. coli (EPEC), Enteroinvasive E. coli (EIEC), Enterohemorrhagic E. coli (EHEC). Uropathogenic E. coli (UPEC). Verotoxin-producing E. coli. E. coli O157:H7. E. coli O104:H4, E. coli 0121, E. coli O104:H21. E. coli Kl, and E. coli NC101. In certain embodiments, the E. coli is E. coli K12. In certain embodiments, the E. coli is E. coli B. In certain embodiments, the E. coli is E. coli C.

In certain embodiments, the E. coli is derived from a strain selected from the group consisting of NCTC 12757, NCTC 12779, NCTC 12790, NCTC 12796, NCTC 12811, ATCC 11229, ATCC 25922, ATCC 8739, DSM 30083, BC 5849, BC 8265, BC 8267, BC 8268, BC 8270, BC 8271, BC 8272, BC 8273, BC 8276, BC 8277, BC 8278, BC 8279, BC 8312, BC 8317, BC 8319, BC 8320, BC 8321, BC 8322, BC 8326, BC 8327, BC 8331, BC 8335, BC 8338, BC 8341, BC 8344, BC 8345, BC 8346, BC 8347, BC 8348, BC 8863, and BC 8864.

In certain embodiments, the E. coli is derived from a strain selected from the group consisting of BC 4734 (O26:H11), BC 4735 (O157:H—), BC 4736, BC 4737 (n.d.), BC 4738 (O157:H7), BC 4945 (O26:H—), BC 4946 (O157:H7), BC 4947 (O111:H—), BC 4948 (O157:H), BC 4949 (O5), BC 5579 (O157:H7), BC 5580 (O157:H7), BC 5582 (O3:H), BC 5643 (O2:H5), BC 5644 (O128), BC 5645 (O55:H—), BC 5646 (O69:H—), BC 5647 (O101:H9), BC 5648 (O103:H2), BC 5850 (O22:H8), BC 5851 (O55:H—), BC 5852 (O48:H21), BC 5853 (O26:H11), BC 5854 (O157:H7), BC 5855 (O157:H—), BC 5856 (O26:H—), BC 5857 (O103:H2), BC 5858 (O26:H11), BC 7832, BC 7833 (O raw form:H—), BC 7834 (ONT:H—), BC 7835 (O103:H2), BC 7836 (O57:H—), BC 7837 (ONT:H—), BC 7838, BC 7839 (O128:H2), BC 7840 (O157:H—), BC 7841 (O23:H—), BC 7842 (O157:H—), BC 7843. BC 7844 (O157:H—), BC 7845 (O103:H2), BC 7846 (O26:H11), BC 7847 (O145:H—), BC 7848 (O157:H—), BC 7849 (O156:H47), BC 7850, BC 7851 (O157:H—), BC 7852 (O157:H—), BC 7853 (O5:H—), BC 7854 (O157:H7), BC 7855 (O157:H7), BC 7856 (O26:H—), BC 7857, BC 7858, BC 7859 (ONT:H—), BC 7860 (O129:H—), BC 7861, BC 7862 (O103:H2), BC 7863, BC 7864 (O raw form:H—). BC 7865, BC 7866 (O26:H—), BC 7867 (O raw form:H—), BC 7868, BC 7869 (ONT:H—), BC 7870) (O113:H—), BC 7871 (ONT:H—), BC 7872 (ONT:H—), BC 7873, BC 7874 (O raw form:H—), BC 7875 (O157:H—), BC 7876 (O111:H—), BC 7877 (O146:H21), BC 7878 (O145:H—), BC 7879 (O22:H8), BC 7880 (O) raw form:H—), BC 7881 (O145:H—), BC 8275 (O157:H7), BC 8318 (O55:K—:H—), BC 8325 (O157:H7), BC 8332 (ONT), and BC 8333.

In certain embodiments, the E. coli is derived from a strain selected from the group consisting of BC 8246 (O152:K−:H—), BC 8247 (O124:K(72):H3), BC 8248 (O124), BC 8249 (O112), BC 8250) (O136:K (78):H—), BC 8251 (O124:H—), BC 8252 (O144:K—:H—), BC 8253 (O143:K:H—), BC 8254 (O143), BC 8255 (O112), BC 8256 (O28a.e), BC 8257 (O124:H—), BC 8258 (O143), BC 8259 (O167:K—:H5), BC 8260) (O128a. c.:H35), BC 8261 (O164), BC 8262 (O164:K—:H—), BC 8263 (O164), and BC 8264 (O124).

In certain embodiments, the E. coli is derived from a strain selected from the group consisting of BC 5581 (O78:H11), BC 5583 (O2:K1), BC 8221 (O118), BC 8222 (O148:H—), BC 8223 (O111). BC 8224 (O110:H—), BC 8225 (O148), BC 8226 (O118), BC 8227 (O25:H42), BC 8229 (O6), BC 8231 (O153:H45), BC 8232 (O9), BC 8233 (O148), BC 8234 (O128), BC 8235 (O118), BC 8237 (O111), BC 8238 (O110:H17), BC 8240 (O148), BC 8241 (O6H16), BC 8243 (O153), BC 8244 (O15:H—), BC 8245 ((20), BC 8269 (O125a.c:H—), BC 8313 (O6:H6), BC 8315 (O153:H—), BC 8329. BC 8334 (O118:H12), and BC 8339.

In certain embodiments, the E. coli is derived from a strain selected from the group consisting of BC 7567 (O86), BC 7568 (O128), BC 7571 (O114), BC 7572 (O119), BC 7573 (O125), BC 7574 (O124), BC 7576 (O127a), BC 7577 (O126), BC 7578 (O142), BC 7579 (O26), BC 7580 (OK26), BC 7581 (O142), BC 7582 (O55), BC 7583 (O158), BC 7584 (O—), BC 7585 (O—), BC 7586 (O—), BC 8330, BC 8550 (O26), BC 8551 (O55), BC 8552 (O158), BC 8553 (O26), BC 8554 (O158), BC 8555 (O86), BC 8556 (O128), BC 8557 (OK26), BC 8558 (O55), BC 8560 (O158), BC 8561 (O158), BC 8562 (O114), BC 8563 (O86), BC 8564 (O128), BC 8565 (O158), BC 8566 (O158), BC 8567 (O158). BC 8568 (O111), BC 8569 (O128), BC 8570 (O114), BC 8571 (O128), BC 8572 (O128), BC 8573 (O158), BC 8574 (O158), BC 8575 (O158), BC 8576 (O158), BC 8577 (O158), BC 8578 (O158), BC 8581 (O158), BC 8583 (O128), BC 8584 (O158), BC 8585 (O128), BC 8586 (O158), BC 8588 (O26), BC 8589 (O86), BC 8590 (O127), BC 8591 (O128), BC 8592 (O114), BC 8593 (O114), BC 8594 (O114), BC 8595 (O125), BC 8596 (O158), BC 8597 (O26), BC 8598 (O26), BC 8599 (O158), BC 8605 (O158), BC 8606 (O158), BC 8607 (O158), BC 8608 (O128), BC 8609 (O55), BC 8610 (O114), BC 8615 (O158), BC 8616 (O128), BC 8617 (O26), BC 8618 (O86), BC 8619, BC 8620, BC 8621, BC 8622, BC 8623, BC 8624 (O158), and BC 8625 (O158).

In certain embodiments, the B. subtilis is derived from Strain 168.

In certain embodiments, the L. lactis is derived from Strain A12.

In certain embodiments, the microorganism is a fungal cell. In certain embodiments, the fungal cell is selected from the group consisting of Aspergillus, Aspergillus nidulans, Aspargillus niger, Aspargillus oryze, Aspergillus melleus, Aspergillus pulverulentus, Aspergillus saitoi, Aspergillus sojea, Aspergillus terreus, Aspergillus pseudoterreus, Aspergillus usamii, Candida rugosa, Issatchenkia orientalis, Kluyveromyces, Kluyveromycesfragilis, Kluyveromyces lactis, Kluyveromyces marxianas, Penicillium, Penicillium camemberti, Penicillium citrinum, Penicillium emersonii, Penicillium roqueforti, Penicillum lilactinum, Penicillum multicolor, Rhodosporidium toruloides, Saccharomyces cerevisiae, Schizosaccharomyces pombe, Trichoderma, Trichoderma longibrachiatum, Trichoderma reesei, Trichoderma viride, Trichosporon penicillaium, Yarrowia lipolytica, and Zygosaccharomyces rouxii.

In certain embodiments, the microorganism is a yeast cell. In certain embodiments, the yeast cell is Saccharomyces cerevisiae.

### 2.4. Exemplary Microorganisms

In certain embodiments, the present disclosure provides a recombinant microorganism comprising an increased production of psicose as compared to a naturally occurring microorganism. In certain embodiments, the recombinant microorganism comprises an exogenous epimerase and an exogenous phosphatase. In certain embodiments, the exogenous epimerase is an allulose-6-phosphate 3-epimerase (AlsE). In certain embodiments, the exogenous phosphatase is hexitol phosphatase B (HxpB). In certain embodiments, the recombinant microorganism is a bacterium. In certain embodiments, the bacterium is E. coli.

In certain embodiments, the present disclosure provides a recombinant microorganism comprising an increased production of psicose as compared to a naturally occurring microorganism. In certain embodiments, the recombinant microorganism comprises an exogenous epimerase, an exogenous phosphatase, and a deletion of one, two, three or four (4) genes. In certain embodiments, the exogenous epimerase is an allulose-6-phosphate 3-epimerase (AlsE). In certain embodiments, the exogenous phosphatase is hexitol phosphatase B (HxpB). In certain embodiments, the four deleted genes are glucose-6-phosphate 1-dehydrogenase, phosphofructokinase-1, allose-6-phosphate isomerase, and mannose-6-phosphate isomerase. In certain embodiments, the recombinant microorganism is a bacterium. In certain embodiments, the bacterium is E. coli.

In certain embodiments, the present disclosure provides a microorganism comprising a recombinant polynucleotide, wherein the microorganism comprises an increased production of psicose as compared to a naturally occurring microorganism. In certain embodiments, the recombinant polynucleotide comprises a nucleotide sequence encoding an exogenous epimerase and an exogenous phosphatase. In certain embodiments, the exogenous epimerase is an allulose-6-phosphate 3-epimerase (AlsE). In certain embodiments, the exogenous phosphatase is hexitol phosphatase B (HxpB). In certain embodiments, the recombinant microorganism is a bacterium. In certain embodiments, the bacterium is E. coli.

In certain embodiments, the present disclosure provides a microorganism comprising a recombinant polynucleotide, wherein the microorganism comprises an increased production of psicose as compared to a naturally occurring microorganism. In certain embodiments, the recombinant polynucleotide comprises a nucleotide sequence encoding an exogenous epimerase and an exogenous phosphatase. In certain embodiments, the exogenous epimerase is an allulose-6-phosphate 3-epimerase (AlsE). In certain embodiments, the exogenous phosphatase is hexitol phosphatase B (HxpB). In certain embodiments, the microorganism further comprises a deletion of a first gene. In certain embodiments, the first gene is glucose-6-phosphate 1-dehydrogenase. In certain embodiments, the microorganism further comprises a deletion of a second gene. In certain embodiments, the second gene is phosphofructokinase-1. In certain embodiments, the microorganism further comprises a deletion of a third gene. In certain embodiments, the third gene is allose-6-phosphate isomerase. In certain embodiments, the microorganism further comprises a deletion of a fourth gene. In certain embodiments, the fourth gene is mannose-6-phosphate isomerase. In certain embodiments, the recombinant microorganism is a bacterium. In certain embodiments, the bacterium is E. coli. In certain embodiments, the bacterium is B. subtilis. In certain embodiments, the bacterium is L. lactis.

In certain embodiments, the present disclosure provides a recombinant microorganism comprising an increased production of psicose as compared to a naturally occurring microorganism. In certain embodiments, the recombinant microorganism comprises an exogenous epimerase, an exogenous phosphatase, an exogenous nuclease. a sgRNA, and a deletion of four (4) genes. In certain embodiments, the exogenous epimerase is an allulose-6-phosphate 3-epimerase (AlsE). In certain embodiments, the exogenous phosphatase is hexitol phosphatase B (HxpB). In certain embodiments, the exogenous nuclease is dCas9. In certain embodiments, the four deleted genes are zwf. pfkA, RpiB. and ManA. In certain embodiments, the sgRNA targets pfkB. In certain embodiments, the recombinant microorganism is a bacterium. In certain embodiments, the bacterium is E. coli. In certain embodiments, the bacterium is B. subtilis. In certain embodiments, the bacterium is L. lactis.

In certain embodiments, the present disclosure provides a microorganism comprising a recombinant polynucleotide, wherein the microorganism comprises an increased production of psicose as compared to a naturally occurring microorganism. In certain embodiments, the recombinant polynucleotide comprises a nucleotide sequence encoding an exogenous epimerase, a nucleotide sequence encoding an exogenous phosphatase. and a nucleotide sequence encoding a nuclease. In certain embodiments, the exogenous epimerase is an allulose-6-phosphate 3-epimerase (AlsE). In certain embodiments, the exogenous phosphatase is hexitol phosphatase B (HxpB). In certain embodiments, the exogenous nuclease is dCas9. In certain embodiments, the microorganism further comprises a deletion of a first gene. In certain embodiments, the first gene is zwf. In certain embodiments, the microorganism further comprises a deletion of a second gene. In certain embodiments, the second gene is pfkA. In certain embodiments, the microorganism further comprises a deletion of a third gene. In certain embodiments, the third gene is rpiB. In certain embodiments, the microorganism further comprises a deletion of a fourth gene. In certain embodiments, the fourth gene is manA. In certain embodiments, the microorganism further comprises a sgRNA. In certain embodiments, the sgRNA targets pfkB. In certain embodiments, the recombinant microorganism is a bacterium. In certain embodiments, the bacterium is E. coli. In certain embodiments, the bacterium is B. subtilis. In certain embodiments, the bacterium is L. lactis.

### 3. Methods for Producing and Generating Microorganisms Psicose

The present disclosure also provides methods for preparing and/or generating any of the microorganisms disclosed herein. Many recombinant techniques commonly known in the art may be used to introduce one or more recombinant polynucleotides of the present disclosure into a microorganism, including without limitation protoplast fusion, transfection, transformation, conjugation, and transduction. These techniques include conventional molecular biology techniques (e.g., recombinant techniques), microbiology, cell biology, and biochemistry, which are within the skill of the art. Additional information on these techniques can be found in Molecular Cloning: A Laboratory Manual, second edition (Sambrook et al., 1989): Oligonucleotide Synthesis (Gait, ed., 1984); Animal Cell Culture (Freshney. ed., 1987): Gene Transfer Vectors for Mammalian Cells (Miller & Calos, eds., 1987); Current Protocols in Molecular Biology (Ausubel et al., eds., 1987): PCR: The Polymerase Chain Reaction, (Mullis et al., eds., 1994); and Current Protocols in Immunology (Coligan et al., eds., 1991).

### 3.1. Recombinant Polynucleotides

In certain embodiments, the recombinant polynucleotides disclosed herein can be stably integrated into a microorganism chromosome. In certain embodiments, the recombinant polynucleotides disclosed herein are stably integrated into a microorganism chromosome using homologous recombination. transposition-based chromosomal integration, recombinase-mediated cassette exchange (RMCE: e.g., using a Cre-lox system), or an integrating plasmid (e.g., a yeast integrating plasmid). A variety of integration techniques suitable for a range of microorganisms are known in the art (see, e.g., Griffiths, A. J. F., Miller, J. H., Suzuki. D. T. et al. An Introduction to Genetic Analysis. 7^{th} ed. New York: W.H. Freeman: 2000). In certain embodiments, the recombinant polynucleotides disclosed herein are maintained in a recombinant microorganism of the present disclosure on an extra-chromosomal plasmid (e.g., an expression plasmid or vector). A variety of extra-chromosomal plasmids suitable for a range of microorganisms are known in the art, including without limitation replicating plasmids (e.g., yeast replicating plasmids that include an autonomously replicating sequence, ARS), centromere plasmids (e.g., yeast centromere plasmids that include an autonomously replicating sequence, CEN), episomal plasmids (e.g., 2-p.m plasmids), and/or artificial chromosomes (e.g., yeast artificial chromosomes, YACs, or bacterial artificial chromosomes. BACs).

### 3.1.1. Vectors

In certain embodiments, the present disclosure provides vectors including the nucleotide sequences disclosed herein. As used herein, the term “vector” refers to a polynucleotide construct designed to introduce nucleic acids into one or more microorganisms. Vectors can include, but without any limitation, cloning vectors, expression vectors, shuttle vectors, plasmids, and cassettes. As used herein, the term “plasmid” refers to a circular double-stranded DNA construct used as a cloning and/or expression vector. In certain embodiments, plasmids can be extrachromosomal self-replicating genetic elements (e.g., episomal plasmids) when introduced into a microorganism. In certain embodiments, plasmids can integrate into a microorganism chromosome. In certain embodiments, vectors can direct the expression of coding regions to which they are operatively linked, e.g. “expression vectors.” These expression vectors allow the expression of exogenous polynucleotides and/or polypeptides in microorganisms. In certain embodiments, the vectors allow the integration of one or more polynucleotides into the genome of a microorganism.

In certain embodiments, a vector disclosed herein includes a promoter. In certain embodiments, the vector is a bacterial or prokaryotic expression vector. In certain embodiments, the vector is a yeast or fungal cell expression vector.

In certain embodiments, a vector discloses herein comprises nucleotide sequences in a single operon.

### 3.1.2. Promoters

In certain non-limiting embodiments, the recombinant polynucleotides disclosed herein include a control sequence, an enhancer, or a promoter. For example, but without any limitation, a nucleotide sequence encoding the alsE gene and/or hpxB gene can be operably linked to a control sequence, enhancer, or promoter.

As used herein, the term “promoter” refers to any nucleotide sequence that regulates the initiation of transcription for a particular coding sequence under its control. Biologically, promoters are not transcribed but coordinate the assembly of components that initiate the transcription of other nucleotide sequences. In addition, promoters can limit this assembly and subsequent transcription to specific prerequisite conditions. For example, but without any limitation, a promoter can allow transcription in response to one or more environmental, temporal, or developmental stimuli. Bacterial and fungal cells possess a multitude of proteins that sense external or internal conditions and initiate signaling cascades ending in the binding of proteins to specific promoters and subsequent initiation of transcription of nucleic acid(s) under the control of the promoters. In certain embodiments, the promoter is endogenous. In certain embodiments, the promoter is exogenous. In certain embodiments, the promoter is artificially designed for expression in a particular species.

In certain embodiments, the promoter is a constitutive promoter. A constitutive promoter is a promoter that drives the expression of a nucleotide sequence continuously and without interruption in response to internal or external stimuli. Constitutive promoters are commonly used in recombinant engineering to ensure the continuous expression of a desired nucleotide sequence. Constitutive promoters result in a robust amount of nucleic acid expression, and, as such, are used in many recombinant engineering applications to achieve a high level of recombinant protein and enzymatic activity. Non-limiting examples of constitutive promoters encompassed by the present disclosure include E. coli promoters P_{spc}, P_{bla}, P_{RNAI}, P_{RNAII}, P₁ and P₂ from rrnB, and the lambda phage promoter PL (Liang, S. T. et al. JMoi. Biol. 292 (1): 19-37 (1999)). In some embodiments, the promoter is active in the stationary phase of the microorganism. Exemplary stationary phase promoters can be found in, e.g., Shimada, et al., JOURNAL OF BACTERIOLOGY, November 2004, p. 7112-7122; Pletnev at el., ACTA NATURAE|VOL. 7 No 4 (27) 2015.

In certain embodiments, the promoter is an inducible promoter. An inducible promoter is a promoter that drives the expression of a nucleotide sequence in response to a stimulus. An inducible promoter drives sustained expression upon exposure to a specific stimulus (e.g., IPTG). In certain embodiments, an inducible promoter drives a graded level of expression correlated with the amount of stimulus. Non-limiting examples of stimuli for inducible promoters include heat shock, exogenous compounds or a lack thereof (e.g., a sugar, metal, drug, or phosphate), salts or osmotic shock, oxygen, and biological stimuli (e.g., a growth factor or pheromone). Non-limiting examples of inducible promoters include the E. coli promoters P_{lac}, P_{taq}), P_{tac}, P_{T7}, P_{BAD}, and P_{Lacuv}.

In certain embodiments, the recombinant polynucleotide can include multiple promoters. In certain embodiments, the multiple promoters can be the same. For example, but without any limitation, the recombinant polynucleotide can include a nucleotide sequence encoding the aslE gene operably linked to a first promoter and a nucleotide sequence encoding the hpxB gene operably linked to a second promoter, wherein the first and second promoter is the same. In certain embodiments, the multiple promoters can be different. For example, but without any limitation, the recombinant polynucleotide can include a nucleotide sequence encoding the alsE gene operably linked to a first promoter and a nucleotide sequence encoding the hpxB gene operably linked to a second promoter, wherein the first and second promoter are different. In certain embodiments, the promoter is a P_{LlacO1} promoter. In certain embodiments, the P_{LlacO1} promoter comprises the nucleotide sequence set forth in SEQ ID NO: 42. In certain embodiments, the P_{LlacO1} promoter consists of the nucleotide sequence set forth in SEQ ID NO: 42. The P_{LlacO1} promoter is a hybrid regulatory region including the promoter PL of phage lambda with the CI binding sites replaced with lacO₁. The hybrid design allows for a strong promotion that can be repressed by LacI, the Lac inhibitor (i.e., repressor) or induced by IPTG.

In certain embodiments, the promoter is a P_{LtetO1} promoter. In certain embodiments, the P_{LTETO1} promoter comprises the nucleotide sequence set forth in SEQ ID NO: 43. In certain embodiments, the P_{LTETO1} promoter consists of the nucleotide sequence set forth in SEQ ID NO: 43.

In certain embodiments, the promoter is a P_{T7} promoter. In certain embodiments, the P_{T7} promoter comprises the nucleotide sequence set forth in SEQ ID NO: 44. In certain embodiments, the P_{T7} promoter consists of the nucleotide sequence set forth in SEQ ID NO: 44.

In certain embodiments, the promoter is a Pₜₑₜ promoter. In certain embodiments, the Pₜₑₜ promoter comprises the nucleotide sequence set forth in SEQ ID NO: 45. In certain embodiments, the Pₜₑₜ promoter consists of the nucleotide sequence set forth in SEQ ID NO: 45.

In certain embodiments, the promoter is a P_{gadB} promoter. In certain embodiments, the P_{gadB} promoter comprises the nucleotide sequence set forth in SEQ ID NO: 46. In certain embodiments, the P_{gadB} promoter consists of the nucleotide sequence set forth in SEQ ID NO: 46.

P_{Llaco1} promoter nucleotide sequence:
(SEQ ID NO: 42)
AATTGTGAGCGGATAACAATTGACATTGTGAGCGGATAACAAGATACTGAGCACATCAGCAGGACGC
ACTGACCGAATTCATTAAAGAGGAGAAAAGATATACC
P_{LtetO1} promoter nucleotide sequence:
(SEQ ID NO: 43)
tccctatcagtgatagagattgacatccctatcagtgatagagatactgagcacatcagcaggacgc
actgaccgaattcattaaagaggagaaaggtacc
P_{T7} promoter nucleotide sequence:
(SEQ ID NO: 44)
taatacgactcactataggggaattgtgagcggataacaattcccctctagaaataattttgtttaa
ctttaagaaggagatatacc
Pₜₑₜ promoter nucleotide sequence:
(SEQ ID NO: 45)
gttgacactctatcgttgatagagttattttaccactccctatcagtgatagagaaaagaattcaaa
agatctaaagaggagaaaggatct
P_{gadB} promoter nucleotide sequence:
(SEQ ID NO: 46)
GTAATAATTTTATAAATGCGTTCAAAATAATAATCAAGTACTAATAGTGATATTTTAAGGTCTGATT
TTTACGTGATAATTCAGGAGACACAGAATGCGCATAAAAATAACAGCATAAAACACCTTACCACCAC
CCAAGAATTTCATATTGTATTGTTTTTCAATGAAAAAATATTATTCGCGTAATATCTCACGATAAAT
AACATTAGGATTTTGTTATTTAAACACGAGTCCTTTGCACTTGCTTACTTTATCGATAAATCCTACT
TTTTTAATGCGATCCAATCATTTTAAGGAGTTTAAAATGGATAAGAAGCAAGTGAATTCATTAAAGA
GGAGAAAAGATATACC

In certain embodiments, the promoter is a stationary phase promoter. As used herein, the term “stationary phase promoter” refers to a promoter upstream of a gene that is transcribed during the stationary phase of a microorganism growth. The life cycle of an E. coli culture includes 5 distinct phases: lag, logarithmic, stationary, death, and long-term stationary phase. The lag phase occurs when cells are inoculated into media and adjust their metabolic processes according to their new environment. The cells will then rapidly grow and divide, entering the logarithmic phase. It is at this time that enzymes related to central carbon metabolism are most important, and the transcription of corresponding genes will be upregulated. Once the cells sense environmental stressors such as scarcity of media nutrients, their growth and division slows, and the culture enters the stationary phase. The use of a stationary phase promoter prevents the production pathway from competing with central carbon metabolism for carbon flux during the logarithmic phase of growth, a time when cells need carbon to rigorously grow and divide.

In certain embodiments, the stationary phase promoter is P_{gadB}. In certain embodiments, the P_{gadB} promoter comprises the nucleotide sequence set forth in SEQ ID NO: 46 or SEQ ID NO: 53. In certain embodiments, the P_{gadB} promoter consists of the nucleotide sequence set forth in SEQ ID NO: 53.

In certain embodiments, the stationary phase promoter is P_{cbpA2}. In certain embodiments, the P_{cbpA2} promoter comprises the nucleotide sequence set forth in SEQ ID NO: 54. In certain embodiments, the P_{cbpA2} promoter consists of the nucleotide sequence set forth in SEQ ID NO: 54.

In certain embodiments, the stationary phase promoter is P_{thfA4}. In certain embodiments, the P_{ihfA4} promoter comprises the nucleotide sequence set forth in SEQ ID NO: 55. In certain embodiments, the P_{infA4} promoter consists of the nucleotide sequence set forth in SEQ ID NO: 55.

In certain embodiments, the stationary phase promoter is P_{dps}. In certain embodiments, the P_{dps} promoter comprises the nucleotide sequence set forth in SEQ ID NO: 56. In certain embodiments, the P_{dps} promoter consists of the nucleotide sequence set forth in SEQ ID NO: 56.

(SEQ ID NO: 53)
GTAATAATTTTATAAATGCGTTCAAAATAATAATCAAGTACTAATAGTGATATTTTAAGGTCTGATT
TTTACGTGATAATTCAGGAGACACAGAATGCGCATAAAAATAACAGCATAAAACACCTTACCACCAC
CCAAGAATTTCATATTGTATTGTTTTTCAATGAAAAAATATTATTCGCGTAATATCTCACGATAAAT
AACATTAGGATTTTGTTATTTAAACACGAGTCCTTTGCACTTGCTTACTTTATCGATAAATCCTACT
TTTTTAATGCGATCCAATCATTTTAAGGAGTTTAAAATGGATAAGAAGCAAGTCGAATTCATTAAAG
AGGAGAAAGGTACCATG
(SEQ ID NO: 54)
TTTGCAGTGCAACTAATTCCATGTATATTACTACCCATATATAGCGTCTATAAAATTTAATAAATAA
TGACGCCCTAGTTAAACTTAAAGTGCCTGGTTCAACTATCAAAAATCGCTCACCCTTTTTCACCTGT
TTAAAATATGTTCAGCAACCCATCTTGATGGCGACCTCCTCTCCGCGATGATTTCAATAACATATTC
TGTGTTGGCATATGAAATTTTGAGGATTACCCTACACTTATAGGAGTTACCTTACAGGGGTTCCTTC
AATTTGTGTTGATTTACGCGAGATAACGCTCGAATTCATTAAAGAGGAGAAAGGTACCATG
(SEQ ID NO: 55)
TATCCGAATGTAAGAAAGTTGGCGTAAATCAGGTAGTTGGCGTAAACTTATTTGACGTGTACCGCGG
TAAGGGTGTTGCGGAGGGGTATAAGAGCCTCGCCATAAGCCTGATCCTGCAAGATACCAGCCGTACA
CTCGAAGAAGAGGAGATTGCCGCTACCGTCGCCAAATGTGTAGAGGCATTAAAAGAGCGATTCCAGG
CATCATTGAGGGATTGAACCTCGAATTCATTAAAGAGGAGAAAGGTACCATG
(SEQ ID NO: 56)
TCATTGAATCTTTATTAGTTTTGTTTTTCACGCTTGTTACCACTATTAGTGTGATAGGAACAGCCAG
AATAGCGGAACACATAGCCGGTGCTATACTTAATCTCGTTAATTACTGGGACATAACATCAAGAGGA
TATGAAATTCGAATTCATTAAAGAGGAGAAAGGTACCATG

### 3.1.3. Genetic Markers

In certain embodiments, the presently disclosed recombinant polynucleotides include genetic markers. These genetic markers allow the selection of microorganisms that have one or more desired polynucleotides (e.g., recombinant polynucleotides). In certain embodiments, the genetic marker is an antibiotic resistance marker selected from the group consisting of Apramycin resistance, Ampicillin resistance, Kanamycin resistance, Spectinomycin resistance, Tetracyclin resistance, Neomycin resistance, Chloramphenicol resistance, Gentamycin resistance, Erythromycin resistance, Carbenicillin resistance, Actinomycin D resistance, Neomycin resistance, Polymyxin resistance, Zeocin resistance, and Streptomycin resistance. In certain embodiments, the genetic marker includes a coding sequence of an antibiotic resistance protein (e.g., a beta-lactamase for certain Ampicillin resistance markers) and a promoter or enhancer element that drives the expression of the coding sequence in a microorganism of the present disclosure. In certain embodiments, a microorganism of the present disclosure is grown under conditions in which an antibiotic resistance marker is expressed and confers resistance to the microorganism, thereby selected for the microorganism with successful integration of the marker. In certain embodiments, the genetic marker is an auxotrophic marker. In certain embodiments, the auxotrophic marker is a gene involved in vitamin, amino acid, fatty acid synthesis, or carbohydrate metabolism. In certain embodiments, the auxotrophic marker is a gene for synthesizing amino acid. In certain embodiments, the auxotrophic marker is a gene for synthesizing glycine, alanine, valine, leucine, isoleucine, proline, phenylalanine, tyrosine, tryptophan, serine, threonine, cysteine, methionine, asparagine, glutamine, lysine, arginine, histidine, aspartate or glutamate. In certain embodiments, the auxotrophic marker is a gene for synthesizing adenosine, biotin, thiamine, leucine, glucose, lactose, or maltose. In certain embodiments, a microorganism of the present disclosure is grown under conditions in which an auxotrophic resistance marker is expressed in an environment or medium lacking the corresponding nutrient and confers growth to the microorganism (lacking an endogenous ability to produce the nutrient), thereby selected for the microorganism with successful integration of the marker.

### 3.2. Deletions and Reduced Expression of Genes

In certain embodiments, the present disclosure also provides methods to introduce a deletion of any of the genes or enzymes disclosed herein. These deletions can be generated by any suitable gene-editing methods. In certain embodiments, the deletion is generated by a method comprising homologous recombination, a Zinc finger nuclease, a meganuclease, a Transcription activator-like effector nuclease (TALEN), a Clustered regularly-interspaced short palindromic repeats (CRISPR) system, or a combination thereof.

In certain embodiments, the deletion is generated by a CRISPR system. Clustered regularly-interspaced short palindromic repeats (CRISPR) system is a genome-editing tool discovered in prokaryotic cells. When utilized for genome editing, the system includes Cas9 (a protein able to modify DNA utilizing crRNA as its guide), CRISPR RNA (crRNA, which contains the RNA used by Cas9) to guide it to the correct section of host DNA along with a region that binds to tracrRNA (generally in a hairpin loop form) forming an active complex with Cas9), trans-activating crRNA (tracrRNA, binds to crRNA and forms an active complex with Cas9), and an optional section of DNA repair template (DNA that guides the cellular repair process allowing insertion of a specific DNA sequence). Multiple crRNA's and the tracrRNA can be packaged together to form a single-guide RNA (sgRNA). This sgRNA can be joined together with the Cas) gene and made into a plasmid in order to be transfected into cells. In certain embodiments, the CRISPR system comprises base editors. In certain embodiments, the CRISPR system comprises transposases/recombinases. In certain embodiments, the CRISPR system comprises prime editors. In certain embodiments, the CRISPR system comprises an epigenetic modulator. In certain embodiments, the CRISPR system comprises a CRISPRoff system. Additional details on the CRISPR systems of the present disclosure can be found in Anzalone et al., Nature biotechnology 38.7 (2020): 824-844 and in Nuñez et al., Cell 184.9 (2021): 2503-2519, and Jiang et al., Appl Environ Microbiol. 2015 April; 81(7):2506-14, the contents of each of which are incorporated by reference in their entireties.

In certain embodiments, the deletion is generated by a zinc-finger nuclease. A zinc-finger nuclease (ZFN) is an artificial restriction enzyme, which is generated by combining a zinc finger DNA-binding domain with a DNA-cleavage domain. A zinc finger domain can be engineered to target specific DNA sequences and allows a zinc-finger nuclease to target desired sequences within genomes. The DNA-binding domains of individual ZFNs typically contain a plurality of individual zinc finger repeats and can each recognize a plurality of base pairs. The most common method to generate a new zinc-finger domain is to combine smaller zinc-finger “modules” of known specificity. The most common cleavage domain in ZFNs is the non-specific cleavage domain from the Type IIs restriction endonuclease FokI.

In certain embodiments, the deletion is generated by a TALEN system. Transcription activator-like effector nucleases (TALEN) are restriction enzymes that can be engineered to cut specific sequences of DNA TALEN system operates on almost the same principle as 7.FNs. They are generated by combining a transcription activator-like effectors DNA-binding domain with a DNA cleavage domain. Transcription activator-like effectors (TALEs) are composed of 33-34 amino acid repeating motifs with two variable positions that have a strong recognition for specific nucleotides. By assembling arrays of these TALEs, the TALE DNA-binding domain can be engineered to bind desired DNA sequence, and thereby guide the nuclease to cut at specific locations in genome.

In certain embodiments, the deletion is generated by a meganuclease. A meganuclease is an endodeoxyribonuclease that recognizes a double-stranded DNA site of approx. 12 to approx. 40 base pairs that occur only once in a genome. Meganucleases are some of the most specific naturally occurring restriction enzymes. Meganucleases are also defined as molecular DNA scissors since they can replace, eliminate or modify sequences in a highly targeted way. Protein engineering allows the modification of their recognition sequence and the targeted sequence.

In certain embodiments, the present disclosure also provides methods to reduce the expression of any of the genes or enzymes disclosed herein. In certain embodiments, the reduced expression of genes and enzymes disclosed herein comprises using oligonucleotides that have complementary sequences to the mRNA of the genes disclosed herein (e.g., zwf, manA, rpiB, pfkA, pfkB, etc.). Non-limiting examples of these oligonucleotides include small interference RNA (siRNA), short hairpin RNA (shRNA), and microRNA (miRNA). In certain embodiments, these oligonucleotides can be at least about 75%, at least about 80%, at least about 85%, at least about 90%, at least about 95%, at least about 96%, at least about 97%, at least about 98%, at least about 99% identical to at least a portion of a zwf mRNA sequence. In certain embodiments, these oligonucleotides can be identical to at least a portion of a zwf mRNA sequence. In certain embodiments, these oligonucleotides can be at least about 75%, at least about 80%, at least about 85%, at least about 90%, at least about 95%, at least about 96%, at least about 97%, at least about 98%, at least about 99% identical to at least a portion of a pfkA mRNA sequence. In certain embodiments, these oligonucleotides can be identical to at least a portion of a pfkA mRNA sequence. In certain embodiments, these oligonucleotides can be at least about 75%, at least about 80%, at least about 85%, at least about 90%, at least about 95%, at least about 96%, at least about 97%, at least about 98%, at least about 99% identical to at least a portion of a pfkB mRNA sequence. In certain embodiments, these oligonucleotides can be identical to at least a portion of a pfkB mRNA sequence. In certain embodiments, these oligonucleotides can be at least about 75%, at least about 80%, at least about 85%, at least about 90%, at least about 95%, at least about 96%, at least about 97%, at least about 98%, at least about 99% identical to at least a portion of a rpiB mRNA sequence. In certain embodiments, these oligonucleotides can be identical to at least a portion of a rpiB mRNA sequence. In certain embodiments, these oligonucleotides can be at least about 75%, at least about 80%, at least about 85%, at least about 90%, at least about 95%, at least about 96%, at least about 97%, at least about 98%, at least about 99% identical to at least a portion of a manA mRNA sequence. In certain embodiments, these oligonucleotides can be identical to at least a portion of a manA mRNA sequence. In certain embodiments, antisense nucleic acid, shRNA, miRNA, or siRNA molecules can include DNA or atypical or non-naturally occurring residues, for example, but not limited to, phosphorothioate residues.

In some embodiments, reduction of expression of genes and enzymes disclosed herein can comprise use of CRISPR, which can mutate the coding sequence or promoter, to lower or remove expression of the gene product, or CRISPRi can be targeted to the genes disclosed herein, thereby reducing expression of one of more of the genes. See, e.g., Arroya-Olarte, et al., Microorganisms, 2021 April; 9(4): 844. Zhang et al., Front. Microbiol. (31 Mar. 2021).

In certain embodiments, the reduced expression of genes and enzymes disclosed herein comprises using a CRISPRi system. CRISPRi systems silence genes at the transcriptional level and can have fewer sequence-specific off-target effects than RNAi. In certain embodiments, the CRISPRi system includes a catalytically dead Cas9 (dCas9). dCas9 is a programmable transcription factor that can be targeted to promoters through sgRNAs, where it can function as repressor. In certain embodiments, dCas9 comprises an amino acid sequence that is at least about 80%, at least about 85%, at least about 90%, at least about 95%, at least about 96%, at least about 97%, at least about 98%, at least about 99%, or at least about 100% identical to the amino acid sequence set forth in SEQ ID NO:

57. In certain embodiments, dCas9 comprises the amino acid sequence set forth in SEQ ID NO: 57. In certain embodiments, dCas9 consists of the amino acid sequence set forth in SEQ ID NO: 57. SEQ ID NO: 57 is provided below:

[SEQ ID NO: 57]
MDKKYSIGLAIGTNSVGWAVITDEYKVPSKKEKVIGNTDRHSIKKNLIGALLFDSGETAEATRLKRT
ARRRYTRRKNRICYLQEIFSNEMAKVDDSFFHRLEESFIVEEDKKHERHPIFGNIVDEVAYHEKYPT
IYHLRKKLVDSTDKADLRLIYLALAHMIKERGHFLIEGDLNPDNSDVDKLFIQLVQTYNQLFEENPI
NASGVDAKAILSARLSKSRRLENLIAQLPGEKKNGLFGNLIALSLGLTPNEKSNEDLAEDAKLQLSK
DTYDDDIDNLLAQIGDQYADLFLAAKNISDAILLSDILRVNTEITKAPISASMIKRYDEHHQDLTIL
KALVRQQLPEKYKEIFFDQSKNGYAGYIDGGASQEEFYKFIKPILEKMDGTEELLVKINREDLLRKQ
RTFDNGSIPHQIHIGELHAILRRQEDFYPFLKDNREKIEKILTFRIPYYVGPLARGNSRFAWMTRKS
EETITPWNFEEVVDKGASAQSFIERMINEDKNLPNEKVIPKHSLLYEYFTVYNELIKVKYVTEGMRK
PAFISGEQKKAIVDLLFKINRKVTVKQLKEDYFKKIECFDSVEISGVEDRFNASLGTYHDLLKIIKD
KDELDNEENEDILEDIVLTLTLFEDREMIEERIKTYAHLEDDKVMKQLKRRRYTGWGRLSRKLINGI
RDKQSGKTILDFIKSDGFANRNFMQLIHDDSLTFKEDIQKAQVSGQGDSLHEHIANLAGSPAIKKGI
LQTVKVVDELVKVMGRAKPENIVIEMARENQTTQKGQKNSREPMKRIEEGIKELGSQILKEHPVENT
QLQNEKLYLYYLQNGRDMYVDQELDINRLSDYDVDAIVPQSFLKDDSIDNKVLTRSDKNRGKSDNVP
SEEVVKKMKNYWRQLLNAKLITQRKFDNLIKAERGGISELDKAGFIKRQLVETRQITKHVAQILDSR
MNTKYDENDKLIREVKVITLKSKLVSDERKDFQFYKVREINNYHHAHDAYLNAVVGTALIKKYPKLE
SEFVYGDYKVYDVRKMIAKSEQEIGKATAKYFFYSNIMNFFKTEITLANGEIRKRPLIETNGETGEI
VWDKGRDFATVRKVISMPQVNIVKKTEVQTGGFSKESILPKRNSDKLIARKKDWDPKKYGGEDSPTV
AYSVLVVAKVEKGKSKKLKSVKELLGITIMERSSFEKNPIDELEAKGYKEVKKDLIIKLPKYSLFEL
ENGRKRMLASAGELQKGNELALPSKYVNFLYLASHYEKLKGSPEDNEQKQLFVEQHKHYLDEIIEQI
SEFSKRVILADANLDKVISAYNKHRDKPIREQAENIIHLFTLTNIGAPAAFKYEDTTIDRKRYTSTK
EVLDATLIHOSITGLYETRIDLSQLGGD

In certain embodiments, the dCas9 is regulated by a promoter (e.g., described in Section 3.1.2). In certain embodiments, the promoter is an inducible promoter. In certain embodiments, the promoter is a stationary phase promoter. In certain embodiments, the CRISPRi system includes a small guide RNAs (sgRNA). In certain embodiments, the sgRNA of the CRISPRi system targets a gene encoding an enzyme of a competing pathway. For example, but without any limitation, the sgRNA can target the zwf gene, the pgm gene, the pfkA gene, the pfkB gene, the ManA gene, or the RpiB gene. In certain embodiments, the sgRNA can target any portion of a gene. For example, but without any limitation, the sgRNA can target a promoter, an operator, or a sequence encoding a protein.

In certain embodiments, the CRISPRi system includes a dCas9 and a sgRNA. In certain embodiments, the dCas9 is regulated by an inducible promoter. In certain embodiments, the inducible promoter is Pₜₑₜ. In certain embodiments, the sgRNA targets a pfkB gene. In certain embodiments, the sgRNA targets a promoter of the pfkB gene.

### 3.3. Transformation and Gene Editing

In certain embodiments, the present disclosure provides the use of transformation of the plasmids and vectors disclosed herein. Vectors and plasmids disclosed herein can be transformed into cells through any known system in the art. For example, but without any limitation, the presently disclosed microorganisms can be transformed by particle bombardment, chemical transformation, Agrobacterium transformation, nano-spike transformation, electroporation, and virus transformation.

In certain embodiments, the vectors of the present disclosure may be introduced into the microorganisms using a variety of techniques, including transformation, transfection, transduction, viral infection, gene guns, or Ti-mediated gene transfer. Non-limiting examples of these methods include calcium phosphate transfection, DEAE-Dextran mediated transfection, lipofection, and electroporation (see, e.g., Davis, L., Dibner, M., Battey, I., 1986 “Basic Methods in Molecular Biology”; Gietz et al., Nucleic Acids Res. 27:69-74 (1992); Ito et al., J. Bacterol. 153:163-168 (1983); and Becker and Guarente, Methods in Enzymology 194:182-187 (1991)). In certain embodiments, transformed microorganisms are referred to as recombinant microorganisms.

In certain embodiments, the present disclosure provides methods for introducing exogenous proteins (e.g., nuclease), RNA (e.g., gRNA), and DNA (e.g., a recombinant polynucleotide disclosed herein) into the microorganism. Various methods for achieving this have been described previously including direct transfection of protein and nucleotide sequence or DNA transformation followed by intracellular expression of RNA and protein (see, e.g., Dicarlo, J. E. et al. “Genome engineering in Saccharomyces cerevisiae using CRISPR-Cas systems.” Nucleic Acids Res (2013). doi: 10.1093/nar/gkt135: Ren, Z. J., Baumann, R. G. & Black, L. W. “Cloning of linear DNAs in vivo by overexpressed T4 DNA ligase: construction of a T4 phage hoc gene display vector.” Gene 195, 303-311 (1997); Lin, S., Staahl. B. T., Alla, R. K. & Doudna, J. A. “Enhanced homology-directed human genome engineering by controlled timing of CRISPR/Cas9 delivery.” Elife 3, e04766 (2014)).

### 3.4. Recombination Systems

In certain embodiments, the present disclosure also provides homologous recombination systems for editing (e.g., insertion, deletion) in a microorganism. In certain embodiments, the homologous recombination system can be native to the host cell or introduced to the cell host. For example, but without any limitation, genes for the homologous recombination system can be introduced on a plasmid, introduced on a linear DNA fragment, introduced as and translated from RNA or set of RNAs, or introduced as a protein or set of proteins. In certain embodiments, the methods include a recombinant polynucleotide disclosed herein. In certain embodiments, the polynucleotide includes sequence homologous (e.g. left and right homology arms) to a region in a nucleic acid (e.g., genome, plasmid, etc.) such that the left and right homology arms are separated by a designed genetic edit (e.g., promoter, insertion, substitution, SNP, terminator, degron, a sequence for a tag, sequence for a degradation signal or deletion). In certain embodiments, the recombinant polynucleotide includes a genetic marker, a counter selectable genetic marker (e.g., SacB or PheS), and an origin of replication (e.g., R6K).

In certain embodiments, the recombinant polynucleotide including the homology arms and sequence for genetic editing is introduced into the microorganism using any of the methods disclosed herein (e.g., transformation via electroporation, conjugation, etc.). In certain embodiments, following transformation, the resulting transformants can be plated on a medium to select for transformants expressing the selectable genetic markers. The recombination of a plasmid comprising homology arms with a targeted locus in a nucleic acid (e.g., genome, plasmid, etc.) can occur at one of the two homology sites targeted by the homology arms present on the plasmid and that flank the designed genetic edit. In certain embodiments, the resulting transformants grow as colonies on the selective medium and can be selected and plated on a second type of selective medium (e.g. counter-selectable medium). In certain embodiments, the second type of selective medium allows the selection of cells that comprise the desired genetic editing.

In certain embodiments, the methods disclosed herein include using proteins from one or more recombination systems. Said recombination systems can be endogenous to the microorganism or can be exogenous. In certain embodiments, the proteins from one or more recombination systems can be introduced as nucleic acids (e.g., as a plasmid, linear DNA or RNA, or integron) and be integrated into the genome of the host cell or be stably expressed from an extrachromosomal element. In certain embodiments, the proteins from one or more recombination systems can be introduced as RNA and be translated by the host cell. In certain embodiments, the proteins from one or more recombination systems can be introduced as proteins into the host cell. Non-limiting examples of recombination systems include lambda red recombination system, RecET recombination system, Red/ET recombination system, any homologs, orthologs, or paralogs of proteins from a lambda red recombination system. RecET recombination system, Red/ET recombination system, lambda red-mediated recombination system, or any combination thereof. Details on the recombination systems from the RecET recombination system can be any of those as described in Zhang Y., Buchholz F., Muyrers J. P. P. and Stewart A. F. “A new logic for DNA engineering using recombination in E. coli.” Nature Genetics 20 (1998) 123-128; Muyrers, J. P. P., Zhang, Y., Testa, G., Stewart, A. F. “Rapid modification of bacterial artificial chromosomes by ET-recombination.” Nucleic Acids Res. 27 (1999) 1555-1557: Zhang Y., Muyrers J. P. P., Testa G. and Stewart A. F. “DNA cloning by homologous recombination in E. coli.” Nature Biotechnology 18 (2000) 1314-1317 and Muyrers J P et al., “Techniques: Recombinogenic engineering—new options for cloning and manipulating DNA” Trends Biochem Sci. 2001 May: 26 (5): 325-31, which are herein incorporated by reference in their entirety.

### 4. Methods for Producing Psicose

The present disclosure also provides methods for producing psicose. Cell-free methods (e.g., in vitro synthesis) have a predicted ΔG° of +5 KJ/mol which renders these thermodynamically unfavorable. In certain embodiments, the presently disclosed methods for producing psicose include culturing microorganisms (e.g., one disclosed in Section 2) and purifying psicose.

### 4.1. Cell Culture

The present disclosure provides methods of culturing microorganisms disclosed herein. As used herein, “culturing” a cell refers to introducing an appropriate culture medium, under appropriate conditions, to promote the growth of a cell. In certain embodiments, culturing is performed using a liquid or solid growth medium. In certain embodiments, culturing occurs under aerobic or anaerobic conditions based on the requirements of the microorganism and desired metabolic state of the same. In certain embodiments, culturing includes specific conditions such as temperature, pressure, light, pH, and cell density.

In certain embodiments, the methods for producing methods of producing psicose include a culture medium for culturing the recombinant bacteria. “Culture medium,” as used herein, refers to any composition or broth that supports the growth of the microorganism disclosed herein. A culture media can be liquid or solid. In certain embodiments, the culture media include nutrients, salts, buffers, elements, and other compounds that support the growth and viability of cells. Additionally, culture media can include sources of nitrogen, carbon, amino acids, carbohydrates, trace elements, vitamins, and minerals. In certain embodiments, the culture media include a complex extract (e.g., yeast extract). In certain embodiments, the culture medium is enriched in order to support rapid growth. In certain embodiments, the culture medium is modified in order to support slower growth. In certain embodiments, the culture medium includes an agent that can inhibit the growth of or kill contaminating organisms (e.g., an antibiotic). In certain embodiments, the culture medium includes an agent that can activate an inducible promoter or enzyme (e.g., IPTG). Non-limiting examples of culture media encompassed by the present disclosure include M9 medium. Lysogeny Broth (LB). Terrific Broth (TB), and YT broth. In certain embodiments, the culture medium comprises a substrate that is converted by the recombinant microorganisms to psicose.

In certain embodiments, the substrate is a sugar (e.g., glucose or fructose) that can be phosphorylated by the bacteria via a kinase (e.g., hexokinase) and converted into fructose-6-phosphate. In certain embodiments, the substrate is glucose. In certain embodiments, glucose can derive from cellulose. C₅ sugars, hemicellulose, and/or xylose. In certain embodiments, the substrate is a constituent of the culture medium. In certain embodiments, the substrate is supplemented with the culture medium. In certain embodiments, the substrate is continuously present in the culture medium. In certain embodiments, the substrate is supplemented during the growth phase. In certain embodiments, the substrate is supplemented during the stationary phase.

### 4.2. Purification of Psicose

In certain embodiments, the methods of the present disclosure further comprise purifying psicose produced by a microorganism of the present disclosure, e.g., from cell culture or cell culture medium. A variety of methods known in the art may be used to purify a product from a microorganism or microorganism culture. In certain embodiments, one or more products may be purified continuously, e.g., from a continuous culture. In certain embodiments, one or more products may be purified separately from fermentation, e.g., from a batch or fed-batch culture. One skilled in the art will appreciate that the specific purification method(s) used may depend upon, inter alia, the microorganism, culture conditions, and/or particular product(s).

In certain embodiments, purifying psicose comprises separating or filtering the microorganisms from a cell culture medium, separating the psicose from the culture medium (e.g., by chromatography), concentration of water (e.g., by evaporation), and lyophilization of the psicose.

### 4.3. Purity

In certain embodiments, the methods of the present disclosure allow for obtaining psicose at a high purity value. As used herein, the term “allulose purity” refers to a percentage value of the concentration of allulose compared to the sum of the concentrations of allulose, mannone, and glucose. In other words, the term allulose purity refers to a relative value of the allulose free from mannose and/or glucose (e.g., extraneous or contaminating sugars). In certain embodiments, the allulose purity is calculated using sugar concentrations and the following equation:

$$\% purity = \frac{[ allu lose ]}{[ allulose ] + [ mannose ] + [ glucose ]} \times 100$$

To determine the purity (e.g., allulose purity) of a sample (e.g., post-production culture media sample, after purification sample), the concentration of glucose, allulose, and mannose can be analyzed using high-performance liquid chromatography (HPLC). Glucose, allulose, and mannose standards of known concentrations can be run on the HPLC, and the area under each corresponding peak can be integrated. For each sugar standard, the peak integrations can be plotted against concentrations, and fitted with a line of best fit. Concurrently with standards, production samples can be run on the HPLC. Standards can be used to identify the corresponding sugar peaks in each production sample (e.g., post-production culture media sample, after purification sample). Each sample peak can be integrated, and their areas recorded. Using the line of best fit, peak integrations can be used to find the concentration of sugars in each sample.

In certain embodiments, the allulose purity is determined after culturing of the microorganisms disclosed herein. In certain embodiments, the allulose purity has a percentage value (%) between about 50% and about 100%. In certain embodiments, the allulose purity has a percentage value at least about 50%, at least about 60%, at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100%. In certain embodiments, the allulose purity has a percentage value of at least about 80%. In certain embodiments, the allulose purity has a percentage value of at least about 90%. In certain embodiments, the allulose purity has a percentage value of at least about 95%. In certain embodiments, the allulose purity has a percentage value of at least about 100%.

In certain embodiments, the allulose purity is determined after purifying psicose. In certain embodiments, the allulose purity has a percentage value (%) between about 50% and about 100%. In certain embodiments, the allulose purity has a percentage value at least about 50%, at least about 60%, at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100%. In certain embodiments, the allulose purity has a percentage value of at least about 80%. In certain embodiments, the allulose purity has a percentage value of at least about 90%. In certain embodiments, the allulose purity has a percentage value of at least about 95%. In certain embodiments, the allulose purity has a percentage value of at least about 100%.

In certain embodiments, the allulose purity meets or exceeds the standards set by the American Chemical Society (ACS) or defined in the U.S. Pharmacopeia (USP).

### 5. Food Products

The present disclosure also provides delivery systems methods for use in food products including the psicose prepared and/or generated by any of the microorganisms disclosed herein.

The term “food product.” as used herein, includes any food product, for example, those set forth in 21 CFR 101.12. Non-limiting examples of such food products include frozen desserts, baked goods, fillings, nutritional drinks, beverages, salad dressing or similar dressing, sauces, icings, puddings and custards, batters, and the like. Various baked goods are disclosed in U.S. Pat. No. 6,536,599, the disclosure of which is herein incorporated by reference in its entirety. Non-limiting examples of bakery goods include cookies, cakes, rolls, pastries, pie dough, brownies, breads, bagels, and the like. The psicose prepared and/or generated by any of the microorganisms disclosed herein are also suitable as a component in frozen foods.

In certain embodiments, the food product is prepared by admixing the psicose in an ingestible vehicle, together with any optional ingredients, to form a uniform mixture. The final compositions are readily prepared using standard methods and apparatus generally known by those skilled in the corresponding arts, such as confectionary arts. The apparatus useful per the presently disclosed subject matter comprises mixing apparatus well known in the art, and therefore the selection of the specific apparatus will be apparent to the artisan.

In certain embodiments, the present application relates to the modified edible food products produced by the methods disclosed herein. In certain embodiments, the food products can be produced by processes for producing comestible products well known to those of ordinary skill in the art.

In certain embodiments, the psicose prepared and/or generated by any of the microorganisms disclosed herein can be dissolved in or dispersed in one of many known comestible acceptable liquids, solids, or other carriers, such as water at neutral, acidic, or basic pH, fruit or vegetable juices, vinegar, marinades, beer, wine, natural water/fat emulsions such as milk or condensed milk, whey or whey products, edible oils and shortenings, fatty acids, certain low molecular weight oligomers of propylene glycol, glyceryl esters of fatty acids, and dispersions or emulsions of such hydrophobic substances in aqueous media, salts such as sodium chloride, vegetable flours, solvents such as ethanol, solid edible diluents such as vegetable powders or flours, and the like, and then combined with precursors of the comestible or medicinal products, or applied directly to the comestible or medicinal products.

Those of ordinary skill in the art of preparing and selling food products are well aware of a large variety of classes, subclasses, and species of the comestible compositions, and utilize well-known and recognized terms of art to refer to those comestible compositions while endeavoring to prepare and sell various of those comestible compositions. Such a list of terms of art is enumerated below, and it is specifically contemplated hereby that the psicose prepared and/or generated by any of the microorganisms disclosed herein can be used to modify or enhance the taste of the following list edible compositions, either singly or in all reasonable combinations or mixtures thereof.

In certain embodiments, the food products to which the psicose prepared and/or generated by any of the microorganisms disclosed herein are admixed with comprise, by way of example, the wet soup category, the dehydrated and culinary food category, the beverage category, the frozen food category, the snack food category, and seasonings or seasoning blends, described herein.

In certain embodiments, the psicose prepared and/or generated by any of the microorganisms disclosed herein are admixed with one or more confectioneries, chocolate confectionery, tablets, countlines, bagged selfmies/softlines, boxed assortments, standard boxed assortments, twist wrapped miniatures, seasonal chocolate, chocolate with toys, allsorts, other chocolate confectionery, mints, standard mints, power mints, boiled sweets, pastilles, gums, jellies and chews, toffees, caramels and nougat, medicated confectionery, lollipops, liquorice, other sugar confectionery, gum, chewing gum, sugarised gum, sugar-free gum, functional gum, bubble gum, bread, packaged/industrial bread, unpackaged/artisanal bread, pastries, cakes, packaged/industrial cakes, unpackaged/artisanal cakes, cookies, chocolate coated biscuits, sandwich biscuits, filled biscuits, savory biscuits and crackers, bread substitutes, breakfast cereals, rte cereals, family breakfast cereals, flakes, muesli, other rte cereals, children's breakfast cereals, hot cereals, ice cream, impulse ice cream, single portion dairy ice cream, single portion water ice cream, multi-pack dairy ice cream, multi-pack water ice cream, take-home ice cream, take-home dairy ice cream, ice cream desserts, bulk ice cream, take-home water ice cream, frozen yoghurt, artisanal ice cream, dairy products, milk, fresh/pasteurized milk, full fat fresh/pasteurized milk, semi skimmed fresh/pasteurized milk, long-life/uht milk, full fat long life/uht milk, semi skimmed long life/uht milk, fat-free long life/uht milk, goat milk, condensed/evaporated milk, plain condensed/evaporated milk, flavored, functional and other condensed milk, flavored milk drinks, dairy only flavored milk drinks, flavored milk drinks with fruit juice, soy milk, sour milk drinks, fermented dairy drinks, coffee whiteners, powder milk, flavored powder milk drinks, cream, cheese, processed cheese, spreadable processed cheese, unspreadable processed cheese, unprocessed cheese, spreadable unprocessed cheese, hard cheese, packaged hard cheese, unpackaged hard cheese, yoghurt, plain/natural yoghurt, flavored yoghurt, fruited yoghurt, probiotic yoghurt, drinking yoghurt, regular drinking yoghurt, probiotic drinking yoghurt, chilled and shelf-stable desserts, dairy-based desserts, soy-based desserts, chilled snacks, fromage frais and quark, plain fromage frais and quark, flavored fromage frais and quark, savory fromage frais and quark, sweet and savory snacks, fruit snacks, chips/crisps, extruded snacks, tortilla/corn chips, popcorn, pretzels, nuts, other sweet and savory snacks, snack bars, granola bars, breakfast bars, energy bars, fruit bars, other snack bars, meal replacement products, slimming products, convalescence drinks, ready meals, canned ready meals, frozen ready meals, dried ready meals, chilled ready meals, dinner mixes, frozen pizza, chilled pizza, soup, canned soup, dehydrated soup, instant soup, chilled soup, uht soup, frozen soup, pasta, canned pasta, dried pasta, chilled/fresh pasta, noodles, plain noodles, instant noodles, cups/bowl instant noodles, pouch instant noodles, chilled noodles, snack noodles, canned food, canned meat and meat products, canned fish/seafood, canned vegetables, canned tomatoes, canned beans, canned fruit, canned ready meals, canned soup, canned pasta, other canned foods, frozen food, frozen processed red meat, frozen processed poultry, frozen processed fish/seafood, frozen processed vegetables, frozen meat substitutes, frozen potatoes, oven baked potato chips, other oven baked potato products, non-oven frozen potatoes, frozen bakery products, frozen desserts, frozen ready meals, frozen pizza, frozen soup, frozen noodles, other frozen food, dried food, dessert mixes, dried ready meals, dehydrated soup, instant soup, dried pasta, plain noodles, instant noodles, cups/bowl instant noodles, pouch instant noodles, chilled food, chilled processed meats, chilled fish/seafood products, chilled processed fish, chilled coated fish, chilled smoked fish, chilled lunch kit, chilled ready meals, chilled pizza, chilled soup, chilled/fresh pasta, chilled noodles, oils and fats, olive oil, vegetable and Seed oil, cooking fats, butter, margarine, spreadable oils and fats, functional spreadable oils and fats, sauces, dressings and condiments, tomato pastes and purees, bouillon/stock cubes, stock cubes, gravy granules, liquid stocks and fonds, herbs and spices, fermented sauces, soy based sauces, pasta sauces, wet sauces, dry sauces/powder mixes, ketchup, mayonnaise, regular mayonnaise, mustard, salad dressings, regular salad dressings, low fat salad dressings, vinaigrettes, dips, pickled products, other sauces, dressings and condiments, baby food, milk formula, standard milk formula, follow-on milk formula, toddler milk formula, hypoallergenic milk formula, prepared baby food, dried baby food, other baby food, spreads, jams and preserves, honey, chocolate spreads, nut-based spreads, and yeast-based spreads.

### 5.1. Chewing-Gum

In certain embodiments, the psicose prepared and/or generated by any of the microorganisms disclosed herein can be used in low-calorie gum formulations and can also be used in sugar chewing gum. Various specifics of chewing gum compositions are disclosed in U.S. Pat. No. 6,899,911, the disclosure of which is incorporated herein by reference in its entirety. The chewing gum composition of the presently disclosed subject matter follows the general pattern outlined below. In general, a chewing gum composition typically contains a chewable gum base portion that is essentially free of water and is water-insoluble, a water-soluble bulk portion, and flavors that are typically water-insoluble. The water-soluble portion dissipates with a portion of the flavor over a period of time during chewing. The gum base portion is retained in the mouth throughout the chew. The insoluble gum base generally comprises elastomers, elastomer solvents, plasticizers, waxes, emulsifiers, and inorganic fillers. Plastic polymers, such as polyvinyl acetate, which behave somewhat as plasticizers, are also often included. Other plastic polymers that can be used include polyvinyl laureate, polyvinyl alcohol, and polyvinyl pyrrolidone. Elastomers can include polyisobutylene, butyl rubber, (isobutylene-isoprene copolymer), and styrene butadiene rubber, as well as natural latexes such as chicle. Elastomer solvents are often resins such as terpene resins. Plasticizers, sometimes called softeners, are typically fats and oils, including tallow, hydrogenated and partially hydrogenated vegetable oils, and cocoa butter. Commonly employed waxes include paraffin, microcrystalline, and natural waxes such as beeswax and carnauba. Microcrystalline waxes, especially those with a high degree of crystallinity, can be considered bodying agents or textural modifiers.

In certain embodiments, the insoluble gum base constitutes between about 5% to about 95% by weight of the gum. More preferably the insoluble gum base comprises between 10% and 50% by weight of the gum and most preferably about 20% to 35% by weight of the gum. The gum base typically also includes a filler component. The filler component can be calcium carbonate, magnesium carbonate, talc, dicalcium phosphate, or the like. The filler can constitute between about 5% and about 60% by weight of the gum base. Preferably the filler comprises about 5% to 50% by weight of the gum base.

Gum bases typically also contain softeners including glycerol monostearate and glycerol triacetate. Gum bases can also contain optional ingredients such as antioxidants, colors, and emulsifiers. The presently disclosed subject matter contemplates employing any commercially acceptable gum base.

The water-soluble portion of the chewing gum can further comprise softeners, sweeteners, flavors, physiological cooling agents, and combinations thereof. The sweeteners often fulfill the role of bulking agents in the gum. The bulking agents typically comprise about 5% to about 95% of the gum composition.

Softeners are added to the chewing gum in order to optimize the chewability and mouth feel of the gum. Softeners, also known in the art as plasticizers or plasticizing agents, generally constitute between about 0.5% to about 15% of the chewing gum. Softeners contemplated by the presently disclosed subject matter include glycerin, lecithin, and combinations thereof. Further, aqueous sweetener solutions such as those containing sorbitol, hydrogenated starch hydrolysate, corn syrup, and combinations thereof can be used as softeners and binding agents in gum.

As mentioned above, the psicose prepared and/or generated by any of the microorganisms disclosed herein can be used in low-calorie gum formulations. However, formulations containing sugar are also within the scope of the invention. Sugar sweeteners generally include saccharide-containing components commonly known in the chewing gum art which comprise, but are not limited to, sucrose, dextrose, maltose, dextrin, dried invert sugar, fructose, galactose, corn syrup solids and the like, alone or in any combination. The psicose prepared and/or generated by any of the microorganisms disclosed herein can also be used in combination with sugarless sweeteners. Generally, sugarless sweeteners include components with sweetening characteristics but which are devoid of the commonly known sugars and comprise, but are not limited to, sugar alcohols such as sorbitol, hydrogenated isomaltulose, mannitol, xylitol, lactitol, erythritol, hydrogenated starch hydrolysate, maltitol and the like alone or in any combination.

Depending on the particular sweetness release profile and shelf stability needed, coated or uncoated high-intensity sweeteners can be used in the chewing gum composition, or can be used in a coating applied to centers made from those gum compositions. High-intensity sweeteners, preferably aspartame, can be used at levels from about 0.01% to about 3.0%. Encapsulated aspartame is a high-intensity sweetener with improved stability and release characteristics, as compared to free aspartame. Free aspartame can also be added, and a combination of some free and encapsulated aspartame is preferred when aspartame is used. Other high-intensity sweeteners that can be used in the gum center are: saccharin, Thaumatin, alitame, saccharin salts, sucralose, Stevia, and acesulfame K. Overall, the chewing gum composition will preferably comprise about 0.5% to about 90% sweetening agents. Most typically the sweetening agents will comprise at least one bulk sweetener and at least one high-intensity sweetener. Optional ingredients such as colors, emulsifiers, and pharmaceutical agents can also be added as separate components of the chewing gum composition, or added as part of the gum base.

Aqueous syrups, such as corn syrup and hydrogenated corn syrup can be used, particularly if their moisture content is reduced. This can preferably be done by co-evaporating the aqueous syrup with a plasticizer, such as glycerin or propylene glycol, to a moisture content of less than 10%. Preferred compositions include hydrogenated starch hydrolysate solids and glycerin. Such syrups and their methods of preparation are discussed in detail in U.S. Pat. No. 4,671,967.

Methods of manufacturing chewing gum according to the presently disclosed subject matter include the sequential addition of the various chewing gum ingredients to any commercially available mixer known in the art. After the ingredients have been thoroughly mixed, the gum is discharged from the mixer and shaped into the desired form such as by rolling into sheets and cutting into sticks, extruding into chunks, or casting into pellets. Generally, the ingredients are mixed by first melting the gum base and adding it to the running mixer. The base can also be melted in the mixer itself. Color or emulsifiers can also be added at this time, along with syrup and a portion of the bulking agent. Further portions of the bulking agent can then be added to the mixer. Flavor systems are typically added with the final portion of the bulking agent. If the flavor system is coated or otherwise modified when incorporated into a delivery system to modify its release rate, it will preferably be added after the final portion of the bulking agent has been added. The entire mixing procedure typically takes from five to twenty minutes, but longer mixing times can sometimes be required. Those skilled in the art will recognize that many variations of the above-described procedures can be followed.

If formed into pellets or balls, the chewing gum composition can be coated. The coating is initially present as a liquid syrup which contains from about 30% to about 80% or 85% sugars or sugar alcohols, and from about 15% or 20% to about 70% of a solvent such as water. In general, the coating process is carried out in conventional panning equipment. Gum center tablets to be coated are placed into the panning equipment to form a moving mass.

The material or syrup that will eventually form the coating is applied or distributed over the gum center tablets. The psicose can be added before, during, and after applying the syrup to the gum centers. Once the coating has dried to form a hard surface, additional syrup additions can be made to produce a plurality of coatings or multiple layers of coating. The psicose can be added to any or none of the coatings and/or layers.

In the panning procedure, syrup is added to the gum center tablets at a temperature range of from about 100° F. to about 240° F. Preferably, the syrup temperature is from about 140° F. to about 200° F. Most preferably, the syrup temperature should be kept constant throughout the process in order to prevent the polyol in the syrup from crystallizing. The syrup can be mixed with, sprayed upon, poured over, or added to the gum center tablets in any way known to those skilled in the art.

In certain embodiments, a soft coating is formed by adding a powder coating after a liquid coating. The powder coating can include natural carbohydrate gum hydrolysates, maltodextrin, gelatin, cellulose derivatives, starches, modified starches, sugars, sugar alcohols, natural carbohydrate gums, and fillers like talc and calcium carbonate.

Each component of the coating on the gum center can be applied in a single layer or a plurality of layers. In general, a plurality of layers is obtained by applying single coats, allowing the layers to dry, and then repeating the process. The amount of solids added by each coating step depends chiefly on the concentration of the coating syrup. Any number of coats can be applied to the gum center tablet. Preferably, no more than about 75 coats are applied to the gum center. More preferably, less than about 60 coats are applied and most preferably, about 30 to about 60 coats are applied. In any event, the presently disclosed subject matter contemplates applying an amount of syrup sufficient to yield a coated chewing gum product containing about 10% to about 65% coating. Preferably, the final product will contain from about 20% to about 50% coating.

Those skilled in the art will recognize that in order to obtain a plurality of coated layers, a plurality of premeasured aliquots of coating syrup can be applied to the gum center. It is contemplated, however, that the volume of aliquots of syrup applied to the gum center can vary throughout the coating procedure.

Once a coating of syrup is applied to the gum center, the syrup is dried in an inert medium. A preferred drying medium comprises air. Preferably, forced drying air contacts the wet syrup coating in a temperature range of from about 70° F. to about 110° F. More preferably, the drying air is in the temperature range of from about 80° F. to about 100° F. The invention also contemplates that the drying air possesses a relative humidity of less than about 15 percent. Preferably, the relative humidity of the drying air is less than about 8%.

The drying air can be passed over and admixed with the syrup coated gum centers in any way commonly known in the art. Preferably, the drying air is blown over and around the syrup coated gum center at a flow rate, for large scale operations, of about 2800 cubic feet per minute. If lower quantities of material are being processed, or if smaller equipment is used, lower flow rates would be used. If a flavor is applied after a syrup coating has been dried, the presently disclosed subject matter contemplates drying the flavor with or without the use of a drying medium.

The amount of psicose employed herein is normally a matter of preference subject to such factors as the type of final chewing gum composition, the individual flavor, the gum base employed, and the strength of flavor desired. Thus, the amount of psicose can be varied in order to obtain the result desired in the final product and such variations are within the capabilities of those skilled in the art without the need for undue experimentation. In gum compositions, the psicose prepared and/or generated by any of the microorganisms disclosed herein is generally present in amounts from about 0.02% to about 5%, and preferably from about 0.1% to about 2%, and more preferably, from about 0.8% to about 1.8%, by weight of the chewing gum composition.

### 5.2. Sugar Confectionery

Another important aspect of the presently disclosed subject matter includes a confectionery composition incorporating the psicose prepared and/or generated by any of the microorganisms disclosed herein and a method for preparing the confectionery compositions. The preparation of confectionery formulations is well-known in the art. Confectionery items have been classified as either “hard” confectionery or “soft” confectionery. The psicose prepared and/or generated by any of the microorganisms disclosed herein can be incorporated into the confections by admixing the compositions of the presently disclosed subject matter into the conventional hard and soft confections.

Hard confectionery can be processed and formulated by conventional means. In general, hard confectionery has a base composed of a mixture of sugar and other carbohydrate bulking agents kept in an amorphous or glassy condition. The hard confectionery can also be sugarless. The hard confectionery can also be low-calorie. This form is considered a solid syrup of sugars generally having from about 0.5% to about 1.5% moisture. Such materials normally contain up to about 92% sugar, up to about 55% corn syrup, and from about 0.1% to about 5% water, by weight of the final composition. The syrup component is generally prepared from sucrose and corn syrups but can include other materials. In certain embodiments, the syrup component includes the psicose prepared and/or generated by any of the microorganisms disclosed herein. Further ingredients such as flavorings, sweetening agents, acidulants, colorants, and so forth can also be added.

Such confectionery can be routinely prepared by conventional methods, including but not limited to methods involving fire cookers, vacuum cookers, and scraped-surface cookers also referred to as high-speed atmospheric cookers. The apparatus useful in accordance with the presently disclosed subject matter comprises cooking and mixing apparatus well known in the confectionery manufacturing arts, and therefore the selection of the specific apparatus will be apparent to the artisan.

Fire cookers involve the traditional method of making a candy base. In this method, the desired quantity of carbohydrate bulking agent is dissolved in water by heating the agent in a kettle until the bulking agent dissolves. Additional bulking agents can then be added and cooked until a final temperature of 145° C. to 156° C. is achieved. The batch is then cooled and worked as a plastic-like mass to incorporate additives such as flavoring agents, colorants, and the like.

A high-speed atmospheric cooker uses a heat-exchanger surface, which involves spreading a film of candy on a heat exchange surface, the candy is heated to 165° C. to 170° C. within a few seconds. The candy is then rapidly cooled to 100° C. to 120° C. and worked as a plastic-like mass enabling incorporation of the additives, such as flavoring agents, colorants, and the like. In vacuum cookers, the carbohydrate bulking agent is boiled to 125° C. to 132° C., vacuum is applied and additional water is boiled off without extra heating. When cooking is complete, the mass is a semi-solid and has a plastic-like consistency. At this point, flavoring agents, colorants, and other additives are admixed in the mass by routine mechanical mixing operations.

The optimum mixing required to uniformly mix the flavoring agent, colorants, and other additives during conventional manufacturing of hard confectionery is determined by the time needed to obtain a uniform distribution of the materials. Generally, mixing times of from 2 to 10 minutes have been found to be acceptable.

Once the candy mass has been properly tempered, it can be cut into workable portions or formed into desired shapes. A variety of forming techniques can be utilized depending upon the shape and size of the final product desired. A general discussion of the composition and preparation of hard confections can be found in H. A. Lieberman, Pharmaceutical Dosage Forms: Tablets, Volume 1 (1989), Marcel Dekker, Inc., New York, N.Y. at pages 419 to 582, which disclosure is incorporated herein by reference.

Compressed tablet confections contain particular materials and are formed into structures under pressure. These confections generally contain sugars in amounts up to about 95%, by weight of the composition, and typical tablet excipients such as binders and lubricants as well as flavoring agents, colorants, and so forth. These confections can also be sugarless.

Similar to hard confectionery, soft confectionery can be utilized in the embodiments of the disclosed subject matter. The preparation of soft confections, such as nougat, involves conventional methods, such as the combination of two primary components, namely (1) a high boiling syrup such as corn syrup, or the like, and (2) a relatively light textured frappe, generally prepared from egg albumin, gum arabic, gelatin, vegetable proteins, such as soy-derived compounds, sugarless milk-derived compounds such as milk proteins, and mixtures thereof. The frappe is generally relatively light, and can, for example, range in density from about 0.5 to about 0.7 grams/cc.

The high boiling syrup, or “bob syrup” of the soft confectionery, is relatively viscous, has a higher density than the frappe component, and frequently contains a substantial amount of carbohydrate bulking agent. Conventionally, the final nougat composition is prepared by the addition of the “bob syrup” to the frappe under agitation, to form the basic nougat mixture. Further ingredients such as flavoring, additional carbohydrate bulking agents, colorants, preservatives, medicaments, mixtures thereof and the like can be added thereafter also under agitation. Soft confectioneries can also be prepared sugarless. A general discussion of the composition and preparation of nougat confections can be found in B. W. Minifie, Chocolate, Cocoa and Confectionery: Science and Technology, 2nd edition. AVI Publishing Co., Inc., Westport, Conn. (1983), at pages 576-580, which disclosure is incorporated herein by reference.

In general, the frappe component is prepared first and thereafter the syrup component is slowly added under agitation at a temperature of at least about 65° C., and preferably at least about 100° C. The mixture of components is continued to be mixed to form a uniform mixture, after which the mixture is cooled to a temperature below 80° C. at which point, the flavor can be added. The mixture is further mixed for an additional period until it is ready to be removed and formed into suitable confectionery shapes.

In accordance with the present disclosure, amounts of the psicose prepared and/or generated by any of the microorganisms disclosed herein can be admixed into the hard and soft confections. The exact amount of psicose employed is normally a matter of preference subject to such factors as the particular type of confection being prepared, the type of bulking agent or carrier employed, the type of flavor employed, and the intensity of breath freshening perception desired. Thus, the amount of psicose can be varied in order to obtain the result desired in the final product and such variations are within the capabilities of those skilled in the art without the need for undue experimentation. In general, the amount of psicose normally present in a hard or soft confection will be from about 0.001% to about 20%, preferably from about 0.01% to about 15%, more preferably from about 0.01% to about 10%, and more preferably from about 0.01% to about 5%, and more preferably 0.01% to about 0.5% by weight of the confection.

The presently disclosed subject matter extends to methods for making the improved confections. The psicose prepared and/or generated by any of the microorganisms disclosed herein can be incorporated into an otherwise conventional hard or soft confection composition using standard techniques and equipment known to those skilled in the art. The apparatus useful in accordance with the presently disclosed subject matter comprises mixing and heating apparatus well known in the confectionery manufacturing arts, and therefore the selection of the specific apparatus will be apparent to the artisan.

In such a method, a composition is made by admixing the psicose into the confectionery composition along with the other ingredients of the final desired composition. Other ingredients will usually be incorporated into the composition as dictated by the nature of the desired composition as well known by those having ordinary skill in the art. The ultimate confectionery compositions are readily prepared using methods generally known in the food technology and pharmaceutical arts. Thereafter the confectionery mixture can be formed into desirable confectionery shapes.

The psicose prepared and/or generated by any of the microorganisms disclosed herein can be formulated with conventional ingredients that offer a variety of textures to suit particular applications. Such ingredients can be in the form of hard and soft confections, tablets, toffee, nougat, chewy candy, chewing gum and so forth, center filled candies, both sugar and sugarless. The acceptable ingredients can be selected from a wide range of materials. Without being limited thereto, such materials include diluents, binders and adhesives, lubricants, disintegrants, bulking agents, humectants, buffers, and adsorbents. The preparation of such confections and chewing gum products is well known.

### 5.3. Chocolates and Fillings

The presently disclosed subject matter is also used with and/or in chocolate products, chocolate-flavored confections, and chocolate flavored compositions. Chocolates also include those containing crumb solids or solids fully or partially made by a crumb process. Various chocolates are disclosed, for example, in U.S. Pat. Nos. 7,968,140 and 8,263,168, the disclosures of which are incorporated herein by reference in their entireties. A general discussion of the composition and preparation of chocolate confections can be found in B. W. Minifie, Chocolate, Cocoa and Confectionery: Science and Technology, 2nd edition, AVI Publishing Co., Inc., Westport, Conn. (1982), which disclosure is incorporated herein by reference.

The term “chocolate” as used herein refers to a solid or semi-plastic food and is intended to refer to all chocolate or chocolate-like compositions containing a fat-based component phase or fat-like composition. The term is intended to include standardized or nonstandardized compositions conforming to the U.S. Standards Of Identity (SOI), CODEX Alimentarius and/or other international standards and compositions not conforming to the U.S. Standards Of Identity or other international standards. The term includes dark chocolate, baking chocolate, sweet chocolate, bittersweet or semisweet chocolate, milk chocolate, buttermilk chocolate, skim milk chocolate, mixed dairy product chocolate, white chocolate, sweet cocoa and vegetable fat coating, sweet chocolate and vegetable fat coating, milk chocolate and vegetable fat coating, vegetable fat based coating, pastels including white chocolate or coating made with cocoa butter or vegetable fat or a combination of these, nutritionally modified chocolate-like compositions (chocolates or coatings made with reduced calorie ingredients) and low fat chocolates, aerated chocolates, compound coatings, non-standardized chocolates and chocolate-like compositions, unless specifically identified otherwise.

Nonstandardized chocolates result when, for example, the nutritive carbohydrate sweetener is replaced partially or completely; or when the cocoa butter, cocoa butter alternative, cocoa butter equivalent, cocoa butter extender, cocoa butter replacer, cocoa butter substitute or milkfat are replaced partially or completely; or when components that have flavors that imitate milk, butter or chocolate are added or other additions or deletions in formula are made outside the FDA standards of identify of chocolate or combinations thereof. Chocolate-like compositions are those fat-based compositions that can be used as substitutes for chocolate in applications such as panning, molding, or enrobing; for example, carob.

In the United States, chocolate is subject to a standard of identity established by the U.S. Food and Drug Administration (FDA) under the Federal Food, Drug and Cosmetic Act. Definitions and standards for the various types of chocolate are well established in the U.S. Nonstandardized chocolates are those chocolates which have compositions that fall outside the specified ranges of the standardized chocolates.

In certain embodiments, the chocolate can contain psicose prepared and/or generated by any of the microorganisms disclosed herein. Additionally, the chocolate can contain a sugar syrup/solids, invert sugar, hydrolyzed lactose, maple sugar, brown sugar, molasses, honey, sugar substitute and the like. Nutritive carbohydrate sweeteners with varying degrees of sweetness intensity can be any of those typically used in the art and include, but are not limited to, sucrose, e.g. from cane or beet, dextrose, fructose, lactose, maltose, glucose syrup solids, corn syrup solids, invert sugar, hydrolyzed lactose, honey, maple sugar, brown sugar, molasses and the like. Sugar substitutes can partially replace the nutritive carbohydrate sweetener. High potency sweeteners include aspartame, cyclamates, saccharin, acesulfame-K, neohesperidin dihydrochalcone, sucralose, alitame, stevia sweeteners, glycyrrhizin, thaumatin and the like and mixtures thereof. The preferred high potency sweeteners are aspartame, cyclamates, saccharin, and acesulfame-K. Examples of sugar alcohols can be any of those typically used in the art and include sorbitol, mannitol, xylitol, maltitol, isomalt, lactitol and the like.

The chocolates can also contain bulking agents. The term “bulking agents” as defined herein can be any of those typically used in the art and include polydextrose, cellulose and its derivatives, maltodextrin, gum arabic, and the like.

The chocolate products can contain emulsifiers. Examples of safe and suitable emulsifiers can be any of those typically used in the art and include lecithin derived from vegetable sources such as soybean, safflower, corn, etc., fractionated lecithins enriched in either phosphatidyl choline or phosphatidyl ethanolamine, or both, mono- and digylcerides, diacetyl tartaric acid esters of mono- and diglycerides (also referred to as DATEM), monosodium phosphate derivatives of mono- and diglycerides of edible fats or oils, sorbitan monostearate, hydroxylated lecithin, lactylated fatty acid esters of glycerol and propylene glycol, polyglycerol esters of fatty acids, propylene glycol mono- and di-esters of fats and fatty acids, or emulsifiers that can become approved for the US FDA-defined soft candy category. In addition, other emulsifiers that can be used include polyglycerol polyricinoleate (PGPR), ammonium salts of phosphatidic acid, (e.g. YN) sucrose esters, oat extract, etc., any emulsifier found to be suitable in chocolate or similar fat/solid system or any blend.

The term “chocolate-flavored confection” refers to food products, excluding “chocolate”, having a chocolate flavor/aroma and comprising a cocoa fraction. These products are stable at ambient temperatures for extended periods of time (e.g., greater than 1 week) and are characterized as microbiologically shelf-stable at 18-30° C. under normal atmospheric conditions. Examples include chocolate-flavored hard candies, chewables, chewing gums, etc.

The term “chocolate-flavored compositions” refers to chocolate-flavored compositions, excluding “chocolate”, containing a cocoa fraction and having a chocolate flavor/aroma. Examples include chocolate-flavored cake mixes, ice creams, syrups, baking goods, etc. The term includes chocolate-flavored compositions (e.g., cakes, nougats, puddings, etc.), as well as compositions not having a chocolate flavor (e.g., caramels, etc.).

### 5.4. Savory Goods and Other Food Products

In certain embodiments, the psicose prepared and/or generated by any of the microorganisms disclosed herein is incorporated into savory goods. In certain embodiments, a savory good is a food product that has savory flavors including, for example, but not limited to, spicy flavor, pepper flavor, dairy flavor, vegetable flavor, tomato flavor, dill flavor, meat flavor, poultry flavor, chicken flavor and reaction flavors that are added or generated during heating of a food product.

In certain embodiments, the psicose prepared and/or generated by any of the microorganisms disclosed herein is incorporated into a wet soup category food product, which comprises wet/liquid soups regardless of concentration or container, including frozen soups. In certain embodiments, the soup food product means a food prepared from meat, poultry, fish, vegetables, grains, fruit, and/or other ingredients, cooked in a liquid which may include visible pieces of some or all of these ingredients. It may be clear (as a broth) or thick (as a chowder), smooth, pureed or chunky, ready-to-serve, semi-condensed or condensed and may be served hot or cold, as a first course or as the main course of a meal or as a between meal snack (sipped like a beverage). Soup may be used as an ingredient for preparing other meal components and may range from broths (consomme) to sauces (cream or cheese-based soups).

In certain embodiments, the psicose prepared and/or generated by any of the microorganisms disclosed herein is incorporated into a dehydrated and culinary food category of food products, which comprises (i) cooking aid products such as: powders, granules, pastes, concentrated liquid products, including concentrated bouillon, bouillon and bouillon like products in pressed cubes, tablets or powder or granulated form, which are sold separately as a finished product or as an ingredient within a product, sauces and recipe mixes (regardless of technology), (ii) meal solutions products such as: dehydrated and freeze dried soups, including dehydrated soup mixes, dehydrated instant soups, dehydrated ready-to-cook soups, dehydrated or ambient preparations of ready-made dishes, meals and single serve entrees including pasta, potato and rice dishes; and (iii) meal embellishment products such as: condiments, marinades, salad dressings, salad toppings, dips, breading, batter mixes, shelf stable spreads, barbecue sauces, liquid recipe mixes, concentrates, sauces or sauce mixes, including recipe mixes for salad, sold as a finished product or as an ingredient within a product, whether dehydrated, liquid or frozen.

In certain embodiments, the psicose prepared and/or generated by any of the microorganisms disclosed herein is incorporated into a meat food product. In certain embodiments, meat food products include food products made by processing the edible remains of any dead animal, including birds, fish, crustaceans, shellfish, and mammals. Meat food products include, without limitation, for example, prepared beef, lamb, pork, poultry, or seafood products. Examples of such meat food products include, for example, bologna, frankfurters, sausage, luncheon, deli slices, loaves, bacon, meatballs, fish sticks, chicken fingers, and ground meats, e.g., meatloaf, meatballs, and hamburgers. A meat food product may be combined with a simulated meat food product. Simulated meat food products include, without limitation, for example, a meat alternative, meat analog, soy burger, soy bologna, soy frankfurter, soy sausage, soy luncheon loaves, soy bacon, and soy meatball. A simulated meat food product may be combined with a meat food product.

In certain embodiments, the psicose prepared and/or generated by any of the microorganisms disclosed herein is incorporated into a snack food category food product. In certain embodiments, snack food products include any food that can be a light informal meal including, but not limited to sweet and savory snacks and snack bars. Examples of snack food include, but are not limited to fruit snacks, chips/crisps, extruded snacks, tortilla/corn chips, popcorn, pretzels, nuts, and other sweet and savory snacks. Examples of snack bars include, but are not limited to granola/muesli bars, breakfast bars, energy bars, fruit bars, and other snack bars.

In certain embodiments, the psicose prepared and/or generated by any of the microorganisms disclosed herein is incorporated into frozen food products, which comprises chilled or frozen food products, for example, but not limited to, ice cream, impulse ice cream, single portion dairy ice cream, single portion water ice cream, multi-pack dairy ice cream, multi-pack water ice cream, take-home ice cream, take-home dairy ice cream, ice cream desserts, bulk ice cream, take-home water ice cream, frozen yogurt, artisanal ice cream, frozen ready meals, frozen pizza, chilled pizza, frozen soup, frozen pasta, frozen processed red meat, frozen processed poultry, frozen processed fish/seafood, frozen processed vegetables, frozen meat substitutes, frozen potatoes, frozen bakery products and frozen desserts.

### 5.4. Pharmaceuticals

The psicose prepared and/or generated by any of the microorganisms disclosed herein can also be in the form of a pharmaceutical. One non-limiting example of a pharmaceutical form is a suspension. Pharmaceutical suspensions can be prepared by conventional compounding methods. Suspensions can contain adjunct materials employed in formulating the suspensions of the art. The suspensions of the presently disclosed subject matter can comprise preservatives, buffers, suspending agents, antifoaming agents, sweetening agents, flavoring agents, coloring or decoloring agents, solubilizers, and combinations thereof.

Flavoring agents such as those flavors well known to the skilled artisan, such as natural and artificial flavors and mints, such as peppermint, menthol, citrus flavors such as orange and lemon, artificial vanilla, cinnamon, and various fruit flavors, both individual and mixed and the like can be utilized in amounts from about 0.01% to about 5%, and more preferably 0.01% to about 0.5% by weight of the suspension.

The pharmaceutical suspensions of the presently disclosed subject matter can be prepared as follows: (i) admix the thickener with water heated from about 40° C. to about 95° C. preferably from about 40° C. to about 70° C., to form a dispersion if the thickener is not water-soluble or a solution if the thickener is water soluble; (ii) admix the psicose prepared and/or generated by any of the microorganisms disclosed herein with water to form a solution; (iii) admix, if desired, a flavoring agent with the thickener-water admixture to form a uniform thickener-flavoring agent; (iv) combine the sweetener solution with the thickener-flavoring agent and mix until uniform; and (v) admix the optional adjunct materials such as coloring agents, flavoring agents, decolorants, solubilizers, anti-foaming agents, buffers and additional water with the mixture of step (iv) to form the suspension.

The psicose prepared and/or generated by any of the microorganisms disclosed herein can also be in chewable form. To achieve acceptable stability and quality as well as good taste and mouth feel in a chewable formulation several considerations are important. These considerations include the amount of active substance per tablet, the flavoring agent employed, the degree of compressibility of the tablet, and additional properties of the composition. Chewable pharmaceutical candy is prepared by procedures similar to those used to make soft confectionery. A general discussion of the lozenge and chewable tablet forms of confectionery can be found in H. A. Lieberman and L. Lachman, Pharmaceutical Dosage Forms: Tablets Volume 1, Marcel Dekker, InC, New York, N.Y. (1989) at pages 367 to 418, which disclosure is incorporated herein by reference. In a typical procedure, a boiled sugar-corn syrup blend is formed to which is added a frappe mixture. The boiled sugar-corn syrup blend can be prepared from sugar and corn syrup blended in parts by weight ratio of about 90:10 to about 10:90. The sugar-corn syrup blend is heated to temperatures above about 120° C. to remove water and to form a molten mass. The frappe is generally prepared from gelatin, egg albumin, milk proteins such as casein, and vegetable proteins such as soy protein, and the like, which are added to a gelatin solution and rapidly mixed at ambient temperature to form an aerated sponge-like mass. The frappe is then added to the molten candy mass and mixed until homogeneous at temperatures between about 65° C. and about 120° C. The psicose prepared and/or generated by any of the microorganisms disclosed herein can then be added to the homogeneous mixture as the temperature is lowered to about 65° C.-95° C. whereupon additional ingredients can then be added such as flavoring agents and coloring agents. The formulation is further cooled and formed into pieces of desired dimensions.

In other pharmaceutical embodiments, the flavoring agent is incorporated into an ingestible topical vehicle which can be in the form of a mouthwash, rinse, ingestible spray, suspension, dental gel, and the like. Typical non-toxic ingestible vehicles known in the pharmaceutical arts can be used in the presently disclosed subject matter. The preferred ingestible vehicles are water, ethanol, and water-ethanol mixtures. The water-ethanol mixtures are generally employed in a weight ratio from about 1:1 to about 20:1, preferably from about 3:1 to about 20:1, and most preferably from about 3:1 to about 10:1, respectively. The pH value of the ingestible vehicle is generally from about 4 to about 7, and preferably from about 5 to about 6.5. An ingestible topical vehicle having a pH value below about 4 is generally irritating to the ingestible cavity and an ingestible vehicle having a pH value greater than about 7 generally results in an unpleasant mouth feel.

The ingestible topical flavoring agents can also contain conventional additives normally employed in those products. Conventional additives include a fluorine-providing compound, a sweetening agent, a flavoring agent, a coloring agent, a humectant, a buffer, and an emulsifier, providing the additives do not interfere with the flavoring properties of the composition. The coloring agents and humectants, and the amounts of these additives to be employed, set out above, can be used in the ingestible topical composition. The flavoring agents (flavors, flavorants) that can be used include those flavors known to the skilled artisan, such as natural and artificial flavors. Suitable flavoring agents include mints, such as peppermint, citrus flavors such as orange and lemon, artificial vanilla, cinnamon, various fruit flavors, both individual and mixed, and the like. The amount of flavoring agent employed in the ingestible topical composition is normally a matter of preference subject to such factors as the type of final ingestible composition, the individual flavor employed, and the strength of flavor desired. Thus, the amount of flavoring can be varied in order to obtain the result desired in the final product and such variations are within the capabilities of those skilled in the art without the need for undue experimentation. The flavoring agents, when used, are generally utilized in amounts that can, for example, range in amounts from about 0.05% to about 6%, by weight of the ingestible topical composition.

### 5.5. Pet Food Products

The psicose prepared and/or generated by any of the microorganisms disclosed herein can be used in a wide variety of pet food products.

As used herein, the terms “pet food” or “pet food product” refer to a product or composition that is intended for consumption by a companion animal, such as cats, dogs, guinea pigs, rabbits, birds and horses. For example, but not by way of limitation, the companion animal can be a “domestic” dog, e.g., Canis lupus familiaris. A “pet food” or “pet food product” includes any food, feed, snack, food supplement, liquid, beverage, treat, toy (chewable and/or consumable toys), meal substitute or meal replacement.

In certain embodiments, the psicose prepared and/or generated by any of the microorganisms disclosed herein is directly added to a pet food product. In certain embodiments, the psicose prepared and/or generated by any of the microorganisms disclosed herein can be added prior to, during or after formulation processing or packaging of the pet food product.

Non-limiting examples of suitable pet food products include wet food products, dry food products, moist food products, pet food supplements (e.g., vitamins), pet beverage products, snack and treats and pet food categories described herein.

In certain embodiments, the pet food product is a dry food product. A dry or low moisture-containing nutritionally-complete pet food product can comprise less than about 15% moisture. In certain embodiments, the pet food product is a wet food product. A wet or high moisture-containing nutritionally-complete pet food product can comprise greater than about 50% moisture. In certain embodiments, the pet food product is a nutritionally complete moist food product. A moist. e.g., semi-moist or semi-dry or soft dry or soft moist or intermediate or medium moisture containing nutritionally-complete pet food product comprises from about 15% to about 50% moisture.

In certain embodiments, the pet food product is a pet food snack product. Non-limiting examples of pet food snack products include snack bars, pet chews, crunchy treats, cereal bars, snacks, biscuits and sweet products.

### EXAMPLES

The presently disclosed subject matter can be better understood by reference to the following. The below examples are exemplary only and should in no way be taken as limiting.

### Example 1—Biosynthesis of Psicose from Glucose

E. coli is naturally capable of producing trace amounts of D-psicose. In the present example, D-psicose production is improved by overexpressing key genes, removing competing pathway genes, and optimizing the production conditions.

Assessing Psicose Production Capabilities in E. coli

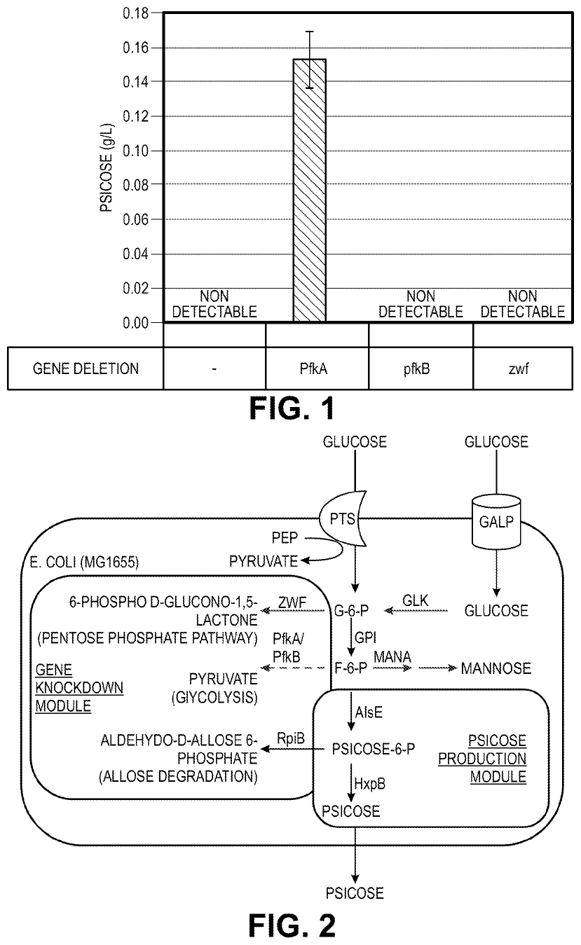

Initially, it was tested if E. coli possesses enzymes capable of producing psicose. Psicose production was tested in the production strain AL3601 depicted in Table 1 below. Cultures were grown on M9P media (M9 minimum media with 5 g/L yeast extract) with 10 g/L glucose at 30° C., with samples of the supernatant taken at 24 hours after inoculation, and then analyzed using HPLC. Notably, psicose was not detected in AL3601 (FIG. 1). A series of single gene knockouts (KOs) were constructed in AL3601. These KOs include ΔpfkA, ΔpfkB, and Δzwf, resulting in strains AL3694, AL3689, and AL3725 respectively (see Table 1). The gene zwf encodes for the enzyme glucose-6-phosphate dehydrogenase (Zwf) (EC 1.1.1.363), which converts glucose-6-phosphate (G6P) to 6-phospho-D-glucono-1,5-lactone as the first committed step in the pentose phosphate pathway (PPP). pfkA and pfkB encode for phosphofructokinase A and phosphofructokinase B (EC 2.7.1.11 and EC 2.7.1.105), which work together to convert fructose-6-phosphare (F6P) to fructose-1,6-bisphosphate (F16BP) as part of the first committed step of glycolysis. The single KO strain containing ΔpfkA produced 0.15 g/L of psicose, indicating that E. coli possesses enzymes capable of producing psicose (FIG. 1). Psicose was not detected in the single KO strains containing Δzwf and ΔpfkB.

TABLE 1
Strain list

| Strain number | Genotype |
|---|---|
| MG1655 | F lambda ilvG rfb-50 rph-1 |
| AL1050 | MG1655 + Z1 fragment (lacl^{q} tetR spec^{R}) |
| AL3601 | AL1050 + ss9::P_{lacuv5}:T7rnap |
| AL3689 | AL3601 ΔpfkB |
| AL3694 | AL3601 ΔpfkA |
| AL3695 | AL3601 ΔrpiB |
| AL3725 | AL3601 Δzwf |
| AL3727 | AL3601 Δzwf ΔpfkA |
| AL3728 | AL3601 Δzwf ΔpfkB |
| AL3729 | AL3601 Δzwf ΔpfkA ΔrpiB |
| AL3730 | AL3601 Δzwf ΔpfkB ΔrpiB |
| AL3756 | AL1050 Δzwf ΔpfkA ΔrpiB |
| AL3757 | AL1050 Δzwf ΔpfkB ΔrpiB |
| AL3989 | AL3601 Δzwf ΔpfkA ΔrpiB ΔmanA |
| AL3990 | AL1050 Δzwf ΔpfkA ΔrpiB ΔmanA |

### Determining Psicose Production Enzymes

Next, it was hypothesized that fructose-6-phosphate (F6P) is an intermediate of psicose in E. coli, since the deletion of pfkA increased psicose production. The production pathway starts with the native assimilation of glucose into E. coli via the phosphotransferase system (PTS) or GalP/Glk, which converts glucose to glucose-6-phosphate (G6P) (FIG. 2). G6P is then isomerized to F6P via glucose-6-phosphate isomerase (Gpi) (EC 5.3.1.9). F6P could be converted to psicose-6-phosphate by an epimerase (FIG. 2). A phosphatase could dephosphorylate psicose-6-phosphate to free psicose, after which it would be excreted from the cell (FIG. 2). A psicose production system using phosphorylation and dephosphorylation steps as system driving forces should be more efficient than the pathway currently used in industrial production of psicose.

By literature search and genome mining, one epimerase and two phosphatase candidates were identified. D-allulose-6-phosphate 3-epimerase (AlsE) has shown activity towards psicose. Two phosphatases (HxpB and YbiV) have shown promiscuity towards a variety of hexoses.

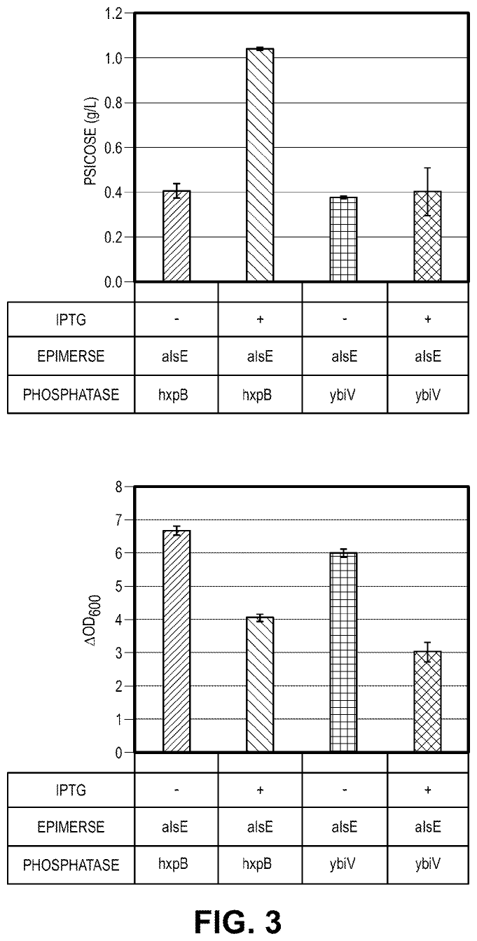

Two plasmids, pAL1946 and pAL1947 (Table 2), were constructed to overexpress genes alsE, and either hxpB or ybiV respectively, under an inducible promoter, P_{T7}. Each plasmid was introduced into AL3601, and psicose production was tested. Cultures were grown on M9P media with 10 g/L glucose at 30° C. and induced with 1 mM IPTG. After 24 hours, induced AL3601/pAL1946 produced 1.0 g/L of psicose, while induced AL3601/pAL1947 produced 0.4 g/L, indicating HxpB is the superior phosphatase for psicose production (FIG. 3).

TABLE 2
Plasmid list

Plasmid

| | |
|---|---|
| pAL1946 | P_{T7}:alsE-yniC, amp^{R}, pBR322 ori |
| pAL1947 | P_{T7}:alsE-ybiV, amp^{R}, pBR322 ori |
| pAL1952 | Pₜₑₜ:dcas9, kan^{R}, p15A ori |
| pAL2001 | P_{LIac01}:alsE-yniC, amp^{R}, ColE1 ori |
| pAL2160 | P_{LIac01}:alsE-yniC, Pⱼ₂₃₁₁₉:empty guide, amp^{R}, ColE1 ori |
| pAL2179 | P_{LIac01}:alsE-yniC, Pⱼ₂₃₁₁₉:pfkB guide, amp^{R}, ColE1 ori |
| pAL2247 | P_{gadB}:alsE-hxpB, amp^{R}, ColE1 ori |
| pAL2274 | P_{LtetO1}:galP-glk, gent^{R}, ColA ori |

### Increasing Psicose Production by Removing Competing Pathways

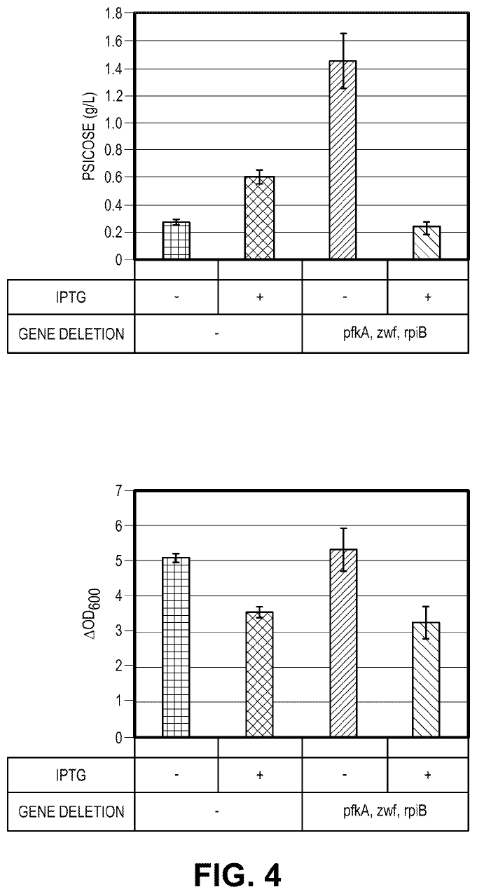

To increase carbon flux through the psicose production pathway, a triple knockout (TKO) strain containing Δzwf, ΔpfkA, and ΔrpiB (see AL3729 in Table 1) was constructed to increase the pool of F6P for psicose production. The gene rpiB encodes for the enzyme allose-6-phosphate isomerase (RpiB), which reassimilates P6P into central carbon metabolism by converting it to aldehydo-D-allose 6-phosphate in the allose degradation pathway (FIG. 2). pAL1946 (Table 2) was introduced into AL3729 (Table 1), and psicose production was tested. Cultures were grown in M9P media supplemented with 10 g/L glucose at 30° C. and induced with 25 μM IPTG. Uninduced AL3729 harboring pAL1946 generated the most psicose at 1.5 g/L after 24 hr, while induced AL3729 harboring pAL1946 produced 0.2 g/L (FIG. 4). Induced AL3601 harboring pAL1946 generated 0.6 g/L of psicose after 24 hr, and uninduced AL3601 harboring pAL1946 produced 0.2 g/L.

### Comparing Expression Systems for Psicose Production Enzymes

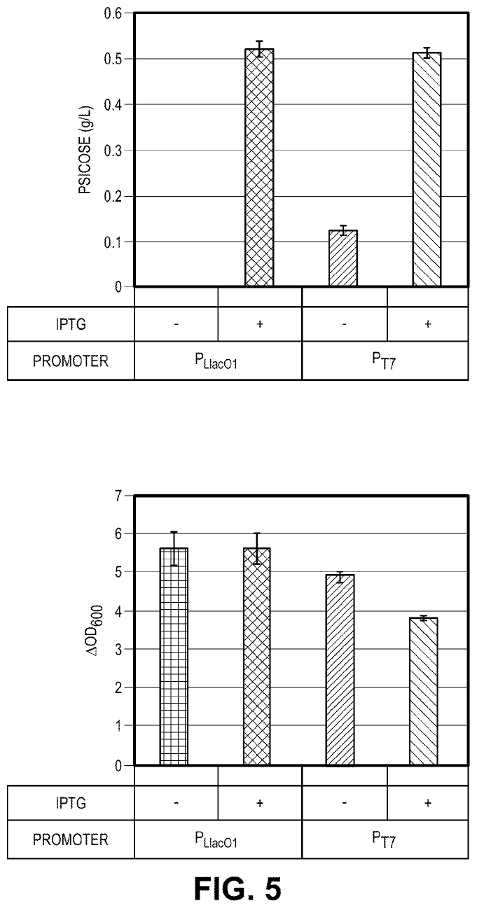

AL3601 possesses P_{lacUV5}:T7RNAP encoding the T7 RNA polymerase (Table 1). The T7 RNAP appears to pose a growth detriment to the tested strains. To eliminate this growth burden, a new psicose production plasmid (see pAL2001 in Table 2) was generated to overexpress alsE and hxpB under the IPTG-inducible promoter P_{Llaco1}, which is weaker than P_{T7}. pAL2001 was introduced into strain AL1050 (see Table 1). Strain AL1050 carries the same genotype as AL3601 but lacks P_{lactUV5}:T7RNAP. Cultures were grown on M9P media with 10 g/L glucose at 30° C. and induced with 1 mM IPTG. After 24 hours, induced AL1050 harboring pAL2001 generated 0.5 g/L of psicose, while induced AL3601 harboring pAL1946 cultures produced 0.5 g/L. Growth between induced and uninduced AL1050 harboring pAL2001 cultures appeared equivalent. In comparison, AL3601 harboring pAL1946 did not grow as well, with induced cultures reaching a much lower culture density (FIG. 5). Based on the result that pAL2001 produced comparable titers of psicose without posing a growth burden, the P_{LlacO1} based expression system was chosen as the psicose production plasmid in the next experiments.

### Comparing Expression Systems for Psicose Production Enzymes in TKO Strains

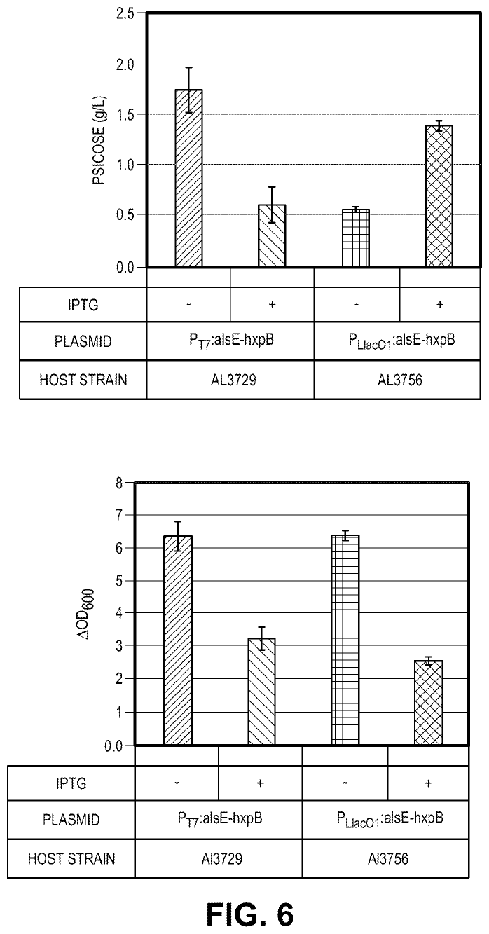

Psicose production was tested in TKO strains AL3756 for P_{LlacO1} and AL3729 for P_{T7} (see Table 1). AL3756 harboring pAL2001 and AL3729 harboring pAL1946 were grown in M9P media with 10 g/L glucose at 30° C. and induced with 1 mM IPTG. After 24 hours, induced AL3756 harboring pAL2001 produced 1.4 g/L of psicose, while uninduced cultures produced 0.6 g/L of psicose. Induced AL3729 harboring pAL1946 cultures produced 0.6 g/L of psicose and uninduced cultures produced 1.8 g/L of psicose (FIG. 6).

### Identification and Reduction of Side Products

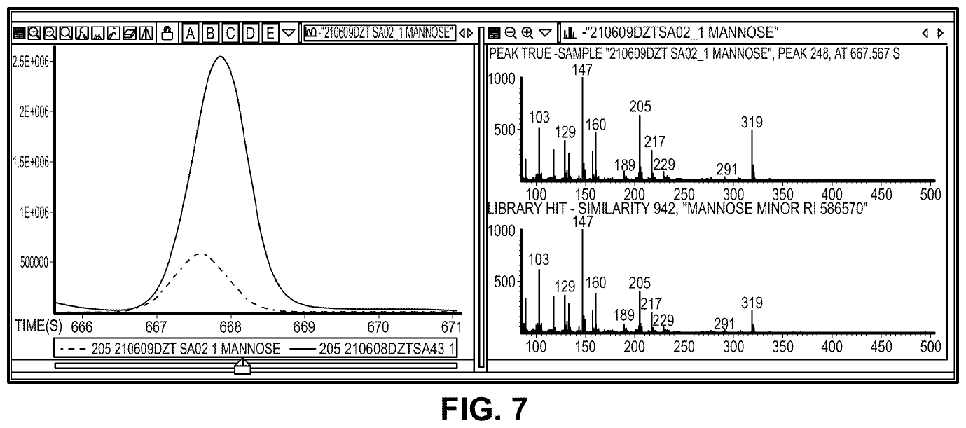

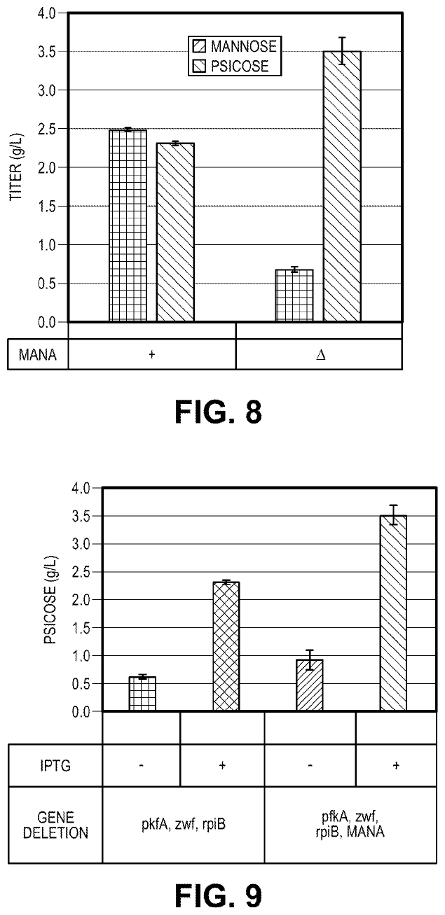

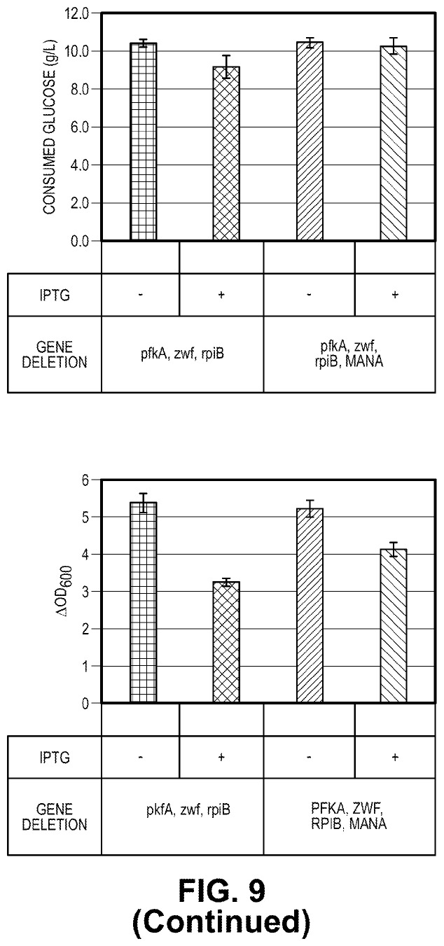

When analyzing samples from the psicose producing strains, it was consistently observed a significant peak on the HPLC chromatogram that did not align with any media component or standard for typical metabolites. It was found that the retention time and mass spectrum of the unknown peak matched the retention time and spectrum of a mannose standard (FIG. 7). To further test the hypothesis that the side product was mannose, manA was knocked out in AL3756, generating the quadruple knock out (QKO) strain AL3990 (see Table 1). The manA gene encodes for the enzyme mannose-6-phosphate isomerase (ManA), which catalyzes the reversible isomerization of mannose-6-phosphate and F6P. pAL2001 (Table 2) was introduced into AL3990 and AL3756 (Table 1), and psicose production was tested (FIG. 8 and FIG. 9). Cultures were grown in M9P media supplemented with 10 g/L glucose at 30° C. and induced with 1 mM IPTG. After 24 hours, the QKO strains generated 0.7 g/L of mannose, but 3.5 g/L psicose, with a yield of 34%. The TKO strains produced 2.5 g/L of mannose and 2.3 g/L of psicose.

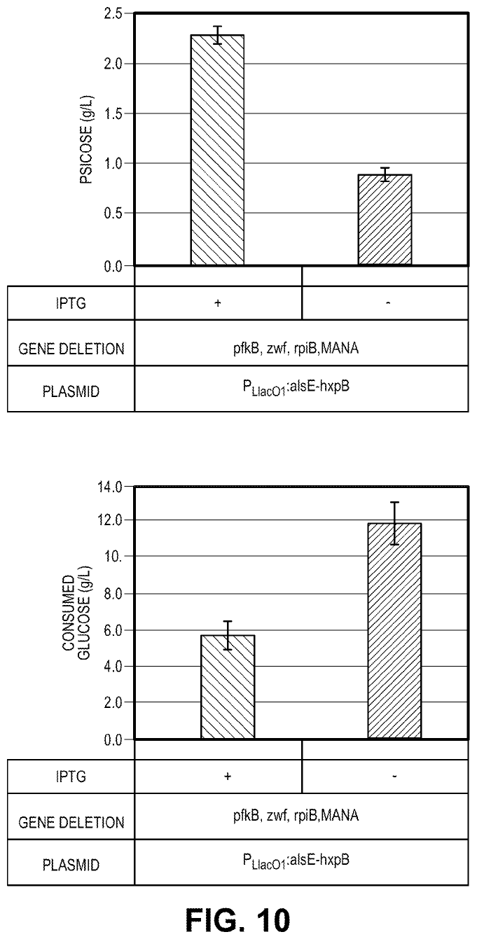

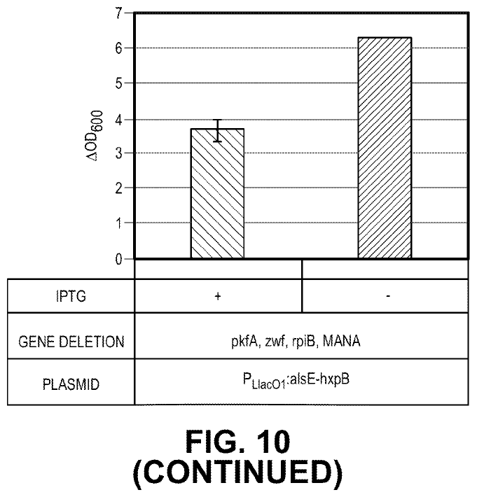

AL3990 harboring pAL2001 was cultured in M9P media supplemented with 15 g/L of glucose, rather than the usual 10 g/L. Cultures were grown at 30° C. and induced with 1 mM of IPTG. After 24 hours, induced strains generated 2.3 g/L of psicose, with a yield of 40.3% (FIG. 10).

### Dynamic Regulation of Production

Balancing carbon flux between glycolysis and production is critical for maximizing both culture health and psicose production. During the logarithmic phase of growth, cells need more energy to rigorously grow and divide. Genes related to glycolysis should be expressed, while genes related to psicose production should be suppressed. When the cells enter the stationary phase and are not actively dividing, carbon flux can be diverted from glycolysis to psicose production.

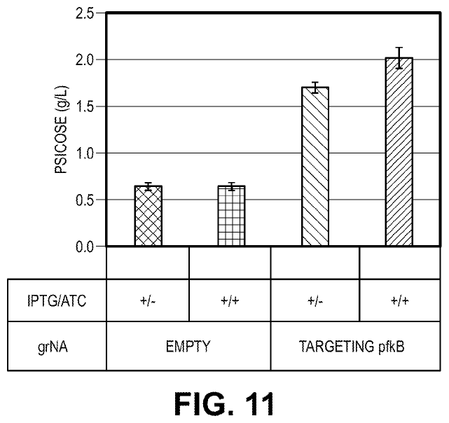

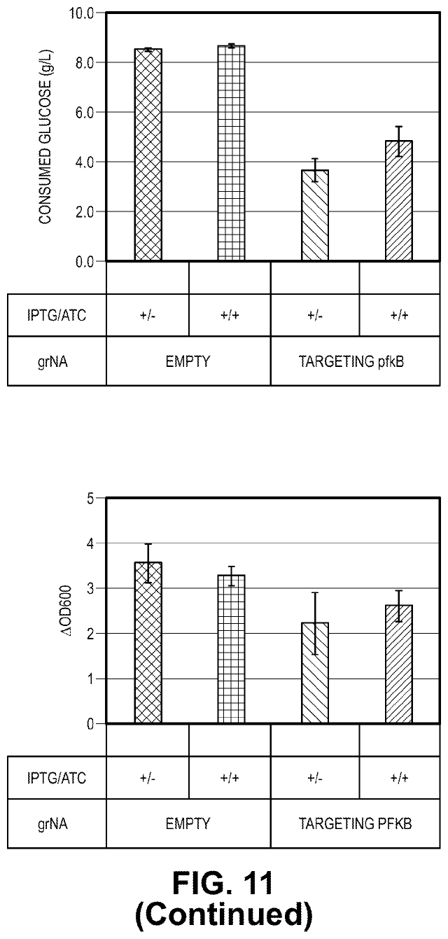

CRISPR Interference (CRISPRi) was used to down regulate pfkB in the QKO strain. The CRISPRi involves an inactivated Cas9 enzyme, dCas9, which when recruited by a single guide RNA scaffold (sgRNA) can target and block transcription initiation by RNA polymerase. Regulation of pfkB was tested using the dCas9 plasmid pAL1952 (see Table 2), which contained gene dCas9 under control of the anhydrotetracycline (aTC)-inducible promoter Pₜₑₜ. A separate production/guide plasmid was constructed, containing P_{LlacO1}:alsE-hxpB and either P_{J23119}:guide (pAL2179) or P_{J23119}:empty guide (pAL2160). The sgRNA guide encoded by pAL2179 directs dCas9 to the promoter region of pfkB. Strain AL3990 was transformed with pAL1952 and either pAL2160 or pAL2179, and psicose production was tested. Cultures were grown in M9P media (10 mL) supplemented with 10 g/L glucose at 37° C. until reaching an OD₆₀₀ of 1.0. The cells were then centrifuged and resuspended in 3.0 mL of fresh M9P media supplemented with 10 g/L of glucose, 1 mM IPTG and 100 ng/ml aTC. After 24 hours of growing at 30° C., aTC and IPTG-induced AL3990 harboring pAL1952 and pAL2179 cultures produced 1.7 g/L of psicose (yield=46%) while aTC-uninduced IPTG-induced cultures produced 2.0 g/L of psicose (yield=42%). aTC-induced AL3990 harboring pAL1952 and pAL2160 cultures produced 0.7 g/L of psicose (yield=8%) and uninduced cultures produced 0.7 g/L of psicose (yield=8%) (FIG. 11).

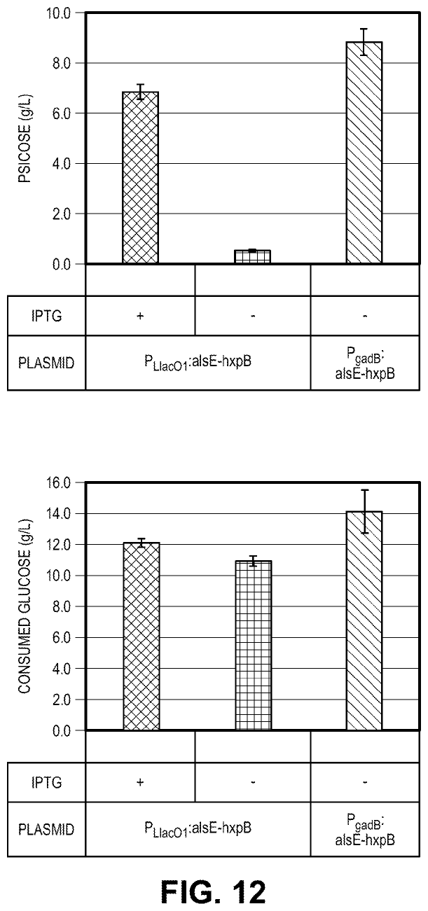

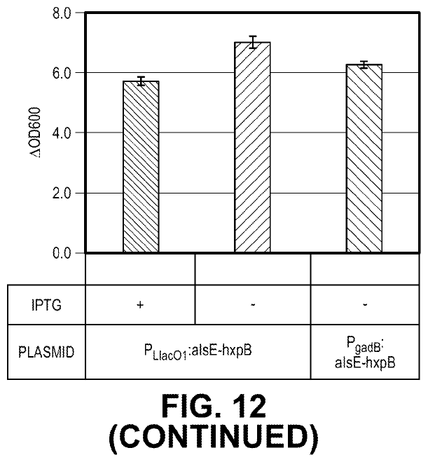

Next, an inducer-free production system using the stationary phase promoter P_{gadB} Was assessed. P_{gadB} controls expression of the gene glutamate decarboxylase B and is annotated in the literature as being primarily active during the stationary phase By controlling expression of genes alsE and hxpB using P_{gadB}, carbon flux can be diverted to production during the stationary phase. Plasmids pAL2001, containing P_{LlacO1}:alsE-hxpB. and pAL2247, containing P_{gadB}:alsE-hxpB (Table 2), were individually introduced into QKO strain AL3990 (Table 1). Cultures were grown in M9P media supplemented with 30 g/L glucose at 30° C. and induced with 1 mM IPTG (only for strains with P_{LlacO1}) for 24 hours. The induced cultures containing pAL2001 generated 6.8 g/L of psicose, with a yield of 56.5%, while cultures containing P_{gadB}:alsE-hxpB produced 8.8 g/L, with a yield of 63% (FIG. 12). P_{gadB} allowed for greater expression of production enzymes and greater flux through allulose pathway (FIG. 12). Collectively, these data suggest that inducer independent promoter is beneficial for controlling the expression of alsE and hpxB genes and for shutting down carbon flux through glycolysis.

### Increasing Glucose Uptake Using Sugar Symporter GalP

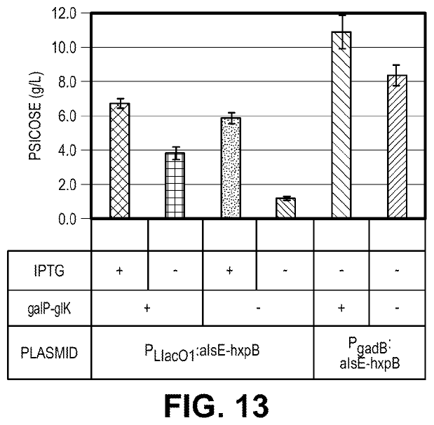

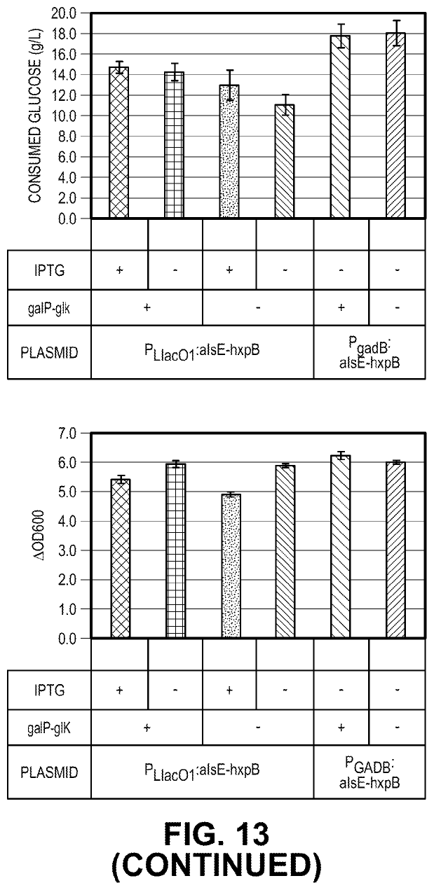

Within E. coli, the favored method for glucose uptake is the phosphotransferase system (PTS). The PTS relies on activation by phosphoenolpyruvate (PEP), which is a downstream product of glycolysis. By inhibiting glycolysis, either through gene knockouts or CRISPRi, we theorize that cellular stock of PEP may become depleted, leading to reduction in glucose consumption. To increase glucose consumption, we introduced extra copies of genes galP and glk, which encode for galactose:H⁺ symporter (GalP) and glucokinase (Glk). GalP transports glucose into the cell where it is phosphorylated to glucose-6-phosphate by Glk and assimilated into central carbon metabolism. Plasmid pAL2274 (Table 2) was constructed to express galP and glk under a P_{LtetO1} promoter. pAL2274 was introduced into QKO strain AL3990 (Table 1), along with either pAL2001 (P_{LlacO1}:alsE-hxpB) or pAL2247 (P_{gadB}:alsE-hxpB), and psicose production was tested. Cultures were grown in M9P media supplemented with 30 g/L glucose at 30° C. and induced with 1 mM IPTG for 24 hours. The strain containing pAL2247 and pAL2274 produced the most psicose, at a titer of 10.7 g/L and yield of 61%. The strain only containing pAL2247 produced 8.3 g/L of psicose, with a yield of 46%. In comparison, IPTG-induced cultures containing pAL2001 and p2274 produced 6.6 g/L of psicose with a yield of 45%, while induced cultures containing only pAL2001 produced 5.8 g/L with 46% yield (FIG. 13). It appears that overexpression of GalP-Glk may supplement glucose import when paired with production plasmid pAL2247.

TABLE 3

| Plasmid | SEQ ID | |
|---|---|---|
| name | NO: | |
| pAL1946 | 60 | taatacgactcactataggggaattgtgagcggataacaattcccct |
| (P_{T7}:alsE- | | ctagaaataattttgtttaactttaagaaggagatataccatgggcc |
| hxpB) | | atcatcatcatcatcatcatcatcatcacagcagcggccatatcgaa |
| | | ggtcgtcatatgaaaatctccccctcgttaatgtgtatggatctgct |
| | | gaaatttaaagaacagatcgaatttatcgacagccatgccgattact |
| | | tccacatcgatatcatggacggtcactttgtccccaatctgacactc |
| | | tcaccgttcttcgtaagtcaggttaaaaaactggcaactaaaccgct |
| | | cgactgtcatctgatggtgacgcggccgcaggattacattgctcaac |
| | | tggcgcgtgcgggagcagatttcatcactctgcatccggaaaccatc |
| | | aacggccaggcgttccgcctgattgatgaaatccgccgtcatgacat |
| | | gaaagtggggctgatccttaacccggagacgccagttgaggccatga |
| | | aatactatatccataaggccgataaaattacggtcatgactgtcgat |
| | | cccggctttgccggacaaccgttcattcctgaaatgctggataaact |
| | | tgccgaactgaaggcatggcgtgaacgagaaggtctggagtacgaaa |
| | | ttgaggtggacggttcctgcaaccaggcaacttacgaaaaactgatg |
| | | gcggcaggggcggatgtctttatcgtcggcacttccggcctgtttaa |
| | | tcatgcggaaaatatcgacgaagcatggagaattatgaccgcgcaga |
| | | ttctggctgcaaaaagcgaggtacagcctcatgcaaaaacagcataa |
| | | ggatccggctgctaacaaaaggagatatacatatgtcaaccccgcgt |
| | | cagattcttgctgcaatttttgatatggatggattacttatcgactc |
| | | agaacctttatgggatcgagccgaactggatgtgatggcaagcctgg |
| | | gggtggatatctcccgtcgtaacgagctgccggacaccttaggttta |
| | | cgcatcgatatggtggtcgatctttggtacgcccggcaaccgtggaa |
| | | tgggccaagccgtcaggaagtagtagaacgggttattgcccgtgcca |
| | | tttcactggttgaagagacacgtccattattaccaggcgtgcgcgaa |
| | | gccgttgcgttatgcaaagaacaaggtttattggtgggactggcctc |
| | | cgcgtcaccactacatatgctggaaaaagtgttgaccatgtttgact |
| | | tacgcgacagtttcgatgccctcgcctcggccgaaaaactgccttac |
| | | agcaagccgcatccgcaagtatatctcgactgcgcagcaaaactggg |
| | | cgttgaccctctgacctgcgtagcgctggaagattcggtaaatggca |
| | | tgatcgcctctaaagcagcccgcatgcgttccatcgtcgttcctgcg |
| | | ccagaagcgcaaaatgatccacgttttgtattagcagacgtcaaact |
| | | ttcatcgctgacagaactcaccgcaaaagaccttctcggttgaggat |
| | | ccggctgctaacaaagcccgaaaggaagctgagttggctgctgccac |
| | | cgctgagcaataactagcataaccccttggggcctctaaacgggtct |
| | | tgaggggttttttgctgaaaggaggaactatatccggatatcccgca |
| | | agaggcccggcagtaccggcataaccaagcctatgcctacagcatcc |
| | | agggtgacggtgccgaggatgacgatgagcgcattgttagatttcat |
| | | acacggtgcctgactgcgttagcaatttaactgtgataaactaccgc |
| | | attaaagcttatcgatgataagctgtcaaacatgagaattcttgaag |
| | | acgaaagggcctcgtgatacgcctatttttataggttaatgtcatga |
| | | taataatggtttcttagacgtcaggtggcacttttcggggaaatgtg |
| | | cgcggaacccctatttgtttatttttctaaatacattcaaatatgta |
| | | tccgctcatgagacaataaccctgataaatgcttcaataatattgaa |
| | | aaaggaagagtatgagtattcaacatttccgtgtcgcccttattccc |
| | | ttttttgcggcattttgccttcctgtttttgctcacccagaaacgct |
| | | ggtgaaagtaaaagatgctgaagatcagttgggtgcacgagtgggtt |
| | | acatcgaactggatctcaacagcggtaagatccttgagagttttcgc |
| | | cccgaagaacgttttccaatgatgagcacttttaaagttctgctatg |
| | | tggcgcggtattatcccgtgttgacgccgggcaagagcaactcggtc |
| | | gccgcatacactattctcagaatgacttggttgagtactcaccagtc |
| | | acagaaaagcatcttacggatggcatgacagtaagagaattatgcag |
| | | tgctgccataaccatgagtgataacactgcggccaacttacttctga |
| | | caacgatcggaggaccgaaggagctaaccgcttttttgcacaacatg |
| | | ggggatcatgtaactcgccttgatcgttgggaaccggagctgaatga |
| | | agccataccaaacgacgagcgtgacaccacgatgcctgcagcaatgg |
| | | Caacaacgttgcgcaaactattaactggcgaactacttactctagct |
| | | tcccggcaacaattaatagactggatggaggcggataaagttgcagg |
| | | accacttctgcgctcggcccttccggctggctggtttattgctgata |
| | | aatctggagccggtgagcgtgggtctcgcggtatcattgcagcactg |
| | | gggccagatggtaagccctcccgtatcgtagttatctacacgacggg |
| | | gagtcaggcaactatggatgaacgaaatagacagatcgctgagatag |
| | | gtgcctcactgattaagcattggtaactgtcagaccaagtttactca |
| | | tatatactttagattgatttaaaacttcatttttaatttaaaaggat |
| | | ctaggtgaagatcctttttgataatctcatgaccaaaatcccttaac |
| | | gtgagttttcgttccactgagcgtcagaccccgtagaaaagatcaaa |
| | | ggatcttcttgagatcctttttttctgcgcgtaatctgctgcttgca |
| | | aacaaaaaaaccaccgctaccagcggtggtttgtttgccggatcaag |
| | | agctaccaactctttttccgaaggtaactggcttcagcagagcgcag |
| | | ataccaaatactgtccttctagtgtagccgtagttaggccaccactt |
| | | caagaactctgtagcaccgcctacatacctcgctctgctaatcctgt |
| | | taccagtggctgctgccagtggcgataagtcgtgtcttaccgggttg |
| | | gactcaagacgatagttaccggataaggcgcagcggtcgggctgaac |
| | | ggggggttcgtgcacacagcccagcttggagcgaacgacctacaccg |
| | | aactgagatacctacagcgtgagctatgagaaagcgccacgcttccc |
| | | gaagggagaaaggcggacaggtatccggtaagcggcagggtcggaac |
| | | aggagagcgcacgagggagcttccagggggaaacgcctggtatcttt |
| | | atagtcctgtcgggtttcgccacctctgacttgagcgtcgatttttg |
| | | tgatgctcgtcaggggggcggagcctatggaaaaacgccagcaacgc |
| | | ggcctttttacggttcctggccttttgctggccttttgctcacatgt |
| | | tctttcctgcgttatcccctgattctgtggataaccgtattaccgcc |
| | | tttgagtgagctgataccgctcgccgcagccgaacgaccgagcgcag |
| | | cgagtcagtgagcgaggaagcggaagagcgcctgatgcggtattttc |
| | | tccttacgcatctgtgcggtatttcacaccgcatatatggtgcactc |
| | | tcagtacaatctgctctgatgccgcatagttaagccagtatacactc |
| | | cgctatcgctacgtgactgggtcatggctgcgccccgacacccgcca |
| | | acacccgctgacgcgccctgacgggcttgtctgctcccggcatccgc |
| | | ttacagacaagctgtgaccgtctccgggagctgcatgtgtcagaggt |
| | | tttcaccgtcatcaccgaaacgcgcgaggcagctgcggtaaagctca |
| | | tcagcgtggtcgtgaagcgattcacagatgtctgcctgttcatccgc |
| | | gtccagctcgttgagtttctccagaagcgttaatgtctggcttctga |
| | | taaagcgggccatgttaagggcggttttttcctgtttggtcactgat |
| | | gcctccgtgtaagggggatttctgttcatgggggtaatgataccgat |
| | | gaaacgagagaggatgctcacgatacgggttactgatgatgaacatg |
| | | cccggttactggaacgtigtgagggtaaacaactggcggtatggatg |
| | | cggcgggaccagagaaaaatcactcagggtcaatgccagcgcttcgt |
| | | taatacagatgtaggtgttccacagggtagccagcagcatcctgcga |
| | | tgcagatccggaacataatggtgcagggcgctgacttccgcgtttcc |
| | | agactttacgaaacacggaaaccgaagaccattcatgttgttgctca |
| | | ggtcgcagacgttttgcagcagcagtcgcttcacgttcgctcgcgta |
| | | tcggtgattcattctgctaaccagtaaggcaaccccgccagcctagc |
| | | cgggtcctcaacgacaggagcacgatcatgcgcacccgtggccagga |
| | | cccaacgctgcccgagatgcgccgcgtgcggctgctggagatggcgg |
| | | acgcgatggatatgttctgccaagggttggtttgcgcattcacagtt |
| | | ctccgcaagaattgattggctccaattcttggagtggtgaatccgtt |
| | | agcgaggtgccgccggcttccattcaggtcgaggtggcccggctcca |
| | | tgcaccgcgacgcaacgcggggaggcagacaaggtatagggcggcgc |
| | | ctacaatccatgccaacccgttccatgtgctcgccgaggcggcataa |
| | | atcgccgtgacgatcagcggtccagtgatcgaagttaggctggtaag |
| | | agccgcgagcgatccttgaagctgtccctgatggtcgtcatctacct |
| | | gcctggacagcatggcctgcaacgcgggcatcccgatgccgccggaa |
| | | gcgagaagaatcataatggggaaggccatccagcctcgcgtcgcgaa |
| | | cgccagcaagacgtagcccagcgcgtcggccgccatgccggcgataa |
| | | tggcctgcttctcgccgaaacgtttggtggvgggaccagtgacgaag |
| | | gcttgagcgagggcgtgcaagattccgaataccgcaagcgacaggcc |
| | | gatcatcgtcgcgctccagcgaaagcggtcctcgccgaaaatgaccc |
| | | agagcgctgccggcacctgtcctacgagttgcatgataaagaagaca |
| | | gtcataagtgcggcgacgatagtcatgccccgcgcccaccggaagga |
| | | gctgactgggttgaaggctctcaagggcatcggtcgagatcccagtg |
| | | cctaatgagtgagctaacttacattaattgcgttgcgctcactgccc |
| | | gctttccagtcgggaaacctgtcgtgccagctgcattaatgaatcgg |
| | | ccaacgcgcggggagaggcggtttgcgtattgggcgccagggtcgtt |
| | | tttcttttcaccagtgagacgggcaacagctgattgcccttcaccgc |
| | | ctggccctgagagagttgcagcaagcggtccacgctggtttgcccca |
| | | gcaggcgaaaatcctgtttgatggtggttaacggcgggatataacat |
| | | gagctgtcttcggtatcgtcgtatcccactaccgagatatccgcacc |
| | | aacgcgcagcccggactcggtaatggcgcgcattgcgcccagcgcca |
| | | tctgatcgttggcaaccagcatcgcagtgggaacgatgccctcattc |
| | | agcatttgcatggtttgttgaaaaccggacatggcactccagtcgcc |
| | | ttcccgttccgctatcggctgaatttgattgcgagtgagatatttat |
| | | gccagccagccagacgcagacgcgccgagacagaacttaatgggccc |
| | | gctaacagcgcgatttgctggtgacccaatgcgaccagatgctccac |
| | | gcccagtcgcgtaccgtcttcatgggagaaaataatactgttgatgg |
| | | gtgtctggtcagagacatcaagaaataacgccggaacattagtgcag |
| | | gcagcttccacagcaatggcatcctggtcatccagcggatagttaat |
| | | gatcagcccactgacgcgttgcgcgagaagattgtgcaccgccgctt |
| | | tacaggcttcgacgccgcttcgttctaccatcgacaccaccacgctg |
| | | gcacccagttgatcggcgcgagatttaatcgccgcgacaatttgcga |
| | | cggcgcgtgcagggccagactggaggtggcaacgccaatcagcaacg |
| | | actgtttgcccgccagttgttgtgccacgcggttgggaatgtaattc |
| | | agctccgccatcgccgcttccactttttcccgcgttttcgcagaaac |
| | | gtggctggcctggttcaccacgcgggaaacggtctgataagagacac |
| | | cggcatactctgcgacatcgtataacgttactggtttcacattcacc |
| | | accctgaattgactctcttccgggcgctatcatgccataccgcgaaa |
| | | ggttttgcgccattcgatggtgtccgggatctcgacgctctccctta |
| | | tgcgactcctgcattaggaagcagcccagtagtaggttgaggccgtt |
| | | gagcaccgccgccgcaaggaatggtgcatgcaaggagatggcgccca |
| | | acagtcccccggccacggggcctgccaccatacccacgccgaaacaa |
| | | gcgctcatgagcccgaagtggcgagcccgatcttccccatcggtgat |
| | | gtcggcgatataggcgccagcaaccgcacctgtggcgccggtgatgc |
| | | cggccacgatgcgtccggcgtagaggatcgagatctcgatcccgcga |
| | | aat |
| pAL1947 | 61 | taatacgactcactataggggaattgtgagcggataacaattcccct |
| (P_{T7}:alsE- | | ctagaaataattttgtttaactttaagaaggagatataccatgggcc |
| ybiV) | | atcatcatcatcatcatcatcatcatcacagcagcggccatatcgaa |
| | | ggtcgtcatatgaaaatctccccctcgttaatgtgtatggatctgct |
| | | gaaatttaaagaacagatcgaatttatcgacagccatgccgattact |
| | | tccacatcgatatcatggacggtcactttgtccccaatctgacactc |
| | | tcaccgttcttcgtaagtcaggttaaaaaactggcaactaaaccgct |
| | | cgactgtcatctgatggtgacgcggccgcaggattacattgctcaac |
| | | tggcgcgtgcgggagcagatttcatcactctgcatccggaaaccatc |
| | | aacggccaggcgttccgcctgattgatgaaatccgccgtcatgacat |
| | | gaaagtggggctgatccttaacccggagacgccagttgaggccatga |
| | | aatactatatccataaggccgataaaattacggtcatgactgtcgat |
| | | cccggctttgccggacaaccgttcattcctgaaatgctggataaact |
| | | tgccgaactgaaggcatggcgtgaacgagaaggtctggagtacgaaa |
| | | ttgaggtggacggttcctgcaaccaggcaacttacgaaaaactgatg |
| | | gcggcaggggcggatgtctttatcgtcggcacttccggcctgtttaa |
| | | tcatgcggaaaatatcgacgaagcatggagaattatgaccgcgcaga |
| | | ttctggctgcaaaaagcgaggtacagcctcatgcaaaaacagcataa |
| | | ggatccggctgctaacaaaaggagatatacatatgagcgtaaaagtt |
| | | atcgtcacagacatggacggtacttttcttaacgacgccaaaacgta |
| | | caaccaaccacgtittatggcgcaatatcaggaactgaaaaagcgcg |
| | | gcattaagttcgttgttgccagcggtaatcagtattaccagcttatt |
| | | tcattctttcctgagctaaaggatgagatctcttttgtcgcggaaaa |
| | | cggcgcactggtttacgaacatggcaagcagttgttccacggcgaac |
| | | tgacccgacatgaatcgcggattgttattggcgagttgctaaaagat |
| | | aagcaactcaattttgtcgcctgcggtctgcaaagtgcatatgtcag |
| | | cgaaaatgcccccgaagcatttgtcgcactgatggcaaaacactacc |
| | | atcgcctgaaacctgtaaaagattatcaggagattgacgacgtactg |
| | | ttcaagttttcgctcaacctgccggatgaacaaatcccgttagtgat |
| | | cgacaaactgcacgtagcgctcgatggcattatgaaacccgttacca |
| | | gtggttttggctttatcgacctgattattcccggtctacataaagca |
| | | aacggtatttcgcggttactgaaacgctgggatctgtcaccgcaaaa |
| | | tgtggtagcgattggcgacagcggtaacgatgcggagatgctgaaaa |
| | | tggcgcgttattcctttgcgatgggcaatgctgcggaaaacattaaa |
| | | caaatcgcccgttacgctaccgatgataataatcatgaaggcgcgct |
| | | gaatgtgattcaggcggtgctggataacacatccccttttaacagct |
| | | gaggatccggctgctaacaaagcccgaaaggaagctgagttggctgc |
| | | tgccaccgctgagcaataactagcataaccccttggggcctctaaac |
| | | gggtcttgaggggttttttgctgaaaggaggaactatatccggatat |
| | | cccgcaagaggcccggcagtaccggcataaccaagcctatgcctaca |
| | | gcatccagggtgacggtgccgaggatgacgatgagcgcattgttaga |
| | | tttcatacacggtgcctgactgcgttagcaatttaactgtgataaac |
| | | taccgcattaaagcttatcgatgataagctgtcaaacatgagaattc |
| | | ttgaagacgaaagggcctcgtgatacgcctatttttataggttaatg |
| | | tcatgataataatggtttcttagacgtcaggtggcactittcgggga |
| | | aatgtgcgcggaacccctatttgtttatttttctaaatacattcaaa |
| | | tatgtatccgctcatgagacaataaccctgataaatgcttcaataat |
| | | attgaaaaaggaagagtatgagtattcaacatttccgtgtcgccctt |
| | | attcccttttttgcggcattttgccttcctgtttttgctcacccaga |
| | | aacgctggtgaaagtaaaagatgctgaagatcagttgggtgcacgag |
| | | tgggttacatcgaactggatctcaacagcggtaagatccttgagagt |
| | | tttcgccccgaagaacgttttccaatgatgagcacttttaaagttct |
| | | gctatgtggcgcggtattatcccgtgttgacgccgggcaagagcaac |
| | | tcggtcgccgcatacactattctcagaatgacttggttgagtactca |
| | | ccagtcacagaaaagcatcttacggatggcatgacagtaagagaatt |
| | | atgcagtgctgccataaccatgagtgataacactgcggccaacttac |
| | | ttctgacaacgatcggaggaccgaaggagctaaccgcttttttgcac |
| | | aacatgggggatcatgtaactcgccttgatcgttgggaaccggagct |
| | | gaatgaagccataccaaacgacgagcgtgacaccacgatgcctgcag |
| | | caatggcaacaacgttgcgcaaactattaactggcgaactacttact |
| | | ctagcttcccggcaacaattaatagactggatggaggcggataaagt |
| | | tgcaggaccacttctgcgctcggcccttccggctggctggtttattg |
| | | ctgataaatctggagccggtgagcgtgggtctcgcggtatcattgca |
| | | gcactggggccagatggtaagccctcccgtatcgtagttatctacac |
| | | gacggggagtcaggcaactatggatgaacgaaatagacagatcgctg |
| | | agataggtgcctcactgattaagcattggtaactgtcagaccaagtt |
| | | tactcatatatactttagattgatttaaaacttcatttttaatttaa |
| | | aaggatctaggtgaagatcctttttgataatctcatgaccaaaatcc |
| | | cttaacgtgagttttcgttccactgagcgtcagaccccgtagaaaag |
| | | atcaaaggatcttcttgagatcctttttttctgcgcgtaatctgctg |
| | | cttgcaaacaaaaaaaccaccgctaccagcggtggtttgtttgccgg |
| | | atcaagagctaccaactctttttccgaaggtaactggcttcagcaga |
| | | gcgcagataccaaatactgtccttctagtgtagccgtagttaggcca |
| | | ccacttcaagaactctgtagcaccgcctacatacctcgctctgctaa |
| | | tcctgttaccagtggctgctgccagtggcgataagtcgtgtcttacc |
| | | gggttggactcaagacgatagttaccggataaggcgcagcggtcggg |
| | | ctgaacggggggttcgtgcacacagcccagcttggagcgaacgacct |
| | | acaccgaactgagatacctacagcgtgagctatgagaaagcgccacg |
| | | cttcccgaagggagaaaggcggacaggtatccggtaagcggcagggt |
| | | cggaacaggagagcgcacgagggagcttccagggggaaacgcctggt |
| | | atctttatagtcctgtcgggtttcgccacctctgacttgagcgtcga |
| | | tttttgtgatgctcgtcaggggggcggagcctatggaaaaacgccag |
| | | caacgcggcctttttacggttcctggccttttgctggccttttgctc |
| | | acatgttctttcctgcgttatcccctgattctgtggataaccgtatt |
| | | accgcctttgagtgagctgataccgctcgccgcagccgaacgaccga |
| | | gcgcagcgagtcagtgagcgaggaagcggaagagcgcctgatgcggt |
| | | attttctccttacgcatctgtgcggtatttcacaccgcatatatggt |
| | | gcactctcagtacaatctgctctgatgccgcatagttaagccagtat |
| | | acactccgctatcgctacgtgactgggtcatggctgcgccccgacac |
| | | ccgccaacacccgctgacgcgccctgacgggcttgtctgctcccggc |
| | | atccgcttacagacaagctgtgaccgtctccgggagctgcatgtgtc |
| | | agaggttttcaccgtcatcaccgaaacgcgcgaggcagctgcggtaa |
| | | agctcatcagcgtggtcgtgaagcgattcacagatgtctgcctgttc |
| | | atccgcgtccagctcgttgagtttctccagaagcgttaatgtctggc |
| | | ttctgataaagcgggccatgttaagggcggttttttcctgtttggtc |
| | | actgatgcctccgtgtaagggggatttctgttcatgggggtaatgat |
| | | accgatgaaacgagagaggatgctcacgatacgggttactgatgatg |
| | | aacatgcccggttactggaacgttgtgagggtaaacaactggcggta |
| | | tggatgcggcgggaccagagaaaaatcactcagggtcaatgccagcg |
| | | cttcgttaatacagatgtaggtgttccacagggtagccagcagcatc |
| | | ctgcgatgcagatccggaacataatggtgcagggcgctgacttccgc |
| | | gtttccagactttacgaaacacggaaaccgaagaccattcatgttgt |
| | | tgctcaggtcgcagacgttttgcagcagcagtcgcttcacgttcgct |
| | | cgcgtatcggtgattcattctgctaaccagtaaggcaaccccgccag |
| | | cctagccgggtcctcaacgacaggagcacgatcatgcgcacccgtgg |
| | | ccaggacccaacgctgcccgagatgcgccgcgtgcggctgctggaga |
| | | tggcggacgcgatggatatgttctgccaagggttggtttgcgcattc |
| | | acagttctccgcaagaattgattggctccaattcttggagtggtgaa |
| | | tccgttagcgaggtgccgccggcttccattcaggtcgaggtggcccg |
| | | gctccatgcaccgcgacgcaacgcggggaggcagacaaggtataggg |
| | | cggcgcctacaatccatgccaacccgttccatgtgctcgccgaggcg |
| | | gcataaatcgccgtgacgatcagcggtccagtgatcgaagttaggct |
| | | ggtaagagccgcgagcgatccttgaagctgtccctgatggtcgtcat |
| | | ctacctgcctggacagcatggcctgcaacgcgggcatcccgatgccg |
| | | ccggaagcgagaagaatcataatggggaaggccatccagcctcgcgt |
| | | cgcgaacgccagcaagacgtagcccagcgcgtcggccgccatgccgg |
| | | cgataatggcctgcttctcgccgaaacgtttggtggcgggaccagtg |
| | | acgaaggcttgagcgagggcgtgcaagattccgaataccgcaagcga |
| | | caggccgatcatcgtcgcgctccagcgaaagcggtcctcgccgaaaa |
| | | tgacccagagcgctgccggcacctgtcctacgagttgcatgataaag |
| | | aagacagtcataagtgcggcgacgatagtcatgccccgcgcccaccg |
| | | gaaggagctgactgggttgaaggctctcaagggcatcggtcgagatc |
| | | ccggtgcctaatgagtgagctaacttacattaattgcgttgcgctca |
| | | ctgcccgctttccagtcgggaaacctgtcgtgccagctgcattaatg |
| | | aatcggccaacgcgcggggagaggcggtttgcgtattgggcgccagg |
| | | gtggtttttcttttcaccagtgagacgggcaacagctgattgccctt |
| | | caccgcctggccctgagagagttgcagcaagcggtccacgctggttt |
| | | gccccagcaggcgaaaatcctgtttgatggtggttaacggcgggata |
| | | taacatgagctgtcttcggtatcgtcgtatcccactaccgagatatc |
| | | cgcaccaacgcgcagcccggactcggtaatggcgcgcattgcgccca |
| | | gcgccatctgatcgttggcaaccagcatcgcagtgggaacgatgccc |
| | | tcattcagcatttgcatggtttgttgaaaaccggacatggcactcca |
| | | gtcgccttcccgttccgctatcggctgaatttgattgcgagtgagat |
| | | atttatgccagccagccagacgcagacgcgccgagacagaacttaat |
| | | gggcccgctaacagcgcgatttgctggtgacccaatgcgaccagatg |
| | | ctccacgcccagtcgcgtaccgtcttcatgggagaaaataatactgt |
| | | tgatgggtgtctggtcagagacatcaagaaataacgccggaacatta |
| | | gtgcaggcagcttccacagcaatggcatcctggtcatccagcggata |
| | | gttaatgatcagcccactgacgcgttgcgcgagaagattgtgcaccg |
| | | ccgctttacaggcttcgacgccgcttcgttctaccatcgacaccacc |
| | | acgctggcacccagttgatcggcgcgagatttaatcgccgcgacaat |
| | | ttgcgacggcgcgtgcagggccagactggaggtggcaacgccaatca |
| | | gcaacgactgtttgcccgccagtigttgtgccacgcggttgggaatg |
| | | taattcagctccgccatcgccgcttccactttttcccgcgttttcgc |
| | | agaaacgtggctggcctggttcaccacgcgggaaacggtctgataag |
| | | agacaccggcatactctgcgacatcgtataacgttactggtttcaca |
| | | ttcaccaccctgaattgactctcttccgggcgctatcatgccatacc |
| | | gcgaaaggttttgcgccattcgatggtgtccgggatctcgacgctct |
| | | cccttatgcgactcctgcattaggaagcagcccagtagtaggttgag |
| | | gccgttgagcaccgccgccgcaaggaatggtgcatgcaaggagatgg |
| | | cgcccaacagtcccccggccacggggcctgccaccatacccacgccg |
| | | aaacaagcgctcatgagcccgaagtggcgagcccgatcttccccatc |
| | | ggtgatgtcggcgatataggcgccagcaaccgcacctgtggcgccgg |
| | | tgatgccggccacgatgcgtccggcgtagaggatcgagatctcgatc |
| | | ccgcgaaat |
| pAL2001 | 62 | aattgtgagcggataacaattgacattgtgagcggataacaagatac |
| (P_{LlacO1}:alSE- | | tgagcacatcagcaggacgcactgaccgaattcattaaagaggagaa |
| hxpB) | | aagatataccatgaaaatctccccctcgttaatgtgtatggatctgc |
| | | tgaaatttaaagaacagatcgaatttatcgacagccatgccgattac |
| | | ttccacatcgatatcatggacggtcactttgtccccaatctgacact |
| | | ctcaccgttcttcgtaagtcaggttaaaaaactggcaactaaaccgc |
| | | tcgactgtcatctgatggtgacgcggccgcaggattacattgctcaa |
| | | ctggcgcgtgcgggagcagatttcatcactctgcatccggaaaccat |
| | | caacggccaggcgttccgcctgattgatgaaatccgccgtcatgaca |
| | | tgaaagtggggctgatccttaacccggagacgccagttgaggccatg |
| | | aaatactatatccataaggccgataaaattacggtcatgactgtcga |
| | | tcccggctttgccggacaaccgttcattcctgaaatgctggataaac |
| | | ttgccgaactgaaggcatggcgtgaacgagaaggtctggagtacgaa |
| | | attgaggtggacggttcctgcaaccaggcaacttacgaaaaactgat |
| | | ggcggcaggggcggatgtctttatcgtcggcacttccggcctgttta |
| | | atcatgcggaaaatatcgacgaagcatggagaattatgaccgcgcag |
| | | attctggctgcaaaaagcgaggtacagcctcatgcaaaaacagcata |
| | | aggatccggctgctaacaaaaggagatatacatatgtcaaccccgcg |
| | | tcagattcttgctgcaatttttgatatggatggattacttatcgact |
| | | cagaacctttatgggatcgagccgaactggatgtgatggcaagcctg |
| | | ggggtggatatctcccgtcgtaacgagctgccggacaccttaggttt |
| | | acgcatcgatatggtggtcgatctttggtacgcccggcaaccgtgga |
| | | atgggccaagccgtcaggaagtagtagaacgggttattgcccgtgcc |
| | | atttcactggttgaagagacacgtccattattaccaggcgtgcgcga |
| | | agccgttgcgttatgcaaagaacaaggtttattggtgggactggcct |
| | | ccgcgtcaccactacatatgctggaaaaagtgttgaccatgtttgac |
| | | ttacgcgacagtttcgatgccctcgcctcggccgaaaaactgcctta |
| | | cagcaagccgcatccgcaagtatatctcgactgcgcagcaaaactgg |
| | | gcgttgaccctctgacctgcgtagcgctggaagattcggtaaatggc |
| | | atgatcgcctctaaagcagcccgcatgcgttccatcgtcgttcctgc |
| | | gccagaagcgcaaaatgatccacgttttgtattagcagacgtcaaac |
| | | tttcatcgctgacagaactcaccgcaaaagaccttctcggttgagat |
| | | ccggctgctaaggtacctaatctagaggcatcaaataaaacgaaagg |
| | | ctcagtcgaaagactgggcctttcgttttatctgttgtttgtcggtg |
| | | aacgctctcctgagtaggacaaatccgccgccctagacctaggcgtt |
| | | cggctgcggcgagcggtatcagctcactcaaaggcggtaatacggtt |
| | | atccacagaatcaggggataacgcaggaaagaacatgtgagcaaaag |
| | | gccagcaaaaggccaggaaccgtaaaaaggccgcgttgctggcgttt |
| | | ttccataggctccgcccccctgacgagcatcacaaaaatcgacgctc |
| | | aagtcagaggtggcgaaacccgacaggactataaagataccaggcgt |
| | | ttccccctggaagctccctcgtgcgctctcctgttccgaccctgccg |
| | | cttaccggatacctgtccgcctttctcccttcgggaagcgtggcgct |
| | | ttctcaatgctcacgctgtaggtatctcagttcggtgtaggtcgttc |
| | | gctccaagctgggctgtgtgcacgaaccccccgttcagcccgaccgc |
| | | tgcgccttatccggtaactatcgtcttgagtccaacccggtaagaca |
| | | cgacttatcgccactggcagcagccactggtaacaggattagcagag |
| | | cgaggtatgtaggcggtgctacagagttcttgaagtggtggcctaac |
| | | tacggctacactagaaggacagtatttggtatctgcgctctgctgaa |
| | | gccagttaccttcggaaaaagagttggtagctcttgatccggcaaac |
| | | aaaccaccgctggtagcggtggtttttttgtttgcaagcagcagatt |
| | | acgcgcagaaaaaaaggatctcaagaagatcctttgatcttttctac |
| | | ggggtctgacgctcagtggaacgaaaactcacgttaagggattttgg |
| | | tcatgactagtgcttggattctcaccaataaaaaacgcccggcggca |
| | | accgagcgttctgaacaaatccagatggagttctgaggtcattactg |
| | | gatctatcaacaggagtccaagcgagctcgtaaacttggtctgacag |
| | | ttaccaatgcttaatcagtgaggcacctatctcagcgatctgtctat |
| | | ttcgttcatccatagttgcctgactccccgtcgtgtagataactacg |
| | | atacgggagggcttaccatctggccccagtgctgcaatgataccgcg |
| | | agacccacgctcaccggctccagatttatcagcaataaaccagccag |
| | | ccggaagggccgagcgcagaagtggtcctgcaactttatccgcctcc |
| | | atccagtctattaattgttgccgggaagctagagtaagtagttcgcc |
| | | agttaatagtttgcgcaacgttgttgccattgctacaggcatcgtgg |
| | | tgtcacgctcgtcgtttggtatggcttcattcagctccggttcccaa |
| | | cgatcaaggcgagttacatgatcccccatgttgtgcaaaaaagcggt |
| | | tagctccttcggtcctccgatcgttgtcagaagtaagttggccgcag |
| | | tgttatcactcatggttatggcagcactgcataattctcttactgtc |
| | | atgccatccgtaagatgcttttctgtgactggtgagtactcaaccaa |
| | | gtcattctgagaatagtgtatgcggcgaccgagttgctcttgcccgg |
| | | cgtcaatacgggataataccgcgccacatagcagaactttaaaagtg |
| | | ctcatcattggaaaacgttcttcggggcgaaaactctcaaggatctt |
| | | accgctgttgagatccagttcgatgtaacccactcgtgcacccaact |
| | | gatcttcagcatcttttactttcaccagcgtttctgggtgagcaaaa |
| | | acaggaaggcaaaatgccgcaaaaaagggaataagggcgacacggaa |
| | | atgttgaatactcatactcttcctttttcaatattattgaagcattt |
| | | atcagggttattgtctcatgagcggatacatatttgaatgtatttag |
| | | aaaaataaacaaataggggttccgcgcacatttccccgaaaagtgcc |
| | | acctgacgtctaagaaaccattattatcatgacattaacctataaaa |
| | | ataggcgtatcacgaggccctttcgtcttcacctcgag |
| pAL1952 | 63 | gttgacactctatcgttgatagagttattttaccactccctatcagt |
| (Pₜₑₜ:dcas9) | | gatagagaaaagaattcaaaagatctaaagaggagaaaggatctatg |
| | | gataagaaatactcaataggcttagctatcggcacaaatagcgtcgg |
| | | atgggcggtgatcactgatgaatataaggttccgtctaaaaagttca |
| | | aggttctgggaaatacagaccgccacagtatcaaaaaaaatcttata |
| | | ggggctcttttatttgacagtggagagacagcggaagcgactcgtct |
| | | caaacggacagctcgtagaaggtatacacgtcggaagaatcgtattt |
| | | gttatctacaggagattttttcaaatgagatggcgaaagtagatgat |
| | | agtttctttcatcgacttgaagagtcttttttggtggaagaagacaa |
| | | gaagcatgaacgtcatcctatttttggaaatatagtagatgaagttg |
| | | cttatcatgagaaatatccaactatctatcatctgcgaaaaaaattg |
| | | gtagattctactgataaagcggatttgcgcttaatctatttggcctt |
| | | agcgcatatgattaagtttcgtggtcattttttgattgagggagatt |
| | | taaatcctgataatagtgatgtggacaaactatttatccagttggta |
| | | caaacctacaatcaattatttgaagaaaaccctattaacgcaagtgg |
| | | agtagatgctaaagcgattctttctgcacgattgagtaaatcaagac |
| | | gattagaaaatctcattgctcagctccccggtgagaagaaaaatggc |
| | | ttatttgggaatctcattgctttgtcattgggtttgacccctaattt |
| | | taaatcaaattttgatttggcagaagatgctaaattacagctttcaa |
| | | aagatacttacgatgatgatttagataatttattggcgcaaattgga |
| | | gatcaatatgctgatttytttttggcagctaagaatttatcagatgc |
| | | tattttactttcagatatcctaagagtaaatactgaaataactaagg |
| | | ctcccctatcagcttcaatgattaaacgctacgatgaacatcatcaa |
| | | gacttgactcttttaaaagctttagttcgacaacaacttccagaaaa |
| | | gtataaagaaatcttttttgatcaatcaaaaaacggatatgcaggtt |
| | | atattgatgggggagctagccaagaagaattttataaatttatcaaa |
| | | ccaattttagaaaaaatggatggtactgaggaattattggtgaaact |
| | | aaatcgtgaagatttgctgcgcaagcaacggacctttgacaacggct |
| | | ctattccccatcaaattcacttgggtgagctgcatgctattttgaga |
| | | agacaagaagacttttatccatttttaaaagacaatcgtgagaagat |
| | | tgaaaaaatcttgacttttcgaattccttattatgttggtccattgg |
| | | cgcgtggcaatagtcgttttgcatggatgactcggaagtctgaagaa |
| | | acaattaccccatggaattttgaagaagttgtcgataaaggtgcttc |
| | | agctcaatcatttattgaacgcatgacaaactttgataaaaatcttc |
| | | caaatgaaaaagtactaccaaaacatagtttgctttatgagtatttt |
| | | acggtttataacgaattgacaaaggtcaaatatgttactgaaggaat |
| | | gcgaaaaccagcatttctttcaggtgaacagaagaaagccattgttg |
| | | atttactcttcaaaacaaatcgaaaagtaaccgttaagcaattaaaa |
| | | gaagattatttcaaaaaaatagaatgttttgatagtgttgaaatttc |
| | | aggagttgaagatagatttaatgcttcattaggtacctaccatgatt |
| | | tgctaaaaattattaaagataaagattttttggataatgaagaaaat |
| | | gaagatatcttagaggatattgttttaacattgaccttatttgaaga |
| | | tagggagatgattgaggaaagacttaaaacatatgctcacctctttg |
| | | atgataaggtgatgaaacagcttaaacgtcgccgttatactggttag |
| | | ggacgtttgtctcgaaaattgattaatggtattagggataagcaatc |
| | | tggcaaaacaatattagattttttgaaatcagatggttttgccaatc |
| | | gcaattttatgcagctgatccatgatgatagtttgacatttaaagaa |
| | | gacattcaaaaagcacaagtgtctggacaaggcgatagtttacatga |
| | | acatattgcaaatttagctggtagccctgctattaaaaaaggtattt |
| | | tacagactgtaaaagttgttgatgaattggtcaaagtaatggggcgg |
| | | cataagccagaaaatatcgttattgaaatggcacgtgaaaatcagac |
| | | aactcaaaagggccagaaaaattcgcgagagcgtatgaaacgaatcg |
| | | aagaaggtatcaaagaattaggaagtcagattcttaaagagcatcct |
| | | gttgaaaatactcaattgcaaaatgaaaagctctatctctattatct |
| | | ccaaaatggaagagacatgtatgtggaccaagaattagatattaatc |
| | | gtttaagtgattatgatgtcgatgccattgttccacaaagtttcctt |
| | | aaagacgattcaatagacaataaggtcttaacgcgttctgataaaaa |
| | | tcgtggtaaatcggataacgttccaagtgaagaagtagtcaaaaaga |
| | | tgaaaaactattggagacaacttctaaacgccaagttaatcactcaa |
| | | cgtaagtttgataatttaacgaaagctgaacgtggaggtttgagtga |
| | | acttgataaagctggttttatcaaacgccaattggttgaaactcgcc |
| | | aaatcactaagcatgtggcacaaattttggatagtcgcatgaatact |
| | | aaatacgatgaaaatgataaacttattcgagaggttaaagtgattac |
| | | cttaaaatctaaattagtttctgacttccgaaaagatttccaattct |
| | | ataaagtacgtgagattaacaattaccatcatgcccatgatgcgtat |
| | | ctaaatgccgtcgttggaactgctttgattaagaaatatccaaaact |
| | | tgaatcggagtttgtctatggtgattataaagtttatgatgttcgta |
| | | aaatgattgctaagtctgagcaagaaataggcaaagcaaccgcaaaa |
| | | tatttcttttactctaatatcatgaacttcttcaaaacagaaattac |
| | | acttgcaaatggagagattcgcaaacgccctctaatcgaaactaatg |
| | | gggaaactggagaaattgtctgggataaagggcgagattttgccaca |
| | | gtgcgcaaagtattgtccatgccccaagtcaatattgtcaagaaaac |
| | | agaagtacagacaggcggattctccaaggagtcaattttaccaaaaa |
| | | gaaattcggacaagcttattgctcgtaaaaaagactgggatccaaaa |
| | | aaatatggtggttttgatagtccaacggtagcttattcagtcctagt |
| | | ggttgctaaggtggaaaaagggaaatcgaagaagttaaaatccgtta |
| | | aagagttactagggatcacaattatggaaagaagttcctttgaaaaa |
| | | aatccgattgactttttagaagctaaaggatataaggaagttaaaaa |
| | | agacttaatcattaaactacctaaatatagtctttttgagttagaaa |
| | | acggtcgtaaacggatgctggctagtgccggagaattacaaaaagga |
| | | aatgagctggctctgccaagcaaatatgtgaattttttatatttagc |
| | | tagtcattatgaaaagttgaagggtagtccagaagataacgaacaaa |
| | | aacaattgtttgtggagcagcataagcattatttagatgagattatt |
| | | gagcaaatcagtgaattttctaagcgtgttattttagcagatgccaa |
| | | tttagataaagttcttagtgcatataacaaacatagagacaaaccaa |
| | | tacgtgaacaagcagaaaatattattcatttatttacgttgacgaat |
| | | cttggagctcccgctgcttttaaatattttgatacaacaattgatcg |
| | | taaacgatatacgtctacaaaagaagttttagatgccactcttatcc |
| | | atcaatccatcactggtctttatgaaacacgcattgatttgagtcag |
| | | ctaggaggtgactaactcgagtaaggatctccaggcatcaaataaaa |
| | | cgaaaggctcagtcgaaagactgggcctttcgttttatctgttgttt |
| | | gtcggtgaacgctctctactagagtcacactggctcaccttcgggtg |
| | | ggcctttctgcgtttatacctagggatatattccgcttcctcgctca |
| | | ctgactcgctacgctcggtcgttcgactgcggcgagcggaaatggct |
| | | tacgaacggggcggagatttcctggaagatgccaggaagatacttaa |
| | | cagggaagtgagagggccgcggcaaagccgtttttccataggctccg |
| | | cccccctgacaagcatcacgaaatctgacgctcaaatcagtggtggc |
| | | gaaacccgacaggactataaagataccaggcgtttccccctggcggc |
| | | tccctcgtgcgctctcctgttcctgcctttcggtttaccggtgtcat |
| | | tccgctgttatggccgcgtttgtctcattccacgcctgacactcagt |
| | | tccgggtaggcagttcgctccaagctggactgtatgcacgaaccccc |
| | | cgttcagtccgaccgctgcgccttatccggtaactatcgtcttgagt |
| | | ccaacccggaaagacatgcaaaagcaccactggcagcagccactggt |
| | | aattgatttagaggagttagtcttgaagtcatgcgccggttaaggct |
| | | aaactgaaaggacaagttttggtgactgcgctcctccaagccagtta |
| | | cctcggttcaaagagttggtagctcagagaaccttcgaaaaaccgcc |
| | | ctgcaaggcggttttttcgttttcagagcaagagattacgcgcagac |
| | | caaaacgatctcaagaagatcatcttattaatcagataaaatatttc |
| | | tagatttcagtgcaatttatctcttcaaatgtagcacctgaagtcag |
| | | ccccatacgatataagttgttactagtgcttggattctcaccaataa |
| | | aaaacgcccggcggcaaccgagcgttctgaacaaatccagatggagt |
| | | tctgaggtcattactggatctatcaacaggagtccaagcgagctcga |
| | | tatcaaatcagaagaactcgtcaagaaggcgatagaaggcgatgcgc |
| | | tgcgaatcgggagcggcgataccgtaaagcacgaggaagcggtcagc |
| | | ccattcgccgccaagctcttcagcaatatcacgggtagccaacgcta |
| | | tgtcctgatagcggtccgccacacccagccggccacagtcgatgaat |
| | | ccagaaaagcggccattttccaccatgatattcggcaagcaggcatc |
| | | gccatgggtcacgacgagatcctcgccgtcgggcatgcgcgccttga |
| | | gcctggcgaacagttcggctggcgcgagcccctgatgctcttcgtcc |
| | | agatcatcctgatcgacaagaccggcttccatccgagtacgtgctcg |
| | | ctcgatgcgatgtttcgcttggtggtcgaatgggcaggtagccggat |
| | | caagcgtatgcagccgccgcattgcatcagccatgatggatactttc |
| | | tcggcaggagcaaggtgagatgacaggagatcctgccccggcacttc |
| | | gcccaatagcagccagtcccttcccgcttcagtgacaacgtcgagca |
| | | cagctgcgcaaggaacgcccgtcgtggccagccacgatagccgcgct |
| | | gcctcgtcctgcagttcattcagggcaccggacaggtcggtcttgac |
| | | aaaaagaaccgggcgcccctgcgctgacagccggaacacggcggcat |
| | | cagagcagccgattgtctgttgtgcccagtcatagccgaatagcctc |
| | | tccacccaagcggccggagaacctgcgtgcaatccatcttgttcaat |
| | | cattttagcttccttagctcctgaaaatctcgataactcaaaaaata |
| | | cgcccggtagtgatcttatttcattatggtgaaagttggaacctctt |
| | | acgtgccgatcaacgtctcattttcgccagatatcgacgtcttaaga |
| | | cccactttcacatttaagttgtttttctaatccgcatatgatcaatt |
| | | caaggccgaataagaaggctggctctgcaccttggtgatcaaataat |
| | | tcgatagcttgtcgtaataatggcggcatactatcagtagtaggtgt |
| | | ttccctttcttctttagcgacttgatgctcttgatcttccaatacgc |
| | | aacctaaagtaaaatgccccacagcgctgagtgcatataatgcattc |
| | | tctagtgaaaaaccttgttggcataaaaaggctaattgattttcgag |
| | | agtttcatactgtttttctgtaggccgtgtacctaaatgtacttttg |
| | | ctccatcgcgatgacttagtaaagcacatctaaaacttttagcgtta |
| | | ttacgtaaaaaatcttgccagctttccccttctaaagggcaaaagtg |
| | | agtatggtgcctatctaacatctcaatggctaaggcgtcgagcaaag |
| | | cccgcttattttttacatgccaatacaatgtaggctgctctacacct |
| | | agcttctgggcgagtttacgggttgttaaaccttcgattccgacctc |
| | | attaagcagctctaatgcgctgttaatcactttacttttatctaatc |
| | | tagacatcattaattcctaattttt |
| pAL2160 | 64 | aattgtgagcggataacaattgacattgtgagcggataacaagatac |
| (P_{LlacO1}:alsE- | | tgagcacatcagcaggacgcactgaccgaattcattaaagaggagaa |
| hxpB, | | aagatataccatgaaaatctccccctcgttaatgtgtatggatctgc |
| no sgRNA) | | tgaaatttaaagaacagatcgaatttatcgacagccatgccgattac |
| | | ttccacatcgatatcatggacggtcactttgtccccaatctgacact |
| | | ctcaccgttcttcgtaagtcaggttaaaaaactggcaactaaaccgc |
| | | tcgactgtcatctgatggtgacgcggccgcaggattacattgctcaa |
| | | ctggcgcgtgcgggagcagatttcatcactctgcatccggaaaccat |
| | | caacggccaggcgttccgcctgattgatgaaatccgccgtcatgaca |
| | | tgaaagtggggctgatccttaacccggagacgccagttgaggccatg |
| | | aaatactatatccataaggccgataaaattacggtcatgactgtcga |
| | | tcccggctttgccggacaaccgttcattcctgaaatgctggataaac |
| | | ttgccgaactgaaggcatggcgtgaacgagaaggtctggagtacgaa |
| | | attgaggtggacggttcctgcaaccaggcaacttacgaaaaactgat |
| | | ggcggcaggggcggatgtctttatcgtcggcacttccggcctgttta |
| | | atcatgcggaaaatatcgacgaagcatggagaattatgaccgcgcag |
| | | attctggctgcaaaaagcgaggtacagcctcatgcaaaaacagcata |
| | | aggatccggctgctaacaaaaggagatatacatatgtcaaccccgcg |
| | | tcagattcttgctgcaatttttgatatggatggattacttatcgact |
| | | cagaacctttatgggatcgagccgaactggatgtgatggcaagcctg |
| | | ggggtggatatctcccgtcgtaacgagctgccggacaccttaggttt |
| | | acgcatcgatatggtggtcgatctttggtacgcccggcaaccgtgga |
| | | atgggccaagccgtcaggaagtagtagaacgggttattgcccgtgcc |
| | | atttcactggttgaagagacacgtccattattaccaggcgtgcgcga |
| | | agccgttgcgttatgcaaagaacaaggtttattggtgggactggcct |
| | | ccgcgtcaccactacatatgctggaaaaagtgttgaccatgtttgac |
| | | ttacgcgacagtttcgatgccctcgcctcggccgaaaaactgcctta |
| | | cagcaagccgcatccgcaagtatatctcgactgcgcagcaaaactgg |
| | | gcgttgaccctctgacctgcgtagcgctggaagattcggtaaatggc |
| | | atgatcgcctctaaagcagcccgcatgcgttccatcgtcgttcctgc |
| | | gccagaagcgcaaaatgatccacgttttgtattagcagacgtcaaac |
| | | tttcatcgctgacagaactcaccgcaaaagaccttctcggttgagat |
| | | ccggctgctaaggtacctaatctagaggcatcaaataaaacgaaagg |
| | | ctcagtcgaaagactgggcctttcgttttatctgttgtttgtcggtg |
| | | aacgctctcctgagtaggacaaatccgccgccctagacctaggcgtt |
| | | cggctgcggcgagcggtatcagctcactcaaaggcggtaatacggtt |
| | | atccacagaatcaggggataacgcaggaaagaacagagcgcctgatg |
| | | cggtattttctccttacgcatctgtgcggtatttcacaccgcatatg |
| | | ctggatccttgacagctagctcagtcctaggtataatactagtgttt |
| | | tagagctagaaatagcaagttaaaataaggctagtccgttatcaact |
| | | tgaaaaagtggcaccgagtcggtgctttttttgaattctctagagtc |
| | | gacctgcagaagcttagatctattaatgtgagcaaaaggccagcaaa |
| | | aggccaggaaccgtaaaaaggccgcgttgctggcgtttttccatagg |
| | | ctccgcccccctgacgagcatcacaaaaatcgacgctcaagtcagag |
| | | gtggcgaaacccgacaggactataaagataccaggcgtttccccctg |
| | | gaagctccctcgtgcgctctcctgttccgaccctgccgcttaccgga |
| | | tacctgtccgcctttctcccttcgggaagcgtggcgctttctcaatg |
| | | ctcacgctgtaggtatctcagttcggtgtaggtcgttcgctccaagc |
| | | tgggctgtgtgcacgaaccccccgttcagcccgaccgctgcgcctta |
| | | tccggtaactatcgtcttgagtccaacccggtaagacacgacttatc |
| | | gccactggcagcagccactggtaacaggattagcagagcgaggtatg |
| | | taggcggtgctacagagttcttgaagtggtggcctaactacggctac |
| | | actagaaggacagtatttggtatctgcgctctgctgaagccagttac |
| | | cttcggaaaaagagttggtagctcttgatccggcaaacaaaccaccg |
| | | ctggtagcggtggtttttttgtttgcaagcagcagattacgcgcaga |
| | | aaaaaaggatctcaagaagatcctttgatctttictacggggtctga |
| | | cgctcagtggaacgaaaactcacgttaagggattttggtcatgacta |
| | | gtgcttggattctcaccaataaaaaacgcccggcggcaaccgagcgt |
| | | tctgaacaaatccagatggagttctgaggtcattactggatctatca |
| | | acaggagtccaagcgagctcgtaaacttggtctgacagttaccaatg |
| | | cttaatcagtgaggcacctatctcagcgatctgtctatttcgttcat |
| | | ccatagttgcctgactccccgtcgtgtagataactacgatacgggag |
| | | ggcttaccatctggccccagtgctgcaatgataccgcgagacccacg |
| | | ctcaccggctccagatttatcagcaataaaccagccagccggaaggg |
| | | ccgagcgcagaagtggtcctgcaactttatccgcctccatccagtct |
| | | attaattgttgccgggaagctagagtaagtagttcgccagttaatag |
| | | tttgcgcaacgttgttgccattgctacaggcatcgtggtgtcacgct |
| | | cgtcgtttggtatggcttcattcagctccggttcccaacgatcaagg |
| | | cgagttacatgatcccccatgttgtgcaaaaaagcggttagctcctt |
| | | cggtcctccgatcgttgtcagaagtaagttggccgcagtgttatcac |
| | | tcatggttatggcagcactgcataattctcttactgtcatgccatcc |
| | | gtaagatgcttttctgtgactggtgagtactcaaccaagtcattctg |
| | | agaatagtgtatgcggcgaccgagttgctcttgcccggcgtcaatac |
| | | gggataataccgcgccacatagcagaactttaaaagtgctcatcatt |
| | | ggaaaacgttcttcggggcgaaaactctcaaggatcttaccgctgtt |
| | | gagatccagttcgatgtaacccactcgtgcacccaactgatcttcag |
| | | catcttttactttcaccagcgtttctgggtgagcaaaaacaggaagg |
| | | caaaatgccgcaaaaaagggaataagggcgacacggaaatgttgaat |
| | | actcatactcttcctttttcaatattattgaagcatttatcagggtt |
| | | attgtctcatgagcggatacatatttgaatgtatttagaaaaataaa |
| | | caaataggggttccgcgcacatttccccgaaaagtgccacctgacgt |
| | | ctaagaaaccattattatcatgacattaacctataaaaataggcgta |
| | | tcacgaggccctttcgtcttcacctcgag |
| pAL2179 | 65 | aattgtgagcggataacaattgacattgtgagcggataacaagatac |
| (P_{LlacO1}:alsE- | | tgagcacatcagcaggacgcactgaccgaattcattaaagaggagaa |
| hxpB, | | aagatataccatgaaaatctccccctcgttaatgtgtatggatctgc |
| pfkB sgRNA) | | tgaaatttaaagaacagatcgaatttatcgacagccatgccgattac |
| | | ttccacatcgatatcatggacggtcactttgtccccaatctgacact |
| | | ctcaccgttcttcgtaagtcaggttaaaaaactggcaactaaaccgc |
| | | tcgactgtcatctgatggtgacgcggccgcaggattacattgctcaa |
| | | ctggcgcgtgcgggagcagatttcatcactctgcatccggaaaccat |
| | | caacggccaggcgttccgcctgattgatgaaatccgccgtcatgaca |
| | | tgaaagtggggctgatccttaacccggagacgccagttgaggccatg |
| | | aaatactatatccataaggccgataaaattacggtcatgactgtcga |
| | | tcccggctttgccggacaaccgttcattcctgaaatgctggataaac |
| | | ttgccgaactgaaggcatggcgtgaacgagaaggtctggagtacgaa |
| | | attgaggtggacggttcctgcaaccaggcaacttacgaaaaactgat |
| | | ggcggcaggggcggatgtctttatcgtcggcacttccggcctgttta |
| | | atcatgcggaaaatatcgacgaagcatggagaattatgaccgcgcag |
| | | attctggctgcaaaaagcgaggtacagcctcatgcaaaaacagcata |
| | | aggatccggctgctaacaaaaggagatatacatatgtcaaccccgcg |
| | | tcagattcttgctgcaatttttgatatggatggattacttatcgact |
| | | cagaacctttatgggatcgagccgaactggatgtgatggcaagcctg |
| | | ggggtggatatctcccgtcgtaacgagctgccggacaccttaggttt |
| | | acgcatcgatatggtggtcgatctttggtacgcccggcaaccgtgga |
| | | atgggccaagccgtcaggaagtagtagaacgggttattgcccgtgcc |
| | | atttcactggttgaagagacacgtccattattaccaggcgtgcgcga |
| | | agccgttgcgttatgcaaagaacaaggtttattggtgggactggcct |
| | | ccgcgtcaccactacatatgctggaaaaagtgttgaccatgtttgac |
| | | ttacgcgacagtttcgatgccctcgcctcggccgaaaaactgcctta |
| | | cagcaagccgcatccgcaagtatatctcgactgcgcagcaaaactgg |
| | | gcgttgaccctctgacctgcgtagcgctggaagattcggtaaatggc |
| | | atgatcgcctctaaagcagcccgcatgcgttccatcgtcgttcctgc |
| | | gccagaagcgcaaaatgatccacgttttgtattagcagacgtcaaac |
| | | tttcatcgctgacagaactcaccgcaaaagaccttctcggttgagat |
| | | ccggctgctaaggtacctaatctagaggcatcaaataaaacgaaagg |
| | | ctcagtcgaaagactgggcctttcgttttatctgttgtttgtcggtg |
| | | aacgctctcctgagtaggacaaatccgccgccctagacctaggcgtt |
| | | cggctgcggcgagcggtatcagctcactcaaaggcggtaatacggtt |
| | | atccacagaatcaggggataacgcaggaaagaacagagcgcctgatg |
| | | cggtattttctccttacgcatctgtgcggtatttcacaccgcatatg |
| | | ctggatccttgacagctagctcagtcctaggtataatactagtttct |
| | | tcactttccgctgattgttttagagctagaaatagcaagttaaaata |
| | | aggctagtccgttatcaacttgaaaaagtggcaccgagtcggtgctt |
| | | tttttgaattctctagagtcgacctgcagaagcttagatctattaat |
| | | gtgagcadaaggccagcaaaaggccaggaaccgtaaaaaggccgcgt |
| | | tgctggcgtttttccataggctccgcccccctgacgagcatcacaaa |
| | | aatcgacgctcaagtcagaggtggcgaaacccgacaggactataaag |
| | | ataccaggcgtttccccctggaagctccctcgtgcgctctcctgttc |
| | | cgaccctgccgcttaccggatacctgtccgcctttctcccttcggga |
| | | agcgtggcgctttctcaatgctcacgctgtaggtatctcagttcggt |
| | | gtaggtcgttcgctccaagctgggctgtgtgcacgaaccccccgttc |
| | | agcccgaccgctgcgccttatccggtaactatcgtcttgagtccaac |
| | | ccggtaagacacgacttatcgccactggcagcagccactggtaacag |
| | | gattagcagagcgaggtatgtaggcggtgctacagagttcttgaagt |
| | | ggtggcctaactacggctacactagaaggacagtatttggtatctgc |
| | | gctctgctgaagccagttaccttcggaaaaagagttggtagctcttg |
| | | atccggcaaacaaaccaccgctggtagcggtggtttttttgtttgca |
| | | agcagcagattacgcgcagaaaaaaaggatctcaagaagatcctttg |
| | | atcttttctacggggtctgacgctcagtggaacgaaaactcacgtta |
| | | agggattttggtcatgactagtgcttggattctcaccaataaaaaac |
| | | gcccggcggcaaccgagcgttctgaacaaatccagatggagttctga |
| | | ggtcattactggatctatcaacaggagtccaagcgagctcgtaaact |
| | | tggtctgacagttaccaatgcttaatcagtgaggcacctatctcagc |
| | | gatctgtctatttcgttcatccatagttgcctgactccccgtcgtgt |
| | | agataactacgatacgggagggcttaccatctggccccagtgctgca |
| | | atgataccgcgagacccacgctcaccggctccagatttatcagcaat |
| | | aaaccagccagccggaagggccgagcgcagaagtggtcctgcaactt |
| | | tatccgcctccatccagtctattaattgttgccgggaagctagagta |
| | | agtagttcgccagttaatagittgcgcaacgttgttgccattgctac |
| | | aggcatcgtggtgtcacgctcgtcgtttggtatggcttcattcagct |
| | | ccggttcccaacgatcaaggcgagttacatgatcccccatgttgtgc |
| | | aaaaaagcggttagctccttcggtcctccgatcgttgtcagaagtaa |
| | | gttggccgcagtgttatcactcatggttatggcagcactgcataatt |
| | | ctcttactgtcatgccatccgtaagatgcttttctgtgactggtgag |
| | | tactcaaccaagtcattctgagaatagtgtatgcggcgaccgagttg |
| | | ctcttgcccggcgtcaatacgggataataccgcgccacatagcagaa |
| | | ctttaaaagtgctcatcattggaaaacgttcttcggggcgaaaactc |
| | | tcaaggatcttaccgctgttgagatccagttcgatgtaacccactcg |
| | | tgcacccaactgatcttcagcatcttttactttcaccagcgtttctg |
| | | ggtgagcaaaaacaggaaggcaaaatgccgcaaaaaagggaataagg |
| | | gcgacacggaaatgttgaatactcatactcttcctttttcaatatta |
| | | ttgaagcatttatcagggttattgtctcatgagcggatacatatttg |
| | | aatgtatttagaaaaataaacaaataggggttccgcgcacatttccc |
| | | cgaaaagtgccacctgacgtctaagaaaccattattatcatgacatt |
| | | aacctataaaaataggcgtatcacgaggccctttcgtcttcacctcg |
| | | ag |
| pAL2247 | 66 | atgcgcataaaaataacagcataaaacaccttaccaccacccaagaa |
| (P_{gadB}:alsE- | | gtaataattttataaatgcgttcaaaataataatcaagtactaatag |
| hxpB) | | tgatattttaaggtctgatttttacgtgataattcaggagacacaga |
| | | tttcatattgtattgtttttcaatgaaaaaatattattcgcgtaata |
| | | tctcacgataaataacattaggattttgttatttaaacacgagtcct |
| | | ttgcacttgcttactttatcgataaatcctacttttttaatgcgatc |
| | | caatcattttaaggagtttaaaatggataagaagcaagtgaattcat |
| | | taaagaggagaaaagatataccatgaaaatctccccctcgttaatgt |
| | | gtatggatctgctgaaatttaaagaacagatcgaatttatcgacagc |
| | | catgccgattacttccacatcgatatcatggacggtcactttgtccc |
| | | caatctgacactctcaccgttcttcgtaagtcaggttaaaaaactgg |
| | | caactaaaccgctcgactgtcatctgatggtgacgcggccgcaggat |
| | | tacattgctcaactggcgcgtgcgggagcagatttcatcactctgca |
| | | tccggaaaccatcaacggccaggcgttccgcctgattgatgaaatcc |
| | | gccgtcatgacatgaaagtggggctgatccttaacccggagacgcca |
| | | gttgaggccatgaaatactatatccataaggccgataaaattacggt |
| | | catgactgtcgatcccggctttgccggacaaccgttcattcctgaaa |
| | | tgctggataaacttgccgaactgaaggcatggcgtgaacgagaaggt |
| | | ctggagtacgaaattgaggtggacggttcctgcaaccaggcaactta |
| | | cgaaaaactgatggcggcaggggcggatgtctttatcgtcggcactt |
| | | ccggcctgtttaatcatgcggaaaatatcgacgaagcatggagaatt |
| | | atgaccgcgcagattctggctgcaaaaagcgaggtacagcctcatgc |
| | | aaaaacagcataaggatccggctgctaacaaaaggagatatacatat |
| | | gtcaaccccgcgtcagattcttgctgcaatttttgatatggatggat |
| | | tacttatcgactcagaacctttatgggatcgagccgaactggatgtg |
| | | atggcaagcctgggggtggatatctcccgtcgtaacgagctgccgga |
| | | caccttaggtttacgcatcgatatggtggtcgatctttggtacgccc |
| | | ggcaaccgtggaatgggccaagccgtcaggaagtagtagaacgggtt |
| | | attgcccgtgccatttcactggttgaagagacacgtccattattacc |
| | | aggcgtgcgcgaagccgttgcgttatgcaaagaacaaggtttattgg |
| | | tgggactggcctccgcgtcaccactacatatgctggaaaaagtgttg |
| | | accatgtttgacttacgcgacagtttcgatgccctcgcctcggccga |
| | | aaaactgccttacagcaagccgcatccgcaagtatatctcgactgcg |
| | | cagcaaaactgggcgttgaccctctgacctgcgtagcgctggaagat |
| | | tcggtaaatggcatgatcgcctctaaagcagcccgcatgcgttccat |
| | | cgtcgttcctgcgccagaagcgcaaaatgatccacgttttgtattag |
| | | cagacgtcaaactttcatcgctgacagaactcaccgcaaaagacctt |
| | | ctcggttgagatccggctgctaaggtacctaatctagaggcatcaaa |
| | | taaaacgaaaggctcagtcgaaagactgggcctttcgttttatctgt |
| | | tgtttgtcggtgaacgctctcctgagtaggacaaatccgccgcccta |
| | | gacctaggcgttcggctgcggcgagcggtatcagctcactcaaaggc |
| | | ggtaatacggttatccacagaatcaggggataacgcaggaaagaaca |
| | | tgtgagcaaaaggccagcaaaaggccaggaaccgtaaaaaggccgcg |
| | | ttgctggcgtttttccataggctccgcccccctgacgagcatcacaa |
| | | aaatcgacgctcaagtcagaggtggcgaaacccgacaggactataaa |
| | | gataccaggcgtttccccctggaagctccctcgtgcgctctcctgtt |
| | | ccgaccctgccgcttaccggatacctgtccgcctttctcccttcggg |
| | | aagcgtggcgctttctcaatgctcacgctgtaggtatctcagttcgg |
| | | tgtaggtcgttcgctccaagctgggctgtgtgcacgaaccccccgtt |
| | | cagcccgaccgctgcgccttatccggtaactatcgtcttgagtccaa |
| | | cccggtaagacacgacttatcgccactggcagcagccactggtaaca |
| | | ggattagcagagcgaggtatgtaggcggtgctacagagttcttgaag |
| | | tggtggcctaactacggctacactagaaggacagtatttggtatctg |
| | | cgctctgctgaagccagttaccttcggaaaaagagttggtagctctt |
| | | gatccggcaaacaaaccaccgctggtagcggtggtttttttgtttgc |
| | | aagcagcagattacgcgcagaaaaaaaggatctcaagaagatccttt |
| | | gatcttttctacggggtctgacgctcagtggaacgaaaactcacgtt |
| | | aagggattttggtcatgactagtgcttggattctcaccaataaaaaa |
| | | cgcccggcggcaaccgagcgttctgaacaaatccagatggagttctg |
| | | aggtcattactggatctatcaacaggagtccaagcgagctcgtaaac |
| | | ttggtctgacagttaccaatgcttaatcagtgaggcacctatctcag |
| | | cgatctgtctatttcgttcatccatagttgcctgactccccgtcgtg |
| | | tagataactacgatacgggagggcttaccatctggccccagtgctgc |
| | | aatgataccgcgagacccacgctcaccggctccagatttatcagcaa |
| | | taaaccagccagccggaagggccgagcgcagaagtggtcctgcaact |
| | | ttatccgcctccatccagtctattaattgttgccgggaagctagagt |
| | | aagtagttcgccagttaatagtttgcgcaacgttgttgccattgcta |
| | | caggcatcgtggtgtcacgctcgtcgtttggtatggcttcattcagc |
| | | tccggttcccaacgatcaaggcgagttacatgatcccccatgttgtg |
| | | caaaaaagcggttagctccttcggtcctccgatccttgtcagaagta |
| | | agttggccgcagtgttatcactcatggttatggcagcactgcataat |
| | | tctcttactgtcatgccatccgtaagatgcttttctgtgactggtga |
| | | gtactcaaccaagtcattctgagaatagtgtatgcggcgaccgagtt |
| | | gctcttgcccggcgtcaatacgggataataccgcgccacatagcaga |
| | | actitaaaagtgctcatcatiggaaaacgttcttcggggcgaaaact |
| | | ctcaaggatcttaccgctgttgagatccagttcgatgtaacccactc |
| | | gtgcacccaactgatcttcagcatcttttactttcaccagcgtttct |
| | | gggtgagcaaaaacaggaaggcaaaatgccgcaaaaaagggaataag |
| | | ggcgacacggaaatgttgaatactcatactcttcctttttcaatatt |
| | | attgaagcatttatcagggttattgtctcatgagcggatacatattt |
| | | gaatgtatttagaaaaataaacaaataggggttccgcgcacatttcc |
| | | Ccgaaaagtgccacctgacgtctaagaaaccattattatcatgacat |
| | | taacctataaaaataggcgtatcacgaggccctttcgtcttcacctc |
| | | gag |
| pAL2274 | 67 | tccctatcagtgatagagattgacatccctatcagtgatagagatac |
| (P_{LtetO1}:galP- | | tgagcacatcagcaggacgcactgaccgaattcattaaagaggagaa |
| glk) | | aggtacaatgcctgacgctaaaaaacaggggcggtcaaacaaggcaa |
| | | tgacgtttttcgtctgcttccttgccgctctggcgggattactcttt |
| | | ggcctggatatcggtgtaattgctggcgcactgccgtttattgcaga |
| | | tgaattccagattacttcgcacacgcaagaatgggtcgtaagctcca |
| | | tgatgttcggtgcggcagtcggtgcggtgggcagcggctggctctcc |
| | | tttaaactcgggcgcaaaaagagcctgatgatcggcgcaattttgtt |
| | | tcttgccggttcgctgttctctgcggctgcgccaaacgttgaagtac |
| | | tgattctttcccgcgttctactggggctggcggtgggtgtggcctct |
| | | tataccgcaccgctgtacctctctgaaattgcgccggaaaaaattcg |
| | | tggcagtatgatctcgatgtatcagttgatgatcactatcgggatcc |
| | | tcggtgcttatctttctgataccgccttcagctacaccggtgcatgg |
| | | cgctggatgctgggtgtgattatcatcccggcaattttgctgctgat |
| | | tggtgtcttcttcctgccagacagcccacgttggtttgccgccaaac |
| | | gccgttttgttgatgccgaacgcgtgctgctacgcctgcgtgacacc |
| | | agcgcggaagcgaaacgcgaactggatgaaatccgtgaaagtttgca |
| | | ggttaaacagagtggctgggcgctgtttaaagagaacagcaacttcc |
| | | gccgcgcggtgttccttggcgtactgttgcaggtaatgcagcaattc |
| | | accgggatgaacgtcatcatgtattacgcgccgaaaatcttcgaact |
| | | ggcgggttataccaacactaccgagcaaatgtgggggaccgtgattg |
| | | tcggcctgaccaacgtacttgccacctttatcgcaatcggccttgtt |
| | | gaccgctggggacgtaaaccaacgctaacgctgggcttcctggtgat |
| | | ggctgctggcatgggcgtactcggtacaatgatgcatatcggtattc |
| | | actctccgtcggcgcagtatttcgccatcgccatgctgctgatgttt |
| | | attgtcggttttgccatgagtgccggtccgctgatttgggtactgtg |
| | | ctccgaaattcagccgctgaaaggccgcgattttggcatcacctgct |
| | | ccactgccaccaactggattaccaacatgatcgttggcgcaacgttc |
| | | ctgaccatgctcaacacgctgggtaacgccaacaccttctgggtgta |
| | | tgcggctctgaacgtactgtttatcctgctgacattgtggctggtac |
| | | cggaaaccaaacacgtttcgctggaacatattgaacgtaatctgatg |
| | | aaaggtcgtaaactgcgcgaaataggcgctcacgattaaggctgcta |
| | | acaaaaggagatatacatatgacaaagtatgcattagtcggtgatgt |
| | | gggcggcaccaacgcacgtcttgctctgtgtgatattaccagtggtg |
| | | aaatctcgcaggctaagacctattcagggcttgattaccccagcctc |
| | | gaagcggtcattcgcgtttatcttgaagaacataaggtcgaggtgaa |
| | | agacggctgtattgccatcgcttgcccaattaccggtgactgggtgg |
| | | cgatgaccaaccatacctgggcgttctcaattgccgaaatgaaaaag |
| | | aatctcggttttagccatctggaaattattaacgattttaccgctgt |
| | | atcgatggcgatcccgatgctgaaaaaagagcatctgattcagtttg |
| | | gtggcgcagaaccggtcgaaggtaagcctattgcggtttacggtgcc |
| | | ggaacggggcttggggttgcgcatctggtccatgtcgataagcgttg |
| | | ggtaagcttgccaggcgaaggcggtcacgttgattttgcgccgaata |
| | | gtgaagaagaggccattatcctcgaaatattgcgtgcggaaattggt |
| | | catgtttcggcggagcgcgtgctttctggccctgggctggtgaattt |
| | | gtatcgcgcaattgtgaaagctgacaaccgcctgccagaaaatctca |
| | | agccaaaagatattaccgaacgcgcgctggctgacagctgcaccgat |
| | | tgccgccgcgcattgtcgctgttttgcgtcattatgggccgttttgg |
| | | cggcaatctggcgctcaatctcgggacatttggcggcgtgtttattg |
| | | cgggcggtatcgtgccgcgcttccttgagttcttcaaagcctccggt |
| | | ttccgtgccgcatttgaagataaagggcgctttaaagaatatgtcca |
| | | tgatattccggtgtatctcatcgtccatgacaatccgggccttctcg |
| | | gttccggtgcacatttacgccagaccttaggtcacattctgtaaacg |
| | | cgtgctagaggcatcaaataaaacgaaaggctcagtcgaaagactgg |
| | | gcctttcgttttatctgttgtttgtcggtgaacgctctcctgagtag |
| | | gacaaatccgccgccctagacctaggtagcgcagaaacgtcctagaa |
| | | gatgccaggaggatacttagcagagagacaataaggccggagcgaag |
| | | ccgtttttccataggctccgcccccctgacgaacatcacgaaatctg |
| | | acgctcaaatcagtggtggcgaaacccgacaggactataaagatacc |
| | | aggcgtttccccctgatggctccctcttgcgctctcctgttcccgtc |
| | | ctgcggcgtccgtgttgtggtggaggctttacccaaatcaccacgtc |
| | | ccgttccgtgtagacagttcgctccaagctgggctgtgtgcaagaac |
| | | cccccgttcagcccgactgctgcgccttatccggtaactatcatctt |
| | | gagtccaacccggaaagacacgacaaaacgccactggcagcagccat |
| | | tggtaactgagaattagtggatttagatatcgagagtcttgaagtgg |
| | | tggcctaacagaggctacactgaaaggacagtatttggtatctgcgc |
| | | tccactaaagccagttaccaggttaagcagttccccaactgacttaa |
| | | ccttcgatcaaaccgcctccccaggcggttttttcgtttacagagca |
| | | ggagattacgacgatcgtaaaaggatctcaagaagatcctttacgga |
| | | ttcccgacaccatcaccagataaaatatttctagatttcagtgcaat |
| | | ttatctcttcaaatgtagcacctgaagtcagccccatacgatataag |
| | | ttgttactagtgcttggattctcaccaataaaaaacgcccggcggca |
| | | accgagcgttctgaacaaatccagatggagttctgaggtcattactg |
| | | gatctatcaacaggagtccaagcgagctctcgaaccccagagtcccg |
| | | ctcattaggtggcggtacttgggtcgatatcaaagtgcatcacttct |
| | | tcccgtatgcccaactttgtatagagagccactgcgggatcgtcacc |
| | | gtaatctgcttgcacgtagatcacataagcaccaagcgcgttggcct |
| | | catgcttgaggagattgatgagcgcggtggcaatgccctgcctccgg |
| | | tgctcgccggagactgcgagatcatagatatagatctcactacgcgg |
| | | ctgctcaaacctgggcagaacgtaagccgcgagagcgccaacaaccg |
| | | cttcttggtcgaaggcagcaagcgcgatgaatgtcttactacggagc |
| | | aagttcccgaggtaatcggagtccggctgatgttgggagtaggtggc |
| | | tacgtctccgaactcacgaccgaaaagatcaagagcagcccgcatgg |
| | | atttgacttggtcagggccgagcctacatgtgcgaatgatgcccata |
| | | cttgagccacctaactttgttttagggcgactgccctgctgcgtaac |
| | | atcgttgctgctgcgtaacatgcgaaacgatcctcatcctgtctctt |
| | | gatcagatcttgatcccctgcgccatcagatccttggcggcaagaaa |
| | | gccatccagtttactttgcagggcttcccaaccttaccagagggcgc |
| | | cccagctggcaattccgacgtctaagaaaccattattatcatgacat |
| | | taacctataaaaataggcgtatcacgaggccctttcgtcttcacctc |
| | | gag |
| pCas | 68 | atggataagaaatactcaataggcttagatatcggcacaaatagcgt |
| (CRISPR | | cggatgggcggtgatcactgatgattataaggttccgtctaaaaagt |
| Cas9 | | tcaaggttctgggaaatacagaccgccacagtatcaaaaaaaatctt |
| plasmid, | | ataggggctcttttatttgacagtggagagacagcggaagcgactcg |
| addgene | | tctcaaacggacagctcgtagaaggtatacacgtcggaagaatcgta |
| plasmid | | tttgttatctacaggagattttttcaaatgagatggcgaaagtagat |
| #62225 | | gatagtttctttcatcgacttgaagagtcttttttggtggaagaaga |
| from | | caagaagcatgaacgtcatcctatttttggaaatatagtagatgaag |
| Multigene | | ttgcttatcatgagaaatatccaactatctatcatctgcgaaaaaaa |
| editing in | | ttggtagattctactgataaagcggatttgcgcttaatctatttggc |
| the | | cttagcgcatatgattaagtttcgtggtcattttttgattgagggag |
| Escherichia | | atttaaatcctgataatagtgatgtggacaaactatttatccagttg |
| coli | | gtacaaacctacaatcaattatttgaagaaaaccctattaacgcaag |
| genome | | tggagtagatgctaaagcgattctttctgcacgattgagtaaatcaa |
| using the | | gacgattagaaaatctcattgctcagctccccggtgagaagaaaaat |
| CRISPR- | | ggcttatttgggaatctcattgctttgtcattgggtttgacccctaa |
| Cas9 | | ttttaaatcaaattttgatttggcagaagatgctaaattacagcttt |
| system. | | caaaagatacttacgatgatgatttagataatttattggcgcaaatt |
| Jiang Y, | | ggagatcaatatgctgatttgtttttggcagctaagaatttatcaga |
| Chen B, | | tgctattttactttcagatatcctaagagtaaatactgaaataacta |
| Duan C, | | aggctcccctatcagcttcaatgattaaacgctacgatgaacatcat |
| Sun B, | | caagacttgactcttttaaaagctttagttcgacaacaacttccaga |
| Yang J, | | aaagtataaagaaatcttttttgatcaatcaaaaaacggatatgcag |
| Yang S. | | gttatattgatgggggagctagccaagaagaattttataaatttatc |
| Appl | | aaaccaattttagaaaaaatggatggtactgaggaattattggtgaa |
| Environ | | actaaatcgtgaagatttgctgcgcaagcaacggacctttgacaacg |
| Microbiol. | | gctctattccccatcaaattcacttgggtgagctgcatgctattttg |
| 2015 | | agaagacaagaagacttttatccatttttaaaagacaatcgtgagaa |
| Jan 30. | | gattgaaaaaatcttgacttttcgaattccttattatgttggtccat |
| pii: | | tggcgcgtggcaatagtcgttttgcatggatgactcggaagtctgaa |
| AEM.04023- | | gaaacaattaccccatggaattttgaagaagttgtcgataaaggtgc |
| 14. | | ttcagctcaatcatttattgaacgcatgacaaactttgataaaaatc |
| 10.1128/ | | ttccaaatgaaaaagtactaccaaaacatagtttgctttatgagtat |
| AEM.04023-14) | | tttacggtttataacgaattgacaaaggtcaaatatgttactgaagg |
| | | aatgcgaaaaccagcatttctttcaggtgaacagaagaaagccattg |
| | | ttgatttactcttcaaaacaaatcgaaaagtaacccttaagcaatta |
| | | aaagaagattatttcaaaaaaatagaatgttttgatagtgttgaaat |
| | | ttcaggagttgaagatagatttaatgcttcattaggtacctaccatg |
| | | atttgctaaaaattattaaagataaagattttttggataatgaagaa |
| | | aatgaagatatcttagaggatattgttttaacattgaccttatttga |
| | | agatagggagatgattgaggaaagacttaaaacatatgctcacctct |
| | | ttgatgataaggtgatgaaacagcttaaacgtcgccgttatactggt |
| | | tggggacgtttgtctcgaaaattgattaatggtattagggataagca |
| | | atctggcaaaacaatattagattttttgaaatcagatggttttgcca |
| | | atcgcaattttatgcagctgatccatgatgatagtttgacatttaaa |
| | | gaagacattcaaaaagcacaagtgtctggacaaggcgatagtttaca |
| | | tgaacatattgcaaatttagctggtagccctgctattaaaaaaggta |
| | | ttttacagactgtaaaagttgttgatgaattggtcaaagtaatgggg |
| | | cggcataagccagaaaatatcgttattgaaatggcacgtgaaaatca |
| | | gacaactcaaaagggccagaaaaattcgcgagagcgtatgaaacgaa |
| | | tcgaagaaggtatcaaagaattaggaagtcagattcttaaagagcat |
| | | cctgttgaaaatactcaattgcaaaatgaaaagctctatctctatta |
| | | tctccaaaatggaagagacatgtatgtggaccaagaattagatatta |
| | | atcgtttaagtgattatgatgtcgatcacattgttccacaaagtttc |
| | | cttaaagacgattcaatagacaataaggtcttaacgcgttctgataa |
| | | aaatcgtggtaaatcggataacgttccaagtgaagaagtagtcaaaa |
| | | agatgaaaaactattggagacaacttctaaacgccaagttaatcact |
| | | caacgtaagtttgataatttaacgaaagctgaacgtggaggtttgag |
| | | tgaacttgataaagctggttttatcaaacgccaattggttgaaactc |
| | | gccaaatcactaagcatgtggcacaaattttggatagtcgcatgaat |
| | | actaaatacgatgaaaatgataaacttattcgagaggttaaagtgat |
| | | taccttaaaatctaaattagittctgacttccgaaaagatttccaat |
| | | tctataaagtacgtgagattaacaattaccatcatgcccatgatgcg |
| | | tatctaaatgccgtcgttggaactgctttgattaagaaatatccaaa |
| | | actigaatcggagtttgtctatggtgattataaagtttatgatgttc |
| | | gtaaaatgattgctaagtctgagcaagaaataggcaaagcaaccgca |
| | | aaatatttcttttactctaatatcatgaacttcttcaaaacagaaat |
| | | tacacttgcaaatggagagattcgcaaacgccctctaatcgaaacta |
| | | atggggaaactggagaaattgtctgggataaagggcgagattttgcc |
| | | acagtgcgcaaagtattgtccatgccccaagtcaatattgtcaagaa |
| | | aacagaagtacagacaggcggattctccaaggagtcaattttaccaa |
| | | aaagaaattcggacaagcttattgctcgtaaaaaagactgggatcca |
| | | aaaaaatatggtggttttgatagtccaacggtagcttattcagtcct |
| | | agtggttgctaaggtggaaaaagggaaatcgaagaagttaaaatccg |
| | | ttaaagagttactagggatcacaattatggaaagaagttcctttgaa |
| | | aaaaatccgattgactttttagaagctaaaggatataaggaagttaa |
| | | aaaagacttaatcattaaactacctaaatatagtctttttgagttag |
| | | aaaacggtcgtaaacggatgctggctagtgccggagaattacaaaaa |
| | | ggaaatgagctggctctgccaagcaaatatgtgaattttttatattt |
| | | agctagtcattatgaaaagttgaagggtagtccagaagataacgaac |
| | | aaaaacaattgtttgtggagcagcataagcattatttagatgagatt |
| | | attgagcaaatcagtgaattttctaagcgtgttattttagcagatgc |
| | | caatttagataaagttcttagtgcatataacaaacatagagacaaac |
| | | caatacatgaacaagcagaaaatattattcatttatttacgttgacg |
| | | aatcttggagctcccgctgcttttaaatattttgatacaacaattga |
| | | tcgtaaacgatatacgtctacaaaagaagttttagatgccactctta |
| | | tccatcaatccatcactggtctttatgaaacacgcattgatttgagt |
| | | cagctaggaggtgactgaagtatattttagatgaagattatttctta |
| | | atctagacatgagcggatacatatttgaatgtatttagaaaaataaa |
| | | caaataggggttccgcgcacatttccccgaaaagtgccacctgcatc |
| | | gatttattatgacaacttgacggctacatcattcactttttcttcac |
| | | aaccggcacggaactcgctcgggctggccccggtgcattttttaaat |
| | | acccgcgagaaatagagttgatcgtcaaaaccaacattgcgaccgac |
| | | ggtggcgataggcatccgggtggtgctcaaaagcagcttcgcctggc |
| | | tgatacgttggtcctcgcgccagcttaagacgctaatccctaactgc |
| | | tggcggaaaagatgtgacagacgcgacggcgacaagcaaacatgctg |
| | | tgcgacgctggcgatatcaaaattgctgtctgccaggtgatcgctga |
| | | tgtactgacaagcctcgcgtacccgattatccatcggtggatggagc |
| | | gactcgttaatcgcttccatgcgccgcagtaacaattgctcaagcag |
| | | atttatcgccagcagctccgaatagcgcccttccccttgcccggcgt |
| | | taatgatttgcccaaacaggtcgctgaaatgcggctggtgcgcttca |
| | | tccgggcgaaagaaccccgtattggcaaatattgacggccagttaag |
| | | ccattcatgccagtaggcgcgcggacgaaagtaaacccactggtgat |
| | | accattcgcgagcctccggatgacgaccgtagtgatgaatctctcct |
| | | ggggggaacagcaaaatatcacccggtcggcaaacaaattctcgtcc |
| | | ctgatttttcaccaccccctgaccgcgaatggtgagattgagaatat |
| | | aacctttcattcccagcggtcggtcgataaaaaaatcgagataaccg |
| | | ttggcctcaatcggcgttaaacccgccaccagatgggcattaaacga |
| | | gtatcccggcagcaggggatcattttgcgcttcagccatacttttca |
| | | tactcccgccattcagagaagaaaccaattgtccatattgcatcaga |
| | | cattgccgtcactgcgtcttttactggctcttctcgctaaccaaacc |
| | | ggtaaccccgcttattaaaagcattctgtaacaaagcgggaccaaag |
| | | ccatgacaaaaacgcgtaacaaaagtgtctataatcacggcagaaaa |
| | | gtccacattgattatttgcacggcgtcacactttgctatgccatagc |
| | | atttttatccataagattagcggatcctacctgacgctttttatcgc |
| | | aactctctactgtttctccatacccgtttttttgggaattcgagctc |
| | | taaggaggttataaaaaatggatattaatactgaaactgagatcaag |
| | | caaaagcattcactaaccccctttcctgttttcctaatcagcccggc |
| | | atttcgcgggcgatattttcacagctatttcaggagttcagccatga |
| | | acgcttattacattcaggatcgtcttgaggctcagagctgggcgcgt |
| | | cactaccagcagctcgcccgtgaagagaaagaggcagaactggcaga |
| | | cgacatggaaaaaggcctgccccagcacctgtttgaatcgctatgca |
| | | tcgatcatttgcaacgccacggggccagcaaaaaatccattacccgt |
| | | gcgtttgatgacgatgttgagtttcaggagcgcatggcagaacacat |
| | | ccggtacatggttgaaaccattgctcaccaccaggttgatattgatt |
| | | cagaggtataaaacgaatgagtactgcactcgcaacgctggctggga |
| | | agctggctgaacgtgtcggcatggattctgtcgacccacaggaactg |
| | | atcaccactcttcgccagacggcatttaaaggtgatgccagcgatgc |
| | | gcagttcatcgcattactgatcgttgccaaccagtacggccttaatc |
| | | cgtggacgaaagaaatttacgcctttcctgataagcagaatggcatc |
| | | gttccggtggtgggcgttgatggctggtcccgcatcatcaatgaaaa |
| | | ccagcagtttgatggcatggactttgagcaggacaatgaatcctgta |
| | | catgccggatttaccgcaaggaccgtaatcatccgatctgcgttacc |
| | | gaatggatggatgaatgccgccgcgaaccattcaaaactcgcgaagg |
| | | cagagaaatcacggggccgtggcagtcgcatcccaaacggatgttac |
| | | gtcataaagccatgattcagtgtgcccgtctggccttcggatttgct |
| | | ggtatctatgacaaggatgaagccgagcgcattgtcgaaaatactgc |
| | | atacactgcagaacgtcagccggaacgcgacatcactccggttaacg |
| | | atgaaaccatgcaggagattaacactctgctgatcgccctggataaa |
| | | acatgggatgacgacttattgccgctctgttcccagatatttcgccg |
| | | cgacattcgtgcatcgtcagaactgacacaggccgaagcagtaaaag |
| | | ctcttggattcctgaaacagaaagccgcagagcagaaggtggcagca |
| | | tgacaccggacattatcctgcagcgtaccgggatcgatgtgagagct |
| | | gtcgaacagggggatgatgcgtggcacaaattacggctcggcgtcat |
| | | caccgcttcagaagttcacaacgtgatagcaaaaccccgctccggaa |
| | | agaagtggcctgacatgaaaatgtcctacttccacaccctgcttgct |
| | | gaggtttgcaccggtgtggctccggaagttaacgctaaagcactggc |
| | | ctggggaaaacagtacgagaacgacgccagaaccctgtttgaattca |
| | | cttccggcgtgaatgttactgaatccccgatcatctatcgcgacgaa |
| | | agtatgcgtaccgcctgctctcccgatggtttatgcagtgacggcaa |
| | | cggccttgaactgaaatgcccgtttacctcccgggatttcatgaagt |
| | | tccggctcggtggtttcgaggccataaagtcagcttacatggcccag |
| | | gtgcagtacagcatgtgggtgacgcgaaaaaatgcctggtactttgc |
| | | caactatgacccgcgtatgaagcgtgaaggcctgcattatgtcgtga |
| | | ttgagcgggatgaaaagtacatggcgagttttgacgagatcgtgccg |
| | | gagttcatcgaaaaaatggacgaggcactggctgaaattggttttgt |
| | | atttggggagcaatggcgatgacgcatcctcacgataatatccgggt |
| | | aggcgcaatcactttcgtctactccgttacaaagcgaggctgggtat |
| | | ttcccggcctttctgttatccgaaatccactgaaagcacagcggctg |
| | | gctgaggagataaataataaacgaggggctgtatgcacaaagcatct |
| | | tctgttgagttaagaacgagtatcgagatggcacatagccttgctca |
| | | aattggaatcaggtttgtgccaataccagtagaaacagacgaagaat |
| | | ccatgggtatggacagatctcaaaaaaagcaccgactcggtgccact |
| | | ttttcaagttgataacggactagccttattttaacttgctatttcta |
| | | gctctaaaacctggtaacaggattagcagatgtgtgaaattgttatc |
| | | cgctcacaattccacacattatacgagccggatgattaattgtcaac |
| | | agctcatttcagaatatttgccagaaccgttatgatgtcggcgcaaa |
| | | aaacattatccagaacgggagtgcgccttgagcgacacgaattatgc |
| | | agtgatttacgacctgcacagccataccacagcttccgatggctgcc |
| | | tgacgccagaagcattggtgcaccgtgcagtcgatgataagctgtca |
| | | aaccagatcaattcgcgctaactcacattaattgcgttgcgctcact |
| | | gcccgctttccagtcgggaaacctgtcgtgccagctgcattaatgaa |
| | | tcggccaacgcgcggggagaggcggtttgcgtattgggcgccagggt |
| | | ggtttttcttttcaccagtgagacgggcaacagctgattgcccttca |
| | | ccgcctggccctgagagagttgcagcaagcggtccacgctggtttgc |
| | | Cccagcaggcgaaaatcctgtttgatggtggttgacggcgggatata |
| | | acatgagctgtcttcggtatcgtcgtatcccactaccgagatatccg |
| | | caccaacgcgcagcccggactcggtaatggcgcgcattgcgcccagc |
| | | gccatctgatcgttggcaaccagcatcgcagtgggaacgatgccctc |
| | | attcagcatttgcatggtttgttgaaaaccggacatggcactccagt |
| | | cgccttcccgttccgctatcggctgaatttgattgcgagtgagatat |
| | | ttatgccagccagccagacgcagacgcgccgagacagaacttaatgg |
| | | gcccgctaacagcgcgatttgctggtgacccaatgcgaccagatgct |
| | | ccacgcccagtcgcgtaccgtcttcatgggagaaaataatactgttg |
| | | atgggtgtctggtcagagacatcaagaaataacgccggaacattagt |
| | | gcaggcagcttccacagcaatggcatcctggtcatccagcggatagt |
| | | taatgatcagcccactgacgcgttgcgcgagaagattgtgcaccgcc |
| | | gctttacaggcttcgacgccgcttcgttctaccatcgacaccaccac |
| | | gctggcacccagttgatcggcgcgagatttaatcgccgcgacaattt |
| | | gcgacggcgcgtgcagggccagactggaggtggcaacgccaatcagc |
| | | aacgactgtttgcccgccagtigttgtgccacgcggitgggaatgta |
| | | attcagctccgccatcgccgcttccactttttcccgcgttttcgcag |
| | | aaacgtggctggcctggttcaccacgcgggaaacggtctgataagag |
| | | acaccggcatactctgcgacatcgtataacgttactggtttcacatt |
| | | caccaccctgaattgactctcttccgggcgctatcatgccataccgc |
| | | gaaaggttttgcaccattcgatggtgtcaacgtaaatgcatgccgct |
| | | tcgccttccatgggtatggacagttttccctttgatatgtaacggtg |
| | | aacagttgttctacttttgtttgttagtcttgatgcttcactgatag |
| | | atacaagagccataagaacctcagatccttccgtatttagccagtat |
| | | gttctctagtgtggttcgttgtttttgcgtgagccatgagaacgaac |
| | | cattgagatcatacttactttgcatgtcactcaaaaattttgcctca |
| | | aaactggtgagctgaatttttgcagttaaagcatcgtgtagtgtttt |
| | | tcttagtccgttacgtaggtaggaatctgatgtaatggttgttggta |
| | | ttttgtcaccattcatttttatctggttgttctcaagttcggttacg |
| | | agatccatttgtctatctagttcaacttggaaaatcaacgtatcagt |
| | | cggggggcctcgcttatcaaccaccaatttcatattgctgtaagtgt |
| | | ttaaatctttacttattggtttcaaaacccattggttaagcctttta |
| | | aactcatggtagttattttcaagcattaacatgaacttaaattcatc |
| | | aaggctaatctctatatttgccttgtgagttttcttttgtgttagtt |
| | | cttttaataaccactcataaatcctcatagagtatttcttttcaaaa |
| | | gacttaacatgttccagattatattttatgaatttttttaactggaa |
| | | aagataaggcaatatctcttcactaaaaactaattctaatttttcgc |
| | | ttgagaacttggcatagtttgtccactggaaaatctcaaagccttta |
| | | accaaaggattcctgatttccacagttctcgtcatcagctctctggt |
| | | tgctttagctaatacaccataagcattttccctactgatgttcatca |
| | | tctgagcgtattggttataagtgaacgataccgtccgttctttcctt |
| | | gtagggttttcaatcgtggggttgagtagtgccacacagcataaaat |
| | | tagcttggtttcatgctccgttaagtcatagcgactaatcgctagtt |
| | | catttgctttgaaaacaactaattcagacatacatctcaattggtct |
| | | aggtgattttaatcactataccaattgagatgggctagtcaatgata |
| | | attactagtccttttcctttgagtigtgggtatctgtaaattctgct |
| | | agacctttgctggaaaacttgtaaattctgctagaccctctgtaaat |
| | | tccgctagacctttgtgtgttttttttgtttatattcaagtggttat |
| | | aatttatagaataaagaaagaataaaaaaagataaaaagaatagatc |
| | | ccagccctgtgtataactcactactttagtcagttccgcagtattac |
| | | aaaaggatgtcgcaaacgctgtttgctcctctacaaaacagacctta |
| | | aaaccctaaaggcttaagtagcaccctcgcaagctcggttgcggccg |
| | | caatcgggcaaatcgctgaatattccttttgtctccgaccatcaggc |
| | | acctgagtcgctgtctttttcgtgacattcagttcgctgcgctcacg |
| | | gctctggcagtgaatgggggtaaatggcactacaggcgccttttatg |
| | | gattcatgcaaggaaactacccataatacaagaaaagcccgtcacgg |
| | | gcttctcagggcgttttatggcgggtctgctatgtggtgctatctga |
| | | ctttttgctgttcagcagttcctgccctctgattttccagtctgacc |
| | | acttcggattatcccgtgacaggtcattcagactggctaatgcaccc |
| | | agtaaggcagcggtatcatcaacggggtctgacgctcagtggaacga |
| | | aaactcacgttaagggattttggtcatgagattatcaaaaaggatct |
| | | tcacctagatccttttaaattaaaaatgaagttttaaatcaatctaa |
| | | agtatatatgagtaaacttggtctgacagttacgtttccacaaccaa |
| | | ttaaccaattctgattagaaaaactcatcgagcatcaaatgaaactg |
| | | caatttattcatatcaggattatcaataccatatttttgaaaaagcc |
| | | gtttctgtaatgaaggagaaaactcaccgaggcagttccataggatg |
| | | gcaagatcctggtatcggtctgcgattccgactcgtccaacatcaat |
| | | acaacctattaatttcccctcgtcaaaaataaggttatcaagtgaga |
| | | aatcaccatgagtgacgactgaatccggtgagaatggcaaaagctta |
| | | tgcatttctttccagacttgttcaacaggccagccattacgctcgtc |
| | | atcaaaatcactcgcatcaaccaaaccgttattcattcgtgattgcg |
| | | cctgagcgagacgaaatacgcgatcgctgttaaaaggacaattacaa |
| | | acaggaatcgaatgcaaccggcgcaggaacactgccagcgcatcaac |
| | | aatattttcacctgaatcaggatattcttctaatacctggaatgctg |
| | | ttttcccggggatcgcagtggtgagtaaccatgcatcatcaggagta |
| | | cggataaaatgcttgatggtcggaagaggcataaattccgtcagcca |
| | | gtttagtctgaccatctcatctgtaacatcattggcaacgctacctt |
| | | tgccatgtttcagaaacaactctggcgcatcgggcttcccatacaat |
| | | cgatagattgtcgcacctgattgcccgacattatcgcgagcccattt |
| | | atacccatataaatcagcatccatgttggaatttaatcgcggcctcg |
| | | agcaagacgtttcccgttgaatatggctcataacaccccttgtatta |
| | | ctgtttatgtaagcagacagttttattgttcatgatgatatattttt |
| | | atcttgtgcaatgtaacatcagagattttgagacacaacgtggcttt |
| | | ccctgcagggtttgcagtcagagtagaatagaagtatcaaaaaaagc |
| | | accgactcggtgccactttttcaagttgataacggactagccttatt |
| | | ttaacttgctatgctgttttgaatggttccaacaagattattttata |
| | | actittataacaaataatcaaggagaaattcaaagaaatttatcagc |
| | | Cataaaacaatacttaatactatagaatgataacaaaataaactact |
| | | ttttaaaagaattttgtgttataatctatttattattaagtattggg |
| | | taatattttttgaagagatattttgaaaaagaaaaattaaagcatat |
| | | taaactaatttcggaggtcattaaaactattattgaaatcatcaaac |
| | | tcattatggatttaatttaaactttttattttaggaggcaaaa |
| pAL1950 | 69 | ttcccgtgcatcggtctgccgttttagagctagaaatagcaagttaa |
| (CRISPR | | aataaggctagtccgttatcaacttgaaaaagtggcaccgagtcggt |
| sgRNA | | gctttttttgaattctctagagtcgacctgcagaagcttagatctat |
| plasmid | | taccctgttatccctactcgagttcatgtgcagctccataagcaaaa |
| targeting | | ggggatgataagtttatcaccaccgactatttgcaacagtgccgttg |
| pfkA) | | atcgtgctatgatcgactgatgtcatcagcggtggagtgcaatgtca |
| | | tgagtattcaacatttccgtgtcgcccttattcccttttttgcggca |
| | | ttttgccttcctgtttttgctcacccagaaacgctggtgaaagtaaa |
| | | agatgctgaagatcagttgggtgcacgagtgggttacatcgaactgg |
| | | atctcaacagcggtaagatccttgagagttttcgccccgaagaacgt |
| | | tttccaatgatgagcactittaaagttctgctatgtggcgcggtatt |
| | | atcccgtgttgacgccgggcaagagcaactcggtcgccgcatacact |
| | | attctcagaatgacttggttgagtactcaccagtcacagaaaagcat |
| | | cttacggatggcatgacagtaagagaattatgcagtgctgccataac |
| | | catgagtgataacactgcggccaacttacttctgacaacgatcggag |
| | | gaccgaaggagctaaccgcttttttgcacaacatgggggatcatgta |
| | | actcgccttgatcgttgggaaccggagctgaatgaagccataccaaa |
| | | cgacgagcgtgacaccacgatgcctgcagcaatggcaacaacgttgc |
| | | gcaaactattaactggcgaactacttactctagcttcccggcaacaa |
| | | ttaatagactggatggaggcggataaagttgcaggaccacttctgcg |
| | | ctcggcccttccggctggctggtttattgctgataaatctggagccg |
| | | gtgagcgtgggtctcgcggtatcattgcagcactggggccagatggt |
| | | aagccctcccgtatcgtagttatctacacgacggggagtcaggcaac |
| | | tatggatgaacgaaatagacagatcgctgagataggtgcctcactga |
| | | ttaagcattggtaagatgccgctcgccagtcgattggctgagctcat |
| | | gaagttcctattccgaagttccgcgaacgcgtaaaggatctaggtga |
| | | agatcctttttgataatctcatgaccaaaatcccttaacgtgagttt |
| | | tcgttccactgagcgtcagaccccgtagaaaagatcaaaggatcttc |
| | | ttgagatcctttttttctgcgcgtaatctgctgcttgcaaacaaaaa |
| | | aaccaccgctaccagcggtggtttgtttgccggatcaagagctacca |
| | | actctttttccgaaggtaactggcttcagcagagcgcagataccaaa |
| | | tactgtccttctagtgtagccgtagttaggccaccacttcaagaact |
| | | ctgtagcaccgcctacatacctcgctctgctaatcctgttaccagtg |
| | | gctgctgccagtggcgataagtcgtgtcttaccgggttggactcaag |
| | | acgatagttaccggataaggcgcagcggtcgggctgaacggggggtt |
| | | cgtgcacacagcccagcttggagcgaacgacctacaccgaactgaga |
| | | tacctacagcgtgagctatgagaaagcgccacgcttcccgaagggag |
| | | aaaggcggacaggtatccggtaagcggcagggtcggaacaggagagc |
| | | gcacgagggagcttccagggggaaacgcctggtatctttatagtcct |
| | | gtcgggtttcgccacctctgacttgagcgtcgatttttgtgatgctc |
| | | gtcaggggggcggagcctatggaaaaacgccagcaacgcggcctttt |
| | | tacggttcctggccttttgctggccttttgctcacatgttctttcct |
| | | gcgttatcccctgattctgtggataaccgtattaccgcctttgagtg |
| | | agctgataccgctcgccgcagccgaacgaccgagcgcagcgagtcag |
| | | tgagcgaggaagcggaagagcgcctgatgcggtattttctccttacg |
| | | catctgtgcggtatttcacaccgcatatgctggatccttgacagcta |
| | | gctcagtcctaggtataatactagt |
| pAL1951 | 70 | aattgaatccggggccatccgtittagagctagaaatagcaagttaa |
| (CRISPR | | aataaggctagtccgttatcaacttgaaaaagtggcaccgagtcggt |
| sgRNA | | gctttttttgaattctctagagtcgacctgcagaagcttagatctat |
| plasmid | | taccctgttatccctactcgagttcatgtgcagctccataagcadaa |
| targeting | | ggggatgataagtttatcaccaccgactatttgcaacagtgccgttg |
| pfkB) | | atcgtgctatgatcgactgatgtcatcagcggtggagtgcaatgtca |
| | | tgagtattcaacatttccgtgtcgcccttattcccttttttgcggca |
| | | ttttgccttcctgtttttgctcacccagaaacgctggtgaaagtaaa |
| | | agatgctgaagatcagttgggtgcacgagtgggttacatcgaactgg |
| | | atctcaacagcggtaagatccttgagagttttcgccccgaagaacgt |
| | | tttccaatgatgagcacttttaaagttctgctatgtggcgcggtatt |
| | | atcccgtgttgacgccgggcaagagcaactcggtcgccgcatacact |
| | | attctcagaatgacttggttgagtactcaccagtcacagaaaagcat |
| | | cttacggatggcatgacagtaagagaattatgcagtgctgccataac |
| | | catgagtgataacactgcggccaacttacttctgacaacgatcggag |
| | | gaccgaaggagctaaccgcttttttgcacaacatgggggatcatgta |
| | | actcgccttgatcgttgggaaccggagctgaatgaagccataccaaa |
| | | cgacgagcgtgacaccacgatgcctgcagcaatggcaacaacgttgc |
| | | gcaaactattaactggcgaactacttactctagcttcccggcaacaa |
| | | ttaatagactggatggaggcggataaagttgcaggaccacttctgcg |
| | | ctcggcccttccggctggctggtttattgctgataaatctggagccg |
| | | gtgagcgtgggtctcgcggtatcattgcagcactggggccagatggt |
| | | aagccctcccgtatcgtagttatctacacgacggggagtcaggcaac |
| | | tatggatgaacgaaatagacagatcgctgagataggtgcctcactga |
| | | ttaagcattggtaagatgccgctcgccagtcgattggctgagctcat |
| | | gaagttcctattccgaagttccgcgaacgcgtaaaggatctaggtga |
| | | agatcctttttgataatctcatgaccaaaatcccttaacgtgagttt |
| | | tcgttccactgagcgtcagaccccgtagaaaagatcaaaggatcttc |
| | | ttgagatcctttttttctgcgcgtaatctgctgcttgcaaacaaaaa |
| | | aaccaccgctaccagcggtggtttgtttgccggatcaagagctacca |
| | | actctttttccgaaggtaactggcttcagcagagcgcagataccaaa |
| | | tactgtccttctagtgtagccgtagttaggccaccacttcaagaact |
| | | ctgtagcaccgcctacatacctcgctctgctaatcctgttaccagtg |
| | | gctgctgccagtggcgataagtcgtgtcttaccgggttggactcaag |
| | | acgatagttaccggataaggcgcagcggtcgggctgaacggggggtt |
| | | cgtgcacacagcccagcttggagcgaacgacctacaccgaactgaga |
| | | tacctacagcgtgagctatgagaaagcgccacgcttcccgaagggag |
| | | aaaggcggacaggtatccggtaagcggcagggtcggaacaggagagc |
| | | gcacgagggagcttccagggggaaacgcctggtatctttatagtcct |
| | | gtcgggtttcgccacctctgacttgagcgtcgatttttgtgatgctc |
| | | gtcaggggggcggagcctatggaaaaacgccagcaacgcggcctttt |
| | | tacggttcctggccttttgctggccttttgctcacatgttctttcct |
| | | gcgttatcccctgattctgtggataaccgtattaccgcctttgagtg |
| | | agctgataccgctcgccgcagccgaacgaccgagcgcagcgagtcag |
| | | tgagcgaggaagcggaagagcgcctgatgcggtattttctccttacg |
| | | catctgtgcggtatttcacaccgcatatgctggatccttgacagcta |
| | | gctcagtcctaggtataatactagt |
| pAL1957 | 71 | gtcgcactggctgttgctgggttttagagctagaaatagcaagttaa |
| (CRISPR | | aataaggctagtccgttatcaacttgaaaaagtggcaccgagtcggt |
| sgRNA | | gctttttttgaattctctagagtcgacctgcagaagcttagatctat |
| plasmid | | taccctgttatccctactcgagttcatgtgcagctccataagcaaaa |
| targeting | | ggggatgataagtttatcaccaccgactatttgcaacagtgccgttg |
| rpiB) | | atcgtgctatgatcgactgatgtcatcagcggtggagtgcaatgtca |
| | | tgagtattcaacatttccgtgtcgcccttattcccttttttgcggca |
| | | ttttgccttcctgtttttgctcacccagaaacgctggtgaaagtaaa |
| | | agatgctgaagatcagttgggtgcacgagtgggttacatcgaactgg |
| | | atctcaacagcggtaagatccttgagagttttcgccccgaagaacgt |
| | | tttccaatgatgagcactittaaagttctgctatgtggcgcggtatt |
| | | atcccgtgttgacgccgggcaagagcaactcggtcgccgcatacact |
| | | attctcagaatgacttggttgagtactcaccagtcacagaaaagcat |
| | | cttacggatggcatgacagtaagagaattatgcagtgctgccataac |
| | | catgagtgataacactgcggccaacttacttctgacaacgatcggag |
| | | gaccgaaggagctaaccgcttttttgcacaacatgggggatcatgta |
| | | actcgccttgatcgttgggaaccggagctgaatgaagccataccaaa |
| | | cgacgagcgtgacaccacgatgcctgcagcaatggcaacaacgttgc |
| | | gcaaactattaactggcgaactacttactctagcttcccggcaacaa |
| | | ttaatagactggatggaggcggataaagttgcaggaccacttctgcg |
| | | ctcggcccttccggctggctggtttattgctgataaatctggagccg |
| | | gtgagcgtgggtctcgcggtatcattgcagcactggggccagatggt |
| | | aagccctcccgtatcgtagttatctacacgacggggagtcaggcaac |
| | | tatggatgaacgaaatagacagatcgctgagataggtgcctcactga |
| | | ttaagcattggtaagatgccgctcgccagtcgattggctgagctcat |
| | | gaagttcctattccgaagttccgcgaacgcgtaaaggatctaggtga |
| | | agatcctttttgataatctcatgaccaaaatcccttaacgtgagttt |
| | | tcgttccactgagcgtcagaccccgtagaaaagatcaaaggatcttc |
| | | ttgagatcctttttttctgcgcgtaatctgctgcttgcaaacaaaaa |
| | | aaccaccgctaccagcggtggtttgtttgccggatcaagagctacca |
| | | actctttttccgaaggtaactggcttcagcagagcgcagataccaaa |
| | | tactgtccttctagtgtagccgtagttaggccaccacttcaagaact |
| | | ctgtagcaccgcctacatacctcgctctgctaatcctgttaccagtg |
| | | gctgctgccagtggcgataagtcgtgtcttaccgggttggactcaag |
| | | acgatagttaccggataaggcgcagcggtcgggctgaacggggggtt |
| | | cgtgcacacagcccagcttggagcgaacgacctacaccgaactgaga |
| | | tacctacagcgtgagctatgagaaagcgccacgcttcccgaagggag |
| | | aaaggcggacaggtatccggtaagcggcagggtcggaacaggagagc |
| | | gcacgagggagcttccagggggaaacgcctggtatctttatagtcct |
| | | gtcgggtttcgccacctctgacttgagcgtcgatttttgtgatgctc |
| | | gtcaggggggcggagcctatggaaaaacgccagcaacgcggcctttt |
| | | tacggttcctggccttttgctggccttttgctcacatgttctttcct |
| | | gcgttatcccctgattctgtggataaccgtattaccgcctttgagtg |
| | | agctgataccgctcgccgcagccgaacgaccgagcgcagcgagtcag |
| | | tgagcgaggaagcggaagagcgcctgatgcggtattttctccttacg |
| | | catctgtgcggtatttcacaccgcatatgctggatccttgacagcta |
| | | gctcagtcctaggtataatactagt |
| pAL1958 | 72 | gcagaagaagtggggatcgagttttagagctagaaatagcaagttaa |
| (CRISPR | | aataaggctagtccgttatcaacttgaaaaagtggcaccgagtcggt |
| sgRNA | | gctttttttgaattctctagagtcgacctgcagaagcttagatctat |
| plasmid | | taccctgttatccctactcgagttcatgtgcagctccataagcaaaa |
| targeting | | ggggatgataagtttatcaccaccgactatttgcaacagtgccgttg |
| zwf) | | atcgtgctatgatcgactgatgtcatcagcggtggagtgcaatgtca |
| | | tgagtattcaacatttccgtgtcgcccttattcccttttttgcggca |
| | | ttttgccttcctgtttttgctcacccagaaacgctggtgaaagtaaa |
| | | agatgctgaagatcagttgggtgcacgagtgggttacatcgaactgg |
| | | atctcaacagcggtaagatccttgagagttttcgccccgaagaacgt |
| | | tttccaatgatgagcacttttaaagttctgctatgtggcgcggtatt |
| | | atcccgtgttgacgccgggcaagagcaactcggtcgccgcatacact |
| | | attctcagaatgacttggttgagtactcaccagtcacagaaaagcat |
| | | cttacggatggcatgacagtaagagaattatgcagtgctgccataac |
| | | catgagtgataacactgcggccaacttacttctgacaacgatcggag |
| | | gaccgaaggagctaaccgcttttttgcacaacatgggggatcatgta |
| | | actcgccttgatcgttgggaaccggagctgaatgaagccataccaaa |
| | | cgacgagcgtgacaccacgatgcctgcagcaatggcaacaacgttgc |
| | | gcaaactattaactggcgaactacttactctagcttcccggcaacaa |
| | | ttaatagactggatggaggcggataaagttgcaggaccacttctgcg |
| | | ctcggcccttccggctggctggtttattgctgataaatctggagccg |
| | | gtgagcgtgggtctcgcggtatcattgcagcactggggccagatggt |
| | | aagccctcccgtatcgtagttatctacacgacggggagtcaggcaac |
| | | tatggatgaacgaaatagacagatcgctgagataggtgcctcactga |
| | | ttaagcattggtaagatgccgctcgccagtcgattggctgagctcat |
| | | gaagttcctattccgaagttccgcgaacgcgtaaaggatctaggtga |
| | | agatcctttttgataatctcatgaccaaaatcccttaacgtgagttt |
| | | tcgttccactgagcgtcagaccccgtagaaaagatcaaaggatcttc |
| | | ttgagatcctttttttctgcgcgtaatctgctgcttgcaaacaaaaa |
| | | aaccaccgctaccagcggtggtttgtttgccggatcaagagctacca |
| | | actctttttccgaaggtaactggcttcagcagagcgcagataccaaa |
| | | tactgtccttctagtgtagccgtagttaggccaccacttcaagaact |
| | | ctgtagcaccgcctacatacctcgctctgctaatcctgttaccagtg |
| | | gctgctgccagtggcgataagtcgtgtcttaccgggttggactcaag |
| | | acgatagttaccggataaggcgcagcggtcgggctgaacggggggtt |
| | | cgtgcacacagcccagcttggagcgaacgacctacaccgaactgaga |
| | | tacctacagcgtgagctatgagaaagcgccacgcttcccgaagggag |
| | | aaaggcggacaggtatccggtaagcggcagggtcggaacaggagagc |
| | | gcacgagggagcttccagggggaaacgcctggtatctttatagtcct |
| | | gtcgggtttcgccacctctgacttgagcgtcgatttttgtgatgctc |
| | | gtcaggggggcggagcctatggaaaaacgccagcaacgcggcctttt |
| | | tacggttcctggccttttgctggccttttgctcacatgttctttcct |
| | | gcgttatcccctgattctgtggataaccgtattaccgcctitgagtg |
| | | agctgataccgctcgccgcagccgaacgaccgagcgcagcgagtcag |
| | | tgagcgaggaagcggaagagcgcctgatgcggtattttctccttacg |
| | | catctgtgcggtatttcacaccgcatatgctggatcttgacagcta |
| | | gctcagtcctaggtataatactagt |
| pAL2178 | 73 | tcggccctcgatagccagcagttttagagctagaaatagcaagttaa |
| (CRISPR | | aataaggctagtccgttatcaacttgaaaaagtggcaccgagtcggt |
| sgRNA | | gctttttttgaattctctagagtcgacctgcagaagcttagatctat |
| plasmid | | taccctgttatccctactcgagttcatgtgcagctccataagcaaaa |
| targeting | | ggggatgataagtttatcaccaccgactatttgcaacagtgccgttg |
| manA) | | atcgtgctatgatcgactgatgtcatcagcggtggagtgcaatgtca |
| | | tgagtattcaacatttccgtgtcgcccttattcccttttttgcggca |
| | | ttttgccttcctgtttttgctcacccagaaacgctggtgaaagtaaa |
| | | agatgctgaagatcagttgggtgcacgagtgggttacatcgaactgg |
| | | atctcaacagcggtaagatccttgagagttttcgccccgaagaacgt |
| | | tttccaatgatgagcacttttaaagttctgctatgtggcgcggtatt |
| | | atcccgtgttgacgccgggcaagagcaactcggtcgccgcatacact |
| | | attctcagaatgacttggttgagtactcaccagtcacagaaaagcat |
| | | cttacggatggcatgacagtaagagaattatgcagtgctgccataac |
| | | catgagtgataacactgcggccaacttacttctgacaacgatcggag |
| | | gaccgaaggagctaaccgcttttttgcacaacatgggggatcatgta |
| | | actcgccttgatcgttgggaaccggagctgaatgaagccataccaaa |
| | | cgacgagcgtgacaccacgatgcctgcagcaatggcaacaacgttgc |
| | | gcaaactattaactggcgaactacttactctagcttcccggcaacaa |
| | | ttaatagactggatggaggcggataaagttgcaggaccacttctgcg |
| | | ctcggcccttccggctggctggtttattgctgataaatctggagccg |
| | | gtgagcgtgggtctcgcggtatcattgcagcactggggccagatggt |
| | | aagccctcccgtatcgtagttatctacacgacggggagtcaggcaac |
| | | tatggatgaacgaaatagacagatcgctgagataggtgcctcactga |
| | | ttaagcattggtaagatgccgctcgccagtcgattggctgagctcat |
| | | gaagttcctattccgaagttccgcgaacgcgtaaaggatctaggtga |
| | | agatcctttttgataatctcatgaccaaaatcccttaacgtgagttt |
| | | tcgttccactgagcgtcagaccccgtagaaaagatcaaaggatcttc |
| | | ttgagatcctttttttctgcgcgtaatctgctgcttgcaaacaaaaa |
| | | aaccaccgctaccagcggtggtttgtttgccggatcaagagctacca |
| | | actctttttccgaaggtaactggcttcagcagagcgcagataccaaa |
| | | tactgtccttctagtgtagccgtagttaggccaccacttcaagaact |
| | | ctgtagcaccgcctacatacctcgctctgctaatcctgttaccagtg |
| | | gctgctgccagtggcgataagtcgtgtcttaccgggttggactcaag |
| | | acgatagttaccggataaggcgcagcggtcgggctgaacggggggtt |
| | | cgtgcacacagcccagcttggagcgaacgacctacaccgaactgaga |
| | | tacctacagcgtgagctatgagaaagcgccacgcttcccgaagggag |
| | | aaaggcggacaggtatccggtaagcggcagggtcggaacaggagagc |
| | | gcacgagggagcttccagggggaaacgcctggtatctttatagtcct |
| | | gtcgggtttcgccacctctgacttgagcgtcgatttttgtgatgctc |
| | | gtcaggggggcggagcctatggaaaaacgccagcaacgcggcctttt |
| | | tacggttcctggccttttgctggccttttgctcacatgttctttcct |
| | | gcgttatcccctgattctgtggataaccgtattaccgcctttgagtg |
| | | agctgataccgctcgccgcagccgaacgaccgagcgcagcgagtcag |
| | | tgagcgaggaagcggaagagcgcctgatgcggtattttctccttacg |
| | | catctgtgcggtatttcacaccgcatatgctggatccttgacagcta |
| | | gctcagtcctaggtataatactagt |

### Example 2—Biosynthesis of Psicose from Glucose

The deadly rise in sedentary lifestyle and access to calorie-dense foods has tripled global obesity rates from 1975 to 2016 (Blüher, Nat. Rev. Endocrinol. 2019 155 15, 288-298 (2019)). Almost 40% of adults world-wide are considered overweight, a major risk factor associated with cardiovascular disease, diabetes, musculoskeletal disorders, and some cancers. In response to this public health crisis, more people are looking to treat or prevent disease by adopting health-conscious, low-calorie diets. The food industry can play a role in helping people make better choices by substituting sucrose and high-fructose corn syrup with zero and low-calorie sugar substitutes. As such, the market for sugar substitutes is expected to reach 20.6 billion USD by 2025 (MarketandMarket. Sugar Substitutes Market by Type (High Fructose Syrup, High-Intensity Sweetener, Low-Intensity Sweetener), Composition, Application (Beverages, Food Products, and Health & Personal Care Products), and Region-Global Forecast to 2025. (2020)).

Rare sugars are monosaccharides uncommonly found in nature, which differ slightly in structure from common sugars such as glucose and fructose. Many rare sugars are non-nutritive and possess potential health benefits, making them intriguing targets as sugar substitutes. The GRAS, zero-calorie, rare sugar D-Psicose is a 3′ epimer of fructose, with 70% the sweetness of sucrose. Marketed to consumers as “allulose”, D-psicose has a desirable flavor profile along with favorable browning, hygroscopic, and solubility properties. Additionally, studies have correlated consumption of D-psicose to antihyperglycemic, antihyperlipidemic, antiparasitic, and antioxidant health benefits3. As with many rare sugars, a limitation to the study of D-psicose and its implementation in industry is the lack of an economic, large-scale production system. D-Psicose is naturally found in some fruits and grains, but only in minute concentrations unviable for extraction (Oshima et al., Food Sci. Technol. Res. 12, 137-143 (2006)).

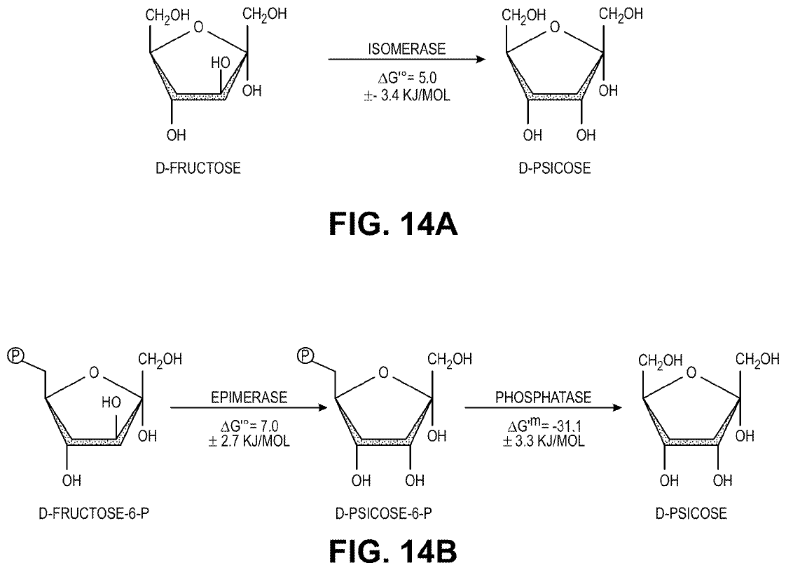

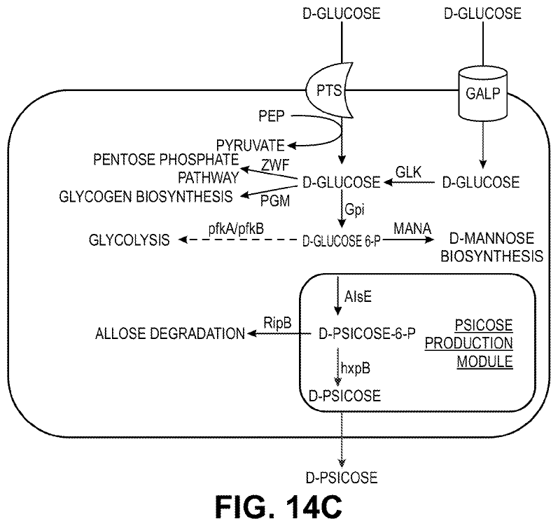

Although synthetic methods for D-psicose production have been proposed, these methods suffer from poor stereoselectivity, yield, and purification (Wang et al., Nature 578, 403-408 (2020)). As such, biosynthesis using the enzyme D-tagatose-3-epimerase (DTEase) and D-psicose-3-epimerase (DPEase) have been the primary focus of D-psicose production (Itoh et al., OUP 58, 2168-2171 (2014): Jiang et al., Front. Bioeng. Biotechnol. 8, 26 (2020): Armetta et al., Synth. Biol. 4, ysz028 (2019)). Both enzymes operate under high temperature and alkaline pH to epimerize the C3 carbon of D-fructose to form D-psicose. Attempts to bolster production have focused on engineering these enzymes for improved catalytic efficiency, thermostability, and ability to perform at lower pHs and temperatures (Hu et al., Compr. Rev. Food Sci. Food Saf. 20, 6012-6026 (2021)). Despite concerted efforts to improve DPEases and DTEases, this method inherently suffers from limited yield due to a lack of thermodynamic driving force, and therefore has not achieved conversion yields greater than 50%. The epimerization of D-fructose to D-psicose is reversible and has a predicted αG° of +5 kJ mol⁻¹, making it thermodynamically unfavorable and more likely to favor D-fructose at equilibrium (FIG. 14A). Low conversion results in a mixed solution of D-fructose and D-psicose, making isolation and purification a major challenge. Methods that achieved conversions greater than 50% relied on the inclusion of toxic or expensive cofactors.

To overcome this barrier, the present example proposes using phosphorylation and dephosphorylation to provide thermodynamic incentive and a driving force towards D-psicose production (Fie 14B). Carrying a predicted ΔG′^{m} of −31.1 kJ mol⁻¹ at typical physiological concentrations of 1 mM, the dephosphorylation of D-psicose-6-phosphate (P6P) to D-psicose is a highly favorable reaction. The enzymatic machinery and cofactors necessary for phosphorylation/dephosphorylation of sugars are readily available within living cells. Furthermore, most organisms, including the model organism Escherichia colim utilize phosphorylation/dephophorylation of sugars as part of sugar consumption and central carbon metabolism. Within E. coli, carbon metabolism begins with the phosphotransferase system (PTS), where D-glucose is simultaneously phosphorylated and transferred across the cell membrane. Alternatively, glucose can be transported across the cell membrane by galactose proton symporter GalP, after which it is phosphorylated by glucokinase Glk13,14. D-glucose-6-phosphate (G6P) can then be isomerized to D-fructose-6-phosphate (F6P) and used in glycolysis. Here, it is theorized that a portion of F6P could be diverted from glycolysis and epimerized to POP, which could then be dephosphorylated to D-psicose and excreted from the cell.

In this example, it was discovered that E. coli natively possesses enzymes capable of completing the D-psicose biosynthetic production pathway proposed above (FIG. 14B). The epimerization of F6P to POP can be accomplished using the D-allulose-6-phosphate 3-epimerase (AlsE), and the dephosphorylation of POP can be accomplished using the phosphatase hexitol-phosphatase B (HxpB). By utilizing E. coli's native genes, the present example successfully generated D-psicose from D-glucose at yields greater than 50%, without the need for heterologous enzyme expression.

The capacity of E. coli to produce D-psicose was improved by additionally expressing alsE and hxpB and removing or regulating competing metabolic pathways, including the pentose phosphate pathway (PPP), glycogen biosynthesis, glycolysis, the D-allose degradation pathway, and the D-mannose degradation pathway. D-glucose import was supplemented by additionally expressing the native galactose proton symporter gene, galP and the glucokinase gene, glk. To further increase production while maintaining cellular viability, multiple strategies for dynamic gene regulation and carbon flux partitioning were explored. During the growth phase, cells need more energy to rigorously grow, and genes related to glycolysis should be expressed. When the cells enter the stationary phase and are not actively growing, carbon flux can be diverted from glycolysis to D-psicose production. To balance carbon partitioning depending on growth phase, two strategies to dynamically regulate the expression of key metabolic and D-psicose production genes were explored: inducer-free stationary-phase promoters and clustered regularly interspaced short palindromic repeats interference (CRISPRi).

### Design of a Thermodynamically Favorable D-Psicose Production Pathway.

Currently, the primary method for production of D-psicose involves the in vitro enzymatic isomerization of D-fructose to D-psicose, a thermodynamically unfavorable process that results in incomplete product formation (˜50%) requires expensive purification (FIG. 14A). To overcome the thermodynamic limitations of this method, the present example proposed a pathway driven by the phosphorylation and dephosphorylation of sugars, a process whose required cofactors and enzymes that are readily available within living organisms (FIGS. 14B and 14C). Therefore, the model organism E. coli was chosen to host the D-psicose biosynthetic pathway.

The proposed pathway begins with the assimilation of D-glucose into E. coli via the PTS, which converts D-glucose to G6P (FIG. 14C). Alternatively, D-glucose can be assimilated by the galactose proton symporter GalP, after which it is phosphorylated to G6P by glucokinase Glk (FIG. 14C). G6P is then isomerized to F6P via glucose 6-phosphate isomerase (Gpi). Here, the proposed pathway diverges from native carbon metabolism. Many enzymes are promiscuous, utilizing a variety of substrates. As such, it was theorized E. coli may natively possess enzymes capable of producing D-psicose given the right conditions and alterations to sugar metabolism. F6P could be epimerized to P6P, which could then be dephosphorylated in a final, thermodynamically favorable step to D-psicose (FIG. 14C). It was theorized that placing this favorable reaction at the end of the biosynthetic pathway drives flux through the production pathway by way of restoring equilibrium. As D-psicose 6-phoshate is favorably dephosphorylated to D-psicose and excreted from the cell, equilibrium is restored by generating more P6P from F6P.

Assessing E. coli's Native D-Psicose Production Capabilities.

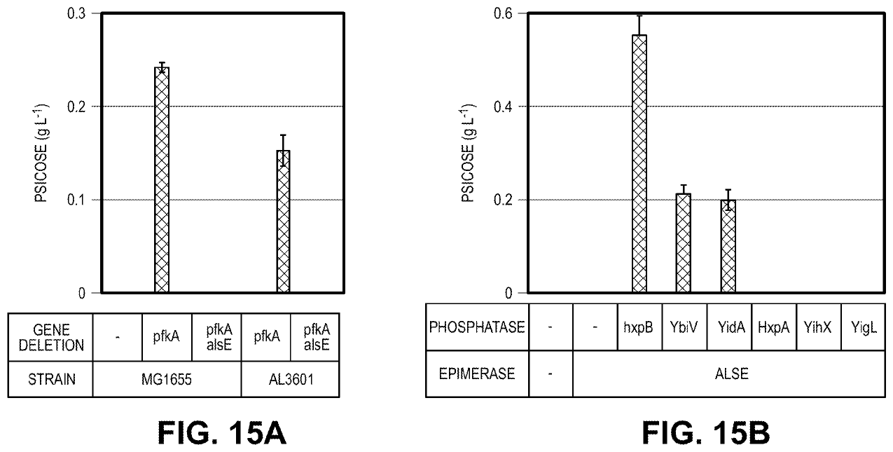

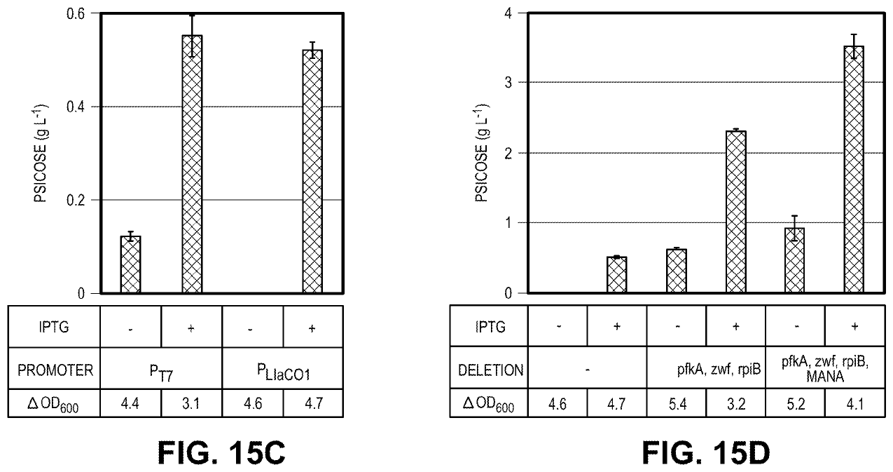

To assess E. coli's native capability of producing D-psicose, production in E. coli MG1655, and MG1655 derived strain AL3601, which carries the gene encoding for T7 RNAP, in M9P media (Table 5, Methods) was tested. Without any genetic manipulations, neither strain produced detectable levels of D-psicose (FIG. 15A).

It was hypothesized that carbon flux must be purposely directed towards D-psicose production by accumulating of the upstream metabolite, F6P. The major metabolic pathway competing for F6P is glycolysis, as F6P is preferentially converted to D-fructose 1,6-bisphosphate by the phosphofructokinase A and B (PfkA and PfkB, FIG. 14C). To build up F6P pools within the cell, the gene encoding PfkA, which accounts for about 90% of phosphofructokinase activity, was deleted in MG1655 and AL3601, generating AL4058 and AL3694 respectively (Table 5). ΔpfkA strains produced D-psicose at 0.24 g L⁻¹ in AL4058 and 0.15 g L⁻¹ in AL3694, indicating that E. coli contains the enzymes necessary for producing D-psicose, most likely from F6P (FIG. 15A). D-psicose production was not detected in cultures grown in M9P media without glucose.

### Elucidating Enzymes Involved in D-Psicose Production.

D-allulose 6-phosphate 3-epimerase (AlsE) was identified as a potential candidate for the conversion of F6P to P6P. AlsE assimilates D-psicose into central carbon metabolism by converting P6P to F6P. Under high concentrations of F6P, AlsE has shown reverse activity and is capable of converting F6P to P6P. To confirm whether AlsE was involved in the production of D-psicose, alsE was deleted in AL3694 and AL4058, resulting in strains AL4063 and AL4082 (Table 5). Neither strain produced detectible levels of D-psicose, suggesting that AlsE is the epimerase involved in the production of D-psicose (FIG. 15A).

E. coli has a number of phosphatase enzymes with potential activity towards POP. The candidate phosphatases were chosen based on a wide range activity towards various sugar substrates and tested hexitol phosphatase B (HxpB), sugar phosphatase YbiV, sugar phosphatase YidA, hexitol phosphatase A (HxpA), α-D-glucose-1-phosphate phosphatase YihX, and phosphosugar phosphatase YigL. To test the activity of each phosphatase for the conversion of P6P to D-psicose, genes for each phosphatase were individually expressed from PT721, along with alsE on an expression plasmid (Table 6). The plasmids were introduced to AL3601 (pfkA+). After 24 h. the strain harboring pAL1946 (P_{T7}:alsE-hxpB) produced the most D-psicose, at 0.55 g L⁻¹, suggesting that HxpB is an excellent phosphatase candidate for D-psicose production (FIG. 15B). The strain harboring pAL1947 (P_{T7}:alsE-ybiV) or pAL2351 (P_{T7}:alsE-yidA) produced 0.21 g L⁻¹ and 0.20 g L⁻¹ of D-psicose respectively, while cultures containing pAL2348 (P_{T7}:alsE-hxpA), pAL2352 (P_{T7}:alsE-yihX), and pAL2349 (P_{T7}:alsE-yigL) did not generate detectable D-psicose (FIG. 15B).

### Identification of Critical P6P Binding Motifs.

A combination of AlphaFold and the Rosetta Molecular Suite were utilized to evaluate predicted binding modes between each of the six phosphatases and P6P. Phosphatases with activity towards P6P (HxpB, YbiV, and YidA) were predicted to form at least two internal hydrogen bonds and one additional hydrogen bond with the terminal hydroxyl group of POP (FIGS. 26A and 26B). Conversely, phosphatases without activity towards P6P (HxpA, YihX, and YigL), were predicted to not form hydrogen bonds with the terminal hydroxyl group of POP. Phosphatase HxpB, which produced the highest D-psicose titer, was predicted to form hydrogen bonds between active site residues and the four hydroxyl groups of POP (FIG. 26B). These predictions show that a minimum of three hydrogen bonds, including a hydrogen bond to the terminal hydroxyl group, is essential for binding P6P in a catalytically competent orientation.

### Comparing Expression System for D-Psicose Production.

In AL3601, the T7 RNA polymerase expression system, which includes the T7 RNA polymerase (RNAP) gene under an IPTG-inducible PlacUV5 promoter, was utilized. However, it was observed decreased growth when IPTG was added to induce the expression of the T7 RNAP expression (FIG. 15C). Thus, the alternative expression system in which the operon of alsE and hxpB was expressed from the IPTG-inducible promoter P_{LlacO1} was tested. Under induced conditions (with IPTG), the strain with P_{LlacO1}:alsE-hxpB produced D-psicose similarly to the strain with P_{T7}:alsE-hxpB (FIG. 15C). Importantly, the strain with P_{LlacO1}:alsE-hxpB got better repression under uninduced conditions (without IPTG) and did not get a growth burden under induced conditions compared to the strain with P_{T7}:alsE-hxpB (FIG. 15C). Thus, the P_{LlacO1} expression system was used for further studies.

### Increasing D-Psicose Production by Removing Competing Pathways.

E. coli relies on two primary glycolytic pathways to metabolize glucose: the PPP and glycolysis (also known as the Embdem Meyerhof Parnas (EMP) pathway). The branching point between D-psicose production and the PPP occurs when glucose-6-phosphate dehydrogenase (Zwf) converts G6P to 6-phospho-D-glucono 1,5-lactone (FIG. 15C). The branching point between D-psicose production and glycolysis occurs when one of two phosphofructokinases, PfkA or PfkB, converts F6P to D-fructose 1,6-bisphosphate (FIG. 15C). The present example established that the deletion of pfkA leads to the development of D-psicose production in comparison to our unmodified base strains (FIG. 15A).

In addition to the PPP and glycolysis, the allose degradation pathway has potential to divert carbon flux away from D-psicose production by reassimilating P6P back into central carbon metabolism. The rpiB gene encodes for allose-6-phosphate isomerase (RpiB), which may be able to convert P6P to aldehydo-D-allose 6-phosphate (FIG. 14C).

To redirect carbon flux away from central carbon metabolism and towards D-psicose production, gene knockouts ΔpfkA, Δzwf, and ΔrpiB were constructed in AL1050, resulting in Strain 1 (Table 4). Those three deletions resulted in a fourfold increase in D-psicose production at 2.31 g L⁻¹ of D-psicose (FIG. 15D).

TABLE 4
List of key strains used in the present example.

| Strain | E. coli | | | |
|---|---|---|---|---|
| no. | strain | Key genotype | Plasmid | Plasmid contents |
| 1 | AL3756 | ΔpfkA Δzwf ΔrpiB | pAL2001 | P_{LlacO1}:alsE-hxpB |
| 2 | AL3990 | 1 + ΔmanA | pAL2001 | P_{LlacO1}:alsE-hxpB |
| 3 | AL3990 | 1 + ΔmanA | pAL2247 | P_{gadB}:alsE-hxpB |
| 4 | AL3990 | 1 + ΔmanA | pAL2247 pAL2264 | P_{gadB}:alsE-hxpB, P_{LlacO1}:galP-glk |
| 5 | AL4186 | 2 + Δpgm | pAL2247 pAL2264 | P_{gadB}:alsE-hxpB, P_{LlacO1}:galP-glk |
| 6 | AL4186 | 2 + Δpgm | pAL2247 pAL2264 | P_{gadB}:alsE-hxpB, P_{LlacO1}:galP-glk, |
| | | | pAL2182 | Pₜₑₜ:dcas9 pTargetF-no target |
| 7 | AL4186 | 2 + Δpgm | pAL2247 pAL2264 | P_{gadB}:alsE-hxpB, P_{LlacO1}:galP-glk, |
| | | | pAL2188 | Pₜₑₜ:dcas9 pTargetF-pfkB |

All strains and plasmids used in this study are listed in Tables 5 and 6, respectively.

TABLE 5
Strains used in the present example.

| Strain | Genotype | Source |
|---|---|---|
| MG1655 | F- lambda- ilvG- rfb-50 rph-1 | Baba et al. 2016 |
| AL1050 | MG1655, but with attB::lacI^{q} tetR spec^{R} | Yoneda et al. 2014 |
| AL3601 | AL1050, but with ss9::P_{lacUV5}:T7rnap | Zhang et al. 2021 |
| AL4058 | MG1655, but with ΔpfkA | The present example |
| AL3694 | AL3601, but with ΔpfkA | The present example |
| AL4063 | AL3694, but with ΔalsE | The present example |
| AL4082 | AL4058, but with ΔalsE | The present example |
| AL3756 | AL1050, but with ΔpfkA Δzwf ΔrpiB | The present example |
| AL3990 | AL3756, but with ΔmanA | The present example |
| AL4121 | AL3990, but with ΔptsG | The present example |
| AL4188 | AL3990, but with ΔptsH | The present example |
| AL4189 | AL4121, but with ΔptsH | The present example |
| AL4186 | AL3990, but with Δpgm | The present example |
| AL4187 | AL4121, but with Δpgm | The present example |
| AL4200 | AL4188, but with Δpgm | The present example |
| AL4201 | AL4189, but with Δpgm | The present example |

TABLE 6
Plasmids used in the present example.

| Plasmid | Description | Source |
|---|---|---|
| pCas | P_{cas}:cas9 P_{araB}:red lacI^{q} Pₜᵣₑ:sgRNA pMB1 | addgene #62225 |
| | repA101(Ts) kan^{R} | |
| pTargetF | P_{J23119}:sgRNA-pmB1, spec^{R}, pMB1 | addgene #62226 |
| pAL421 | P_{LlacO1}:sfgfp, amp^{R}, ColE1 | Zhang et al. 2021 |
| pAL1494 | P_{T7}, amp^{R}, ColE1 | The present example |
| pAL1759 | P_{LlacO1}, amp^{R}, ColE1 | Zhang et al. 2021 |
| pAL1851 | pTargetF-lacZ, amp^{R}, ColE1 | Zhang et al. 2021 |
| pAL1874 | Pₜₑₜ:dcas9, cm^{R}, p15A | addgene #44249 |
| pAL1946 | P_{T7}:alsE-hxpB, amp^{R}, ColE1 | The present example |
| pAL1947 | P_{T7}:alsE-ybiV, amp^{R}, ColE1 | The present example |
| pAL1950 | pTargetF-pfkA, amp^{R}, ColE1 | The present example |
| pAL1952 | Pₜₑₜ:dcas9, kan^{R}, p15A | The present example |
| PAL1957 | pTargetF-rpiB, amp^{R}, ColE1 | The present example |
| pAL1958 | pTargetF-zwf, amp^{R}, ColE1 | The present example |
| pAL1974 | P_{dps}:sfgfp, amp^{R}, ColE1 | The present example |
| pAL1975 | P_{ihfA4}:sfgfp, amp^{R}, ColE1 | The present example |
| pAL1976 | P_{cbpA2}:sfsfp, amp^{R}, ColE1 | The present example |
| pAL1977 | P_{gadB}:sfgfp, amp^{R}, ColE1 | The present example |
| pAL2001 | P_{LlacO1}:alsE-hxpB, amp^{R}, ColE1 | The present example |
| pAL2036 | pTargetF-ptsG, amp^{R}, ColE1 | The present example |
| pAL2038 | pTargetF-pgm, amp^{R}, ColE1 | The present example |
| pAL2062 | P_{LlacO1}:sfgfp, pTargetF-pfkA, amp^{R}, ColE1 | The present example |
| pAL2063 | P_{LlacO1}:sfgfp, pTargetF-no target, amp^{R}, ColE1 | The present example |
| pAL2066 | P_{LlacO1}:sfgfp, pTargetF-sgRNA A*, amp^{R}, ColE1 | The present example |
| pAL2124 | pTargetF-pfkB, amp^{R}, ColE1 | The present example |
| pAL2158 | pTargetF-no target, amp^{R}, ColE1 | The present example |
| pAL2173 | P_{LlacO1}:sfgfp, pTargetF-sgRNA B*, amp^{R}, ColE1 | The present example |
| pAL2174 | P_{LlacO1}:sfgfp, pTargetF-sgRNA C*, amp^{R}, ColE1 | The present example |
| pAL2178 | pTargetF-manA, amp^{R}, ColE1 | The present example |
| pAL2182 | Pₜₑₜ:dcas9, pTargetF-no target, kan^{R}, p15A | The present example |
| pAL2188 | Pₜₑₜ:dcas9, pTargetF-pfkB, kan^{R}, p15A | The present example |
| pAL2233 | pTargetF-alsE, amp^{R}, ColE1 | The present example |
| pAL2247 | P_{gadB}:alsE-hxpB, amp^{R}, ColE1 | The present example |
| pAL2256 | P_{LlacO1}:galP-glk, kan^{R}, ColA | The present example |
| pAL2264 | P_{LlacO1}:galP-glk, gent^{R}, ColA | The present example |
| pAL2318 | pTargetF-ptsH, amp^{R}, ColE1 | The present example |
| pAL2348 | P_{T7}:alsE-hxpA, amp^{R}, ColE1 | The present example |
| pAL2349 | P_{T7}:alsE-yigL, amp^{R}, ColE1 | The present example |
| pAL2351 | P_{T7}:alsE-yidA, amp^{R}, ColE1 | The present example |
| pAL2352 | P_{T7}:alsE-yihX, amp^{R}, ColE1 | The present example |

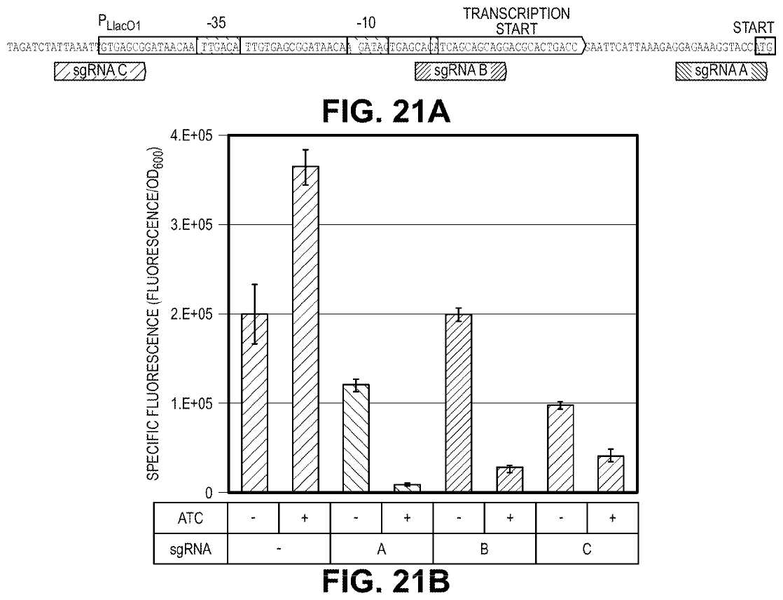

*See FIGS. 21A and 21B.

Through knocking out pfkA, zwf, and rpiB, carbon flux was channeled into the D-psicose production pathway. In particular, knocking out pfkA and zwf should lead to an increase in F6P availability within cells. These results reaffirmed the suspicions that F6P plays a key role as precursor to P6P, and that accumulation of F6P is necessary to drive carbon flux through the production pathway.

### Identification of a Side Product.

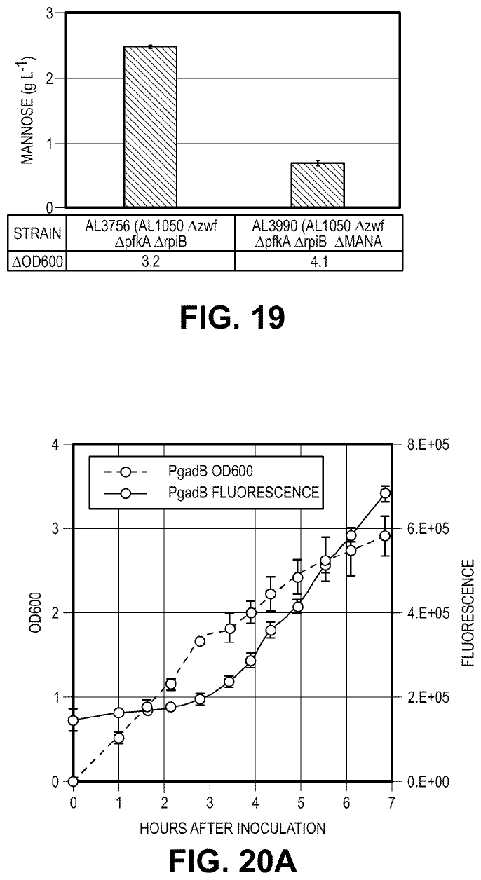

When analyzing samples from Strain 1, it was observed a significant peak on the High-performance liquid chromatography (HPLC) chromatogram that did not align with D-glucose, D-fructose, or D-psicose standards, or any media component. Using gas chromatography-mass spectrometry (GC-MS) analysis, it was found the retention time and mass spectrum of the unknown peak matched the retention time and spectrum of D-mannose (FIG. 19).

Under standard conditions, the D-mannose pathway involves the assimilation of D-mannose 6-phosphate (M6P) into central carbon metabolism by the reversible isomerization of M6P to F6P via mannose-6-phosphate isomerase (ManA). An accumulation of F6P could cause this reaction to run in reverse, generating M6P and D-mannose as a result. To test the hypothesis that this side product was D-mannose, manA was deleted in Strain 1, resulting in Strain 2 (Table 4).

The deletion of manA resulted in a considerable decrease in D-mannose production, and Strain 2 with IPTG generated only 0.69 g L⁻¹ of D-mannose compared to Strain 1 with IPTG, which produced 2.49 g L⁻¹ of D-mannose (FIG. 19). Complementary to the reduction in D-mannose production, the production of D-psicose in Strain 2 was 1.5 times higher than in Strain 1 (FIG. 15D). While not fully eliminated, the unwanted production of D-mannose was greatly decreased by the deletion of manA. Subsequent production was therefore carried out in strains containing ΔpfkA, Δzwf, ΔrpiB, and ΔmanA.

Although the D-mannose pathway was eliminated, there can be other D-fructose epimer pathways continuing to compete for carbon flux. Eliminating the PPP and limiting glycolysis can cause a substantial increase in cellular F6P pools, which if not funneled efficiently into D-psicose biosynthesis, may be acted upon by other epimerases or isomerases. In short, increased F6P availability may allow enzymes not normally observed to engage with a substrate and generate other sugar products.

### Utilization of a Stationary Phase Promoter.

Constructing production pathways within a microorganism requires a careful carbon partitioning between essential metabolic processes and production, especially when working around central carbon metabolism. Within the presently disclosed system, dynamically balancing carbon flux between glycolysis and the D-psicose pathway can help maximize both cellular viability and D-psicose production.

The life cycle of an E. coli culture includes 5 distinct phases: lag, logarithmic, stationary, death, and long-term stationary phase. The lag phase occurs when cells are inoculated into media and adjust their metabolic processes according to their new environment. The cells will then rapidly grow and divide, entering the logarithmic phase. It is at this time that enzymes related to central carbon metabolism are most important, and the transcription of corresponding genes will be upregulated. Once the cells sense environmental stressors such as scarcity of media nutrients, their growth and division slows, and the culture enters the stationary phase. Here, culture density plateaus and genes related stress response are expressed. The transcription of these genes is regulated in part by the 038-subunit of RNA polymerase, which recognizes the promoter region of a gene.

E. coli's native gene regulatory system was utilized to balance carbon flux by affixing the D-psicose production genes, alsE and hxpB, downstream of a stationary phase-active promoter. This prevented the production pathway from competing with central carbon metabolism for carbon flux during the logarithmic phase of growth, a time when cells need carbon to rigorously grow and divide.

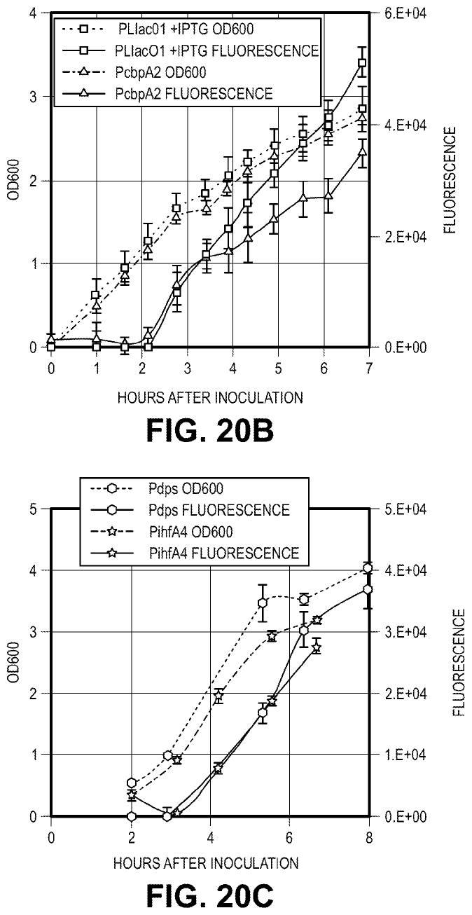

Four promoters previously shown to have activity during the stationary phase were chosen for testing using Green Fluorescent Protein (GFP) as a reporter: P_{gadB}, P_{cbpA2}, P_{ihfA4}, and P_{dps}. Each promoter was cloned upstream of sfgfp on an expression plasmid (Table 6). The strongest promoter, P^{gadB}, had an expression strength 100 times as strong as IPTG-induced promoter P_{LlacO1}, in addition to having an expression time correlating to late logarithmic or early stationary phase (FIG. 20A). The second strongest promoter, P_{cbpA2}, closely followed the expression timing of induced P_{LlacO1}, but with approximately ⅔ the expression strength (FIG. 20B). P_{ihfA4} and P_{dps} also followed the expression timing of P_{LlacO1}, but with lower expression strength than P^{chpA2}. (FIG. 20C)

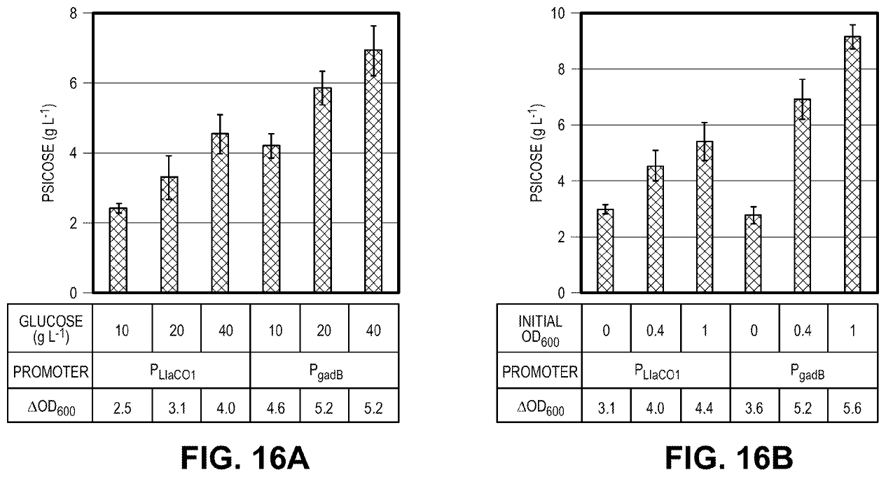

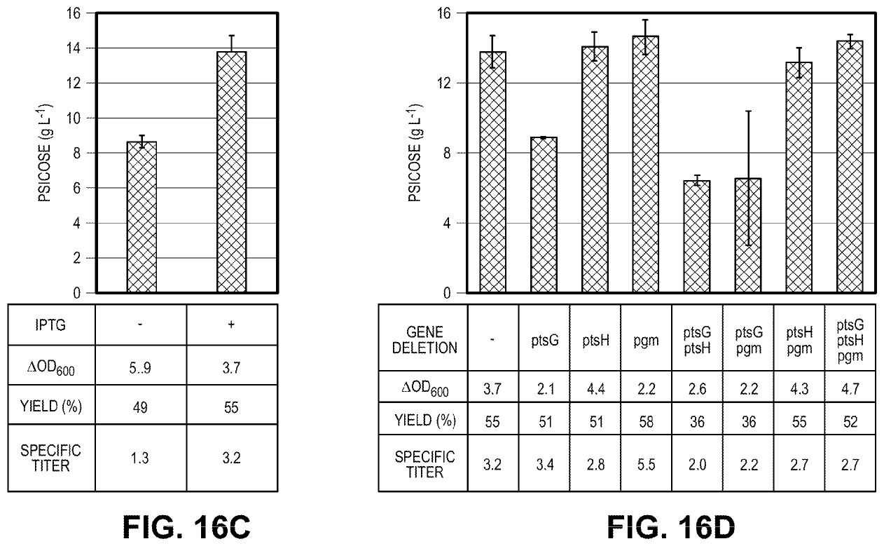

Due to its strong expression during the stationary phase, P_{gadB} was used to express the operon of alsE and hxpB (pAL2247, Table 6). To evaluate the effects of the initial glucose concentration, D-psicose production was tested with 10, 20, and 40 g L⁻¹. Under glucose concentrations (10, 20, and 40 g L⁻¹) Strain 3 (P_{gadB}:alsE-hxpB, Table 4) consistently produced greater titers of D-psicose and grew with a great ΔOD₆₀₀, which represents the difference in optical density at 600 nm (OD₆₀₀) at 0) and 24 h, compared to Strain 2 (P_{LlacO1}:alsE-hxpB, FIG. 16A). The highest D-psicose titers were achieved at a glucose concentration of 40 g L⁻¹, which allowed Strain 3 to produce 6.92 g L⁻¹ of D-psicose and grow with an ΔOD₆₀₀ of 5.2, while Strain 2 produced 4.55 g L⁻¹ and grew with an ΔOD₆₀₀ of 4.0. The initial glucose concentration of 40 g L⁻¹ was used for further studies.

Next, the impact of the timing of the shift from 37° C. to 30° C. on D-psicose was tested. Preliminary tests showed that 37° C. was suitable for cell growth while 30° C. was suitable for production. To determine the optimum timing of the shift from 37° C. to 30° C., cultures were grown to an OD₆₀₀ of ˜0 (no culturing at 37° C.), ˜0.4, or ˜1 at 37° C., then grown at 30° C. and induced as necessary. Across all strains, cultures moved to 30° C. at a later OD₆₀₀ produced higher titers of D-psicose and grew with a greater ΔOD₆₀₀ (FIG. 3b). When moved at an OD₆₀₀ ˜1, Strain 3 produced the highest titers of D-psicose, at 9.13 g L⁻¹, and grew with an ΔOD₆₀₀ of 5.6. Strain 2 produced 5.42 g L⁻¹ of D-psicose and grew with an ΔOD₆₀₀ of 4.4.

It was theorized that despite being a stronger promoter than P_{LlacO1}, the expression timing of P_{gadB} prevents D-psicose production enzymes from siphoning carbon away from central metabolism during a key period of growth. This allows for cultures to grow more robustly and produce higher titers of D-psicose. The use of endogenous, growth phase-associated promoters eliminates the need for costly chemical inducers and allows cultures to self-regulate pathway expression depending on the growth and cellular viability. P_{gadB}:alsE-hxpB (pAL2247, Table 6) was thereafter used in further production experiments.

### Supplementing Glucose Import Using GalP and Glk

Continuous glucose import, especially during the stationary phase of growth, is useful to the production of D-psicose. One consequence of limiting carbon flux through glycolysis by knocking out pfkA is the reduction in downstream metabolites, such as phosphoenolpyruvate (PEP). PEP is of particular concern, as it is utilized by the PTS to import and phosphorylate glucose. A reduction in PEP availability due to decreased flux through glycolysis may have an impact on the ability to assimilate glucose and produce D-psicose. Additionally, it has been shown that increased G6P or F6P pools in the ΔpfkA mutant leads to degradation of ptsG mRNA that encodes for membrane receptor IICBGlc of the PTS complex.

To enhance glucose import without the use of the PTS in the ΔpfkA background, galP and glk were additionally expressed from a plasmid. The galP gene encodes for the galactose proton symporter GalP, which is capable of importing glucose. The glk gene encodes for glucokinase Glk, which phosphorylates glucose to G6P (FIG. 14C). These genes were cloned downstream of PLlacO1, generating plasmid pAL2264 (Table 6). Induction of the expression of galP-glk in Strain 4 (Strain 3 with P_{LlacO1}:galP-glk. Table 4) substantially increased D-psicose production and allowed the strain to produce 13.81 g L⁻¹ with a specific titer of 3.2 g L⁻¹ OD₆₀₀⁻¹ and yield of 55% (FIG. 16C). In comparison, Strain 4 without IPTG produced 8.67 g L⁻¹ of D-psicose, with a specific titer of 1.3 g L⁻¹ OD₆₀₀⁻¹ and a yield of 49%. Yield was calculated based on the possibility that for every 1 mol of D-glucose consumed, 1 mol of D-psicose could be produced, leading to a theoretical maximum yield of 100%. Interestingly, expression of galP and glk resulted in lower ΔOD₆₀₀ (FIG. 16C). It has been shown that overexpression of membrane proteins such as GalP poses a growth burden on cells, potentially due to competition for membrane translocation machinery.

### Manipulating Glucose Utilization and Metabolism

It has been shown that the PTS utilizes ˜50% of PEP to transport and phosphory late glucose. To further balance cellular PEP supply and promote glucose import using GalP-Glk, it was attempted to incapacitate the PTS by knocking out genes ptsG, encoding for membrane receptor IICBGlc, and ptsH, encoding for phosphocarrier protein HPr, in AL3990 (Table 5).

Another source of glucose siphoning away from the D-psicose pathway is through glycogen biosynthesis. Glycogen is stored for usage during times of starvation, which for the presently disclosed subject matter is unnecessary. The deletion of pgm, which encodes for phosphoglucomutase Pgm, prevents E. coli from producing glycogen. As such, pgm was deleted in the production strains (Table 5).

While Strain 4 with ΔptsG appeared detrimental to the psicose production (ΔptsG, ΔptsG ΔptsH and ΔptsG Δpgm), Strain 4 with ΔptsH has no effects on the psicose production (FIG. 16D). Δpgm was beneficial for the psicose production, particularly at specific titers (FIG. 16D) Strain 5 (Strain 4 with Δpgm, Table 4) produced 14.66 g L⁻¹ of D-psicose, with a specific titer of 5.5 g L⁻¹ OD₆₀₀⁻¹ and yield of 58% (FIG. 16D).

Inhibiting glycogen biosynthesis by knocking out pgm removes a source of carbon to central metabolism which would normally be used to help cells grow under periods of starvation. This combined effect of impaired growth and enhanced production lead to an increase in specific titer going from Strain 4 to Strain 5 (FIG. 16D).

Although some studies have shown the elimination of the PTS through gene knockouts helps to restore intracellular PEP balance and rescue growth, here it was found knocking out ptsG or ptsH to be deleterious or neutral to the D-psicose production. Phosphorylated and dephosphorylated forms of IICBGlc and HPr take part in signaling cascades related not only to carbon metabolism, but also global gene expression through expression of RNA polymerase sigma subunits, including the aforementioned σ38-subunit and logarithmic phase-associated σ70-subunit. Given the use of P_{gadB} to express alsE and hxpB, eliminating portions of the PTS may reduce the expression of the D-psicose production pathway genes.

### Dynamic Regulation of Glycolysis Using CRISPRi

While the deletion of pfkA successfully redirected carbon flux towards D-psicose production, glycolysis remained active through PfkB. Completely shutting down glycolysis by deleting pfkB did not allow cells to grow under our culture conditions, so we attempted to dynamically limit pfkB expression to times it was essential. Cells require the carbon flux through glycolysis when building biomass during the logarithmic phase of growth. During the stationary phase, carbon flux can be reduced through glycolysis and redirected to D-psicose production. In order to dynamically regulate the expression of pfkB, it was implemented a CRISPRi system targeting pfkB on the genome.

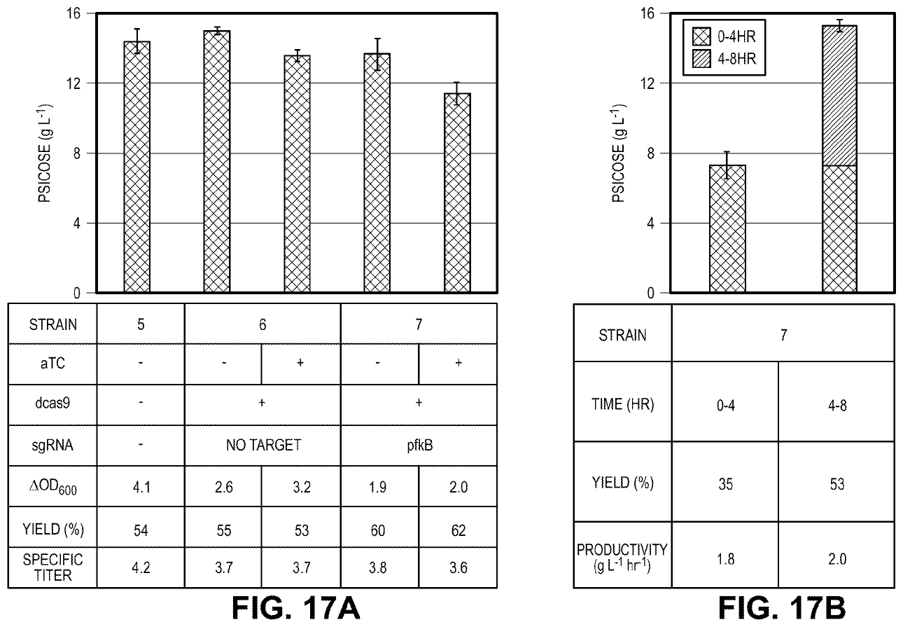

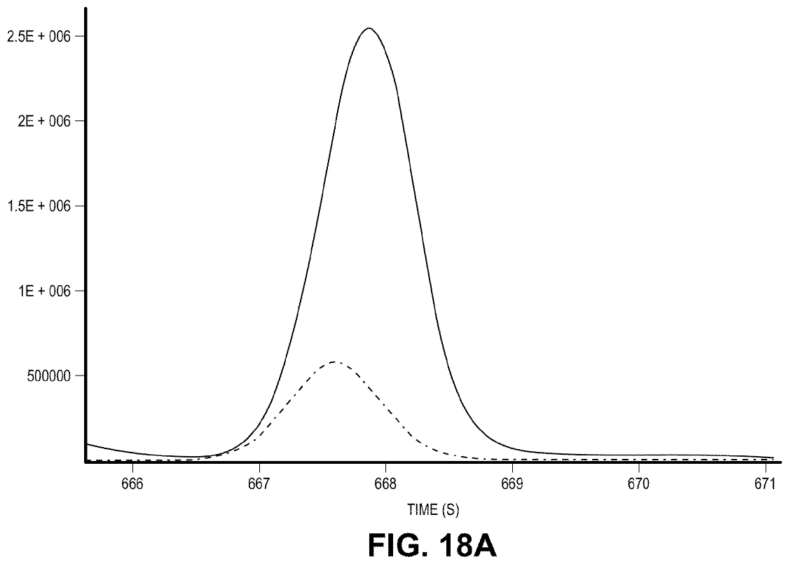

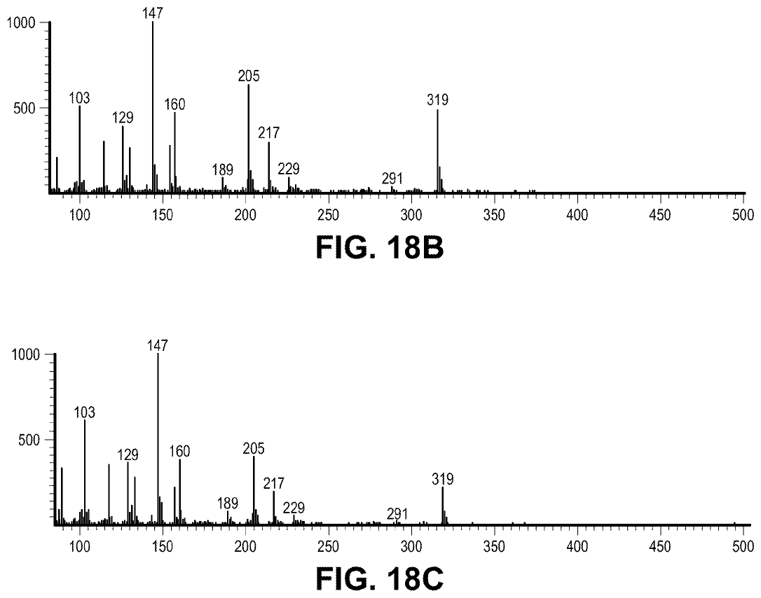

The CRISPRi system utilizes an inactivated Cas9, dCas9, which when recruited by a single guide RNA scaffold (sgRNA) can precisely target and block transcription initiation by RNA polymerase. The dcas9 gene was cloned under an aTc-inducible promoter. Pₜₑₜ, along with a constitutively expressed sgRNA sequence targeting a gene of interest. To confirm functionality of the CRISPRi system and determine where the sgRNA should target in order to achieve the greatest inhibition of expression, three different sgRNA sequences were designed to repress the expression of sfGFP under P_{LlacO1} (FIG. 17A). The sgRNAs targeted the upstream, middle, and downstream sequence of P_{LlacO1} (FIG. 17A). Here, it was found the sgRNA targeting the middle of P_{LlacO1} led to greatest difference in fluorescence. Successive sgRNA targeting the promoter region of pfkB was designed with homology to the middle of the promoter sequence.

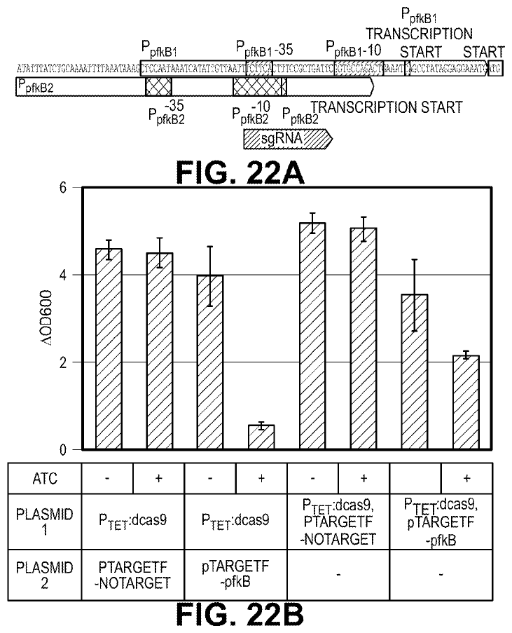

Further exploration of the CRISPRi system involved expressing deas9 and sgRNA from either the same or separate plasmids (FIGS. 22A and 22B). In the single-plasmid system. Pₜₑₜ:dcas9 and a constitutively expressed sgRNA sequence targeting the promoter region of pfkB or no targeting sequence were cloned onto the same plasmid (Table 6). For the separate-plasmid system, Pₜₑₜ:dcas9 was cloned onto one plasmid, and the constitutively expressed sgRNA sequence targeting the promoter region of pfkB or no targeting sequence were cloned onto a different plasmid (Table 6). Each CRISPRi system was introduced into AL4186 (Table 5) and growth was measured 24 hr after induction with 100 ng mL⁻¹ aTc. The separate-plasmid system caused a greater inhibition of growth, most likely because the sgRNA was expressed from a high copy number plasmid, rather than a low copy number plasmid as was the case in the single-plasmid system. When production was tested using the separate-plasmid CRISPRi system in Strain AL3990 with pAL2247 (Tables 5 and 6), very little D-psicose was produced, regardless of aTc induction. As such, the single-plasmid CRISPRi system was used for production.

The single-plasmid CRISPRi system was installed to Strain 5, generating Strain 6 (empty guide) and Strain 7 (sgRNA targeting pfkB. Table 4). With CRISPRi (Strain 7), the cell growth was reduced, and the specific titer was greatly improved with or without aTC, although the titer of Strain 7 was lower than those of Strain 5 and 6 (FIG. 17A). Strain 7 with 100 ng mL⁻¹ aTc produced 11.40 g L⁻¹ of D-psicose with a specific titer of 3.6 g L⁻¹ OD₆₀₀⁻¹ and yield of 62%. Strain 7 without aTc produced 13.65 g L⁻¹ of allulose with a specific titer of 3.8 g L⁻¹ OD₆₀₀⁻¹ and yield of 60%.

### D-Psicose Production Under High Culture Density Conditions

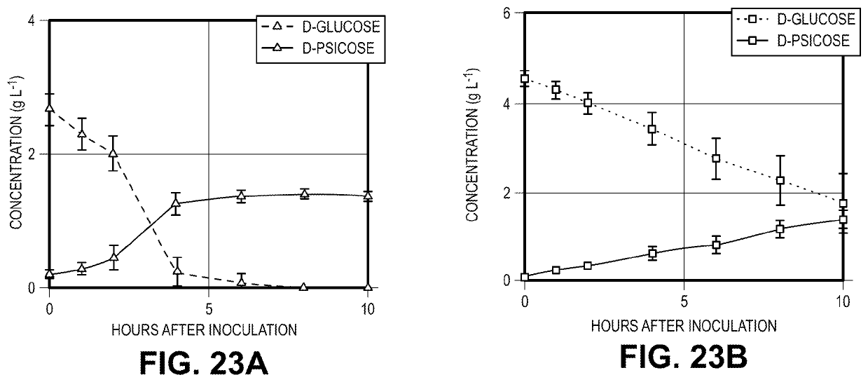

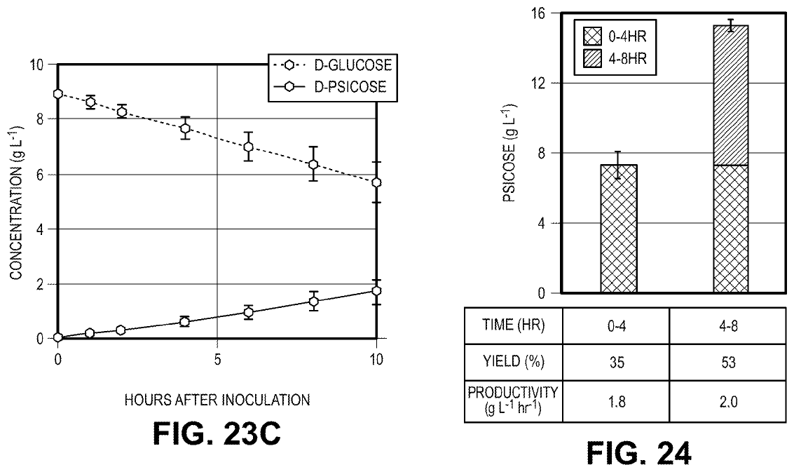

To study the rate of D-glucose consumption and D-psicose production of Strain 7 (Table 4), substrate concentrations were monitored for 10 hr at media glucose concentrations of 3, 5, and 10 g L⁻¹ of glucose (FIGS. 23A-23C). It was found the rate of D-psicose production to be similar across cultures, independent of media glucose concentrations. Cultures fed 3 g L⁻¹ of glucose consumed all glucose over the course of the experiment, implying an advantage for easier extraction and purification of D-psicose from the media in an industrial setting.

To decouple growth and production, and minimize limitations on production by glucose availability, Strain 7 was cultured under high cell density conditions in an excess of available D-glucose over a shorter period of time (FIG. 17B). Strain 7 (Table 4) was grown to an OD₆₀₀ of ˜1 before being induced with 100 ng mL⁻¹ aTc and 1 mM IPTG and grown for a further 30 min. Cells were then pelleted and resuspended to an OD₆₀₀ of ˜10 with M9P containing 40 g L⁻¹ glucose, 100 ng mL⁻¹ aTc, and 1 mM IPTG. Samples were taken and analyzed at 0, 4, and 8 h. Over the course of 8 hr, Strain 7 produced 15.3 g L⁻¹ of D-psicose with a specific titer of 1.4 g L⁻¹ OD₆₀₀⁻¹, yield of 43%, and productivity of 1.9 g L⁻¹ hr⁻¹ (FIG. 17B). Although D-psicose titers produced at 0-4 hr and 4-8 hr were similar, at 7.3 and 8.0 g L⁻¹, the yield at 4-8 hr (53%) was higher than that at 0-4 hr (35%). Higher yield could be the result of cells responding to their high-density culture conditions, as the presently disclosed production system relies on the activation of stationary phase promoter P_{gadB}. Overall, the production system achieved industrially relevant production with high yield (>0.5 g_{product} g_{substrate}⁻¹) and high productivity (>1 g L⁻¹ hr⁻¹) comparable to bioethanol, despite being cultured in unoptimized test tube conditions

### Conclusion

Within the present disclosure, whole-cell catalysis was applied as a strategy for producing the industrially relevant, rare sugar D-psicose. Living cells possess the capability of assembling stereo/regioselective enzymes, providing necessary cofactors, and secreting products for easy purification, all under environmentally friendly production conditions. Whole-cell catalysis technology and infrastructure is already established industrially, and the model organism E. coli can be fed feedstocks that do not compete with commercial food production.

The elimination of competing pathways and additional expression of native E. coli genes (alsE, hxpB, galp, and glk) along with the use of static and dynamic gene regulation strategies led to a strain capable of producing D-psicose using a thermodynamically favorable biosynthetic pathway from a readily available feedstock. The highest titer of D-psicose produced was 16.59 g L⁻¹ with a specific titer of 5.0 g L⁻¹ OD₆₀₀⁻¹ under test tube conditions. The highest yield achieved was 62%, surpassing current industrial standards. Furthermore, the strain's ability to consume all D-glucose present in media greatly simplifies downstream purification requirements. Overall, the engineered strain represents a helpful step in producing D-psicose in a cost-effective manner, offering the food industry a viable source for creating the low-glycemic index products desired by consumers.

Overall, the engineered strain represents an important step in producing D-psicose and other rare sugars in an efficient, cost-effective manner. The ability to produce rare sugars in bulk will help address rising global obesity rates by providing low-calorie sugar alternatives for ultra-processed foods. Increased production of rare sugars will also grant access to sustainable pesticides for the agricultural industry, and medicinally relevant monosaccharides for the pharmaceutical industry. The strategy developed in this study has the potential to drive transformative changes in our ability to produce, measure, and control the human-food relationship, developing a world where easily attainable metabolic health leads to happier, healthier, longer lives for everyone.

### Methods

Reagents. All enzymes involved in the molecular cloning experiments were purchased from New England Biolabs (NEB). All synthetic oligonucleotides were synthesized by Integrated DNA Technologies. Sanger Sequencing was provided by Genewiz from Azenta Life Sciences. D-Psicose and D-Mannose were purchased from Sigma-Aldrich. D-Glucose was purchased from Fisher Scientific.

Strains and Plasmids. All strains and plasmids used in this study are listed in Tables 5 and 6, respectively. All oligonucleotides are listed in Table 7. Plasmids for D-psicose production were constructed using sequence and ligation independent cloning (SLIC). The constructed plasmids were verified via Sanger Sequencing. A guide to the construction of plasmids used in this study is detailed in Table 8.

TABLE 7
Oligonucleotides used in the present example.

| | SEQ ID | | Plasmid(s) or |
|---|---|---|---|
| Name | NO: | Sequence 5′ to 3′ | fragment(s) produced |
| AZ0195 | 74 | GCACATCAGCGTTTTAGAGCTAGAAATAGC | pAL2173 |
| AZ0196 | 75 | TCAGTATCTTACTAGTATTATACCTAGGAC | pAL2173 |
| AZ0466 | 76 | GCCATATCGAAGGTCGTCATATGAAAATCTC | pAL1946, pAL1947 |
| | | CCCCTCGTTAATG | |
| AZ0467 | 77 | ATCTCCTTTTGTTAGCAGCCGGATCCTTATGC | pAL1946, pAL1947 |
| | | TGTTTTTGCATGAGGCTG | |
| AZ0468 | 78 | GGCTGCTAACAAAAGGAGATATACATATGTC | pAL1946 |
| | | AACCCCGCGTCAGATTCTTGC | |
| AZ0469 | 79 | GCTTTGTTAGCAGCCGGATCCTCAACCGAGA | pAL1946 |
| | | AGGTCTTTTGCGGTG | |
| AZ0472 | 80 | GGCTGCTAACAAAAGGAGATATACATATGAG | pAL1947 |
| | | CGTAAAAGTTATCGTCAC | |
| AZ0473 | 81 | GCTTTGTTAGCAGCCGGATCCTCAGCTGTTAA | pAL1947 |
| | | AAGGGGATGTG | |
| AZ0475 | 82 | GGATCCGGCTGCTAACAAAGCCCGAAAGGA | pAL1946, pAL1947 |
| | | AGCTGAGTTGGC | |
| AZ0482 | 83 | CTGACCTGAATCAATTCAGCAGGAAGTGATT | ΔpfkA repair fragment |
| | | GTTATACTATTTGCACATTCGTTGGAT | (RF) |
| AZ0483 | 84 | TCTGTTGCCGGAAGTCTTCTTGCACATCGAAG | ΔpfkA RF |
| | | TGATCCAACGAATGTGCAAATAGTAT | |
| AZ0484 | 85 | AGACTTCCGGCAACAGATTTCATTTTGCATTC | ΔpfkA RF |
| | | CAAAGTTCAGAGGTAGTCTGATTTCG | |
| AZ0485 | 86 | TGTCATCGGTTTCAGGGTAAAGGAATCTGCC | ΔpfkA RF |
| | | TTTTTCCGAAATCAGACTACCTCTGAAC | |
| AZ0486 | 87 | ACCCTGAAACCGATGACAGAAGCAAAAATG | ΔpfkA RF |
| | | CCTGATGCGCTTCGCTTATCAGGCCTACAT | |
| AZ0487 | 88 | CCTACAAAAGTTTGCAAATTCAATAAATTGC | ΔpfkA RF |
| | | AGAATTCATGTAGGCCTGATAAGCGA | |
| AZ0502 | 89 | CAAGCGAGCTCGATATCAAATCAGAAGAACT | pAL1952 |
| | | CGTCAAGAAGGC | |
| AZ0503 | 90 | CAGGAGCTAAGGAAGCTAAAATGATTGAAC | pAL1952 |
| | | AAGATGGATTGCACGCAGG | |
| AZ0504 | 91 | TTTAGCTTCCTTAGCTCCTGAAAATCTCGATA | pAL1952 |
| | | ACTC | |
| AZ0505 | 92 | TTTGATATCGAGCTCGCTTGGACTCC | pAL1952 |
| AZ0520 | 93 | TCGGTCTGCCGTTTTAGAGCTAGAAATAGC | pAL1950 |
| AZ0521 | 94 | TGCACGGGAAACTAGTATTATACCTAGGAC | pAL1950 |
| AZ0522 | 95 | CTGTTGCTGGGTTTTAGAGCTAGAAATAGC | pAL1957 |
| AZ0523 | 96 | CCAGTGCGACACTAGTATTATACCTAGGAC | pAL1957 |
| AZ0526 | 97 | CGCTGTCAGATGTAACTCTGTAAAACAGATC | ΔrpiB RF |
| | | AGGAAGGCGTA | |
| AZ0527 | 98 | TGTGATGTTAATGAATTAAAAACACCCAAAA | ΔrpiB RF |
| | | TCGATGAATTACGCCTTCCTGATCTGTT | |
| AZ0528 | 99 | GGGTGTTTTTAATTCATTAACATCACAAATGT | ΔrpiB RF |
| | | TTTTTGATTGTGAAGTTTTGCACGGACG | |
| AZ0529 | 100 | CTCATCCATGCAAGTAGTGGATGAATCTCAT | ΔrpiB RF |
| | | CTTCCCCGTCCGTGCAAAACTTCACAAT | |
| AZ0530 | 101 | CCACTACTTGCATGGATGAGTAATGATTAAT | ΔrpiB RF |
| | | GTGGATAGAGTTTCTTTTTGAGGTTGGCT | |
| AZ0531 | 102 | AGCGGAAAGCGTTTCATTAGCCAACCTCAAA | ΔrpiB RF |
| | | AAGAAACTCTA | |
| AZ0532 | 103 | ATGAAACGCTTTCCGCTATTTCTTTTATTCAC | ΔrpiB RF |
| | | CCTGCTCACGCTGTCCACCGTTC | |
| AZ0533 | 104 | CGGAACGGTGGACAGCGTGAGC | ΔrpiB RF |
| AZ0534 | 105 | TGGGGATCGAGTTTTAGAGCTAGAAATAGC | pAL1958 |
| AZ0535 | 106 | CTTCTTCTGCACTAGTATTATACCTAGGAC | pAL1958 |
| AZ0538 | 107 | TAATCGCACGGGTGGATAAGCGTTTACAGTT | Δzwf RF |
| | | TTCGCAAGCTCGTAAAAGCAGTACAGTGC | |
| AZ0539 | 108 | CGGTACTTAAGCCAGGGTATACTTGTAATTTT | Δzwf RF |
| | | CTTACGGTGCACTGTACTGCTTTTACGA | |
| AZ0540 | 109 | ACCCTGGCTTAAGTACCGGGTTAGTTAACTT | Δzwf RF |
| | | AAGGAGAATGACTATCTGCGCTTATCCT | |
| AZ0541 | 110 | GCGCAAGATCATGTTACCGGTAAAATAACCA | Δzwf RF |
| | | TAAAGGATAAGCGCAGATAGTCATT | |
| AZ0542 | 111 | CGGTAACATGATCTTGCGCAGATTGTAGAAC | Δzwf RF |
| | | AATTTTTACACTTTCAGGCCTCGTGCGGA | |
| AZ0543 | 112 | CAGTCAGTGTAATAAAAAAAGCCTCGTGGGT | Δzwf RF |
| | | GAATCCGCACGAGGCCTGAA | |
| AZ0666 | 113 | AAGAGGAGAAAAGATATACCATGAAAATCT | pAL2001, pAL2247 |
| | | CCCCCTCGTTAATGTG | |
| AZ0667 | 114 | GGTACCTTAGCAGCCGGATCTCAACCGAGAA | pAL2001, pAL2247 |
| | | GGTCTTTT | |
| AZ0668 | 115 | GATCCGGCTGCTAAGGTACCTAATCTAGAGG | pAL2001, pAL2247 |
| | | CATC | |
| AZ0669 | 116 | GGTATATCTTTTCTCCTCTTTAATGAATTCGG | pAL2001, pAL2247 |
| | | TCAGTGCG | |
| AZ0711 | 117 | ACAATCGTGCGTTTTAGAGCTAGAAATAGC | pAL2038 |
| AZ0712 | 118 | GGATTGCCATACTAGTATTATACCTAGGAC | pAL2038 |
| AZ0743 | 119 | CTGAATTATTTTACTCTGTGTAATAAATAAAG | ΔptsG RF |
| | | GGCGCTTAGATGCCCTGTACACGGCGA | |
| AZ0744 | 120 | ACGCGTGGCAAGGGGGGAGAGCCTCGCCGT | ΔptsG RF |
| | | GTACAGGGCATCTAAGCG | |
| AZ0745 | 121 | CCTTGCCACGCGTGAGAACGTAAAAAAAGCA | ΔptsG RF |
| | | CCCATACTCAGGAGCACTCTCAATTTCCG | |
| AZ0746 | 122 | GCCATCTGGCTGCCTTAGTCTCCCCAACGTCT | ΔptsG RF |
| | | TACGGAAATTGAGAGTGCTCCT | |
| AZ0747 | 123 | GGCAGCCAGATGGCTGCCTTTTTTACAGGTG | ΔptsG RF |
| | | TTATTCAGAATTGATACGTGCCGGTAATG | |
| AZ0748 | 124 | GTGGAAGGTTCTATCGTCTACGGCACACCGC | ΔptsG RF |
| | | GTAATTTCAGCATTACCGGCACGTATCAA | |
| AZ0751 | 125 | AACCTGCAAAGTTTTAGAGCTAGAAATAGC | pAL2036 |
| AZ0752 | 126 | AGCAAATGCAACTAGTATTATACCTAGGAC | pAL2036 |
| AZ0763 | 127 | CAACACCAACACCGGCCGTAAATGCAGCATG | Δpgm RF |
| | | ATCGAACACATCATGCAGTC | |
| AZ0764 | 128 | TCCGCAAACCTTCTCAATCAATTCCGCCGGG | Δpgm RF |
| | | AATTGCATCGACTGCATGATGTGTTCGA | |
| AZ0765 | 129 | GATTGAGAAGGTTTGCGGAACTATCTAAAAC | Δpgm RF |
| | | GTTGCAGACAAAGGACAAAGCAACACATT | |
| AZ0766 | 130 | AAGGGCGATCTTGCGACCGCCCTTTTTTTATT | Δpgm RF |
| | | AAATGTGTTGCTTTGTCCTTTGTCT | |
| AZ0767 | 131 | CGCAAGATCGCCCTTTTTTACGTATGACAAA | Δpgm RF |
| | | CACAGAATTGCCTGATGCGCTACGCTTAT | |
| AZ0768 | 132 | TCGCTTAAATTCAATATATTGCACCATCCTCG | Δpgm RF |
| | | TAGGCCTGATAAGCGTAGCGCATCAG | |
| AZ0936 | 133 | ATGACGACCTTCGATATGGCCGCTGCTGTG | pAL1946, pAL1947 |
| AZ1025 | 134 | AAATTGTGAGGTTTTAGAGCTAGAAATAGC | pAL2174 |
| AZ1026 | 135 | AATAGATCTAACTAGTATTATACCTAGGAC | pAL2174 |
| AZ1041 | 136 | AATATCCTACACACTTTTTTAACAAAAACTG | ΔmanA RF |
| | | AGACTAGTACGACTTTTTGCGGCTCC | |
| AZ1042 | 137 | CCCACTATTAAAGCAAGAATCCTACGGGAAG | ΔmanA RF |
| | | TAACCTGGAGCCGCAAAAAGTCG | |
| AZ1043 | 138 | AGGATTCTTGCTTTAATAGTGGGATTAATTTC | ΔmanA RF |
| | | CACATTAAAACAGGGATTGATCGAGCT | |
| AZ1044 | 139 | GCCTTTAATAAGCTTAGCAAGAGATGTTAAT | ΔmanA RF |
| | | TTTTTCAGTAAGCTCGATCAATCCCTGT | |
| AZ1045 | 140 | TCTCTTGCTAAGCTTATTAAAGGCTTATAACA | ΔmanA RF |
| | | CCTTCAGGCGGCCAGTCCGCCTGATTTC | |
| AZ1046 | 141 | CTACCGCTACCAGCGATTTATTCATAATGATT | ΔmanA RF |
| | | ATCCATAAAATGAAATCAGGCGGACTGG | |
| AZ1051 | 142 | TCCTCGCTCACTGACTCGCTTCCTTACGCATC | pAL2182 |
| | | TGTGCGGTATTTCACACC | |
| AZ1052 | 143 | GCAGTCGAACGACCGAGCGTGGATAACAGG | pAL2182 |
| | | GTAATAGATCTAAGCTTCTG | |
| AZ1053 | 144 | ACGCTCGGTCGTTCGACTGCGG | pAL2182 |
| AZ1054 | 145 | AGCGAGTCAGTGAGCGAGGAAGCGGAATAT | pAL2182 |
| | | ATCC | |
| AZ1055 | 146 | GTTTTAGAGCTAGAAATAGCAAG | pAL2063, pAL2158 |
| DS071 | 147 | TCATGACGGCACTAGTATTATACCTAGGACT | pAL2233 |
| | | GAGCTAG | |
| DS072 | 148 | CATGAAAGTGGTTTTAGAGCTAGAAATAGCA | pAL2233 |
| | | AGTTAAAATAAG | |
| DS075 | 149 | CGCCATCATTGGCGGCACCAGTTTCTTCGGC | ΔalsE RF |
| | | GGCAAGGGGCGCATTTTCTCT | |
| DS076 | 150 | GTTGTTGATGGTGCCGATGATCAACCCGCCA | ΔalsE RF |
| | | ATCACCACAGAGAAAATGCGCCCCT | |
| DS077 | 151 | TCGGCACCATCAACAACGGTCTGAATATTTT | ΔalsE RF |
| | | GCAGGTACAAACCTATTACCAACTGGTGG | |
| DS078 | 152 | CAAGGGCGACAGCCGCGATAATTAATCCGCC | ΔalsE RF |
| | | CATCACCACCAGTTGGTAATAGGTTTG | |
| DS079 | 153 | GGCTGTCGCCCTTGACCGTCTTATCAGTAAGT | ΔalsE RF |
| | | AAGGAATTGAACCGTCGTAGCGGGCGTG | |
| DS080 | 154 | TGCTGTCCGCAGACAAAAGCGGATATGCGTT | ΔalsE RF |
| | | GCCCCCATATCCACGCCCGCTACG | |
| DS081 | 155 | TGTCTGCGGACAGCAGAAGGTGAAACGCTAC | ΔalsE RF |
| | | ACTGCGAAAAAAAGCGGACCGCAGAAGTC | |
| DS082 | 156 | AGGCCGGGAGCAATGACTTCTGCGGTCCGCT | ΔalsE RF |
| | | TTTTTTCGCAGTG | |
| DS083 | 157 | TTGCTCCCGGCCTGGTGTCGGGTATCGGCGA | ΔalsE RF |
| | | AATGATTGACGAGCAACTCAGGCGCTTTA | |
| DS084 | 158 | CCGGAAATCCCATCACCAGACCATGACAGCG | ΔalsE RF |
| | | AGCGTTAAAGCGCCTGAGTTGCTCGT | |
| DS126 | 159 | TGAGCGGGACTCTGGGGTTCGAG | pAL2264 |
| DS127 | 160 | GCGAAACGATCCTCATCCTGTCTCTTG | pAL2264 |
| DS128 | 161 | TGAGGATCGTTTCGCATGTTACGCAGCAGCA | pAL2264 |
| | | ACGATG | |
| DS129 | 162 | CCAGAGTCCCGCTCATTAGGTGGCGGTACTT | pAL2264 |
| | | GGGTC | |
| DS157 | 163 | GTTTAAACAGACTAGTATTATACCTAGGAC | pAL2318 |
| DS158 | 164 | TGCAGACTCTGTTTTAGAGCTAGAAATAGC | pAL2318 |
| DS159 | 165 | GGACTGTATTGCGCTCTTCGTGCGTCGCGTCT | ΔptsH RF |
| | | GTTAA | |
| DS160 | 166 | GCCTGGCGGTTCGACTTTAGCCTGTATTGTTA | ΔptsH RF |
| | | GCGCCAGTTTTTAACAGACGCGACGCAC | |
| DS161 | 167 | CGAACCGCCAGGCTAGACTTTAGTTCCACAA | ΔptsH RF |
| | | CACTAAACCTAT | |
| DS162 | 168 | CCGGGAAATGTATTTCCCCAACTTATAGGTTT | ΔptsH RF |
| | | AGTGTTGTGGAACTAAAGTCTA | |
| DS163 | 169 | GGGGAAATACATTTCCCGGGTTCTTTTAAAA | ΔptsH RF |
| | | ATCAGTCACAAGTAAGGTAGGGTTATGA | |
| DS164 | 170 | CCGAAAGCGATACCCGGGGATGCTAAAATGC | ΔptsH RF |
| | | CTGAAATCATAACCCTACCTTACTTGTGA | |
| DS165 | 171 | CGGGTATCGCTTTCGGTAAAGCTCTGCTTCTG | ΔptsH RF |
| | | AAAGAAGACGAAATTGTCATTGACCGGA | |
| DS166 | 172 | CCTGGTCGGCAGAAATTTTTTTCCGGTCAATG | ΔptsH RF |
| | | ACAATTTCGTCTT | |
| DS303 | 173 | CGGATCCATCTCCTTTTGTTAGCAG | pAL2348, pAL2349, |
| | | | pAL2351, pAL2352 |
| DS304 | 174 | GCTGCTAACAAAGCCCGAAAGGAAG | pAL2348, pAL2349, |
| | | | pAL2351, pAL2352 |
| DS305 | 175 | AACAAAAGGAGATGGATCCGGTGCGGTGCA | pAL2348 |
| | | AAGGTTTTCTG | |
| DS306 | 176 | TTTCGGGCTTTGTTAGCAGCTCACTGAATAAT | pAL2348 |
| | | AACATCGC | |
| DS307 | 177 | AACAAAAGGAGATGGATCCGATGTACCAGGT | pAL2349 |
| | | TGTTGCGTC | |
| DS308 | 178 | TTTCGGGCTTTGTTAGCAGCTTACGATAAATA | pAL2349 |
| | | GAGTTTAC | |
| DS311 | 179 | AACAAAAGGAGATGGATCCGATGGCTATTAA | pAL2351 |
| | | ACTCATTGC | |
| DS312 | 180 | TTTCGGGCTTTGTTAGCAGCTTAATTCAGCAC | pAL2351 |
| | | ATACTTCT | |
| DS313 | 181 | AACAAAAGGAGATGGATCCGATGCTCTATAT | pAL2352 |
| | | CTTTGATTTAG | |
| DS314 | 182 | TTTCGGGCTTTGTTAGCAGCTTAGCATAACAC | pAL2352 |
| | | CTTCGCG | |
| JET003 | 183 | CTCGAGGTGAAGACGAAAGGGCCTCGTGATA | pAL1974, pAL1975, |
| | | CG | pAL1976, pAL1977 |
| JET004 | 184 | CGAATTCATTAAAGAGGAGAAAGGTACCATG | pAL1974, pAL1975, |
| | | GGTCATCACC | pAL1976, pAL1977 |
| JET005 | 185 | TTCGTCTTCACCTCGAGTATCCGAATGTAAGA | pAL1974 |
| | | AAGTTGGC | |
| JET006 | 186 | CCTCTTTAATGAATTCGAGGTTCAATCCCTCA | pAL1974 |
| | | ATGATGCC | |
| JET007 | 187 | CGTCTTCACCTCGAGTCATTGAATCTTTATTA | pAL1975 |
| | | GTTTTGTTTTTCACGC | |
| JET008 | 188 | CCTCTTTAATGAATTCGAATTTCATATCCTCT | pAL1975 |
| | | TGATGTTATGTCCC | |
| JET009 | 189 | CACCTCGAGTTTGCAGTGCAACTAATTCC | pAL1976 |
| JET010 | 190 | CCTCTTTAATGAATTCGAGCGTTATCTCGC | pAL1976 |
| JET011 | 191 | GCCCTTTCGTCTTCACCTCGAGGTAATAATTT | pAL1977, pAL2247 |
| | | TATAAATGCG | |
| JET012 | 192 | CCTCTTTAATGAATTCGACTTGCTTCTTATCC | pAL1977, pAL2247 |
| | | ATTTTAAACTCC | |
| JET018 | 113 | AAGAGGAGAAAAGATATACCATGAAAATCT | pAL2247 |
| | | CCCCCTCGTTAATGTG | |
| JET067 | 193 | ATAGCCAGCAGTTTTAGAGCTAGAAATAGC | pAL2178 |
| JET068 | 194 | CGAGGGCCGAACTAGTATTATACCTAGGAC | pAL2178 |
| JET087 | 195 | TCCGCTGATTGTTTTAGAGCTAGAAATAGC | pAL2124 |
| JET087 | 195 | TCCGCTGATTGTTTTAGAGCTAGAAATAGC | pAL2188 |
| JET088 | 196 | AAGTGAAGAAACTAGTATTATACCTAGGACT | pAL2124 |
| | | GAGC | |
| JET088 | 196 | AAGTGAAGAAACTAGTATTATACCTAGGACT | pAL2188 |
| | | GAGC | |
| JET134 | 197 | GGTATATCTTTTCTCCTCTTTAATGAATTCAC | pAL2247 |
| | | TTGCTTCTTATCCATTTTAAACTCC | |
| JG190 | 198 | GAAAGGTACCGTTTTAGAGCTAGAAATAGC | pAL2066 |
| JG191 | 199 | TCCTCTTTAAACTAGTATTATACCTAGGAC | pAL2066 |
| MM57 | 200 | ACTAGTATTATACCTAGGACTGAGC | pAL2063, pAL2158 |

TABLE 8
Plasmid construction guide.

| | Vector PCR | | | Insert(s) PCR | | | |
|---|---|---|---|---|---|---|---|
| | Primer | Primer | | Primer | Primer | | Insert |
| Plasmid | (F) | (R) | Template | (F) | (R) | Template | description |
| pAL1946 | AZ0475 | AZ936 | pAL1494 | AZ0466 | AZ0467 | MG1655 | alsE |
| | | | | AZ0468 | AZ0469 | MG1655 | hxpB |
| pAL1947 | AZ0475 | AZ936 | pAL1494 | AZ0466 | AZ0467 | MG1655 | alsE |
| | | | | AZ0472 | AZ0473 | MG1655 | ybiV |
| pAL1950* | AZ0520 | AZ0521 | pAL1851 | | | | |
| pAL1952 | AZ0504 | AZ0505 | pAL1874 | AZ0502 | AZ0503 | pAL1949 | kanR |
| pAL1957* | AZ0523 | AZ0522 | pAL1851 | | | | |
| pAL1958* | AZ0534 | AZ0535 | pAL1851 | | | | |
| pAL1974 | JET004 | JET003 | pAL421 | JET005 | JET006 | MG1655 | P_{ihfA4} |
| pAL1975 | JET004 | JET003 | pAL421 | JET007 | JET008 | MG1655 | P_{dps} |
| pAL1976 | JET004 | JET003 | pAL421 | JET009 | JET010 | MG1655 | P_{cbpA2} |
| pAL1977 | JET004 | JET003 | pAL421 | JET011 | JET012 | MG1655 | P_{gadB} |
| pAL2001 | AZ0668 | pAL669 | pAL1759 | AZ0666 | AZ0667 | pAL1946 | alsE-hxpB |
| pAL2036* | AZ0751 | AZ0752 | pAL1851 | | | | |
| pAL2038* | AZ0771 | AZ0712 | pAL1851 | | | | |
| pAL2063* | AZ1055 | MMM57 | pAL2062 | | | | |
| pAL2066* | JG190 | JG191 | pAL2062 | | | | |
| pAL2124* | JET087 | JET088 | pAL1851 | | | | |
| pAL2158* | AZ1055 | MMM57 | pAL1851 | | | | |
| pAL2173* | AZ195 | AZ196 | pAL2066 | | | | |
| pAL2174* | AZ1025 | AZ1016 | pAL2066 | | | | |
| pAL2178 | JET167 | JET168 | pAL1851 | | | | |
| pAL2182 | AZ1053 | AZ1054 | pAL1952 | AZ1051 | AZ1052 | pAL2063 | pTargetF-no target |
| pAL2188* | JET087 | JET088 | pAL2182 | | | | |
| pAL2233* | DS072 | DS071 | pAL1851 | | | | |
| pAL2247 | AZ0666 | JET018 | pAL2001 | JET011 | JET134 | pAL1977 | P_{gadB} |
| pAL2264 | DS127 | DS126 | pAL2256 | DS129 | DS128 | pAL2257 | gent^{R} |
| pAL2318* | DS158 | DS157 | pAL1851 | | | | |
| pAL2348 | DS304 | DS303 | pAL1946 | DS305 | DS306 | genome | hxpA |
| pAL2349 | DS304 | DS303 | pAL1946 | DS307 | DS308 | genome | yigL |
| pAL2351 | DS304 | DS303 | pAL1946 | DS311 | DS312 | genome | yidA |
| pAL2352 | DS304 | DS303 | pAL1946 | DS313 | DS314 | genome | yihx |

*Q5-site directed mutagenesis (NEB)

Genome modifications such as gene deletion and gene insertion were constructed using CRISPR-Cas9-mediated homologous recombination. Linear DNA repair fragments for gene deletions and insertions were constructed by amplifying genomic or plasmid DNA via PCR assembly. Plasmids encoding sgRNA for CRISPR-Cas9-mediated homologous recombination were constructed using Q5 site-directed mutagenesis (New England Biolabs) using pTargetF plasmid (Addgene #62226) as a template. All genomic modifications were verified via Sanger Sequencing. A guide to the CRISPR-Cas9-mediated gene modifications used in this study is detailed in Table 9.

TABLE 9
Guide for CRISPR-Cas9-mediate gene deletions and insertions

| | pTargetF | | | Linear repair fragment |
|---|---|---|---|---|
| Modification | Plasmid | SEQ ID NO: | 20 bp sgRNA targeting sequence 5′ to 3′ | assembly PCR primers |
| ΔpfkA | pAL1950 | 201 | TTCCCGTGCATCGGTCTGCC | AZ0482(F), AZ0483(R), |
| | | | | AZ0484(F), AZ0485(R), |
| | | | | AZ0486(F), AZ0487(R) |
| ΔalsE | pAL2233 | \|202 | GCCGTCATGACATGAAAGTG | DS075(F), DS076(R), |
| | | | | DS078(F), DS079(R), |
| | | | | DS080(F), DS081(R), |
| | | | | DS082(F), DS083(R) |
| Δzwf | pAL1958 | 203 | GCAGAAGAAGTGGGGATCGA | AZ0538(F), AZ0539(R), |
| | | | | AZ0540(F), AZ0541(R), |
| | | | | AZ0542(F), AZ0543(R) |
| ΔrpiB | pAL1957 | 204 | GTCGCACTGGCTGTTGCTGG | AZ0526(F), AZ0527(R), |
| | | | | AZ0528(F), AZ0529(R), |
| | | | | AZ0530(F), AZ0531(R), |
| | | | | AZ0532(F), AZ0533(R) |
| ΔmanA | pAL2178 | 205 | TCGGCCCTCGATAGCCAGCA | AZ1041(F), AZ1042(R), |
| | | | | AZ1043(F), AZ1044(R), |
| | | | | AZ1045(F), AZ1046(R) |
| ΔptsG | pAL2036 | 206 | TGCATTTGCTAACCTGCAAA | AZ0743(F), AZ0744(R), |
| | | | | AZ0745(F), AZ0746(R), |
| | | | | AZ0747(F), AZ0748(R) |
| ΔptsH | pAL2318 | 207 | CTGTTTAAACTGCAGACTCT | DS159(F), DS160(R), |
| | | | | DS161(F), DS162(R), |
| | | | | DS163(F), DS164(R), |
| | | | | DS165(F), DS166(R) |
| Δpgm | pAL2038 | 208 | ATGGCAATCCACAATCGTGC | AZ0763(F), AZ0764(R), |
| | | | | AZ0765(F), AZ0766(R), |
| | | | | AZ0767(F), AZ0768(R) |

Culturing media. Overnight cultures were grown at 37° C. in 3 mL of Luria-Bertani (LB) media with appropriate antibiotics. Antibiotic concentrations were as follows: spectinomycin (50 μg mL⁻¹), ampicillin (200 μg mL⁻¹), kanamycin (50 μg mL⁻¹), gentamycin (3.75 μg mL⁻¹). M9 minimal media consists of 33.7 mM Na₂HPO₄, 22 mM KH₂PO₄, 8.6 mM NaCl, 9.4 mM NH₄Cl, 2 mM MgSO₄, 0.1 mM CaCl₂), A5 trace metals mix (2.86 mg H₃BO₃, 1.81 mg MnCl₂·4H₂O, 0.079 mg CuSO₄·5H₂O, 49.4 μg Co (NO₃)₂·6H₂O), varying concentrations of glucose, and appropriate antibiotics. M9P media for the psicose production consists of M9 minimal media supplemented with 5 g L⁻¹ of yeast extract and appropriate antibiotics. No D-psicose production was detected when cultures were grown in M9P media without glucose. Inducer concentrations are as follows: isopropyl-β-D-1-thiogalactopyranoside (IPTG) (1 mM), anhydrotetracycline (aTc) (100 ng mL⁻¹). OD₆₀₀ was measured with a Synergy H1 hybrid plate reader (BioTek Instruments, Inc.).

Fluorescence assays. Overnight cultures were inoculated at 1% into 300 μL of LB media in a 96-well black-walled fluorescence assay plate. Cells were grown at 37° C., 250 rpm, until OD₆₀₀ ˜0.4. Cultures were then induced with IPTG if necessary and grown at 37° C., 250 rpm, for 24 h. Fluorescence was measured at an excitation wavelength of 485 nm and emission wavelength of 510 nm with a Synergy H1 hybrid plate reader (BioTek Instruments, Inc.).

D-Psicose production. For regular cell density production experiments, overnight cultures were inoculated at 1% into 3 mL of M9P media. Cells were grown at 37° C. until the OD₆₀₀ described, then induced with IPTG and aTc if necessary, and grown at 30° C. for 24 h. For high cell density production experiments in M9P media, overnight cultures were inoculated at 2% into 50 mL of M9P media. Cells were grown at 37° C. until OD₆₀₀ ˜1. Cultures were then induced with IPTG and aTc if necessary and grown for a further 30 min. Cultures were centrifuged at 5,000 g for 15 min and resuspended in M9P media with IPTG and aTc if necessary to a target OD₆₀₀. Cultures were grown at 30° C. for 24 h.

HPLC analysis. Analysis of D-psicose, glucose, and mannose concentrations was performed using high performance liquid chromatography (HPLC) (Shimadzu) equipped with a refractive index detector (RID) 10 A and Rezex™ RCU-USP sugar alcohol column (Phenomenex). Mobile phase was comprised of 100% MilliQ water. Samples were run with an injection volume of 1 μL at a flow rate of 0.5 mL min⁻¹ for 7 min, with the column oven at 83° C. and RID cell temperature of 40° C. To prepare samples for HPLC analysis, 300 μL of culture was centrifuged at 17,000 g for 5 min. Supernatants were applied to a 0.2 μm PVDF hydrophilic membrane 96 well filter plate and centrifuged at 17,000 g or 2 min into a polystyrene 96 well.

GC-MS analysis. GC-MS analysis was performed by the UC Davis West Coast Metabolomics Center. Chemical standards (D-psicose, D-mannose. D-glucose, D-galactose, D-erythrose, D-tagatose, and D-threose) were purchased from Sigma Aldrich. To analyze via GC-MS, 4 μL of spun-down culture supernatant were dried down and derivatized by adding 10 μL of 40 mg mL⁻¹ methoxyamine hydrochloride (Sigma-Aldrich) in pyridine (Sigma-Aldrich) and shaking at 30° C. for 1.5 h. Subsequently, 90 μL of N-tert-Butyldimethylsilyl-N-methyltrifluoroacetamide (MTBSTFA) (Sigma-Aldrich) was added with 13 fatty-acid methyl esters (FAMEs) as retention index markers and shaken at 80° C. for 30 min. Samples were immediately transferred to crimp top vials and injected onto each GC-MS instrument. A LECO Pegasus IV TOF MS was coupled to an Agilent 7890 GC system installed with a Restek RTX-5Sil MS column (29.70 m length, 0.25 mm i.d, 0.25 μM df, 95% dimethyl/5% diphenyl polysiloxane film) with an additional 10 m guard column. 1 μL of the derivatized sample was injected into the GC in splitless mode at an injection temperature of 275° C. and a constant flow of 1 mL min⁻¹. The initial oven temperature was held at 50° C. for 1 min and ramped at a rate of 20° C. min⁻¹ to 330° C. that was maintained for 5 min for a total run time of 20 min. The mass spectrometer was used under electron ionization mode at +70 eV. Mass spectra were acquired from 85 to 500 m/z at a scan rate of 17 Hz and 250° C. source temperature. Binbase was used for metabolite annotation and reporting.

### Example 3—Biosynthesis of Psicose from Glucose in High-Density Conditions

An additional source of glucose siphoning away from the D-psicose pathway is through glycogen biosynthesis, which produces glycogen for use during times of starvation. The deletion of pgm, which encodes for phosphoglucomutase Pgm, prevents E. coli from producing glycogen (Eydallin, G. et al. Genome-wide screening of genes affecting glycogen metabolism in Escherichia coli K-12. FEBS Lett. 581, 2947-2953 (2007)). As such, pgm was deleted in the production strain AL3990, generating AL4186 (MG1655 ΔpfkA Δzwf ΔrpiB ΔmanA Δpgm).

The rate of D-glucose consumption and D-psicose production of Strain 7 (see Table 4) was monitored for 10 h at media glucose concentrations of 3, 5, and 10 g L⁻¹. It was found the rate of D-glucose consumption and D-psicose production to be similar across cultures, independent of media glucose concentrations. Cultures fed 3 g L⁻¹ of glucose consumed all glucose over the course of the experiment.

To decouple growth and production, and minimize limitations on production by glucose availability, Strain AL4186 transformed with plasmids pAL2247 (P_{gadB}:alsE-hxpB), pAL2264 (P_{LlacO1}:galP-glk), and pAL2188 (Pₜₑₜ:dcas9 pTargetF-pfkB) (also identified as Strain 7 in Table 4) was cultured under high cell density conditions in an excess of available D-glucose over a shorter period of time. Cultures were grown in M9P media with 40 g L⁻¹ glucose at 37° C. to an OD₆₀₀ of ˜1 before being induced with 100 ng mL⁻¹ aTc and 1 mM IPTG and grown for a further 30 min. Cells were then pelleted and resuspended to an OD₆₀₀ of ˜10 with M9P containing 40 g L⁻¹ glucose, 100 ng mL⁻¹ aTc, and 1 mM IPTG. Samples were taken and analyzed at 0, 4, and 8 h.

Over the course of 8 h, cultures produced 15.3 g L⁻¹ of D-psicose with a specific titer of 1.4 g L⁻¹ OD₆₀₀⁻¹, yield of 43%, and productivity of 1.9 g L⁻¹ hr⁻¹. See FIG. 24. Although D-psicose titers produced at 0-4 h and 4-8 h were similar, at 7.3 and 8.0 g L⁻¹, the yield at 4-8 hr (53%) was higher than that at 0-4 h (35%). The productivity at 4-8 h was 2.0 g L⁻¹ h⁻¹. Higher yield can be the result of cells responding to their high-density culture conditions, as the presently disclosed production system relies on the activation of stationary phase promoter P_{gadB}. Overall, the system achieved industrially relevant production with high yield (>0.5 g_{product} g_{substrate}⁻¹) and high productivity (>1 g L⁻¹ h⁻¹) comparable to bioethanol, despite being cultured in unoptimized test tube conditions.

Next, to determine the purity of the sample, the concentration of glucose, allulose, and mannose was analyzed using high performance liquid chromatography (HPLC). Glucose, allulose, and mannose standards of known concentrations were run on the HPLC, and the area under each corresponding peak was integrated. For each sugar standard, the peak integrations were plotted against concentrations, and fitted with a line of best fit. Concurrently with standards, production samples were run on the HPLC. Standards were used to identify the corresponding sugar peaks in each production sample. Each sample peak was integrated, and their areas recorded. Using the line of best fit, peak integrations were used to find the concentration of sugars in each sample. Allulose purity was calculated using sugar concentrations and the following equation:

$$\% purity = \frac{[ allu lose ]}{[ allulose ] + [ mannose ] + [ glucose ]} \times 100$$

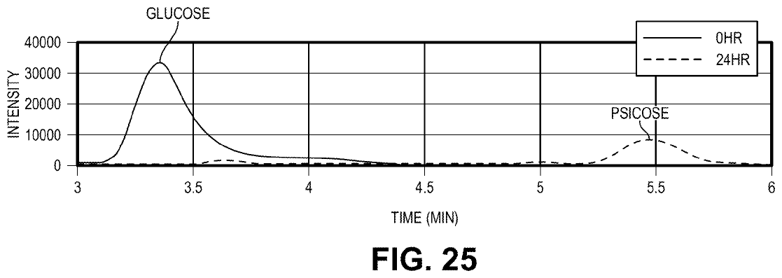

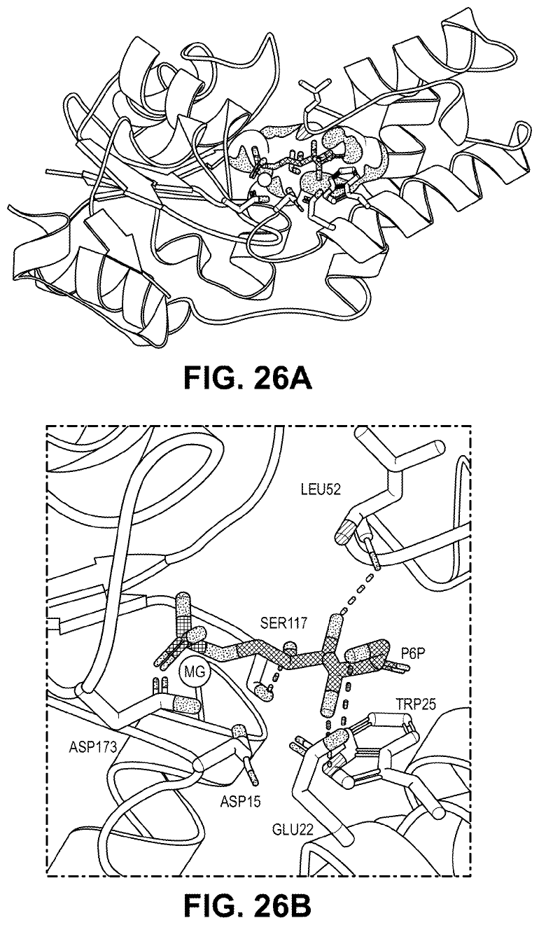

Strain 7 was able to consume all media glucose at 24 h (FIG. 25) indicating that the purity of allulose was approximately 100%. Full consumption of substrate is a desirable property of microbial production, as it is costly to separate D-psicose from mixtures of glucose and/or fructose. Depleting glucose from the media allows for easier extraction and purification of D-psicose in an industrial setting.

Although the presently disclosed subject matter and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the presently disclosed subject matter, processes, machines, manufacture, compositions of matter, means, methods, or steps, presently existing or later to be developed that perform substantially the same function or achieve substantially the same result as the corresponding embodiments described herein can be utilized according to the presently disclosed subject matter. Accordingly, the appended claims are intended to include within their scope such processes, machines, manufacture, compositions of matter, means, methods, or steps.

Patents, patent applications, publications, product descriptions and protocols are cited throughout this application the disclosures of which are incorporated herein by reference in their entireties for all purposes.

1. A recombinant microorganism comprising an exogenous epimerase and an exogenous phosphatase, wherein the recombinant microorganism produces an increased amount of psicose as compared to a naturally occurring microorganism.

2-3. (canceled)

4. The recombinant microorganisms of claim 1, wherein the epimerase comprises an amino acid sequence at least about 80% identical to the amino acid sequence set forth in SEQ ID NO: 1.

5-8. (canceled)

9. The recombinant microorganisms of claim 1, wherein the phosphatase comprises an amino acid sequence at least about 80% identical to the amino acid sequence set forth in SEQ ID NO: 3 or SEQ ID NO: 4.

10-11. (canceled)

12. The recombinant microorganism of claim 1, wherein the recombinant microorganism further comprises exogenous galactose:H⁺ symporter (GalP) and glucokinase (Glk), wherein the GalP comprises an amino acid sequence at least about 80% identical to the amino acid sequence set forth in SEQ ID NO: 38 and the Glk comprises an amino acid sequence at least about 80% identical to the amino acid sequence set forth in SEQ ID NO: 40.

13-15. (canceled)

16. The recombinant microorganism of claim 1 further comprising a mutation of a gene encoding for an enzyme of the pentose phosphate pathway compared to a naturally occurring microorganism.

17. The recombinant microorganism of claim 16, wherein enzyme of the pentose phosphate pathway is glucose-6-phosphate 1-dehydrogenase (Zwf).

18. The recombinant microorganism of claim 1 further comprising a mutation of a gene encoding for an enzyme of the glycolysis compared to a naturally occurring microorganism.

19. The recombinant microorganism of claim 18, wherein the enzyme of the glycolysis is phosphofructokinase-1 (PfkA), phosphofructokinase-2 (PfkB), or pyruvate kinase (PykF).

20. The recombinant microorganism of claim 18, wherein the enzyme of the glycolysis is phosphofructokinase-1 (PfkA).

21. The recombinant microorganism of claim 1 further comprising a mutation of a gene encoding for an enzyme of allose degradation pathway.

22. The recombinant microorganism of claim 21, wherein the enzyme of allose degradation pathway is allose-6-phosphate isomerase (RpiB).

23. The recombinant microorganism of claim 1 further comprising a mutation of a gene encoding for an enzyme of mannose biosynthesis pathway.

24. The recombinant microorganism of claim 23, wherein the enzyme of mannose biosynthesis pathway is mannose-6-phosphate isomerase (ManA).

25-30. (canceled)

31. The recombinant microorganism of claim 1, further comprising a mutation of a gene encoding for an enzyme of glycogen biosynthesis selected from the group consisting of phosphoglucomutase (Pgm), UDP-glucose pyrophosphorylase, glycogen synthase, glycogen branching enzyme, and glycogenin.

32.-60. (canceled)

61. The recombinant microorganism of claim 1, comprising:
a) a recombinant polynucleotide encoding an epimerase and a phosphatase;
b) a mutation of a gene encoding for an enzyme of the pentose phosphate pathway;
c) a mutation of a gene encoding for an enzyme of the glycolysis;
d) a mutation of a gene encoding for an enzyme of allose degradation pathway; and
e) a mutation of a gene encoding for an enzyme of mannose biosynthesis pathway; and
f) optionally a recombinant polynucleotide encoding GalP, Glk, or both.

62. The recombinant microorganism of claim 1, comprising:
a) a recombinant polynucleotide encoding an allulose-6-phosphate 3-epimerase (AlsE) and a hexitol phosphatase B (HxpB);
b) a mutation of glucose-6-phosphate 1-dehydrogenase (Zwf);
c) a mutation of phosphofructokinase-1 (PfkA);
d) a mutation of allose-6-phosphate isomerase (RpiB);
e) a mutation of mannose-6-phosphate isomerase (ManA); and
f) optionally a recombinant polynucleotide encoding GalP, Glk, or both.

63. the recombinant microorganism of claim 1, comprising:
a) a recombinant polynucleotide encoding an allulose-6-phosphate 3-epimerase (AlsE);
b) a recombinant polynucleotide encoding a hexitol phosphatase B (HxpB);
c) a mutation of glucose-6-phosphate 1-dehydrogenase (Zwf);
d) a mutation of phosphofructokinase-1 (PfkA);
e) a mutation of allose-6-phosphate isomerase (RpiB);
f) a mutation of mannose-6-phosphate isomerase (ManA); and
g) optionally a recombinant polynucleotide encoding GalP, Glk, or both.

64-65. (canceled)

66. The microorganism of claim 1, wherein the microorganism is E. coli, Bacillus subtilis or Lactococcus lactis.

67-68. (canceled)

69. A method for producing psicose comprising culturing the microorganism of claim 1 under conditions suitable for converting a substrate to psicose.

70-76. (canceled)

77. A method of making a food product comprising psicose comprising
a) culturing the microorganism of claim 1 under conditions suitable for converting a substrate to psicose;
b) purifying the psicose; and
c) admixing the psicose with a food product to form a food product comprising psicose.

78-79. (canceled)
